(19) United States
(12) Patent Application Publication
(10) Pub. No.: US 2024/0016055 A1
(43) Pub. Date: Jan. 11, 2024

(54) AMINE-CONTAINING COMPOUND, LIGHT-EMITTING DEVICE INCLUDING THE SAME, ELECTRONIC APPARATUS INCLUDING THE LIGHT-EMITTING DEVICE, AND ELECTRONIC DEVICE INCLUDING THE ELECTRONIC APPARATUS

(71) Applicant: Samsung Display Co., Ltd., Yongin-si (KR)

(72) Inventors: Dongjun Kim, Yongin-si (KR); Chaeyeong Kim, Yongin-si (KR); Hankyu Pak, Yongin-si (KR); Byeongwook Yoo, Yongin-si (KR); Seowon Cho, Yongin-si (KR); Sohee Jo, Yongin-si (KR)

(21) Appl. No.: 18/331,790

(22) Filed: Jun. 8, 2023

(30) Foreign Application Priority Data
Jul. 7, 2022 (KR) 10-2022-0083816

(51) Int. Cl. H10K 85/60; C07C 211/54

(52) U.S. Cl. CPC H10K 85/633; H10K 85/615; C07C 211/54; H10K 50/15

(57) ABSTRACT

Provided is a light-emitting device including a first electrode, a second electrode facing the first electrode, an interlayer between the first electrode and the second electrode and including an emission layer, and an amine-containing compound represented by Formula 1, wherein the detailed description of Formula 1 is the same as described in the present specification:

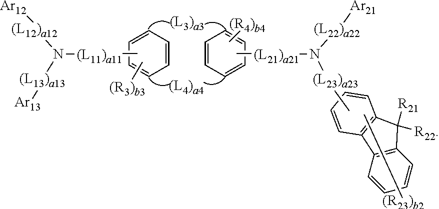

### CROSS-REFERENCE TO RELATED APPLICATION

This application claims priority to and the benefit of Korean Patent Application No. 10-2022-0083816, filed on Jul. 7, 2022, in the Korean Intellectual Property Office, the entire content of which is incorporated by reference herein.

### BACKGROUND

### 1. Field

One or more embodiments of the present disclosure relate to an amine-containing compound, a light-emitting device including the same, an electronic apparatus including the light-emitting device, and an electronic device including the electronic apparatus.

### 2. Description of the Related Art

Organic light-emitting devices may have wide viewing angles, high contrast ratios, and short response times, as compared with inorganic light-emitting devices. The organic light-emitting devices may include a first electrode, a hole transport region, an emission layer, an electron transport region, and a second electrode that are sequentially arranged. Holes injected from the first electrode may move to the emission layer through the hole transport region. Electrons injected from the second electrode may move to the emission layer through the electron transport region. Carriers, such as the holes and the electrons, may recombine in the emission layer. The carriers may combine to produce excitons. The excitons move (e.g., transition) from an excited state to a ground state, thereby generating light.

### SUMMARY

One or more embodiments include an amine-containing compound having improved hole transporting characteristics, and a light-emitting device having a low driving voltage, high luminance, high luminescence efficiency, and a long lifespan by including the amine-containing compound. In addition, one or more embodiments include a high-quality electronic apparatus and electronic device including the light-emitting device.

Additional aspects of embodiments will be set forth in part in the description which follows and, in part, will be apparent from the description, or may be learned by practice of the presented embodiments of the disclosure.

According to one or more embodiments, a light-emitting device includes a first electrode, a second electrode facing the first electrode, an interlayer between the first electrode and the second electrode and including an emission layer, and an amine-containing compound represented by Formula 1.

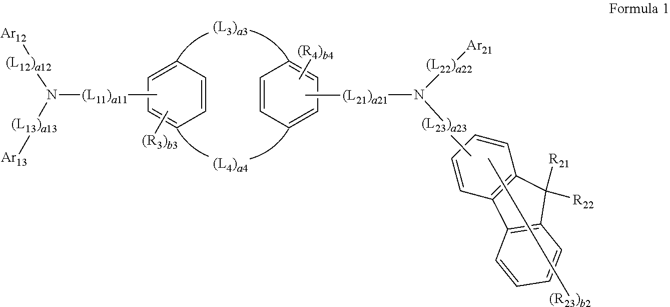

In Formula 1,
L₁₁ to L₁₃, L₂₁ to L₂₃, Ar₁₂, Ar₁₃, and Ar₂₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a11 to a13 and a21 to a23 may each independently be an integer from 0 to 5, wherein, i) when a11 is 0, a group represented by *-(L₁₁)ₐ₁₁-*′ may be a single bond, ii) when a12 is 0, a group represented by *-(L₁₂)ₐ₁₂-*′ may be a single bond, iii) when a13 is 0, a group represented by *-(L₁₃)ₐ₁₃*′ may be a single bond, iv) when a21 is 0, a group represented by *-(L₂₁)ₐ₂₁-*′ may be a single bond, v) when a22 is 0, a group represented by *-(L₂₂)ₐ₂₂-*′ may be a single bond, vi) when a23 is 0, a group represented by *-(L₂₃)ₐ₂₃-*′ may be a single bond, vii) when a11 is one of 2 to 5, a plurality of L₁₁(s) may be identical to or different from each other, viii) when a12 is one of 2 to 5, a plurality of L₁₂(s) may be identical to or different from each other, ix) when a13 is one of 2 to 5, a plurality of L₁₃(s) may be identical to or different from each other, x) when a21 is one of 2 to 5, a plurality of L₂₁(s) may be identical to or different from each other, xi) when a22 is one of 2 to 5, a plurality of L₂₂(s) may be identical to or different from each other, and xii) when a23 is one of 2 to 5, a plurality of L₂₃(s) may be identical to or different from each other,
L₃ and L₄ may each independently be *—O—*′, *—S—*′, *—N(Q₁)*′, or *—C(Q₁)(Q₂)-*′
a3 and a4 may each independently be an integer from 0 to 4, wherein, i) when a3 is 0, a group represented by *-(L₃)ₐ₃*′ may be a single bond, ii) when a4 is 0, a group represented by *-(L₄)ₐ₄*′ may be a single bond, iii) when a3 is one of 2 to 4, a plurality of L₃(s) may be identical to or different from each other, and iv) when a4 is one of 2 to 4, a plurality of L₄(s) may be identical to or different from each other,
R₃, R₄, R₂₁, R₂₂, and R₂₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₂₁ and R₂₂ may optionally be bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
b2 may be an integer from 0 to 7, wherein, when b2 is one of 2 to 7, a plurality of R₂₃(s) may be identical to or different from each other,
b3 may be an integer from 0 to 3, wherein, when b3 is one of 2 and 3, a plurality of R₃(s) may be identical to or different from each other,
b4 may be an integer from 0 to 3, wherein, when b4 is one of 2 and 3, a plurality of R₄(s) may be identical to or different from each other,
R₁₀ₐ may be:
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group, or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof, and
* and *′ each indicate a binding site to a neighboring atom.

According to one or more embodiments, an electronic apparatus includes the light-emitting device.

According to one or more embodiments, an electronic device includes the electronic apparatus.

According to one or more embodiments, provided is the amine-containing compound represented by Formula 1.

### BRIEF DESCRIPTION OF THE DRAWINGS

The above and other aspects and features of certain embodiments of the disclosure will be more apparent from the following description taken in conjunction with the accompanying drawings, in which:

FIG. 1 is a schematic view of a light-emitting device according to an embodiment;

FIG. 2 is a schematic view of an electronic apparatus according to an embodiment;

FIG. 3 is a schematic view of an electronic apparatus according to another embodiment;

FIG. 4 is a schematic perspective view of an electronic device including a light-emitting device, according to an embodiment;

FIG. 5 is a schematic view of the exterior of a vehicle as an electronic device including a light-emitting device, according to an embodiment; and

FIGS. 6A to 6C are schematic views of the interior of a vehicle according to various embodiments.

### DETAILED DESCRIPTION

Reference will now be made in more detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of embodiments of the present description. As used herein, the term “and/or” includes any and all combinations of one or more of the associated listed items. Throughout the disclosure, the expression “at least one of a, b or c” indicates only a, only b, only c, both a and b, both a and c, both b and c, all of a, b, and c, or variations thereof.

According to an aspect of embodiments of the disclosure, provided is a light-emitting device including a first electrode, a second electrode facing the first electrode, an interlayer between the first electrode and the second electrode and including an emission layer, and an amine-containing compound represented by Formula 1.

In an embodiment, the first electrode may be an anode. The second electrode may be a cathode. The emission layer may include a dopant and a host, and may emit light. The dopant and the host are the same as described below.

The term “interlayer,” as used herein, refers to a single layer and/or all of a plurality of layers between the first electrode and the second electrode of the light-emitting device.

In an embodiment, the interlayer may further include a hole transport region between the first electrode and the emission layer and an electron transport region between the emission layer and the second electrode. The hole transport region may include a hole injection layer, a hole transport layer, an emission auxiliary layer, an electron blocking layer, or any combination thereof. The electron transport region may include a buffer layer, a hole blocking layer, an electron control layer, an electron transport layer, an electron injection layer, or any combination thereof.

In an embodiment, the amine-containing compound may be included in the interlayer.

In an embodiment, the amine-containing compound may be included in the hole transport region.

For example, the amine-containing compound may be included in the hole injection layer, the hole transport layer, the emission auxiliary layer, the electron blocking layer, or any combination thereof.

In an embodiment, the amine-containing compound may be included in the hole transport layer, and the hole transport layer may be in direct contact (e.g., physical contact) with the emission layer.

In an embodiment, the light-emitting device may further include a capping layer outside the first electrode, and the amine-containing compound may be included in the capping layer. In an embodiment, the amine-containing compound may be included in a capping layer outside an anode. In an embodiment, the amine-containing compound may be included in a capping layer outside a cathode.

In an embodiment, the light-emitting device may further include a first capping layer outside the first electrode and a second capping layer outside the second electrode, and the amine-containing compound may be included in the first capping layer or the second capping layer. In an embodiment, the amine-containing compound may be included in the first capping layer from among the first capping layer, the first electrode, and the interlayer that are sequentially arranged. In an embodiment, the amine-containing compound may be included in the second capping layer from among the interlayer, the second electrode, and the second capping layer that are sequentially arranged. The amine-containing compound may be included in both the first capping layer and the second capping layer.

According to another aspect of embodiments of the disclosure, provided is an electronic apparatus including the light-emitting device.

In an embodiment, the electronic apparatus may further include a thin-film transistor electrically connected to the light-emitting device, a color filter, a color conversion layer, a touch screen layer, a polarization layer, or any combination thereof. In an embodiment, the electronic apparatus may include the light-emitting device, the thin-film transistor, and the color filter. In an embodiment, the electronic apparatus may include the light-emitting device, the thin-film transistor, the color filter, and the color conversion layer.

According to another aspect of embodiments of the disclosure, provided is an electronic device including the electronic apparatus. The electronic device may be one of a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater and/or stadium screen, a phototherapy device, and/or a signboard.

According to another aspect of embodiments of the disclosure, provided is the amine-containing compound represented by Formula 1:

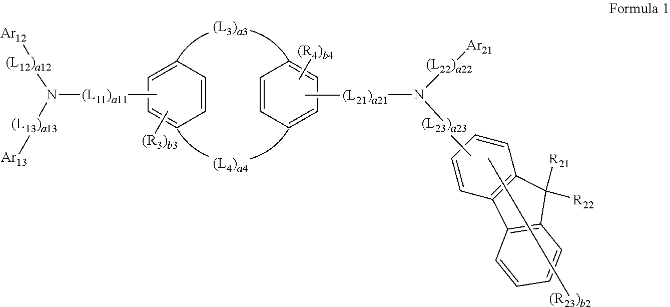

wherein, in Formula 1,
L₁₁ to L₁₃, L₂₁ to L₂₃, Ar₁₂, Ar₁₃, and Ar₂₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
a11 to a13 and a21 to a23 may each independently be an integer from 0 to 5, wherein, i) when a11 is 0, a group represented by *-(L₁₁)ₐ₁₁-*′ may be a single bond, ii) when a12 is 0, a group represented by *-(L₁₂)ₐ₁₂-*′ may be a single bond, iii) when a13 is 0, a group represented by *-(L₁₃)ₐ₁₃*′ may be a single bond, iv) when a21 is 0, a group represented by *-(L₂₁)ₐ₂₁-*′ may be a single bond, v) when a22 is 0, a group represented by *-(L₂₂)ₐ₂₂-*′ may be a single bond, vi) when a23 is 0, a group represented by *-(L₂₃)ₐ₂₃-*′ may be a single bond, vii) when a11 is one of 2 to 5, a plurality of L₁₁(s) may be identical to or different from each other, viii) when a12 is one of 2 to 5, a plurality of L₁₂(s) may be identical to or different from each other, ix) when a13 is one of 2 to 5, a plurality of L₁₃(s) may be identical to or different from each other, x) when a21 is one of 2 to 5, a plurality of L₂₁(s) may be identical to or different from each other, xi) when a22 is one of 2 to 5, a plurality of L₂₂(s) may be identical to or different from each other, and xii) when a23 is one of 2 to 5, a plurality of L₂₃(s) may be identical to or different from each other,
L₃ and L₄ may each independently be *—O—*′, *—S—*′, *—N(Q₁)-*′, or *—C(Q₁)(Q₂)-*′,
a3 and a4 may each independently be an integer from 0 to 4, wherein, i) when a3 is 0, a group represented by *-(L₃)ₐ₃*′ may be a single bond, ii) when a4 is 0, a group represented by *-(L₄)ₐ₄*′ may be a single bond, iii) when a3 is one of 2 to 4, a plurality of L₃(s) may be identical to or different from each other, and iv) when a4 is one of 2 to 4, a plurality of L₄(s) may be identical to or different from each other,
R₃, R₄, R₂₁, R₂₂, and R₂₃ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b2 may be an integer from 0 to 7, wherein, when b2 is one of 2 to 7, a plurality of R₂₃(s) may be identical to or different from each other,
b3 may be an integer from 0 to 3, wherein, when b3 is one of 2 and 3, a plurality of R₃(s) may be identical to or different from each other,
b4 may be an integer from 0 to 3, wherein, when b4 is one of 2 and 3, a plurality of R₄(s) may be identical to or different from each other,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ may each independently be:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆ alkoxy group, a phenyl group, a biphenyl group, or any combination thereof, and
* and *′ each indicate a binding site to a neighboring atom.

In an embodiment, R₃ and R₄ may each not be —N(Q₁)(Q₂).

In an embodiment, R₃ and R₄ may each independently be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, —Si(Q₁)(Q₂)(Q₃), —B(Q₁)(Q₂), —C(═O)(Q₁), —S(═O)₂(Q₁), or —P(═O)(Q₁)(Q₂).

In an embodiment, Ar₂₁ may be a cyclohexane group, a cycloheptane group, a cyclooctane group, an adamantane group, a norbornane group, a benzene group, a biphenyl group, a naphthalene group, a phenanthrene group, a fluorene group, a spiro-bifluorene group, a dibenzofuran group, or a dibenzothiophene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a biphenyl group, a naphthyl group, a phenanthrenyl group, a fluorenyl group, a spiro-bifluorenyl group, a dibenzofuranyl group, a dibenzothiophenyl group, or any combination thereof.

In an embodiment, L₁₁ to L₁₃ and L₂₁ to L₂₃ may each independently be a group represented by one of Formulae 2-1 to 2-28:

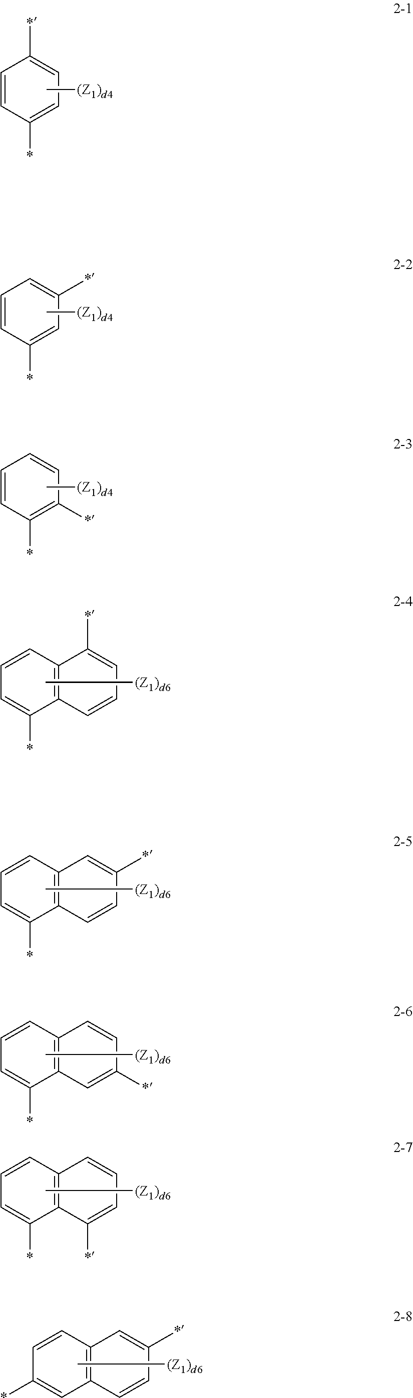

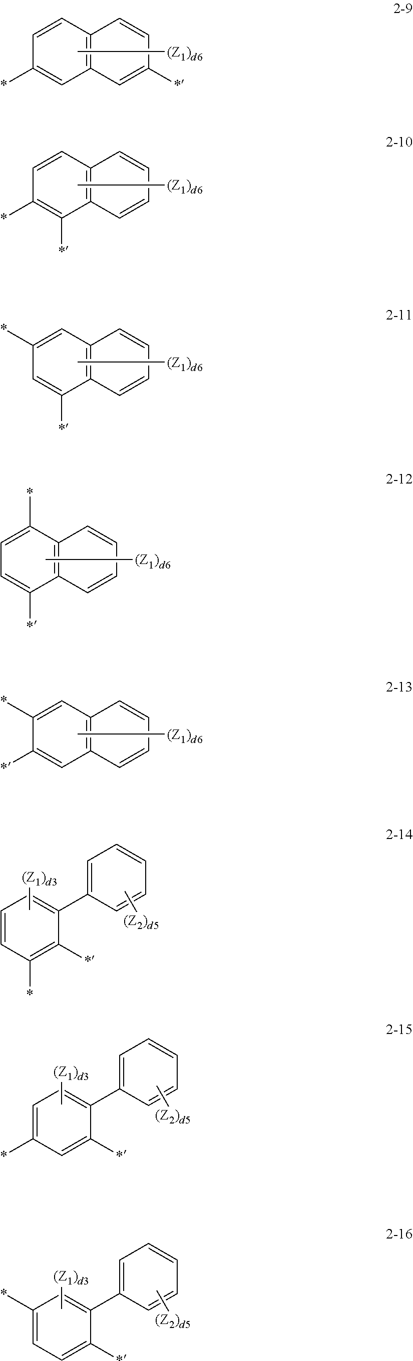

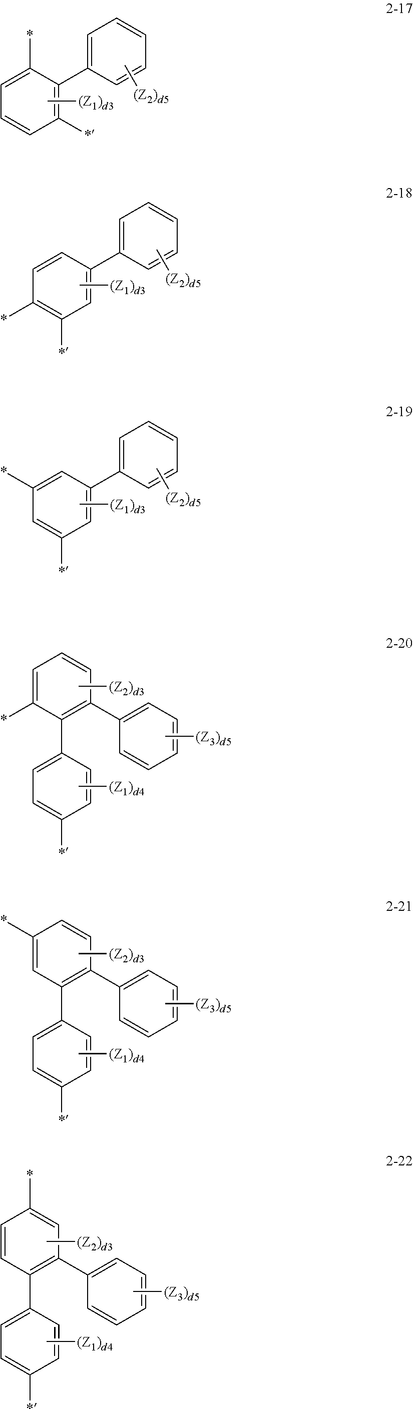

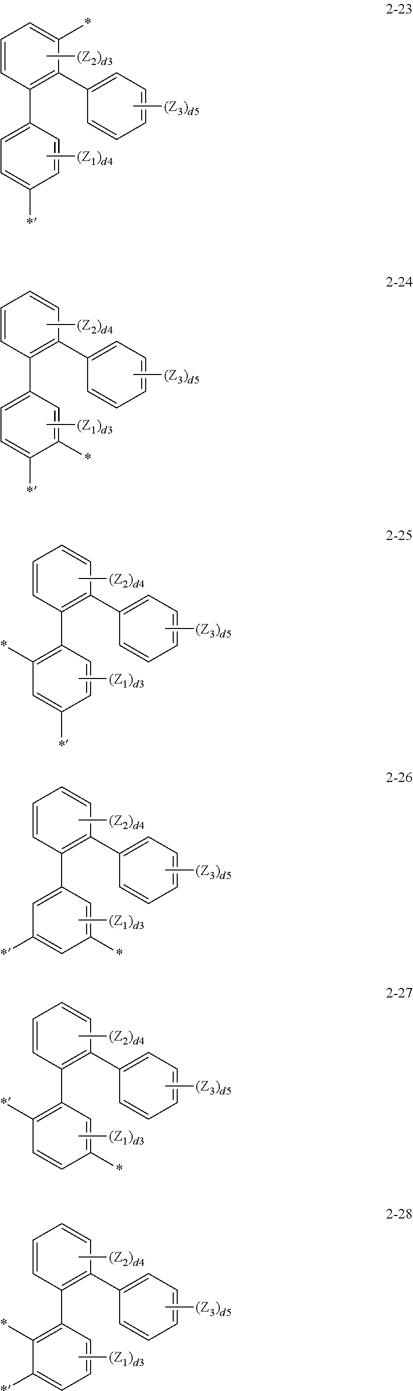

wherein, in Formulae 2-1 to 2-28,
Z₁ to Z₃ are each the same as described in connection with R₁₀ₐ,
d3 may be 0, 1, 2, or 3,
d4 may be 0, 1, 2, 3, or 4,
d5 may be 0, 1, 2, 3, 4, or 5, and
d6 may be 0, 1, 2, 3, 4, 5, or 6.

In an embodiment, Ar₂₁ may be a benzene group or a naphthalene group, each substituted with a cyclohexyl group, a cycloheptyl group, a cyclooctyl group, an adamantanyl group, a norbornanyl group, a phenyl group, or any combination thereof.

In an embodiment, a11 to a13 and a21 to a23 may each independently be 0 or 1.

In an embodiment, Ar₁₂ and Ar₁₃ may each independently be a cyclohexane group, a cycloheptane group, a cyclooctane group, an adamantane group, a norbornane group, a benzene group, a naphthalene group, or a phenanthrene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, or any combination thereof.

In an embodiment, at least one of Ar₂₁, Ar₁₂, and Ar₁₃ may be a benzene group, a naphthalene group, or a phenanthrene group, each substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, or any combination thereof.

In an embodiment, Ar₂₁ may not be

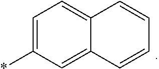 [SMILES: *c1ccc2ccccc2c1]

In an embodiment, when b2 is 0 or 1 and b2 is 1, R₂₃ may be a phenyl group or a naphthyl group, each unsubstituted or substituted with at least one R₁₀ₐ.

In an embodiment, R₂₁ and R₂₂ may each independently be hydrogen, deuterium, or a C₁-C₆₀ alkyl group, and R₂₁ and R₂₂ may optionally be bonded to each other to form a cyclopentane group or a cyclohexane group, each unsubstituted or substituted with at least one R₁₀ₐ.

In an embodiment, at least one of *-(L₃)-*′ and *-(L₄)-*′ may be *—CH₂CH₂—*′.

In an embodiment, the sum of a3 and a4 may be an integer of 2 or less.

In an embodiment, the amine-containing compound may be one of Compounds 1 to 20, 22 to 60, and 62 to 239:

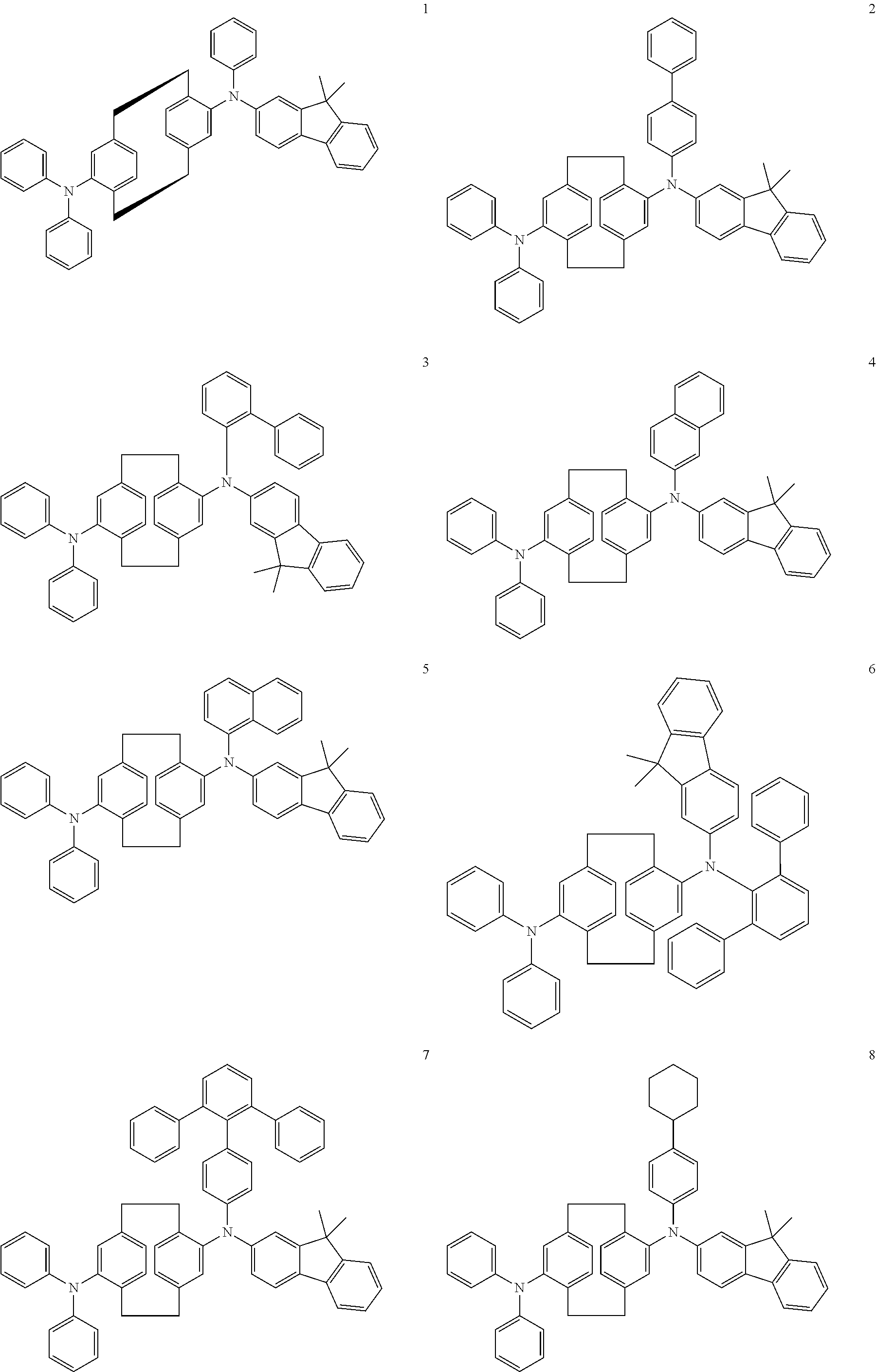

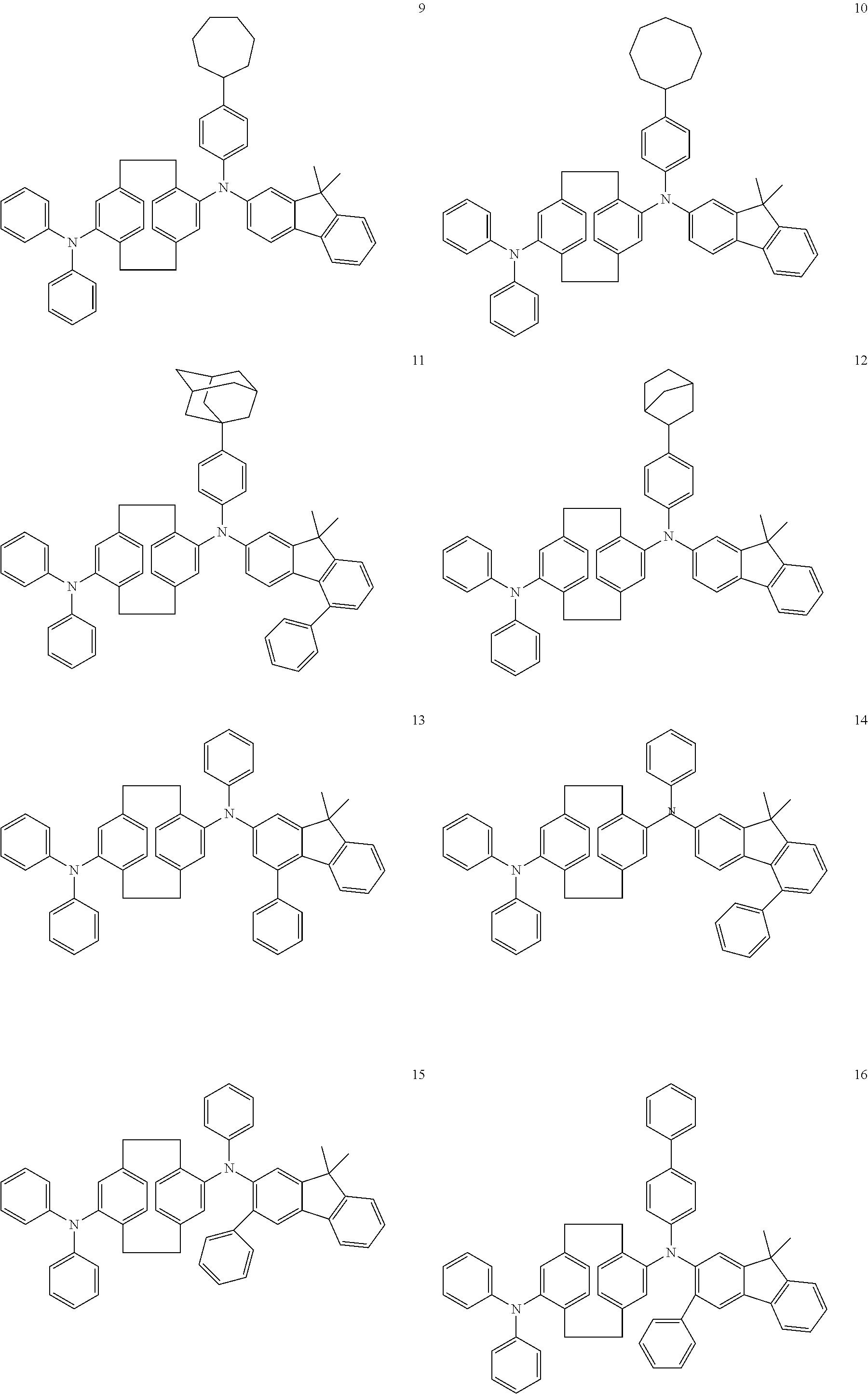

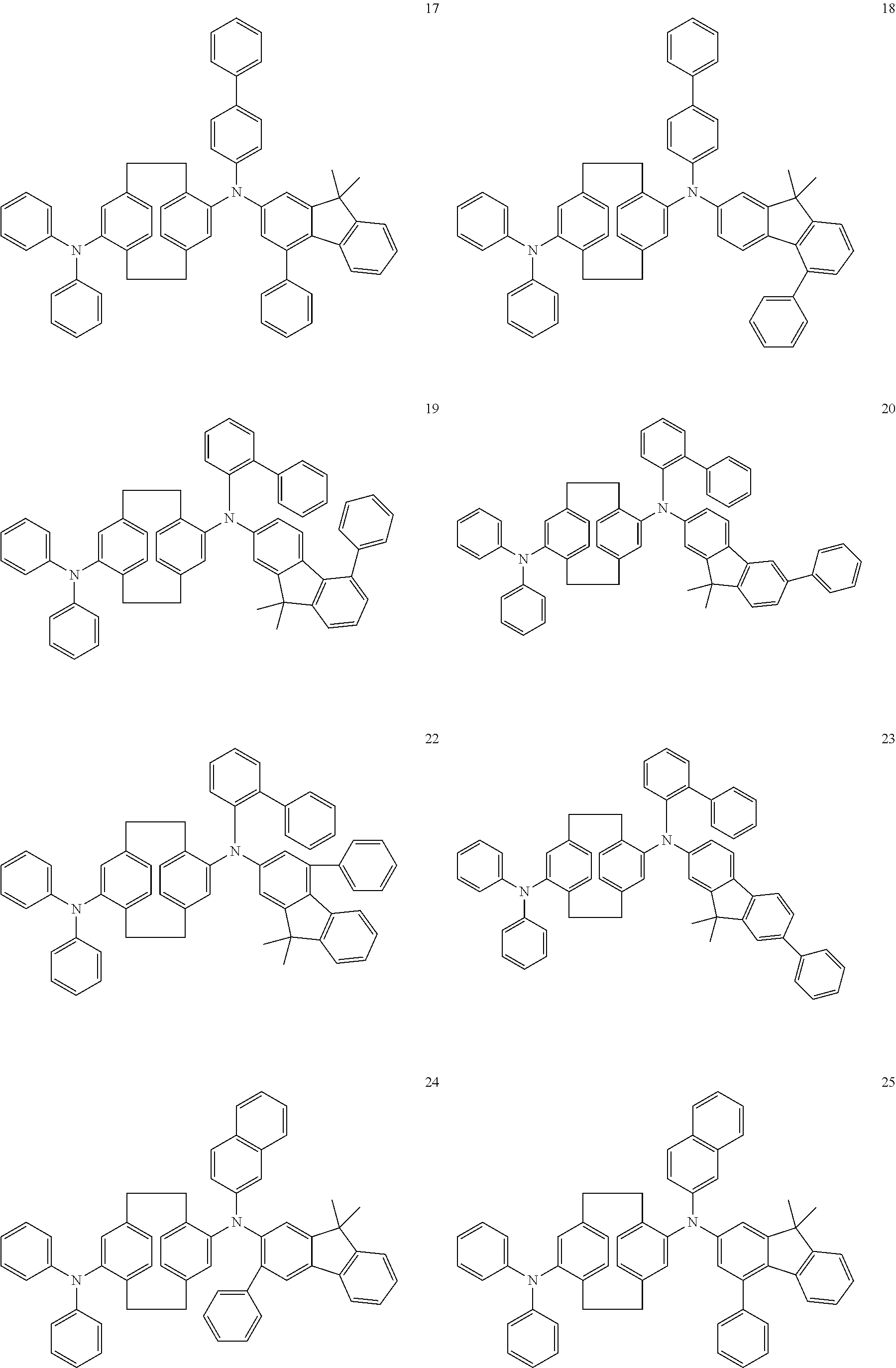

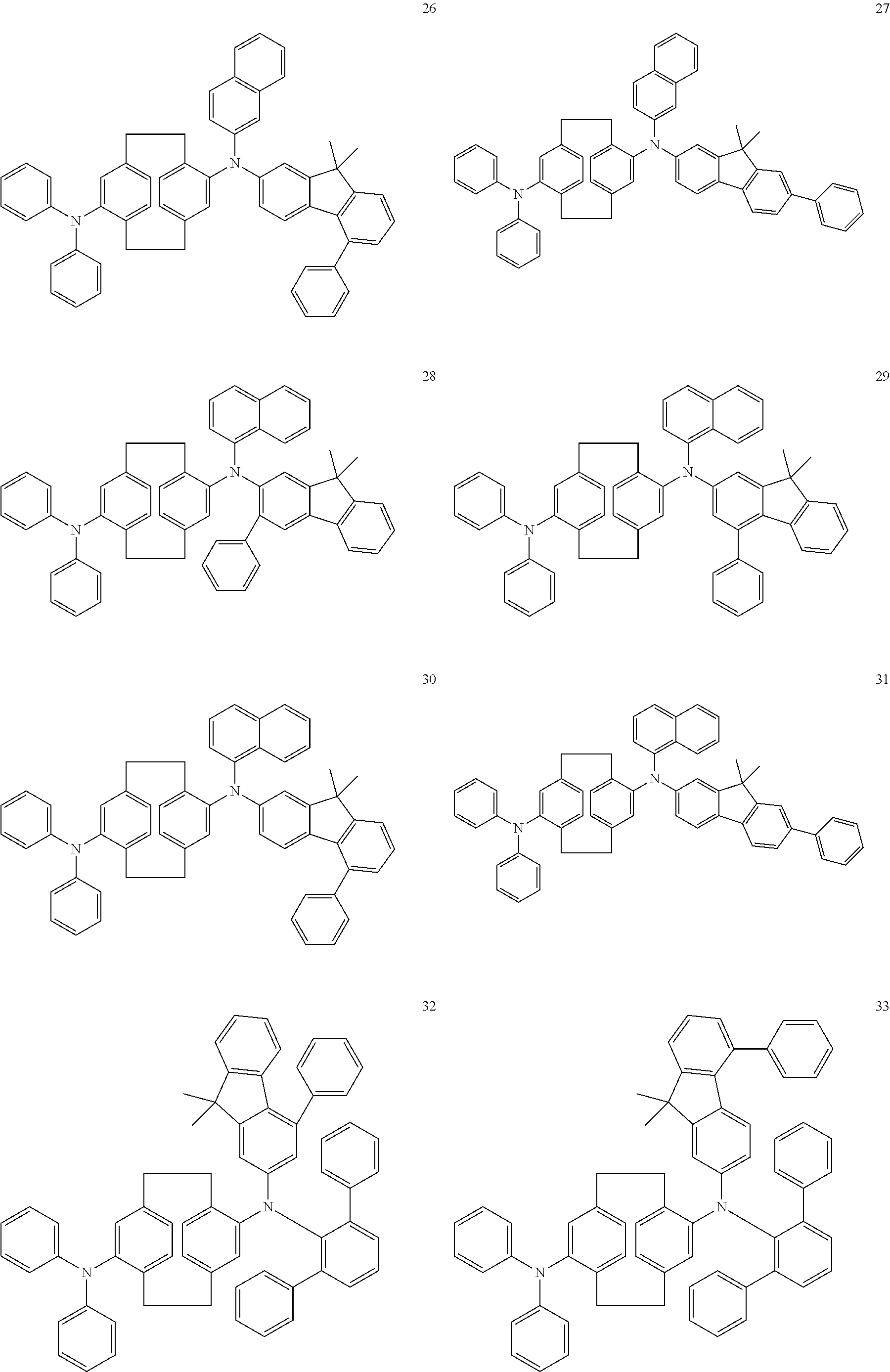

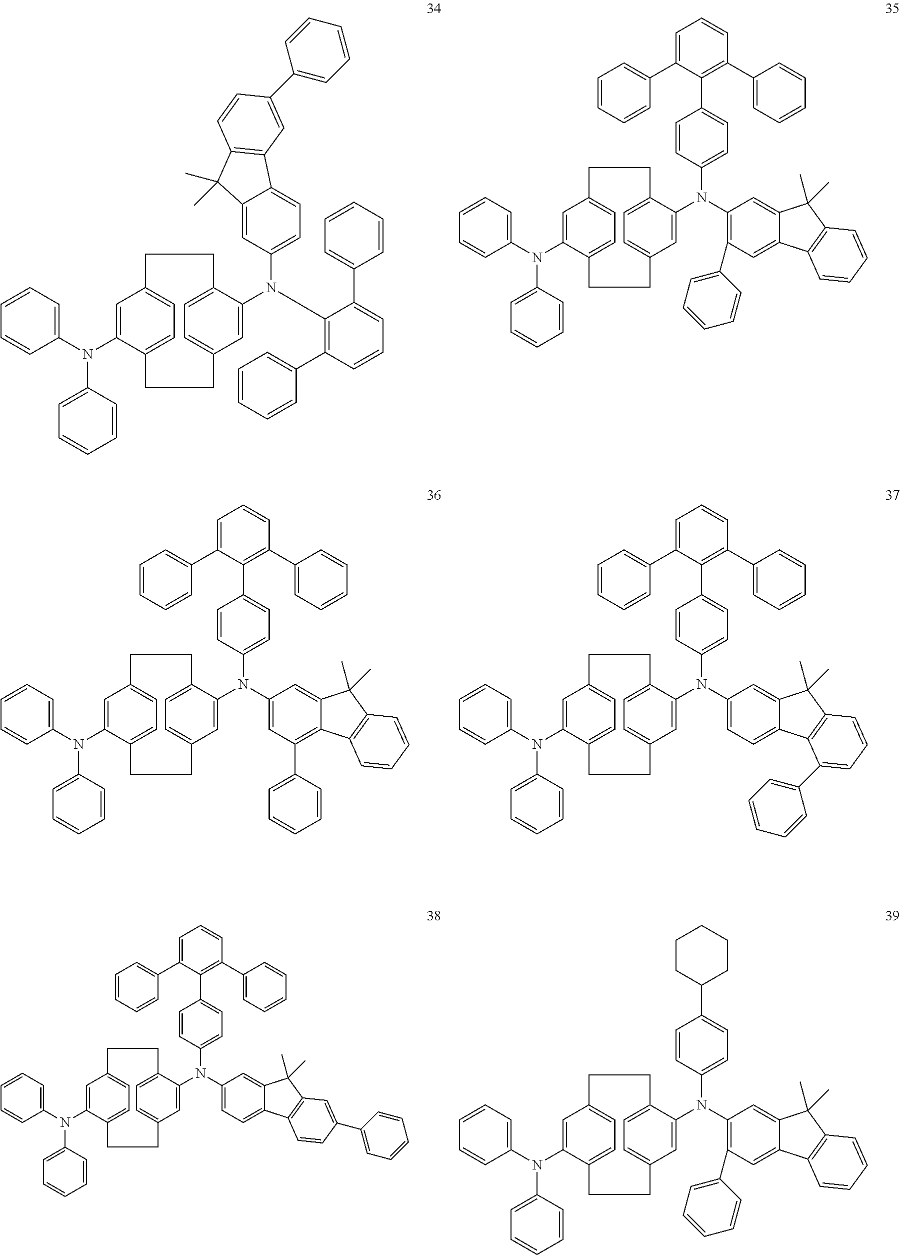

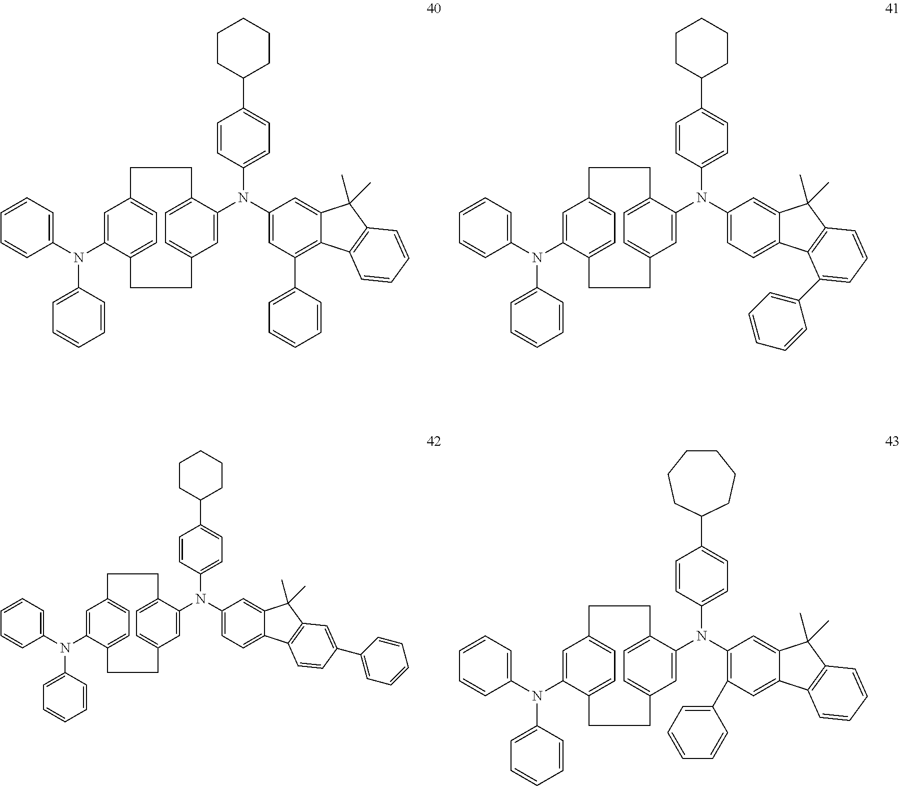 [SMILES: CC1(C)c2ccccc2-c2c(-c3ccccc3)cc(N(c3ccc(C4CCCCC4)cc3)c3cc4ccc3CCc3ccc(c(N(c5ccccc5)c5ccccc5)c3)CC4)cc21 ; CC1(C)c2cc(N(c3ccc(C4CCCCC4)cc3)c3cc4ccc3CCc3ccc(c(N(c5ccccc5)c5ccccc5)c3)CC4)ccc2-c2c(-c3ccccc3)cccc21 ; CC1(C)c2cc(-c3ccccc3)ccc2-c2ccc(N(c3ccc(C4CCCCC4)cc3)c3cc4ccc3CCc3ccc(c(N(c5ccccc5)c5ccccc5)c3)CC4)cc21 ; CC1(C)c2ccccc2-c2cc(-c3ccccc3)c(N(c3ccc(C4CCCCCC4)cc3)c3cc4ccc3CCc3ccc(c(N(c5ccccc5)c5ccccc5)c3)CC4)cc21]

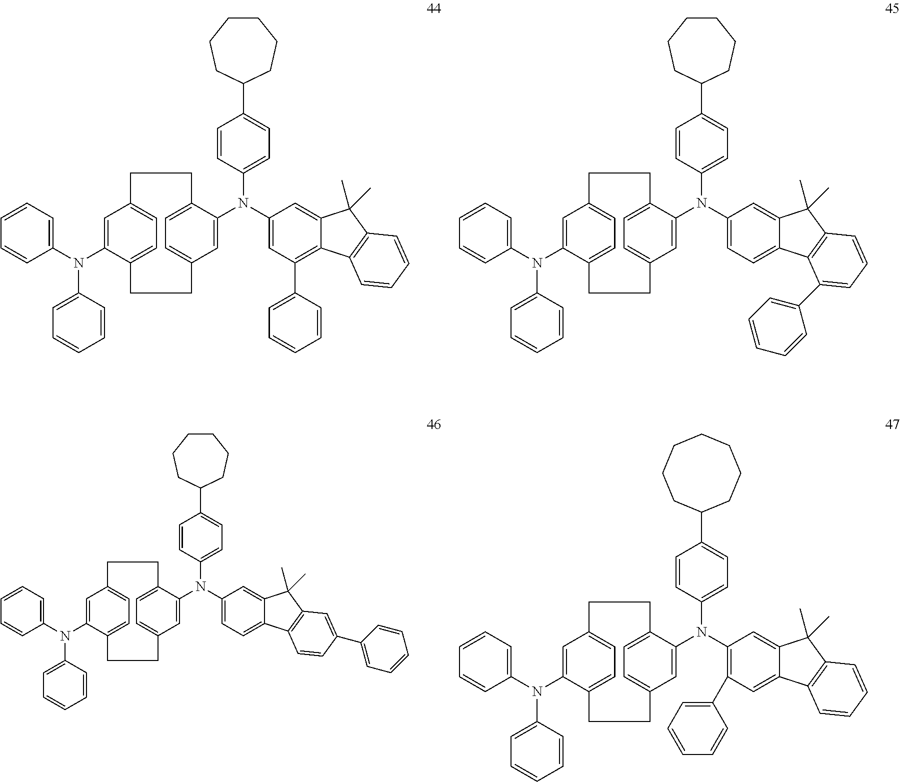 [SMILES: CC1(C)c2ccccc2-c2c(-c3ccccc3)cc(N(c3ccc(C4CCCCCC4)cc3)c3cc4ccc3CCc3ccc(c(N(c5ccccc5)c5ccccc5)c3)CC4)cc21 ; CC1(C)c2cc(N(c3ccc(C4CCCCCC4)cc3)c3cc4ccc3CCc3ccc(c(N(c5ccccc5)c5ccccc5)c3)CC4)ccc2-c2c(-c3ccccc3)cccc21 ; CC1(C)c2cc(-c3ccccc3)ccc2-c2ccc(N(c3ccc(C4CCCCCC4)cc3)c3cc4ccc3CCc3ccc(c(N(c5ccccc5)c5ccccc5)c3)CC4)cc21 ; CC1(C)c2ccccc2-c2cc(-c3ccccc3)c(N(c3ccc(C4CCCCCCC4)cc3)c3cc4ccc3CCc3ccc(c(N(c5ccccc5)c5ccccc5)c3)CC4)cc21]

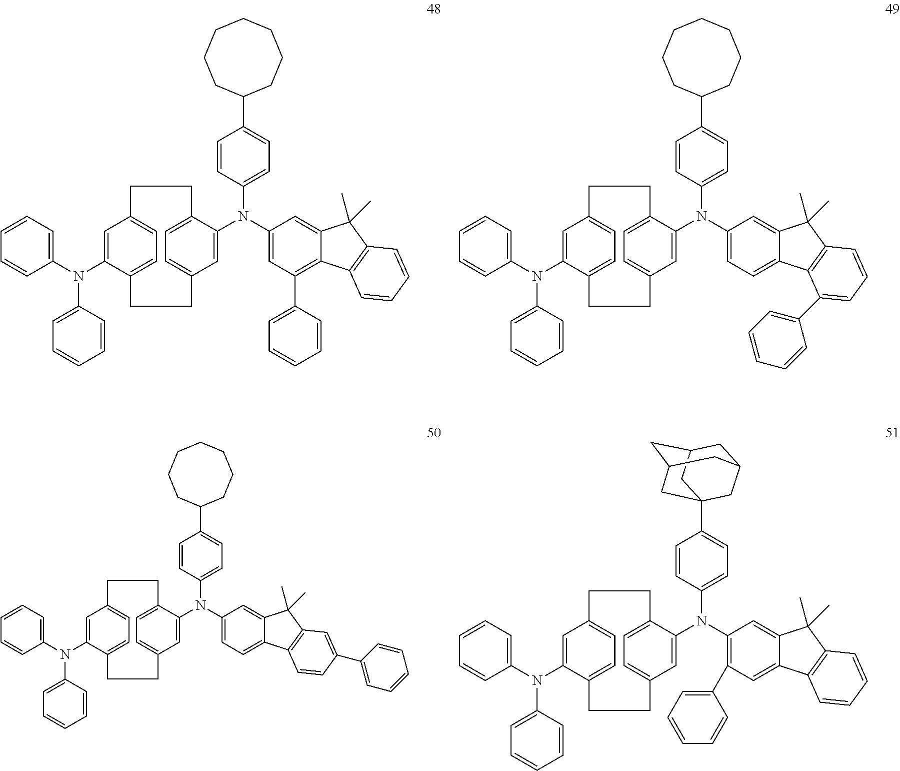 [SMILES: CC1(C)c2ccccc2-c2c(-c3ccccc3)cc(N(c3ccc(C4CCCCCCC4)cc3)c3cc4ccc3CCc3ccc(c(N(c5ccccc5)c5ccccc5)c3)CC4)cc21 ; CC1(C)c2cc(N(c3ccc(C4CCCCCCC4)cc3)c3cc4ccc3CCc3ccc(c(N(c5ccccc5)c5ccccc5)c3)CC4)ccc2-c2c(-c3ccccc3)cccc21 ; CC1(C)c2cc(-c3ccccc3)ccc2-c2ccc(N(c3ccc(C4CCCCCCC4)cc3)c3cc4ccc3CCc3ccc(c(N(c5ccccc5)c5ccccc5)c3)CC4)cc21 ; CC1(C)c2ccccc2-c2cc(-c3ccccc3)c(N(c3ccc(C45CC6CC(CC(C6)C4)C5)cc3)c3cc4ccc3CCc3ccc(c(N(c5ccccc5)c5ccccc5)c3)CC4)cc21]

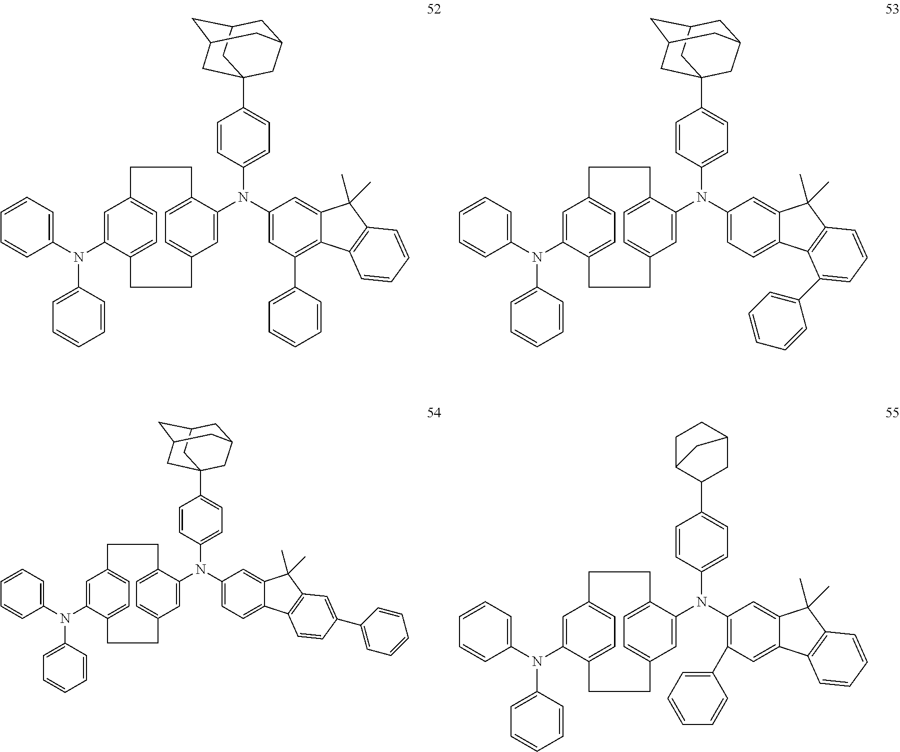 [SMILES: CC1(C)c2ccccc2-c2c(-c3ccccc3)cc(N(c3ccc(C45CC6CC(CC(C6)C4)C5)cc3)c3cc4ccc3CCc3ccc(c(N(c5ccccc5)c5ccccc5)c3)CC4)cc21 ; CC1(C)c2cc(N(c3ccc(C45CC6CC(CC(C6)C4)C5)cc3)c3cc4ccc3CCc3ccc(c(N(c5ccccc5)c5ccccc5)c3)CC4)ccc2-c2c(-c3ccccc3)cccc21 ; CC1(C)c2cc(-c3ccccc3)ccc2-c2ccc(N(c3ccc(C45CC6CC(CC(C6)C4)C5)cc3)c3cc4ccc3CCc3ccc(c(N(c5ccccc5)c5ccccc5)c3)CC4)cc21 ; CC1(C)c2ccccc2-c2cc(-c3ccccc3)c(N(c3ccc(C4CC5CCC4C5)cc3)c3cc4ccc3CCc3ccc(c(N(c5ccccc5)c5ccccc5)c3)CC4)cc21]

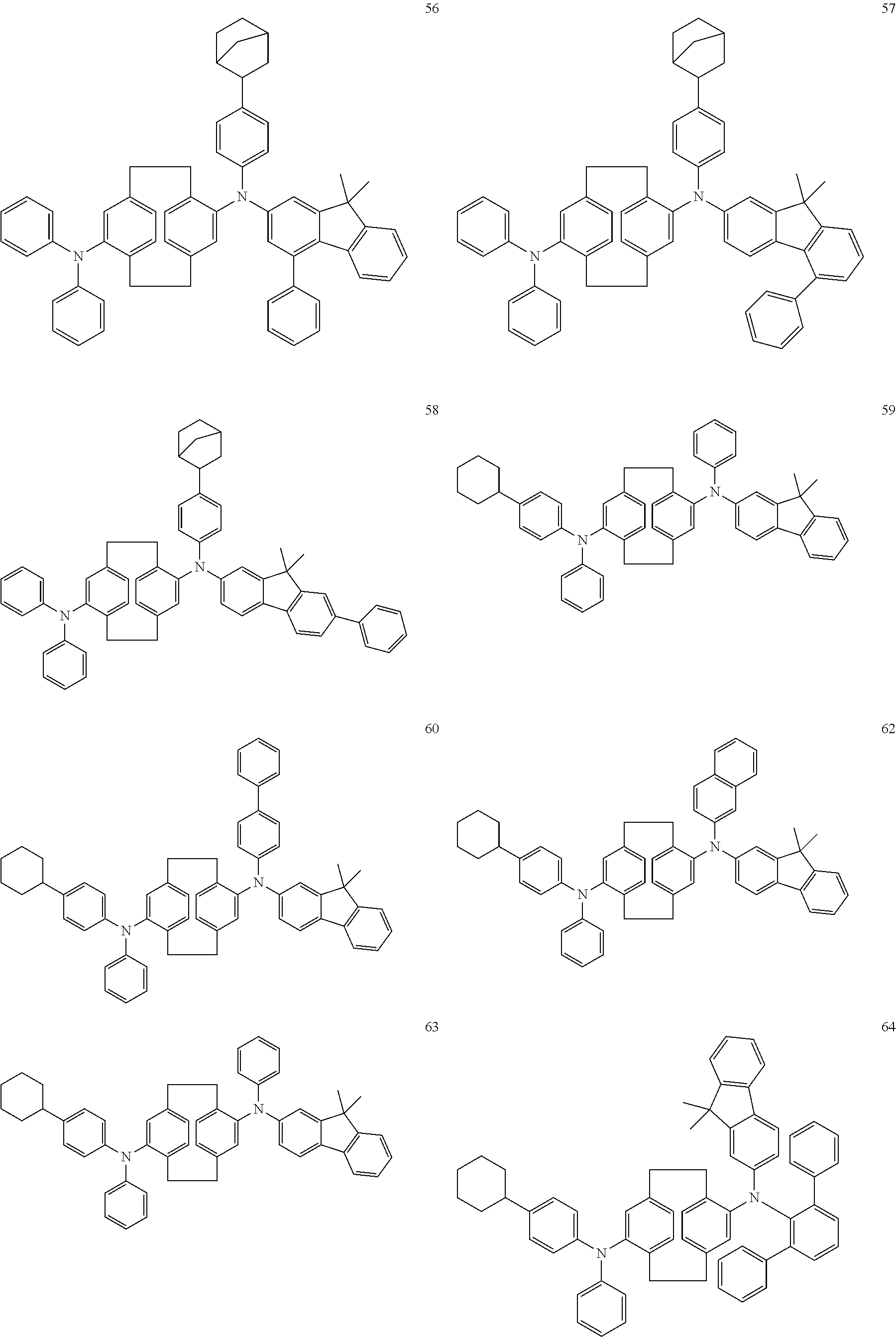

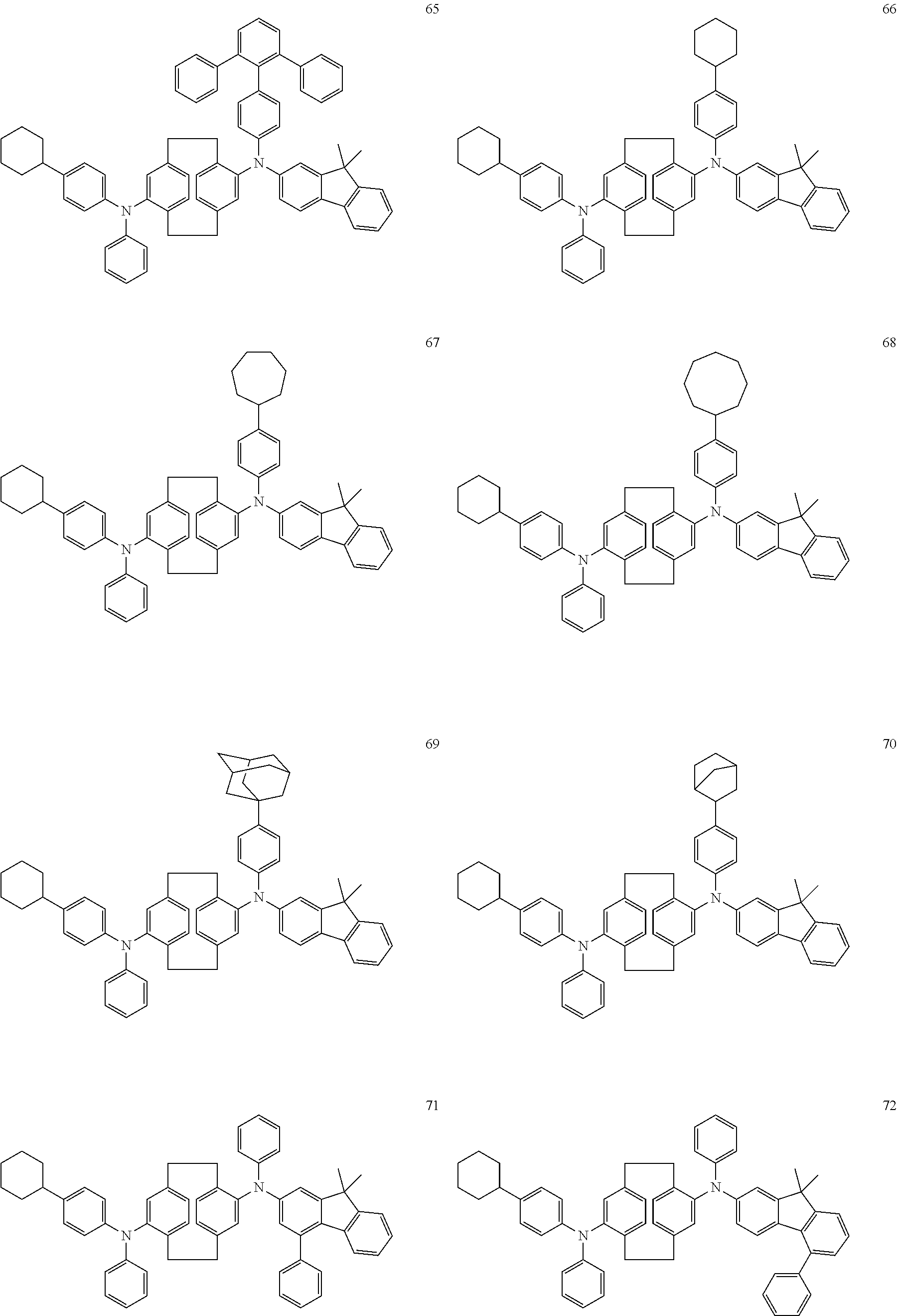

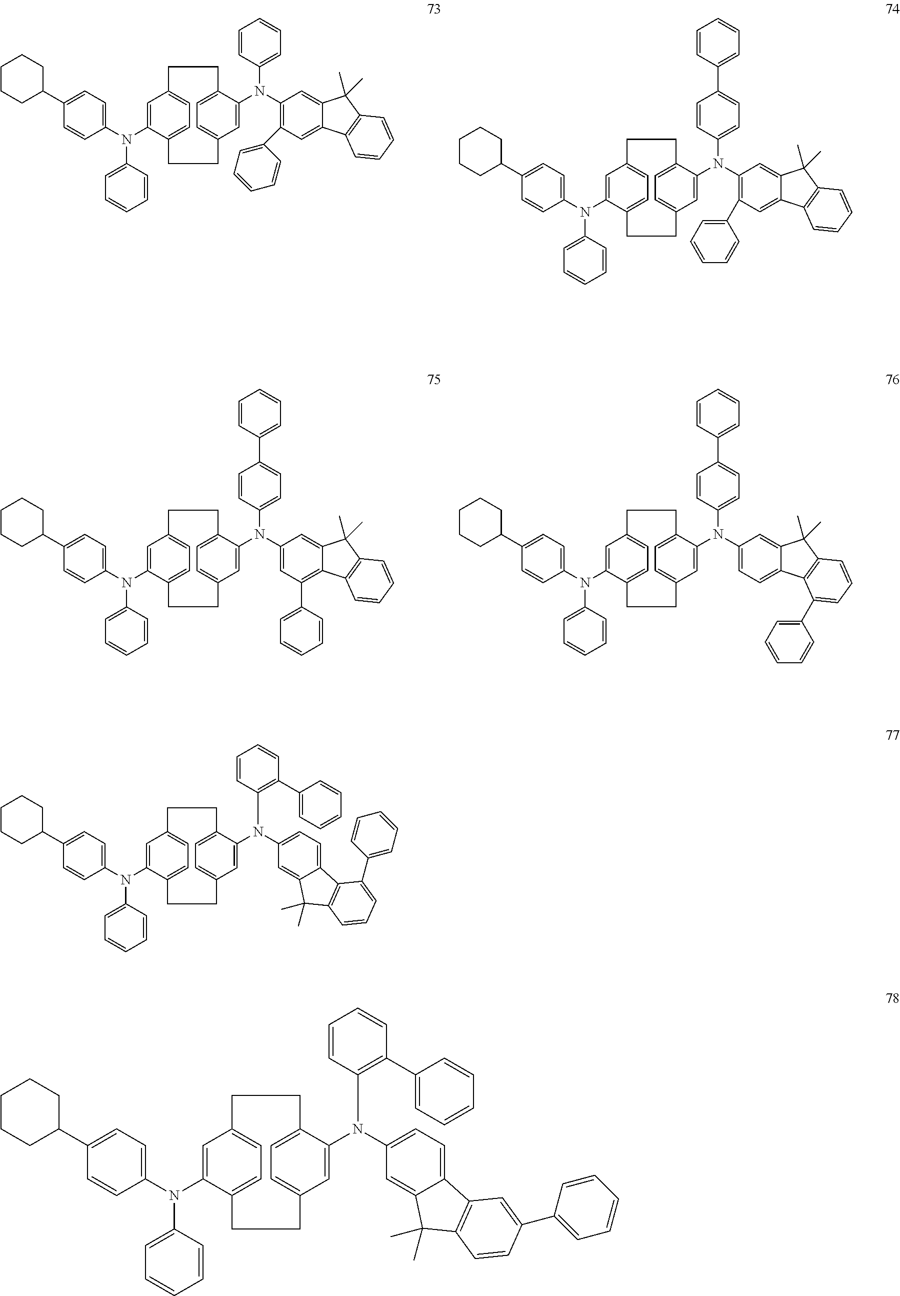

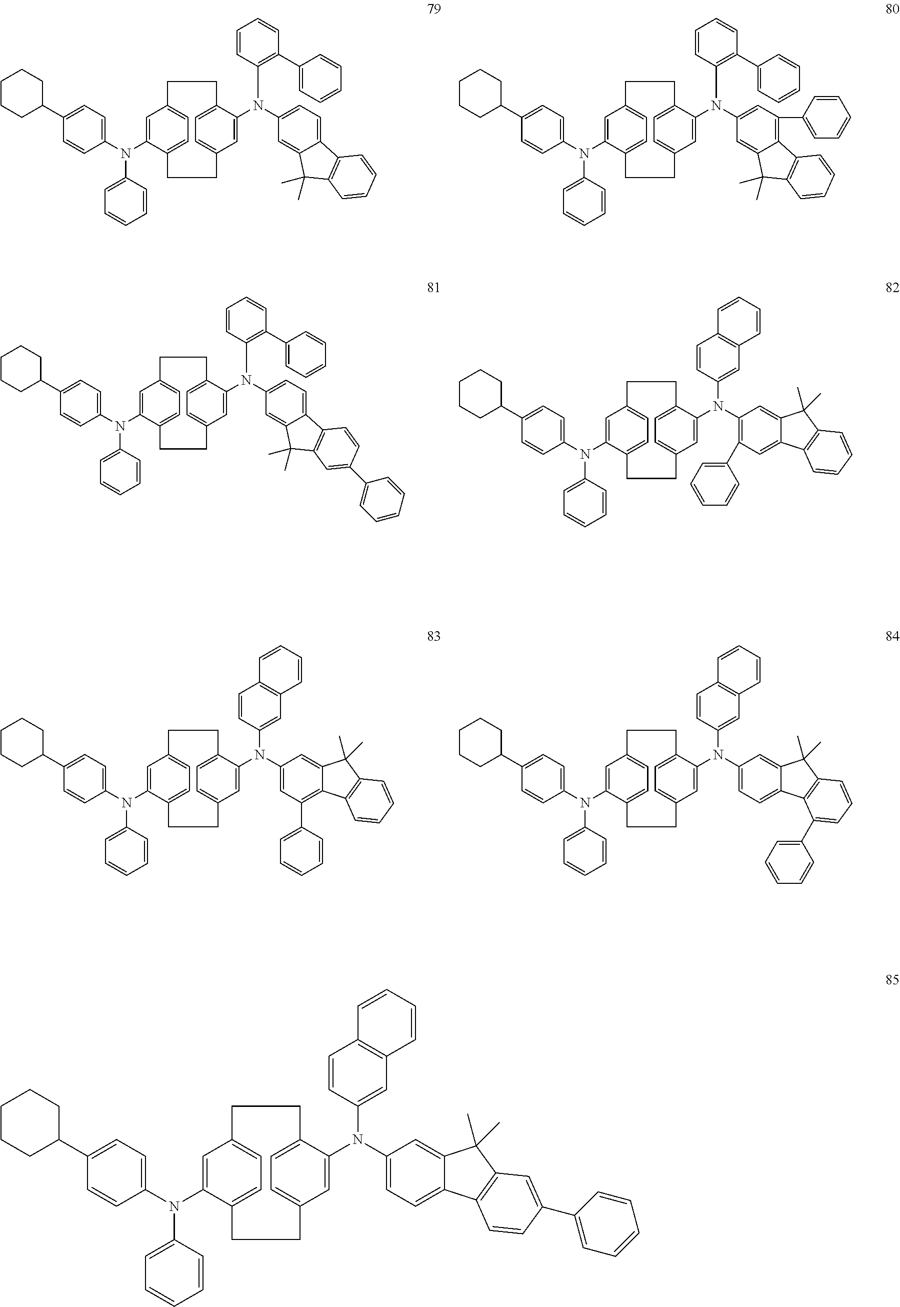

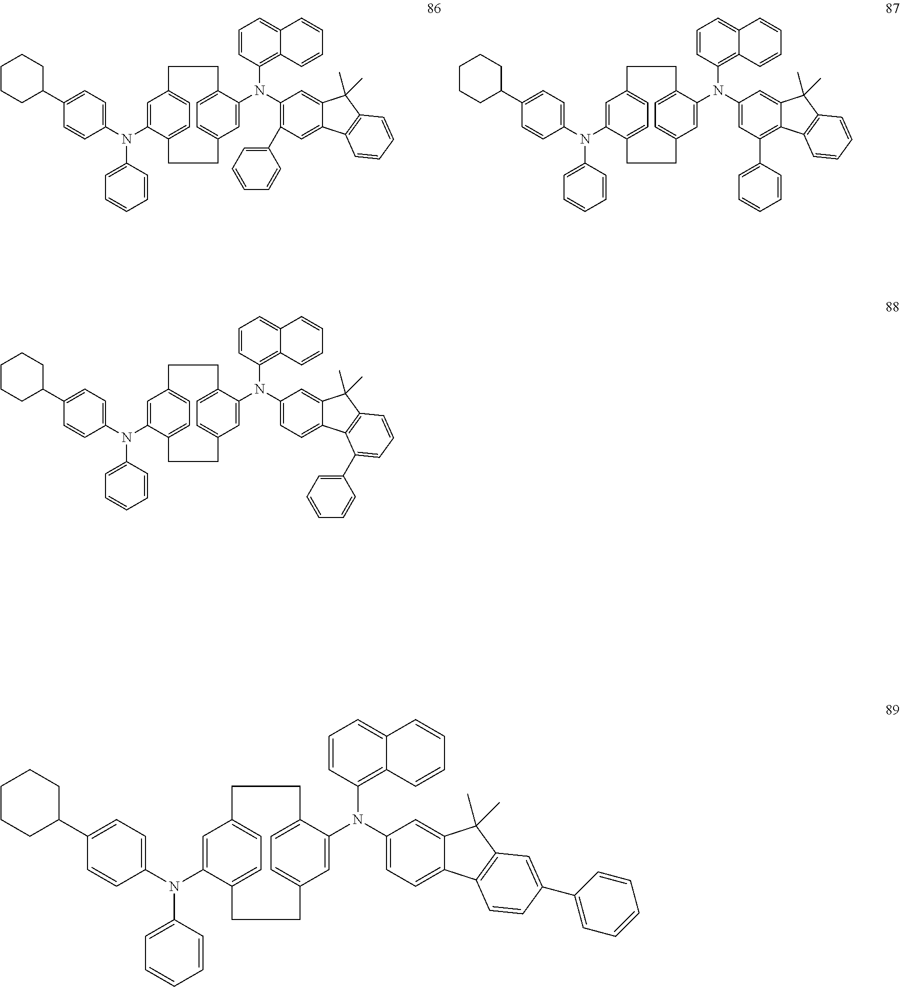 [SMILES: CC1(C)c2ccccc2-c2cc(-c3ccccc3)c(N(c3cc4ccc3CCc3ccc(c(N(c5ccccc5)c5ccc(C6CCCCC6)cc5)c3)CC4)c3cccc4ccccc34)cc21 ; CC1(C)c2ccccc2-c2c(-c3ccccc3)cc(N(c3cc4ccc3CCc3ccc(c(N(c5ccccc5)c5ccc(C6CCCCC6)cc5)c3)CC4)c3cccc4ccccc34)cc21 ; CC1(C)c2cc(N(c3cc4ccc3CCc3ccc(c(N(c5ccccc5)c5ccc(C6CCCCC6)cc5)c3)CC4)c3cccc4ccccc34)ccc2-c2c(-c3ccccc3)cccc21 ; CC1(C)c2cc(-c3ccccc3)ccc2-c2ccc(N(c3cc4ccc3CCc3ccc(c(N(c5ccccc5)c5ccc(C6CCCCC6)cc5)c3)CC4)c3cccc4ccccc34)cc21]

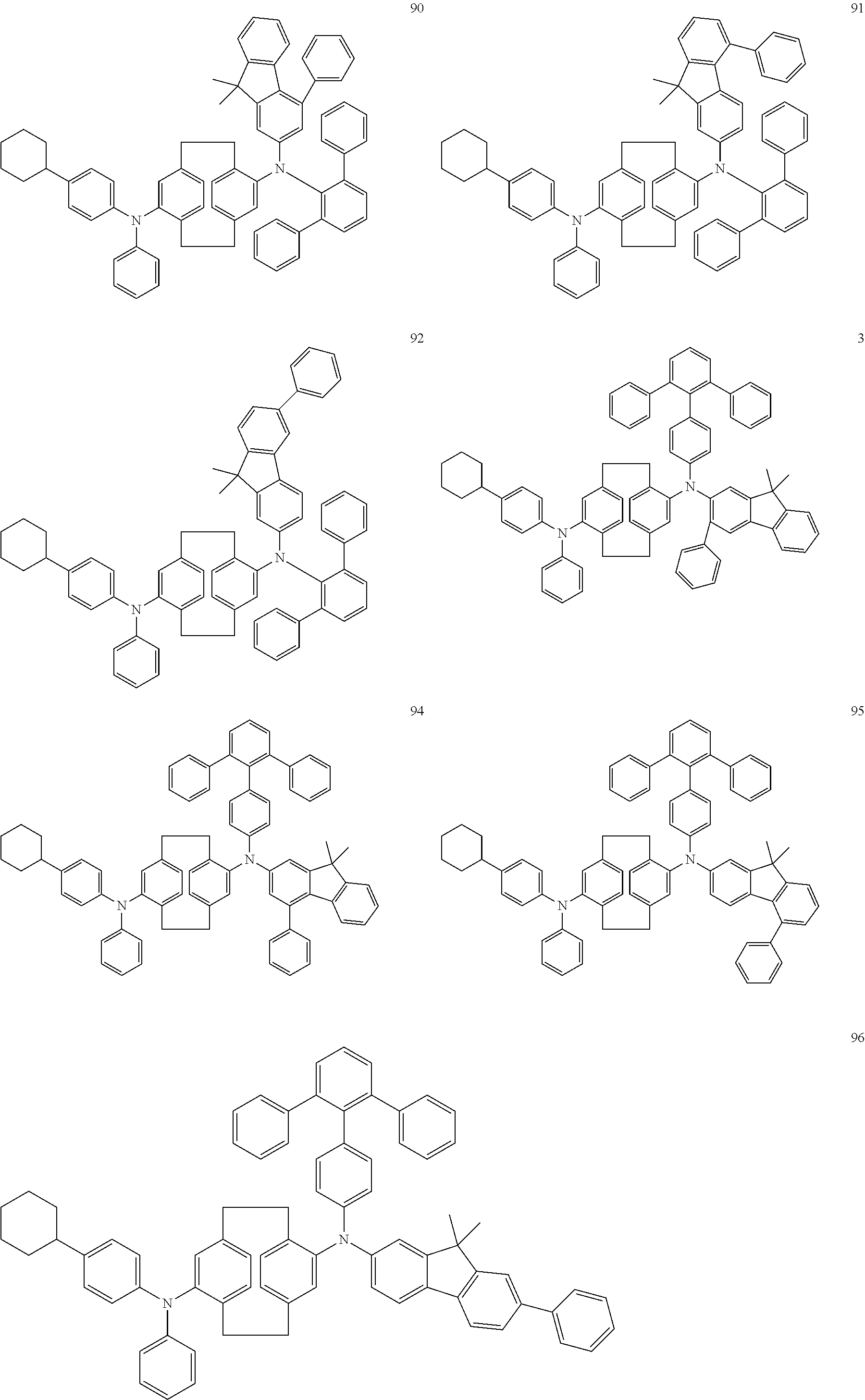

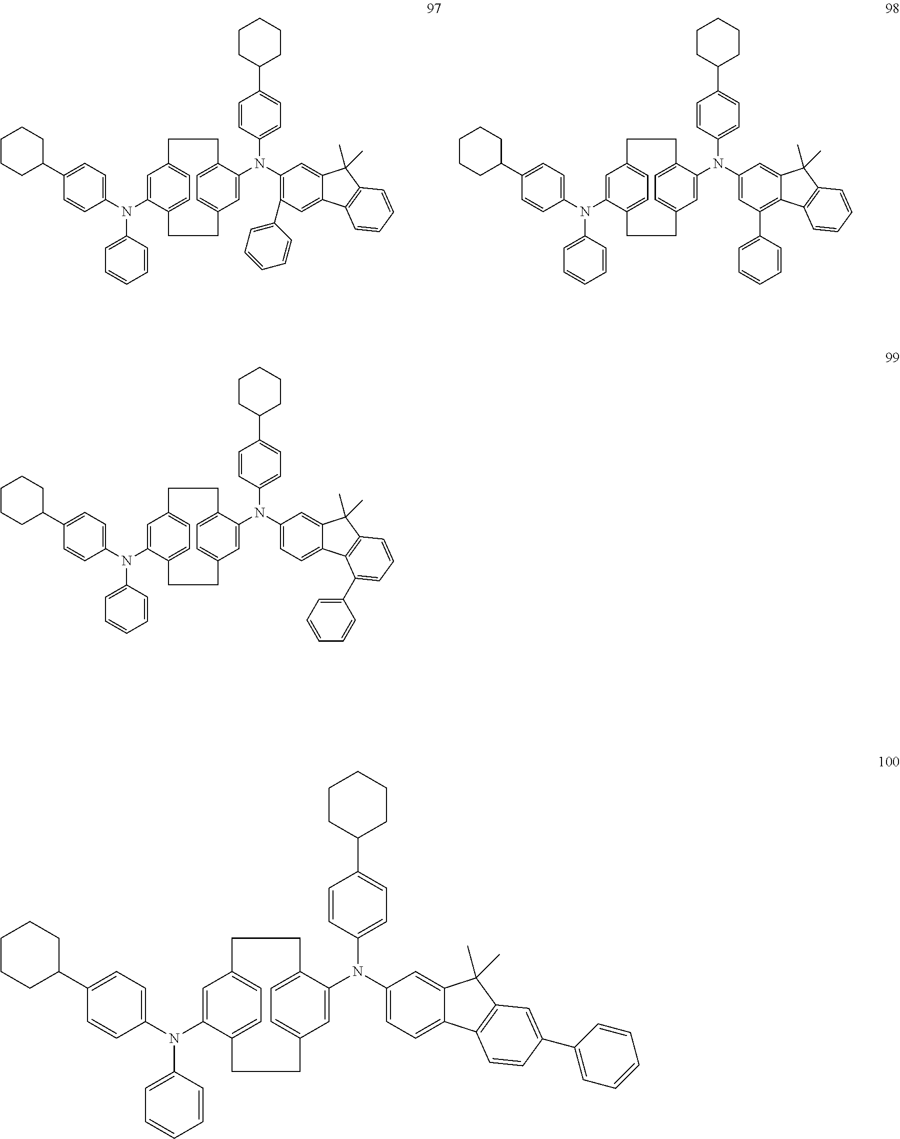 [SMILES: CC1(C)c2ccccc2-c2cc(-c3ccccc3)c(N(c3ccc(C4CCCCC4)cc3)c3cc4ccc3CCc3ccc(c(N(c5ccccc5)c5ccc(C6CCCCC6)cc5)c3)CC4)cc21 ; CC1(C)c2ccccc2-c2c(-c3ccccc3)cc(N(c3ccc(C4CCCCC4)cc3)c3cc4ccc3CCc3ccc(c(N(c5ccccc5)c5ccc(C6CCCCC6)cc5)c3)CC4)cc21 ; CC1(C)c2cc(N(c3ccc(C4CCCCC4)cc3)c3cc4ccc3CCc3ccc(c(N(c5ccccc5)c5ccc(C6CCCCC6)cc5)c3)CC4)ccc2-c2c(-c3ccccc3)cccc21 ; CC1(C)c2cc(-c3ccccc3)ccc2-c2ccc(N(c3ccc(C4CCCCC4)cc3)c3cc4ccc3CCc3ccc(c(N(c5ccccc5)c5ccc(C6CCCCC6)cc5)c3)CC4)cc21]

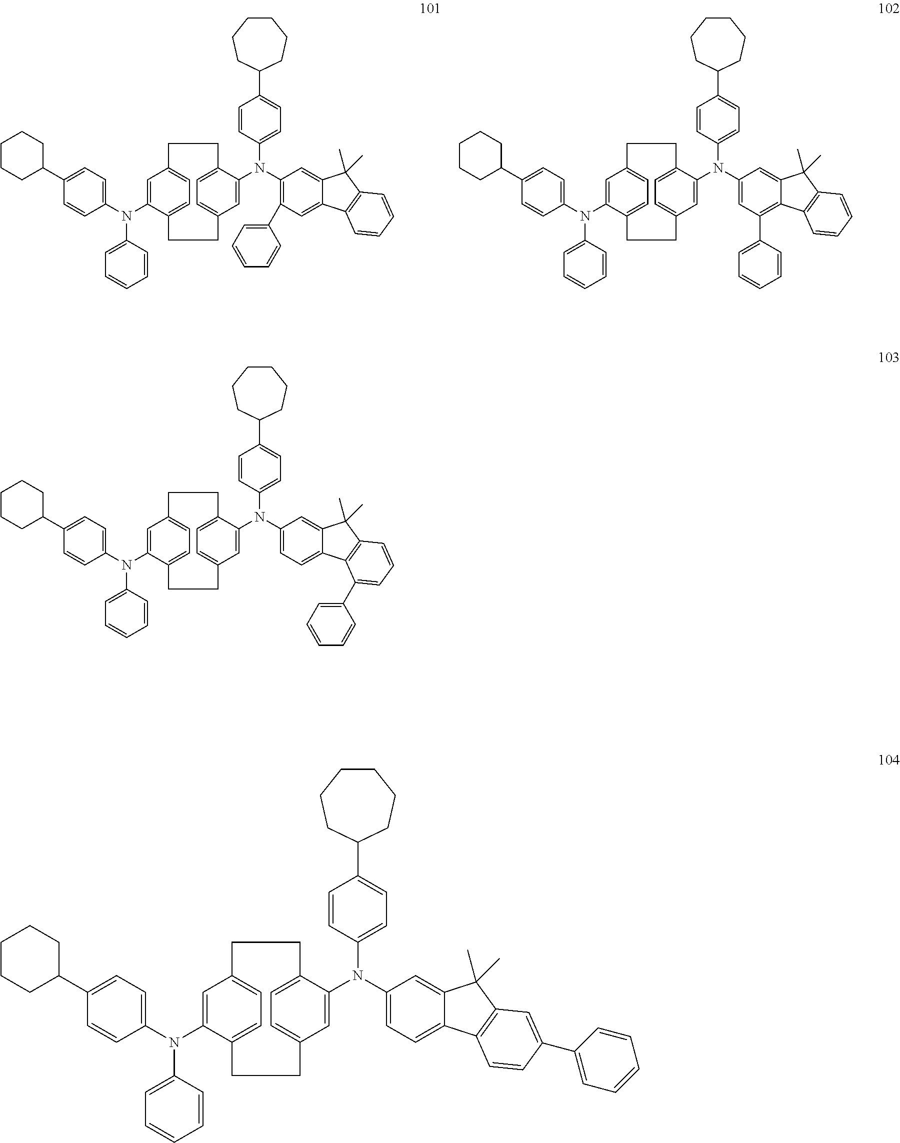 [SMILES: CC1(C)c2ccccc2-c2cc(-c3ccccc3)c(N(c3ccc(C4CCCCCC4)cc3)c3cc4ccc3CCc3ccc(c(N(c5ccccc5)c5ccc(C6CCCCC6)cc5)c3)CC4)cc21 ; CC1(C)c2ccccc2-c2c(-c3ccccc3)cc(N(c3ccc(C4CCCCCC4)cc3)c3cc4ccc3CCc3ccc(c(N(c5ccccc5)c5ccc(C6CCCCC6)cc5)c3)CC4)cc21 ; CC1(C)c2cc(N(c3ccc(C4CCCCCC4)cc3)c3cc4ccc3CCc3ccc(c(N(c5ccccc5)c5ccc(C6CCCCC6)cc5)c3)CC4)ccc2-c2c(-c3ccccc3)cccc21 ; CC1(C)c2cc(-c3ccccc3)ccc2-c2ccc(N(c3ccc(C4CCCCCC4)cc3)c3cc4ccc3CCc3ccc(c(N(c5ccccc5)c5ccc(C6CCCCC6)cc5)c3)CC4)cc21]

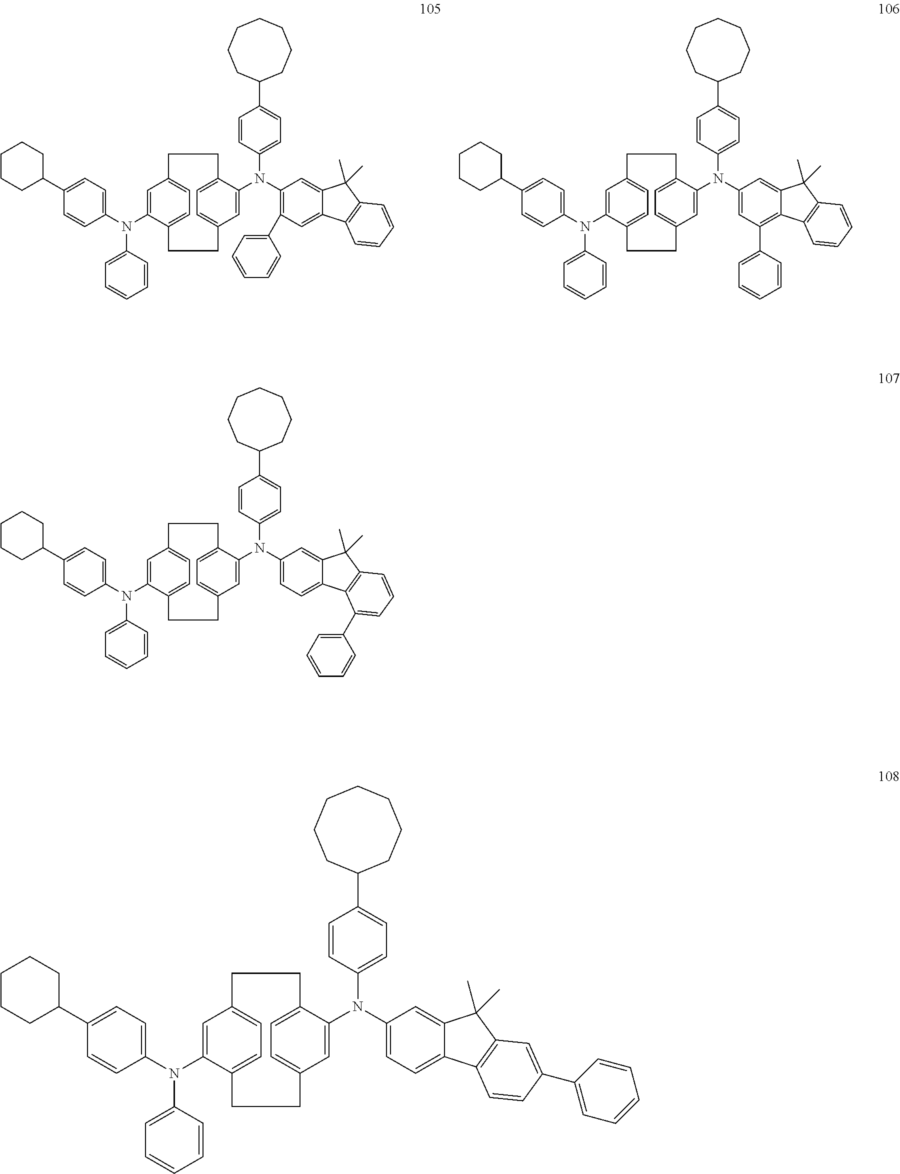 [SMILES: CC1(C)c2ccccc2-c2cc(-c3ccccc3)c(N(c3ccc(C4CCCCCCC4)cc3)c3cc4ccc3CCc3ccc(c(N(c5ccccc5)c5ccc(C6CCCCC6)cc5)c3)CC4)cc21 ; CC1(C)c2ccccc2-c2c(-c3ccccc3)cc(N(c3ccc(C4CCCCCCC4)cc3)c3cc4ccc3CCc3ccc(c(N(c5ccccc5)c5ccc(C6CCCCC6)cc5)c3)CC4)cc21 ; CC1(C)c2cc(N(c3ccc(C4CCCCCCC4)cc3)c3cc4ccc3CCc3ccc(c(N(c5ccccc5)c5ccc(C6CCCCC6)cc5)c3)CC4)ccc2-c2c(-c3ccccc3)cccc21 ; CC1(C)c2cc(-c3ccccc3)ccc2-c2ccc(N(c3ccc(C4CCCCCCC4)cc3)c3cc4ccc3CCc3ccc(c(N(c5ccccc5)c5ccc(C6CCCCC6)cc5)c3)CC4)cc21]

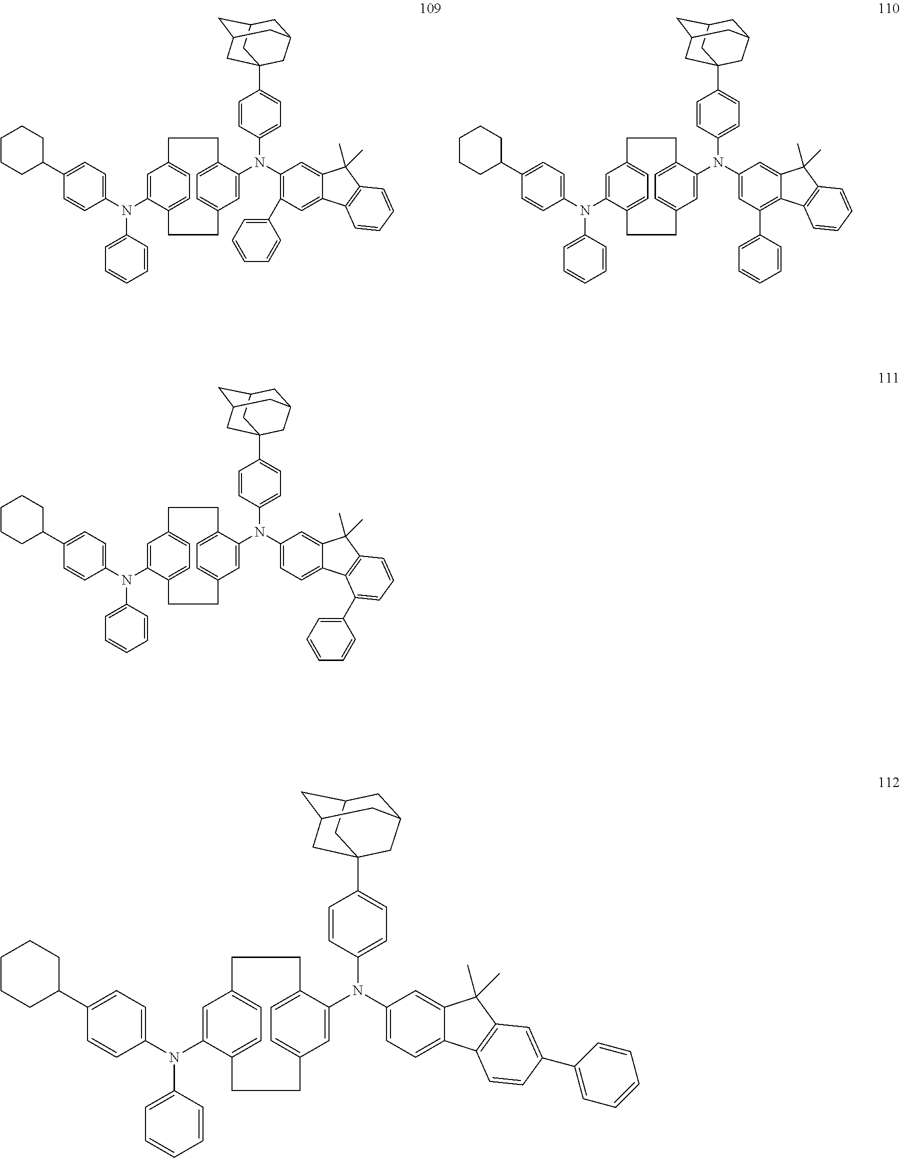

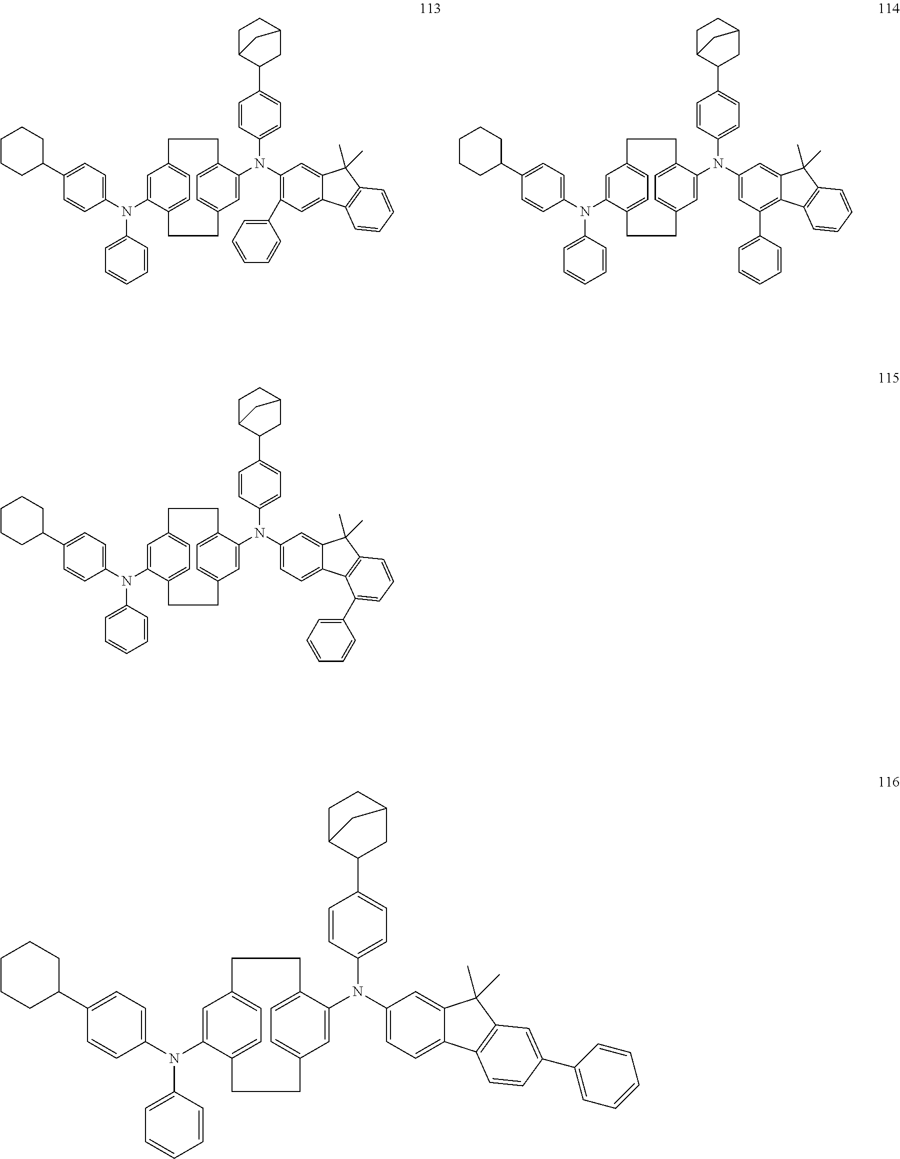 [SMILES: CC1(C)c2ccccc2-c2cc(-c3ccccc3)c(N(c3ccc(C4CC5CCC4C5)cc3)c3cc4ccc3CCc3ccc(c(N(c5ccccc5)c5ccc(C6CCCCC6)cc5)c3)CC4)cc21 ; CC1(C)c2ccccc2-c2c(-c3ccccc3)cc(N(c3ccc(C4CC5CCC4C5)cc3)c3cc4ccc3CCc3ccc(c(N(c5ccccc5)c5ccc(C6CCCCC6)cc5)c3)CC4)cc21 ; CC1(C)c2cc(N(c3ccc(C4CC5CCC4C5)cc3)c3cc4ccc3CCc3ccc(c(N(c5ccccc5)c5ccc(C6CCCCC6)cc5)c3)CC4)ccc2-c2c(-c3ccccc3)cccc21 ; CC1(C)c2cc(-c3ccccc3)ccc2-c2ccc(N(c3ccc(C4CC5CCC4C5)cc3)c3cc4ccc3CCc3ccc(c(N(c5ccccc5)c5ccc(C6CCCCC6)cc5)c3)CC4)cc21]

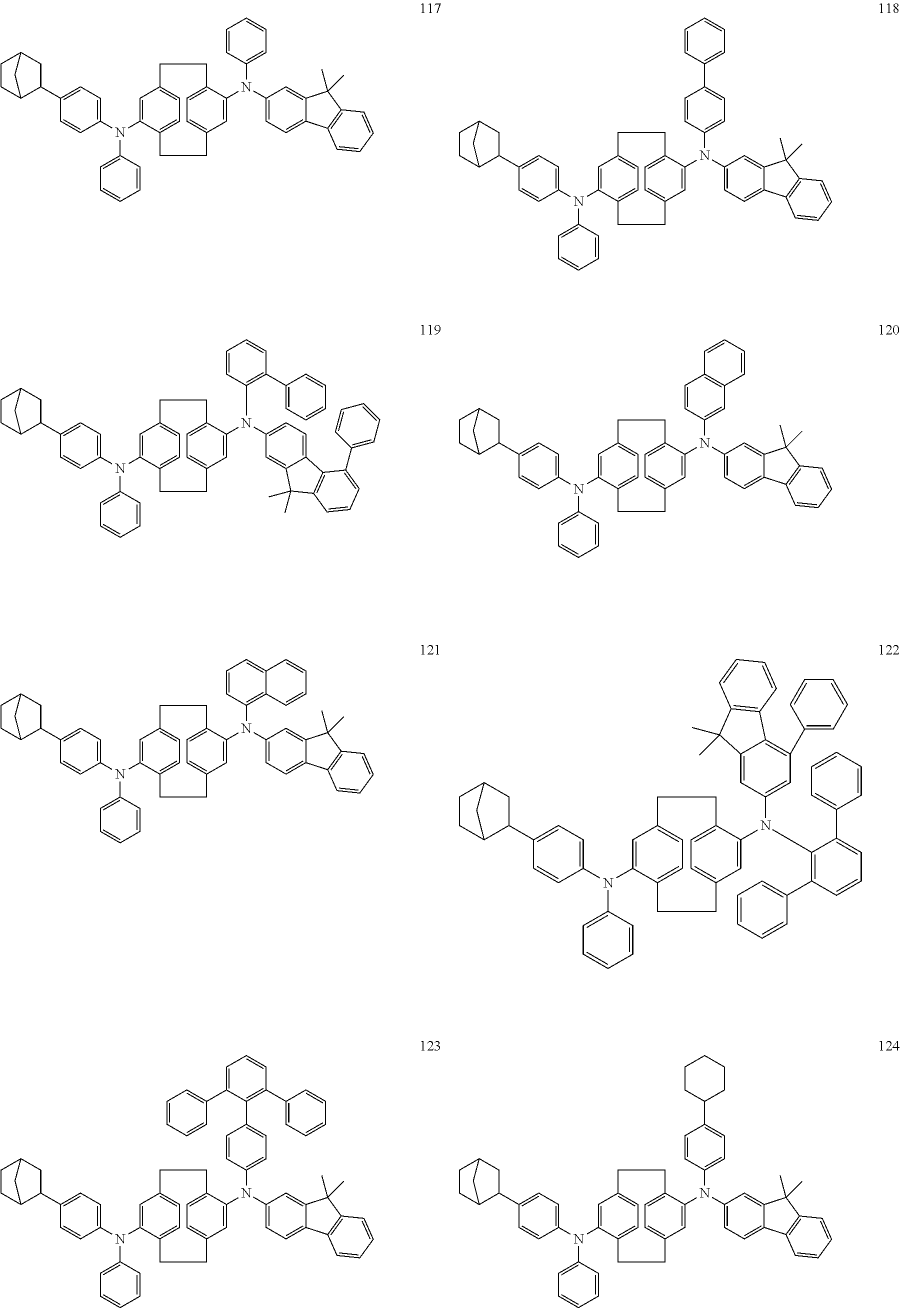

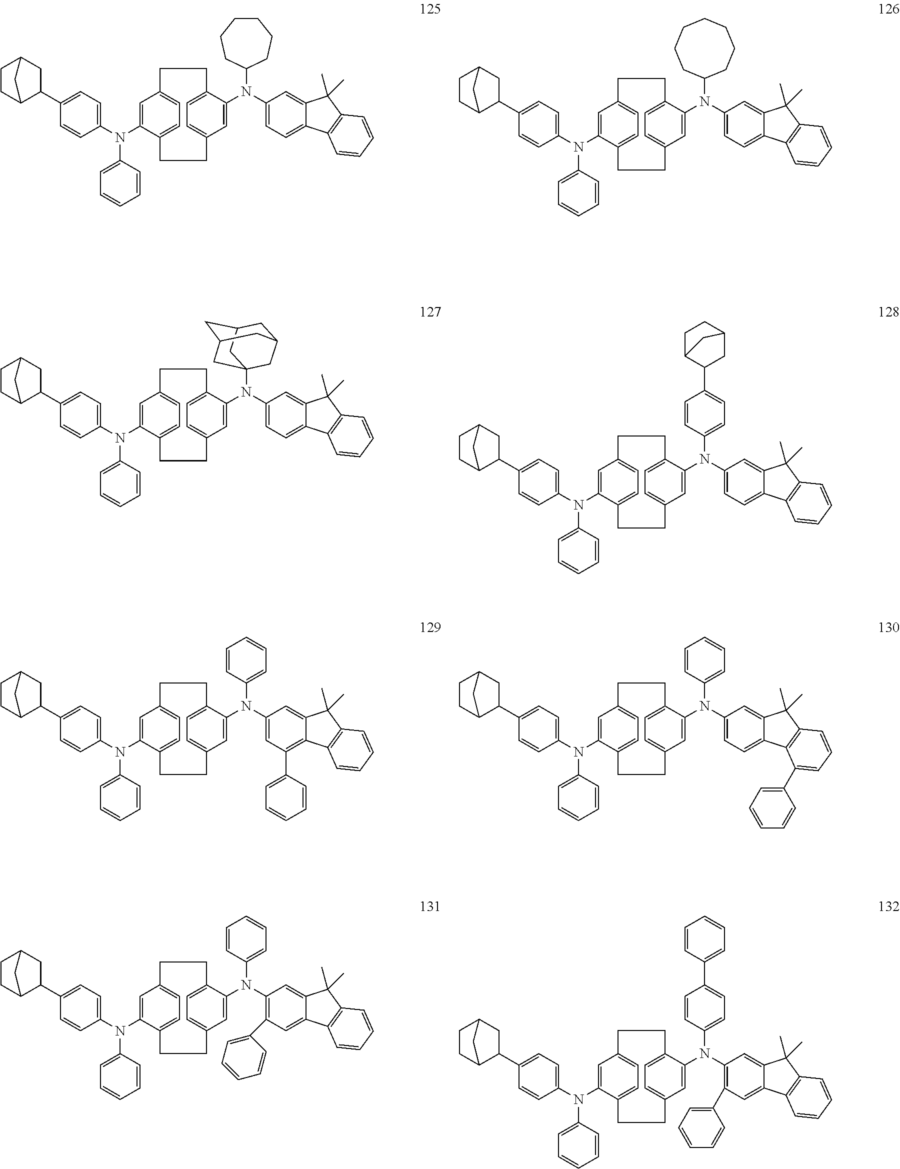

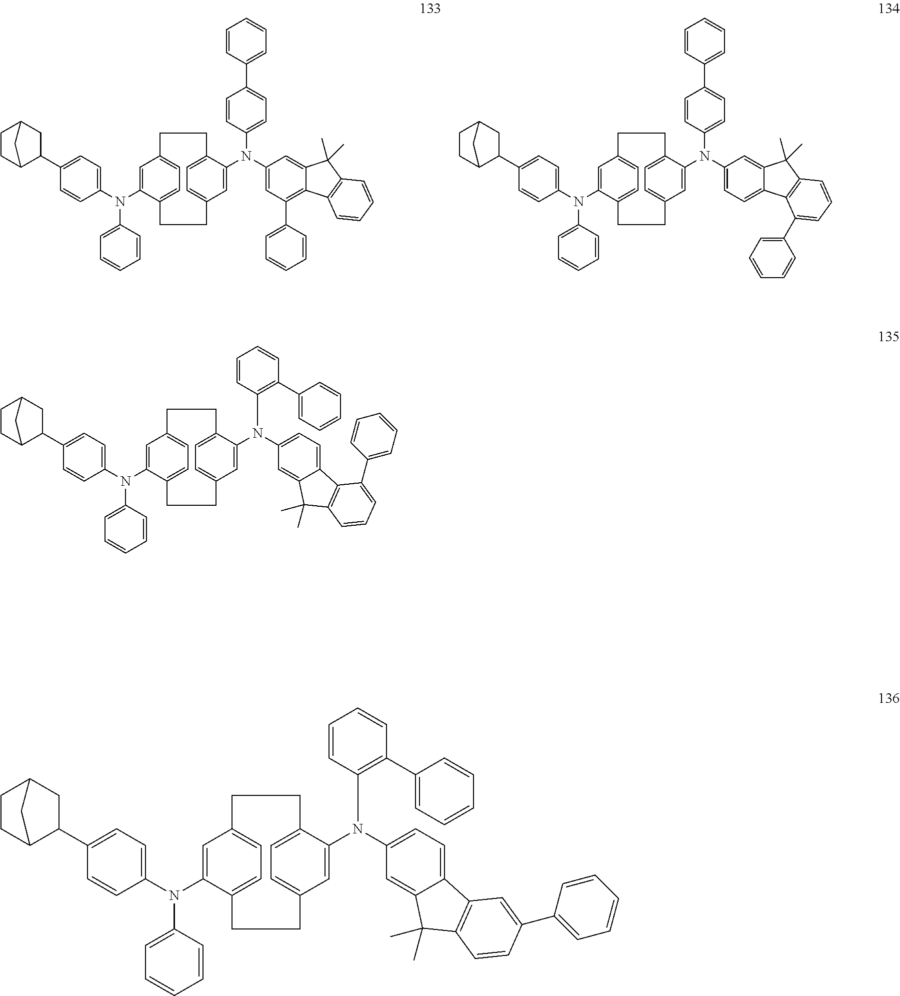 [SMILES: CC1(C)c2ccccc2-c2c(-c3ccccc3)cc(N(c3ccc(-c4ccccc4)cc3)c3cc4ccc3CCc3ccc(c(N(c5ccccc5)c5ccc(C6CC7CCC6C7)cc5)c3)CC4)cc21 ; CC1(C)c2cc(N(c3ccc(-c4ccccc4)cc3)c3cc4ccc3CCc3ccc(c(N(c5ccccc5)c5ccc(C6CC7CCC6C7)cc5)c3)CC4)ccc2-c2c(-c3ccccc3)cccc21 ; CC1(C)c2cc(N(c3cc4ccc3CCc3ccc(c(N(c5ccccc5)c5ccc(C6CC7CCC6C7)cc5)c3)CC4)c3ccccc3-c3ccccc3)ccc2-c2c(-c3ccccc3)cccc21 ; CC1(C)c2ccc(-c3ccccc3)cc2-c2ccc(N(c3cc4ccc3CCc3ccc(c(N(c5ccccc5)c5ccc(C6CC7CCC6C7)cc5)c3)CC4)c3ccccc3-c3ccccc3)cc21]

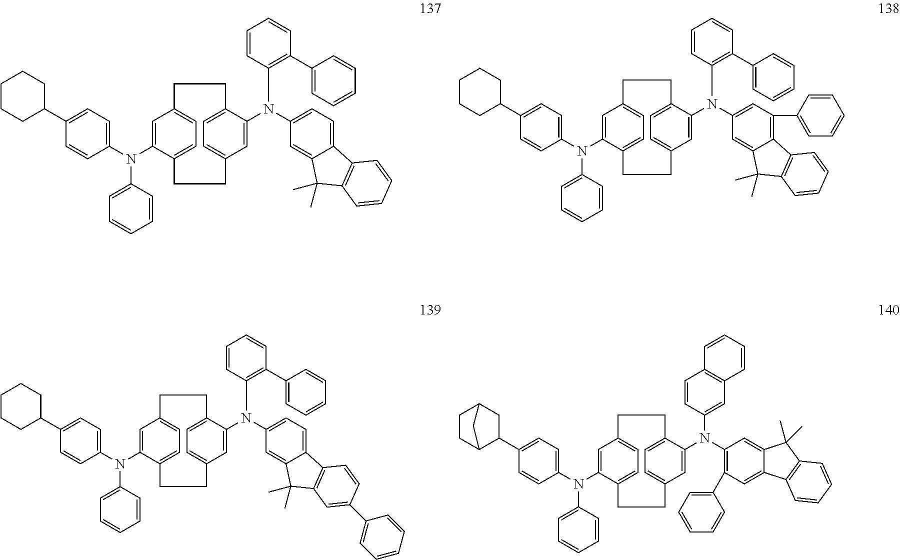 [SMILES: CC1(C)c2ccccc2-c2ccc(N(c3cc4ccc3CCc3ccc(c(N(c5ccccc5)c5ccc(C6CCCCC6)cc5)c3)CC4)c3ccccc3-c3ccccc3)cc21 ; CC1(C)c2ccccc2-c2c(-c3ccccc3)cc(N(c3cc4ccc3CCc3ccc(c(N(c5ccccc5)c5ccc(C6CCCCC6)cc5)c3)CC4)c3ccccc3-c3ccccc3)cc21 ; CC1(C)c2cc(-c3ccccc3)ccc2-c2ccc(N(c3cc4ccc3CCc3ccc(c(N(c5ccccc5)c5ccc(C6CCCCC6)cc5)c3)CC4)c3ccccc3-c3ccccc3)cc21 ; CC1(C)c2ccccc2-c2cc(-c3ccccc3)c(N(c3ccc4ccccc4c3)c3cc4ccc3CCc3ccc(c(N(c5ccccc5)c5ccc(C6CC7CCC6C7)cc5)c3)CC4)cc21]

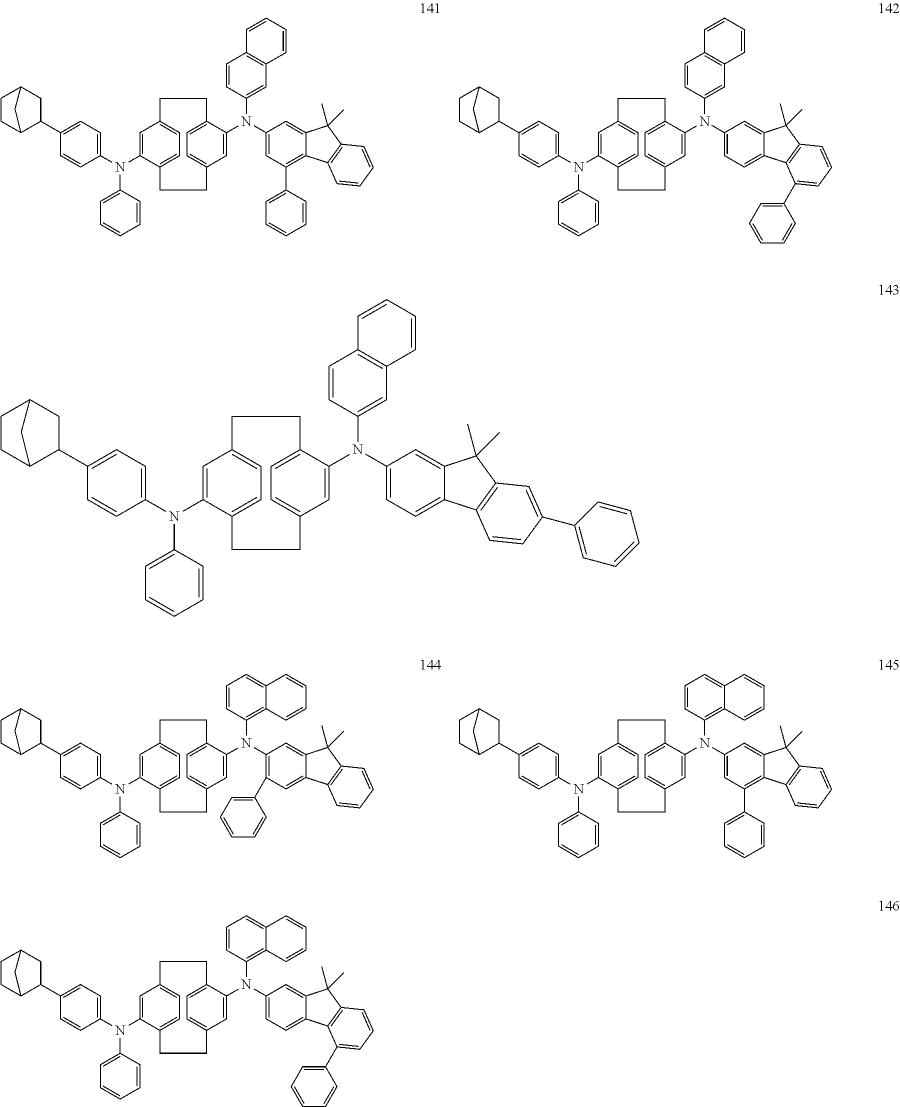

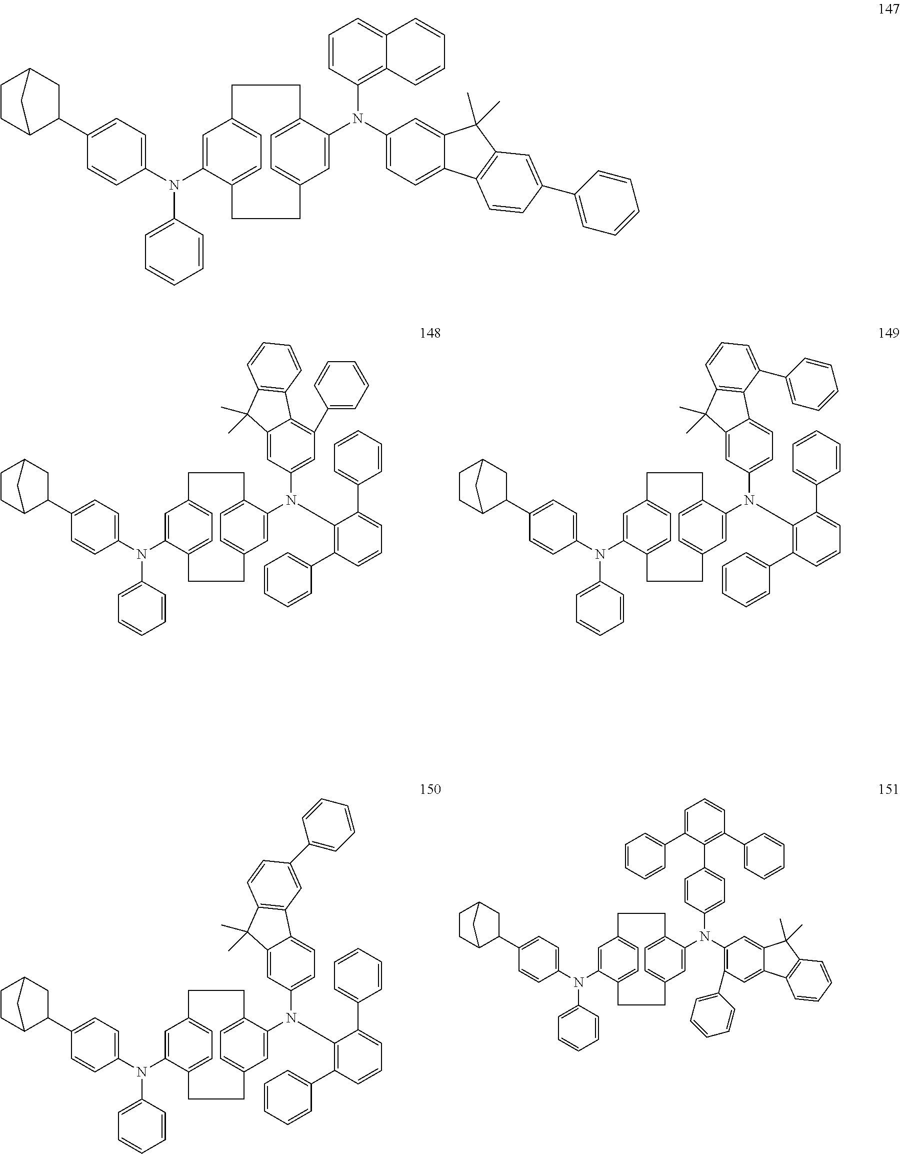

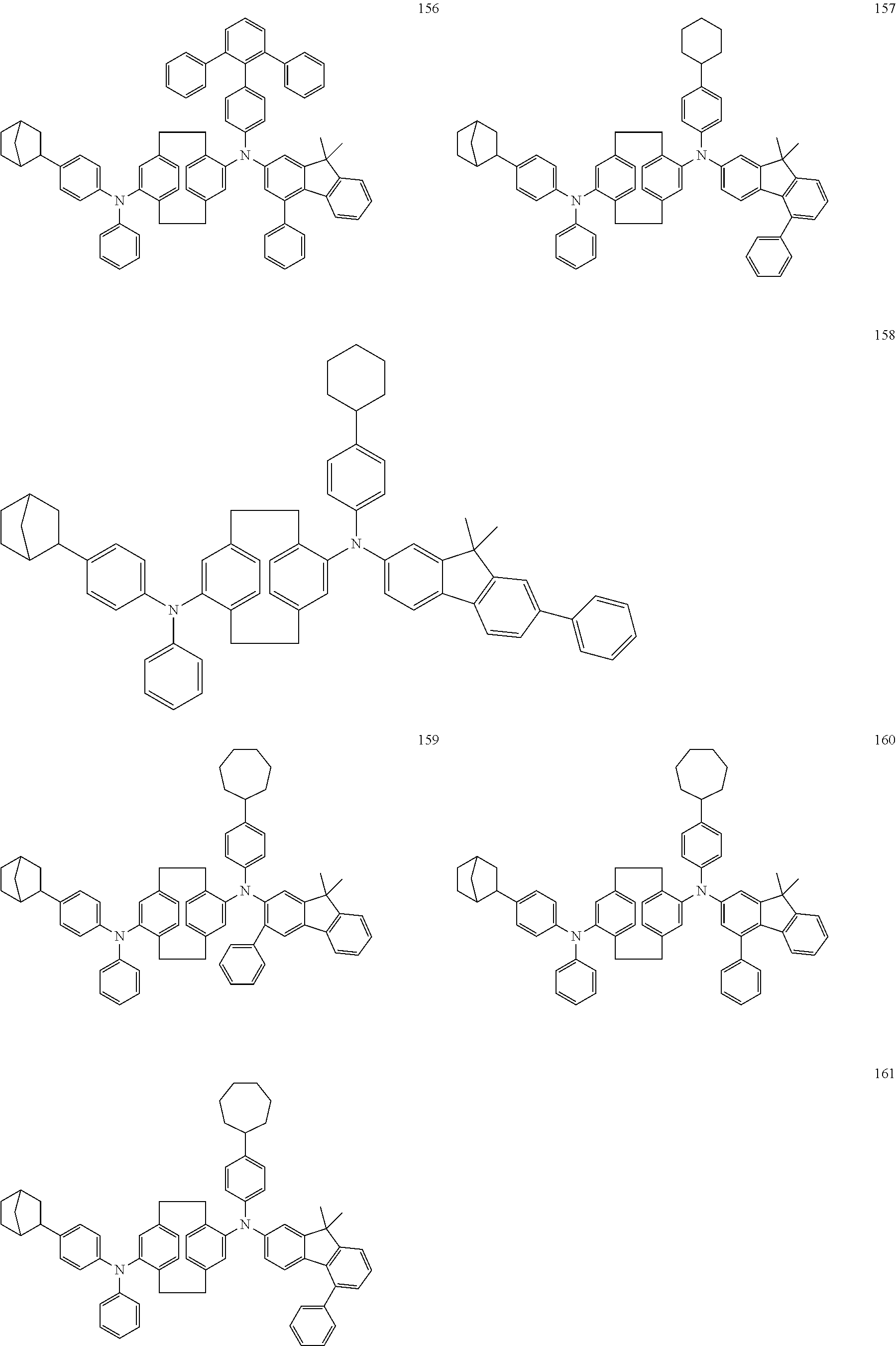

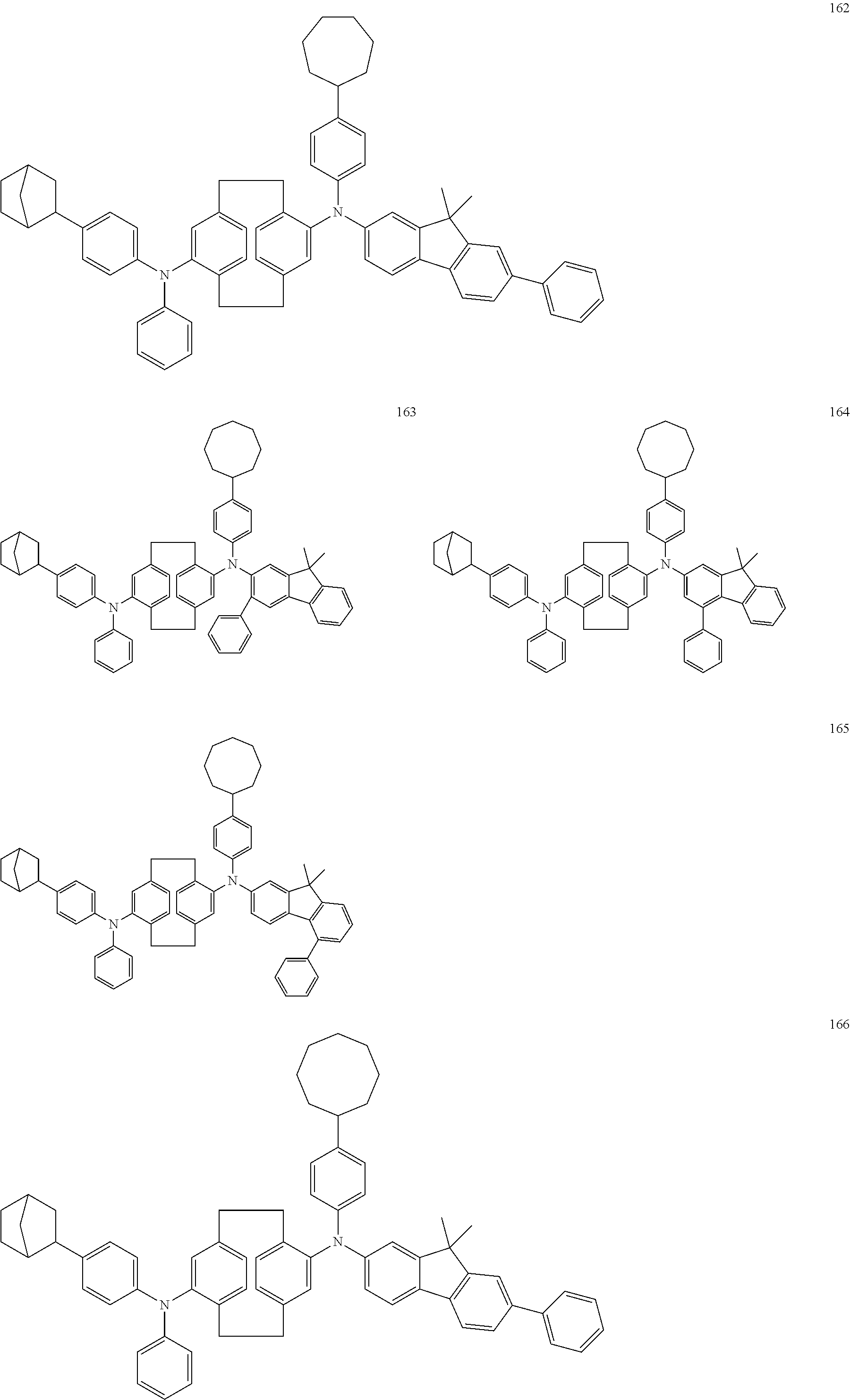

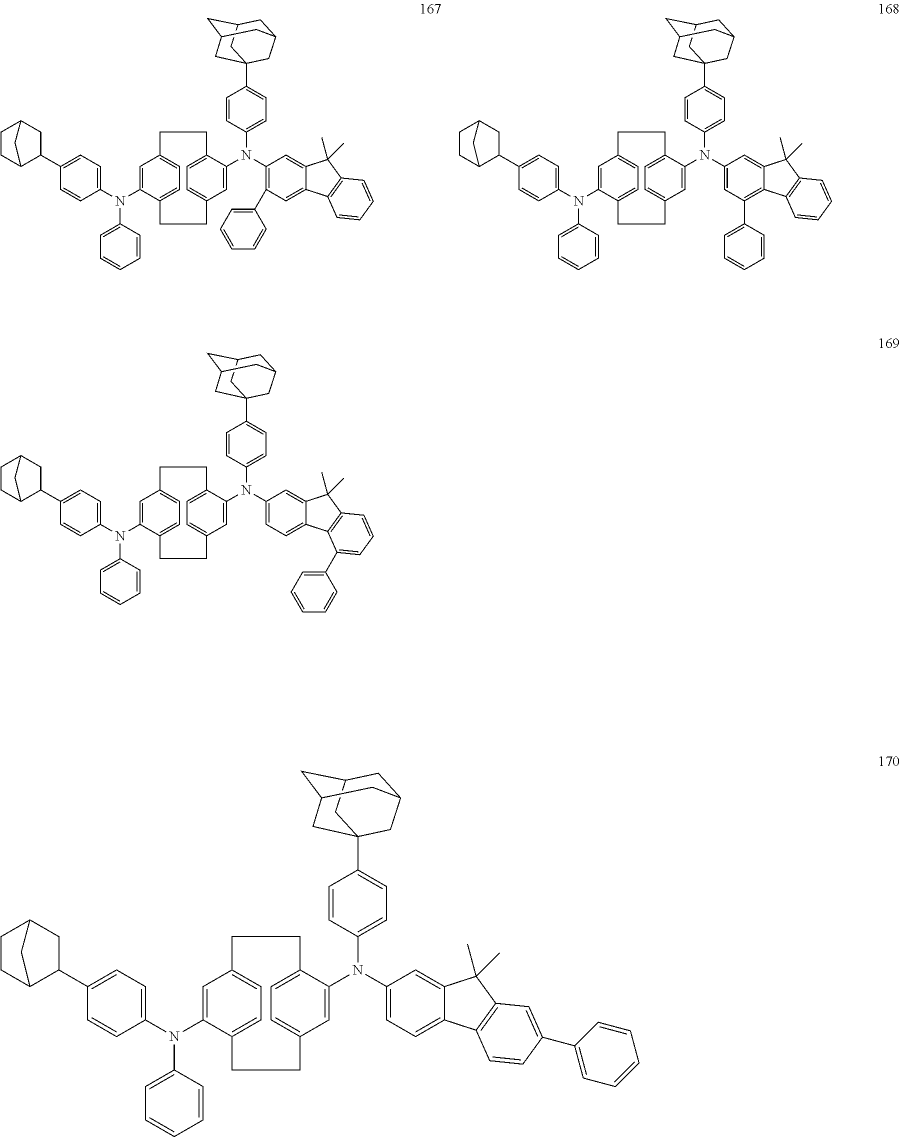

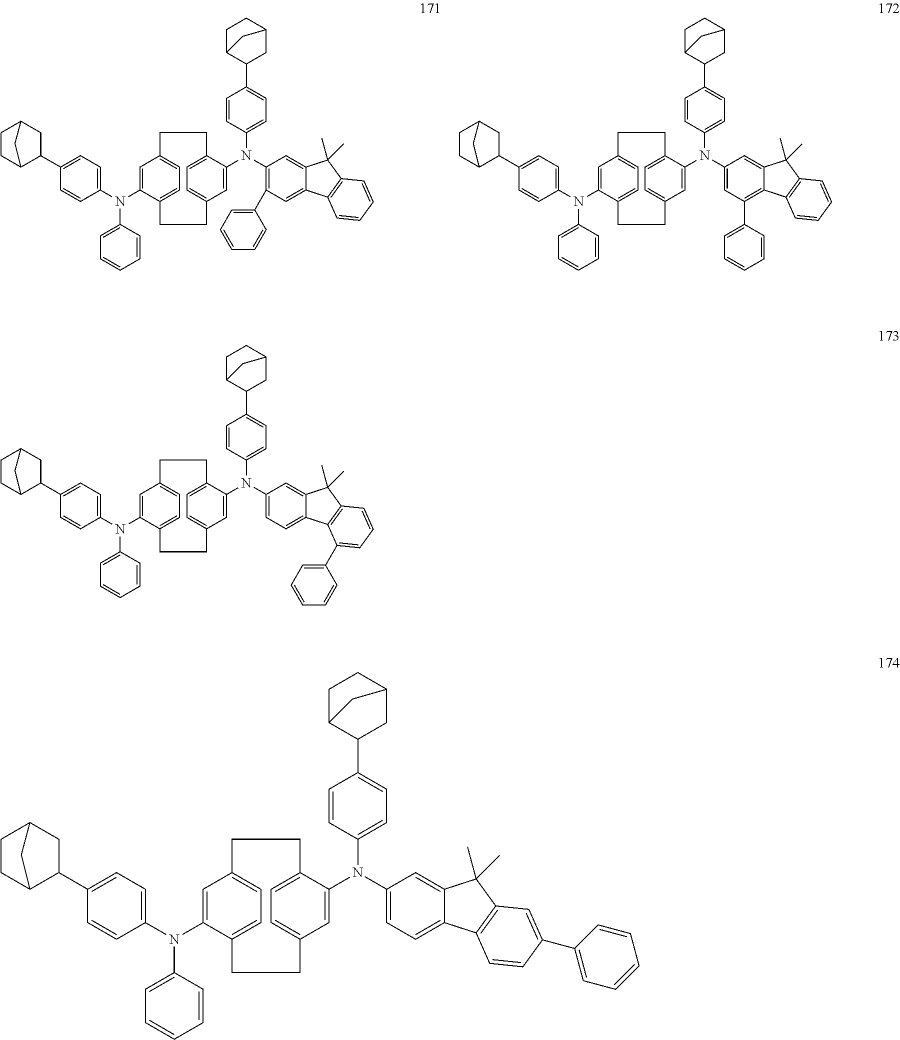 [SMILES: CC1(C)c2ccccc2-c2cc(-c3ccccc3)c(N(c3ccc(C4CC5CCC4C5)cc3)c3cc4ccc3CCc3ccc(c(N(c5ccccc5)c5ccc(C6CC7CCC6C7)cc5)c3)CC4)cc21 ; CC1(C)c2ccccc2-c2c(-c3ccccc3)cc(N(c3ccc(C4CC5CCC4C5)cc3)c3cc4ccc3CCc3ccc(c(N(c5ccccc5)c5ccc(C6CC7CCC6C7)cc5)c3)CC4)cc21 ; CC1(C)c2cc(N(c3ccc(C4CC5CCC4C5)cc3)c3cc4ccc3CCc3ccc(c(N(c5ccccc5)c5ccc(C6CC7CCC6C7)cc5)c3)CC4)ccc2-c2c(-c3ccccc3)cccc21 ; CC1(C)c2cc(-c3ccccc3)ccc2-c2ccc(N(c3ccc(C4CC5CCC4C5)cc3)c3cc4ccc3CCc3ccc(c(N(c5ccccc5)c5ccc(C6CC7CCC6C7)cc5)c3)CC4)cc21]

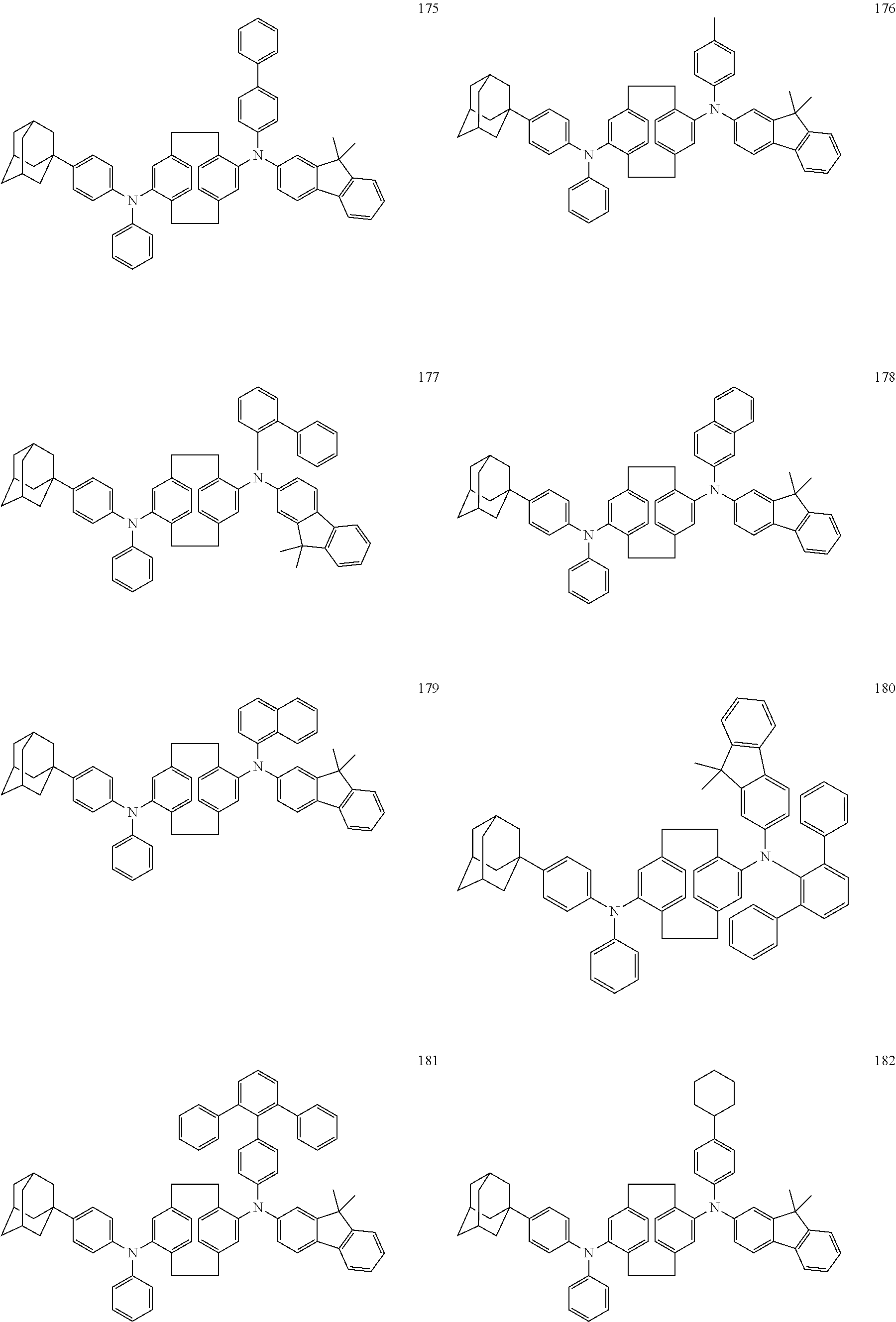

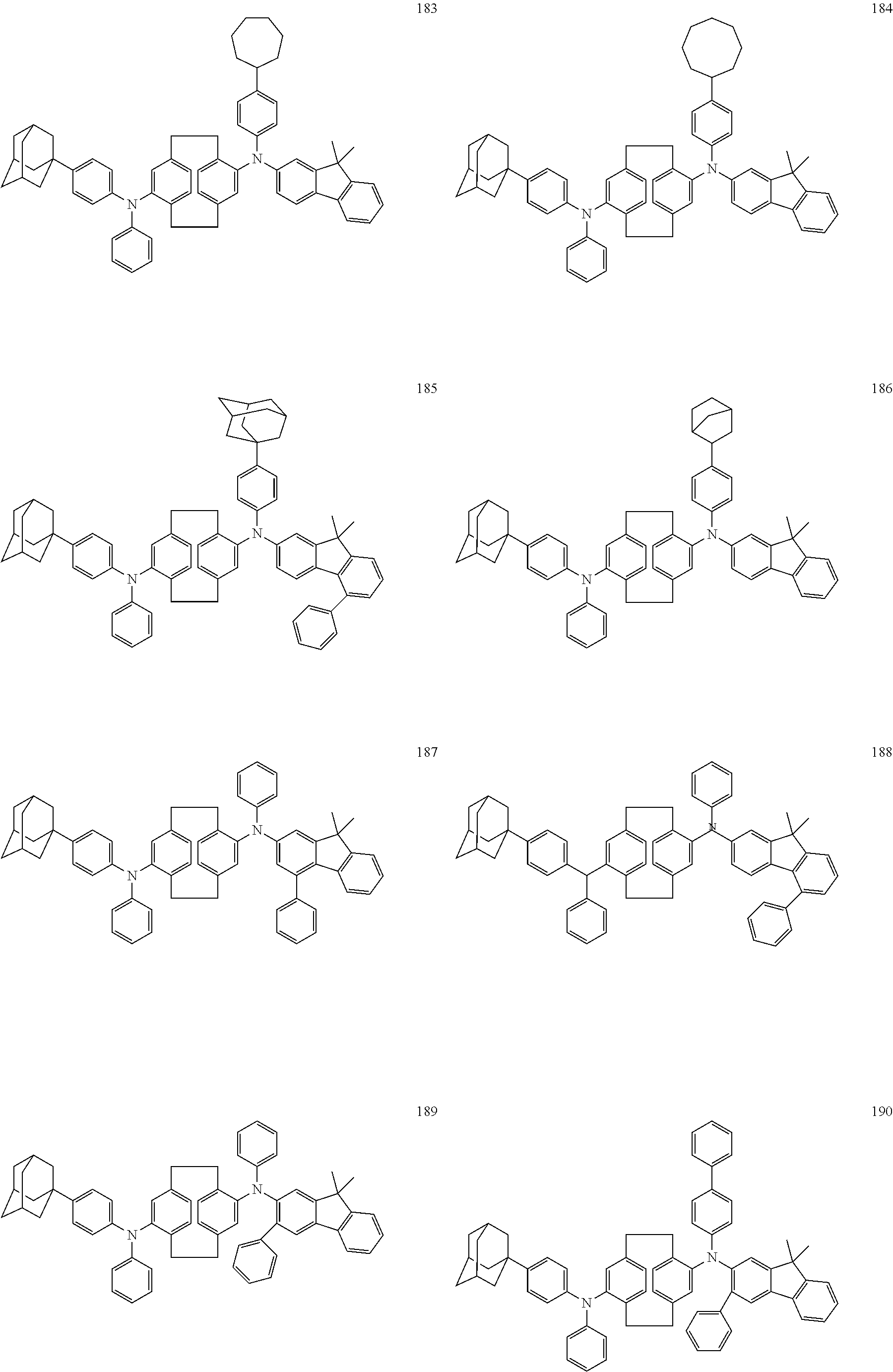

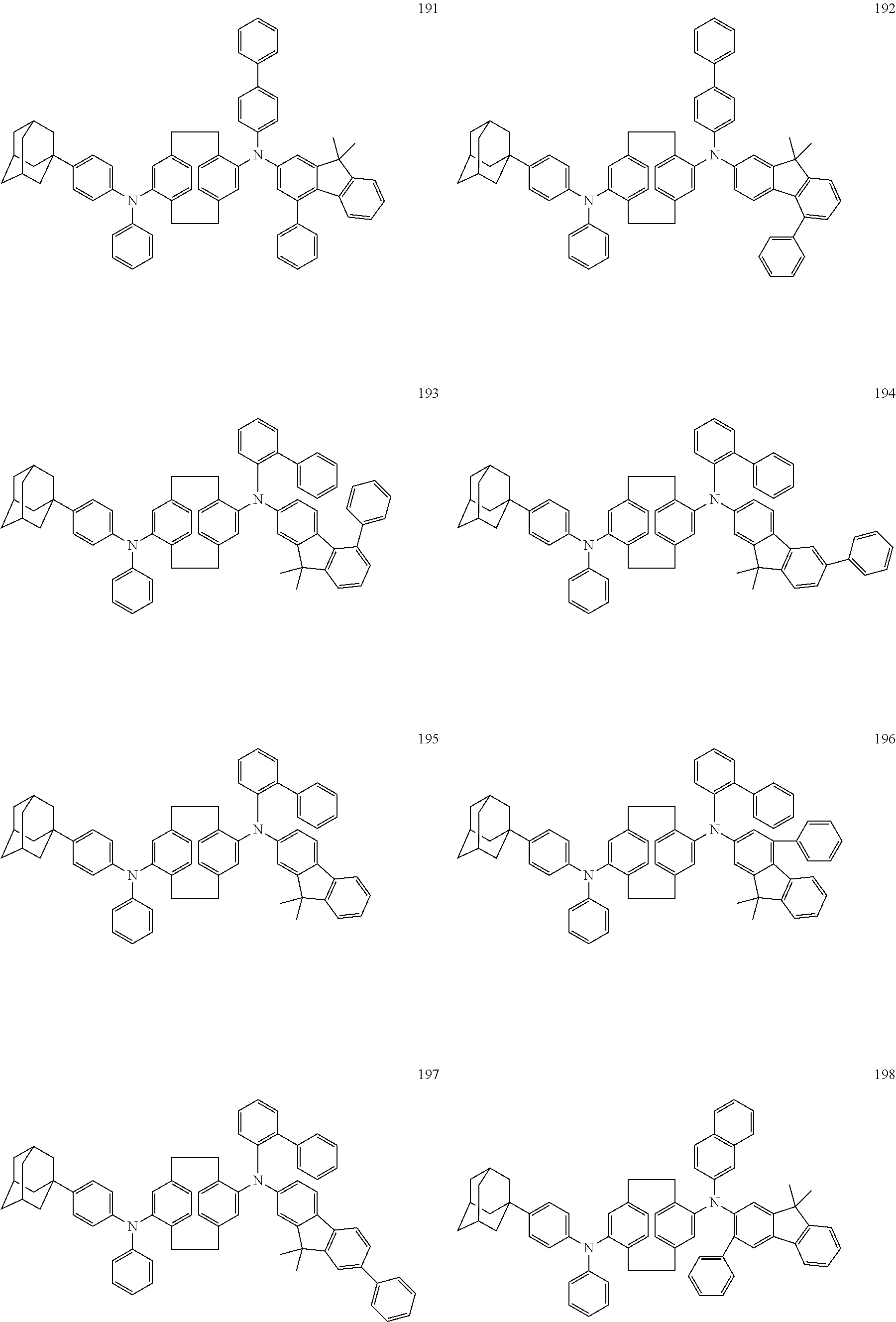

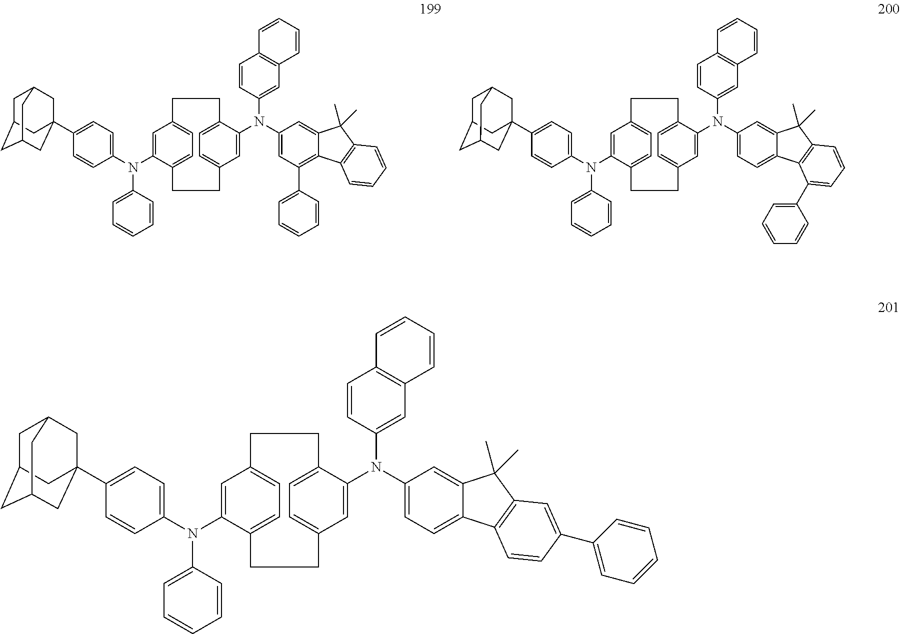 [SMILES: CC1(C)c2ccccc2-c2c(-c3ccccc3)cc(N(c3ccc4ccccc4c3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc4ccccc4c3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21 ; CC1(C)c2cc(-c3ccccc3)ccc2-c2ccc(N(c3ccc4ccccc4c3)c3cc4ccc3CCc3ccc(c(N(c5ccccc5)c5ccc(C67CC8CC(CC(C8)C6)C7)cc5)c3)CC4)cc21]

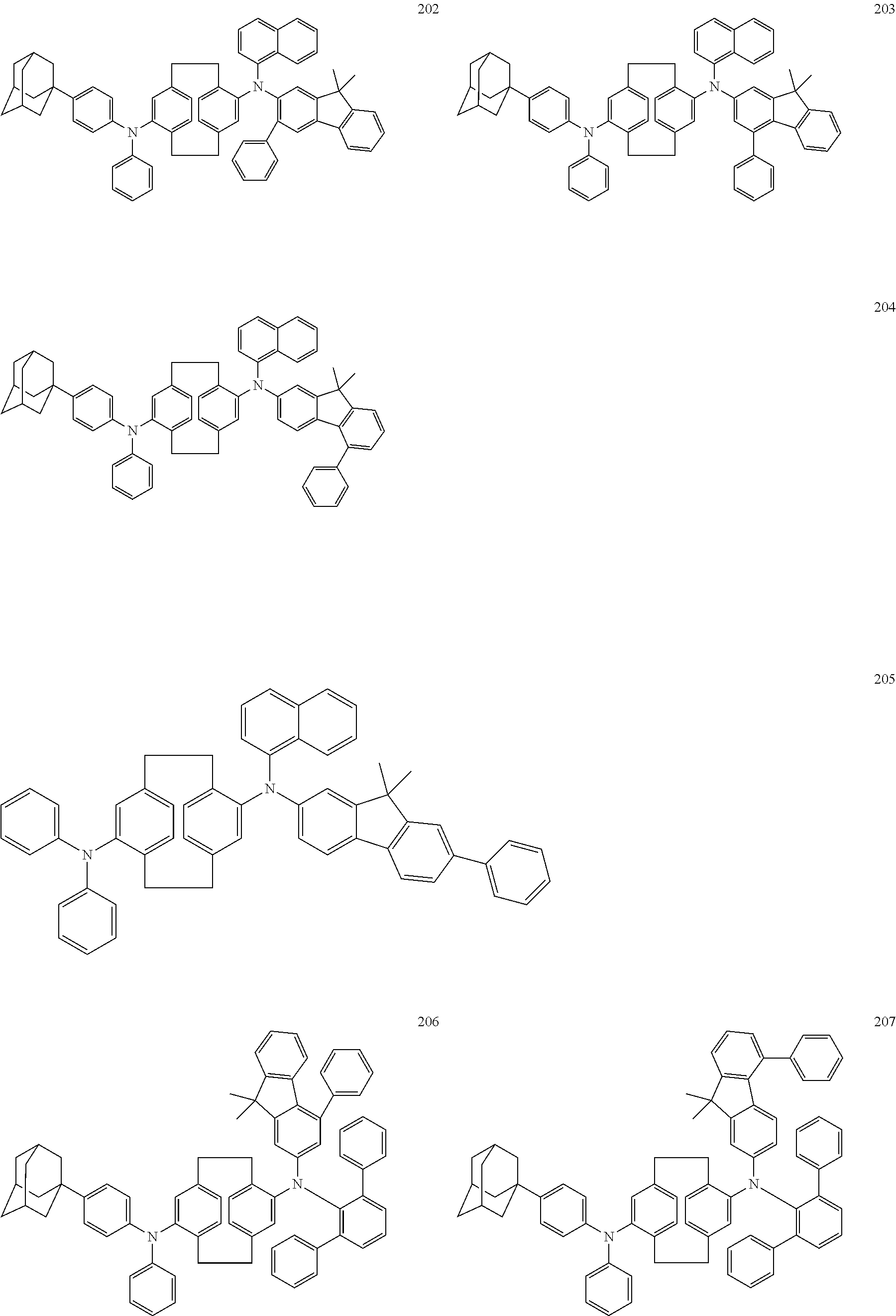

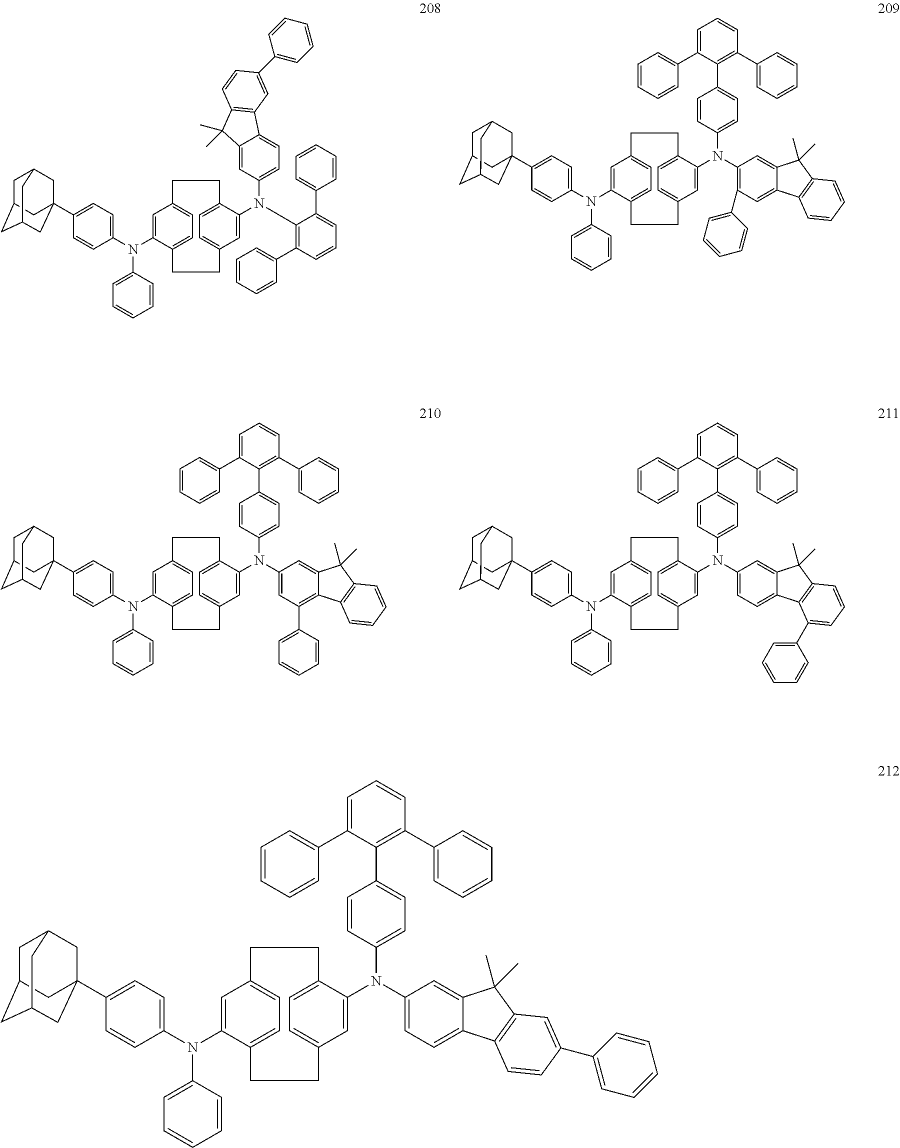

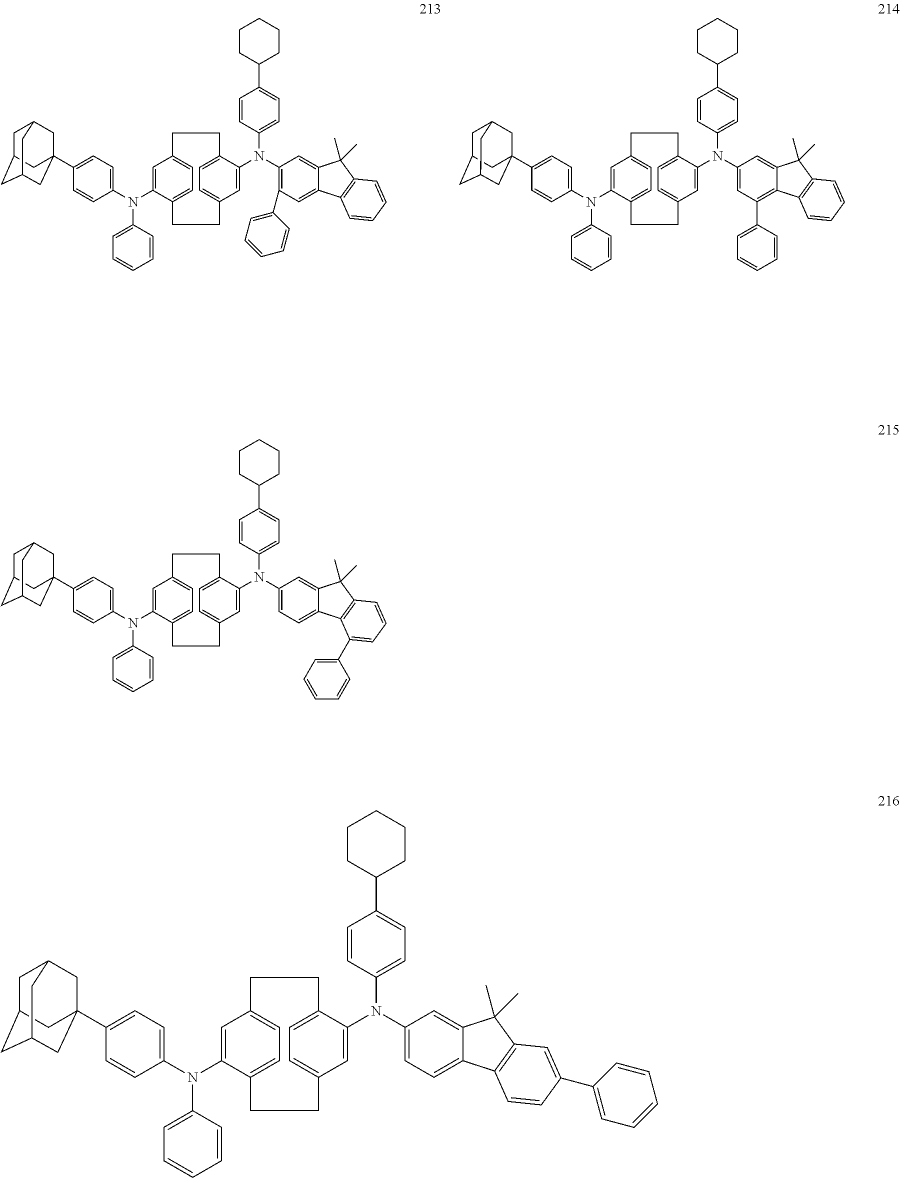

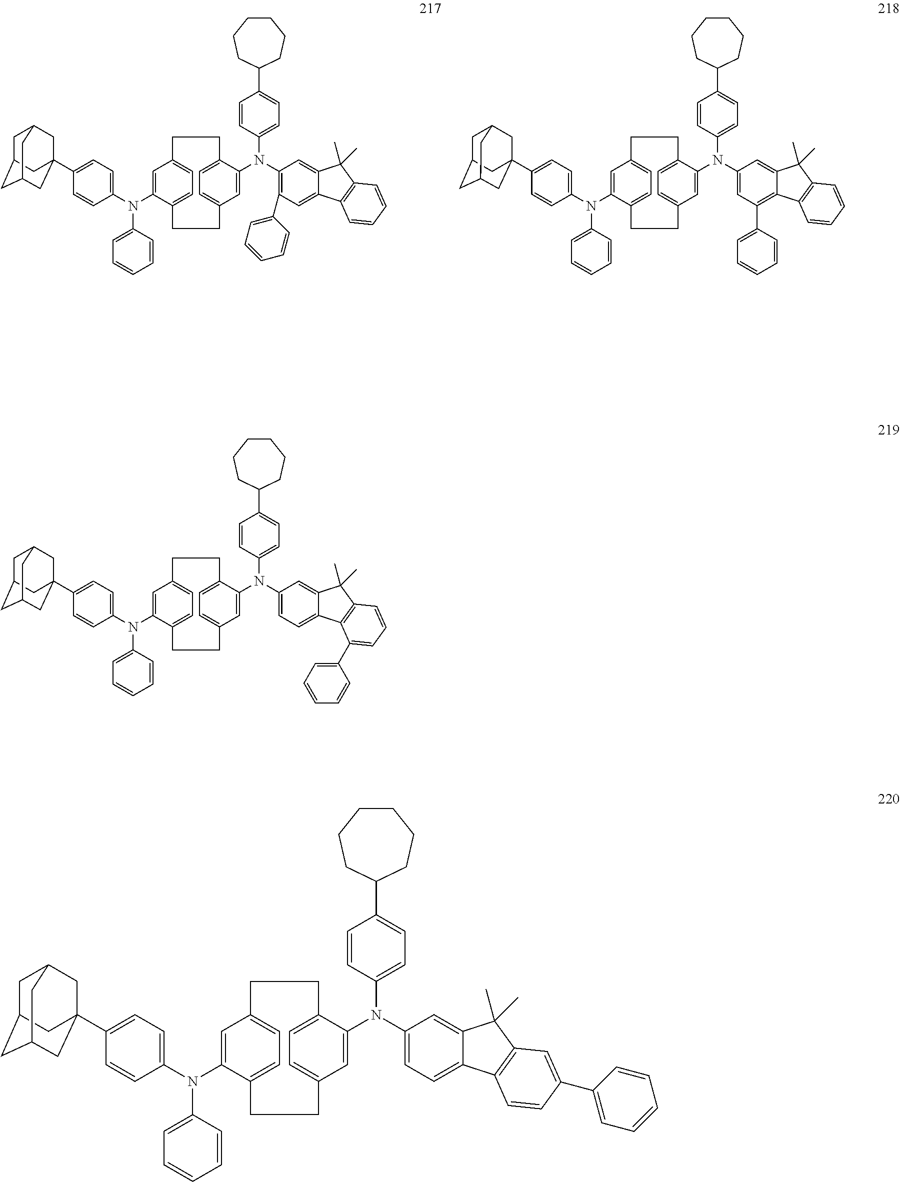

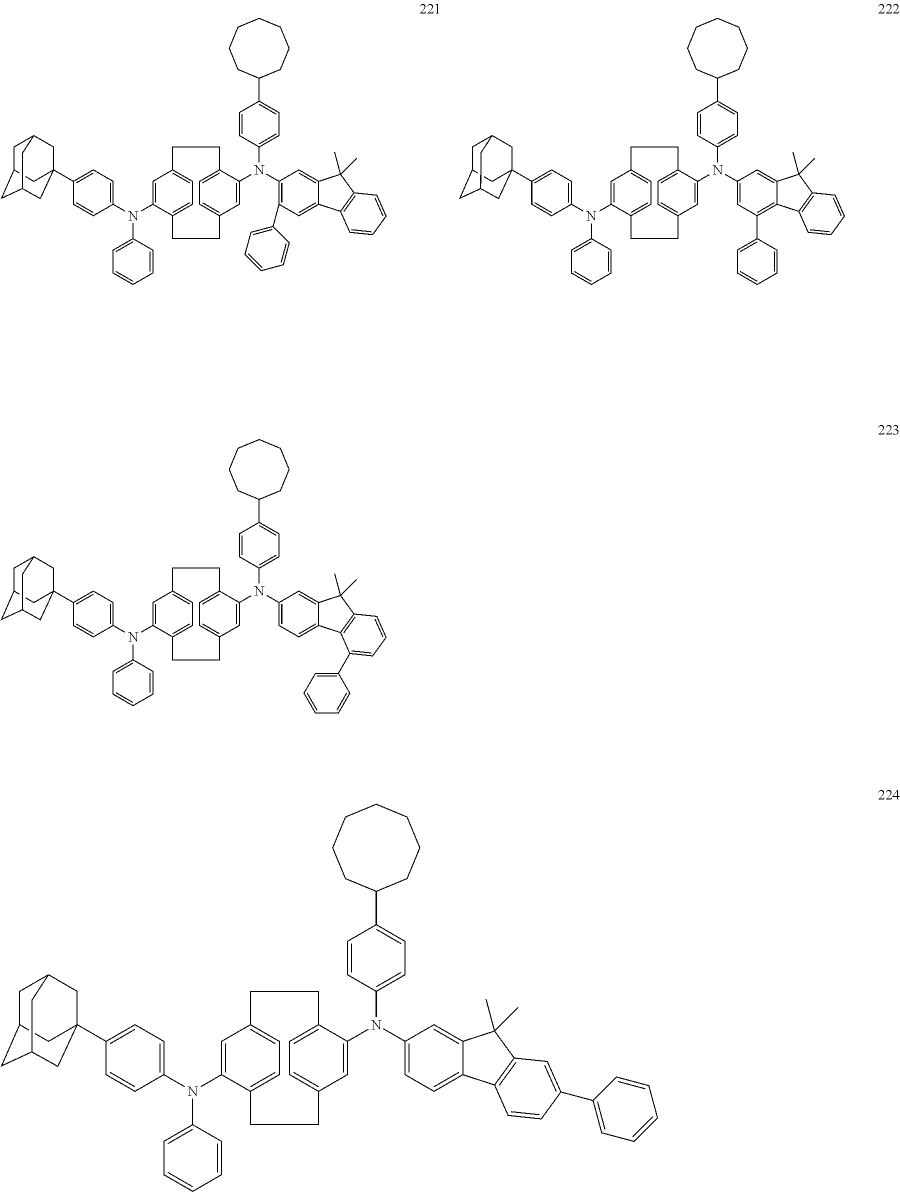

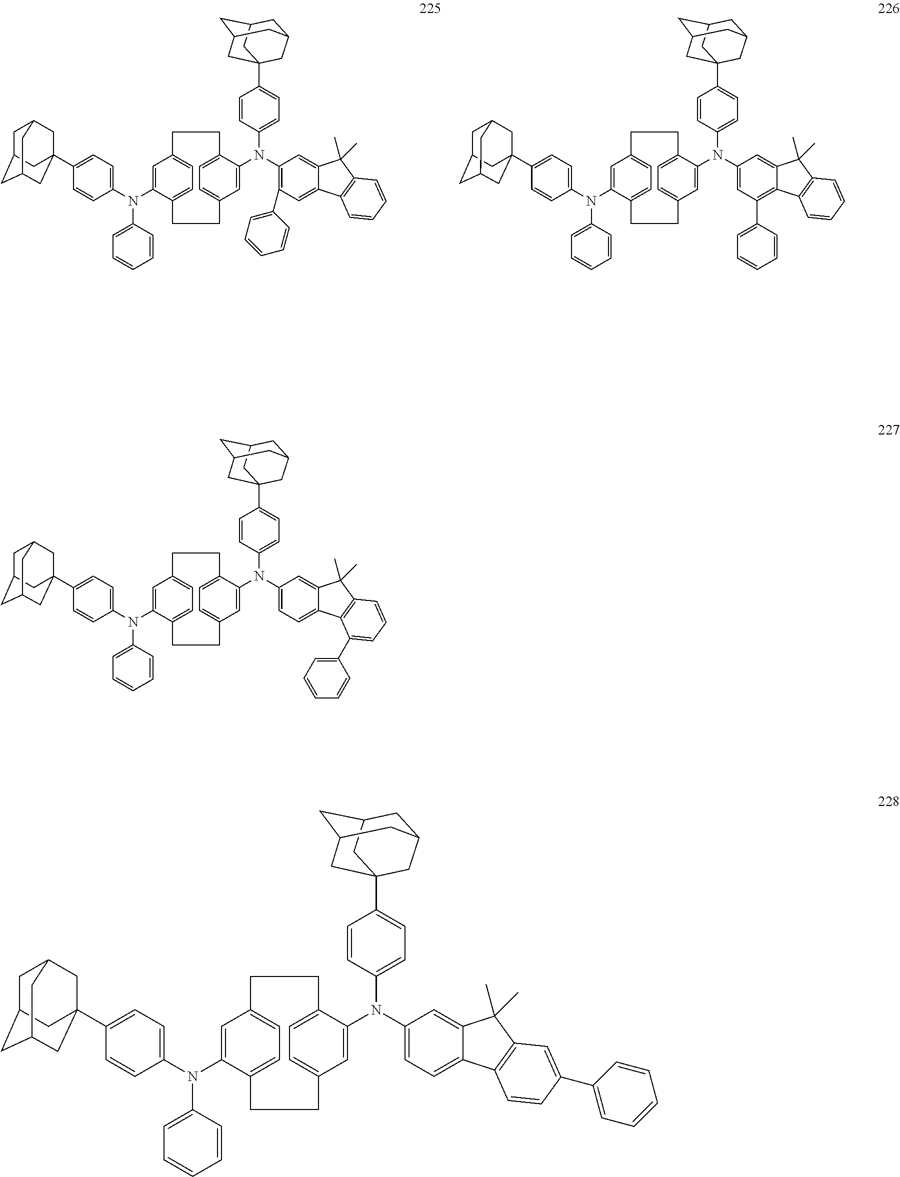

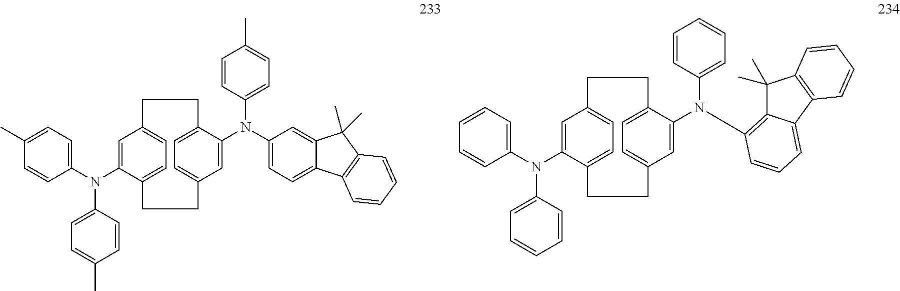 [SMILES: Cc1ccc(N(c2ccc(C)cc2)c2cc3ccc2CCc2ccc(c(N(c4ccc(C)cc4)c4ccc5c(c4)C(C)(C)c4ccccc4-5)c2)CC3)cc1 ; CC1(C)c2ccccc2-c2cccc(N(c3ccccc3)c3cc4ccc3CCc3ccc(c(N(c5ccccc5)c5ccccc5)c3)CC4)c21]

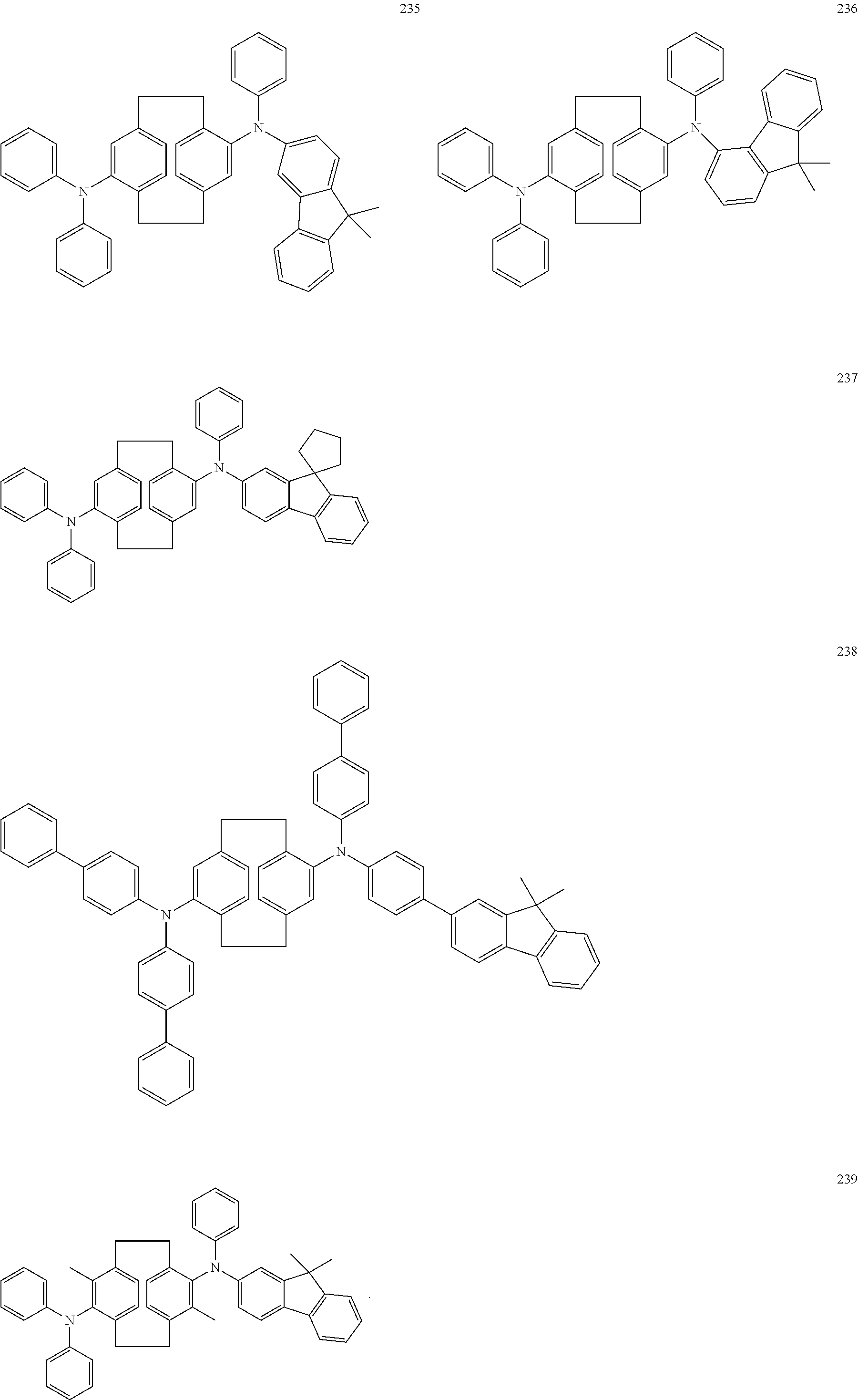 [SMILES: CC1(C)c2ccccc2-c2cc(N(c3ccccc3)c3cc4ccc3CCc3ccc(c(N(c5ccccc5)c5ccccc5)c3)CC4)ccc21 ; CC1(C)c2ccccc2-c2c(N(c3ccccc3)c3cc4ccc3CCc3ccc(c(N(c5ccccc5)c5ccccc5)c3)CC4)cccc21 ; c1ccc(N(c2ccccc2)c2cc3ccc2CCc2ccc(c(N(c4ccccc4)c4ccc5c(c4)C4(CCCC4)c4ccccc4-5)c2)CC3)cc1 ; CC1(C)c2ccccc2-c2ccc(-c3ccc(N(c4ccc(-c5ccccc5)cc4)c4cc5ccc4CCc4ccc(c(N(c6ccc(-c7ccccc7)cc6)c6ccc(-c7ccccc7)cc6)c4)CC5)cc3)cc21 ; Cc1c2ccc(c1N(c1ccccc1)c1ccccc1)CCc1ccc(c(N(c3ccccc3)c3ccc4c(c3)C(C)(C)c3ccccc3-4)c1C)CC2]

The specific structure of the bonds between the phenylene groups in the compounds 2 to 20, 22 to 60, and 62 to 239 is the same as that in the compound 1.

The amine-containing compound represented by Formula 1 is a diamine type (e.g., is a diamine compound), and includes a fluorenyl group bonded to an amine. L₁₁ to L₁₃, L₂₁ to L₂₃, Ar₁₂, Ar₁₃, and Ar₂₁ in Formula 1 may each independently be a C₃-C₆₀ carbocyclic group unsubstituted or substituted with at least one R₁₀ₐ or a C₁-C₆₀ heterocyclic group unsubstituted or substituted with at least one R₁₀ₐ. As a result, as Ar₁₂, Ar₁₃, and Ar₂₁ are changed, a highest occupied molecular orbital (HOMO) energy level of the amine-containing compound represented by Formula 1 may be adjusted. Accordingly, an energy barrier of a layer (e.g., an emission layer) that is adjacent to a layer including the amine-containing compound represented by Formula 1 (e.g., a hole transport layer) may be easily lowered, and thus, exciton generation efficiency in an emission layer may be increased. In addition, a light-emitting device including the amine-containing compound represented by Formula 1 may have low driving voltage, high luminance, high luminescence efficiency, and long lifespan characteristics.

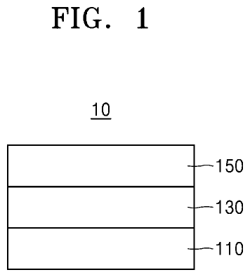

### Description of FIG. 1

FIG. 1 is a schematic view of a light-emitting device 10 according to an embodiment. The light-emitting device 10 may include a first electrode 110, an interlayer 130, and a second electrode 150.

Hereinafter, the structure of the light-emitting device 10 according to an embodiment and a method of manufacturing the light-emitting device 10 will be described with reference to FIG. 1.

### First Electrode 110

In FIG. 1, a substrate may be under the first electrode 110 and/or on the second electrode 150. The substrate may be a glass substrate and/or a plastic substrate. The substrate may be a flexible substrate. For example, the flexible substrate may include plastic having excellent heat resistance and durability, such as polyimide, polyethylene terephthalate (PET), polycarbonate, polyethylene naphthalate, polyarylate (PAR), polyetherimide, or any combination thereof.

The first electrode 110 may be formed by depositing and/or sputtering a material for forming the first electrode 110 on the substrate. When the first electrode 110 is an anode, the material for forming the first electrode 110 may be a high work function material that facilitates injection of holes.

The first electrode 110 may be a reflective electrode, a semi-transmissive electrode, or a transmissive electrode. When the first electrode 110 is a transmissive electrode, the material for forming the first electrode 110 may include indium tin oxide (ITO), indium zinc oxide (IZO), tin oxide (SnO₂), zinc oxide (ZnO), or any combination thereof. When the first electrode 110 is a semi-transmissive electrode or a reflective electrode, the material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layered structure consisting of a single layer or a multi-layered structure including a plurality of layers. For example, the first electrode 110 may have a three-layered structure of ITO/Ag/ITO.

### Interlayer 130

The interlayer 130 may be on the first electrode 110. The interlayer 130 may include an emission layer.

The interlayer 130 may further include a hole transport region between the first electrode 110 and the emission layer and an electron transport region between the emission layer and the second electrode 150.

The interlayer 130 may further include, in addition to various suitable organic materials, a metal-containing compound, such as an organometallic compound, an inorganic material, such as a quantum dot, and/or the like.

The interlayer 130 may include i) two or more emitting units sequentially stacked between the first electrode 110 and the second electrode 150 and ii) a charge generation layer between the two or more emitting units. When the interlayer 130 includes the two or more emitting units and the charge generation layer, the light-emitting device 10 may be a tandem light-emitting device.

### Hole Transport Region in Interlayer 130

The hole transport region may have i) a single-layered structure consisting of a single layer consisting of a single material, ii) a single-layered structure consisting of a single layer including a plurality of different materials, or iii) a multi-layered structure including a plurality of layers including different materials.

The hole transport region may include a hole injection layer, a hole transport layer, an emission auxiliary layer, an electron blocking layer, or any combination thereof.

For example, the hole transport region may have a multi-layered structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure, the layers of each structure being stacked sequentially from the first electrode 110.

The hole transport region may include a compound represented by Formula 201, a compound represented by Formula 202, or any combination thereof:

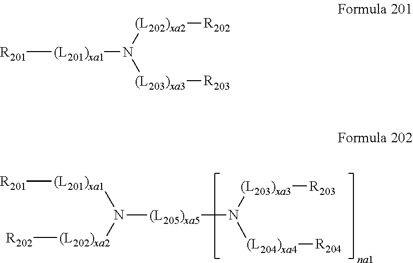

wherein, in Formulae 201 and 202,
L₂₀₁ to L₂₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
L₂₀₅ may be *—O—*′, *—S*′, *—N(Q₂₀₁)-*′, a C₁-C₂₀ alkylene group unsubstituted or substituted with at least one R₁₀ₐ, a C₂-C₂₀ alkenylene group unsubstituted or substituted with at least one R₁₀ₐ, a C₃-C₆₀ carbocyclic group unsubstituted or substituted with at least one R₁₀ₐ, or a C₁-C₆₀ heterocyclic group unsubstituted or substituted with at least one R₁₀ₐ,
xa1 to xa4 may each independently be an integer from 0 to 5,
xa5 may be an integer from 1 to 10,
R₂₀₁ to R₂₀₄ and Q₂₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
R₂₀₁ and R₂₀₂ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group (e.g., a carbazole group, etc.) unsubstituted or substituted with at least one R₁₀ₐ (e.g., Compound HT16),
R₂₀₃ and R₂₀₄ may optionally be linked to each other via a single bond, a C₁-C₅ alkylene group unsubstituted or substituted with at least one R₁₀ₐ, or a C₂-C₅ alkenylene group unsubstituted or substituted with at least one R₁₀ₐ to form a C₈-C₆₀ polycyclic group unsubstituted or substituted with at least one R₁₀ₐ, and
na1 may be an integer from 1 to 4.

For example, each of Formulae 201 and 202 may include at least one of groups represented by Formulae CY201 to CY217:

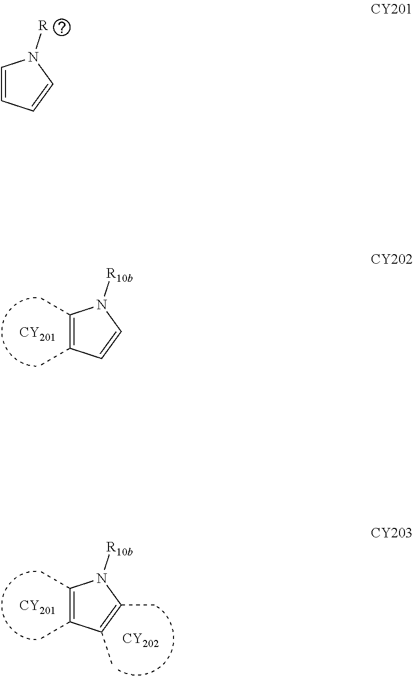

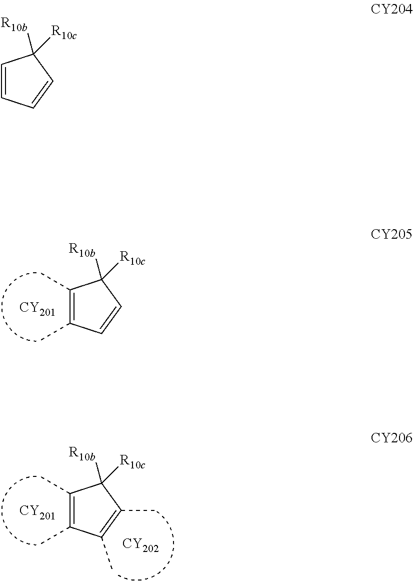

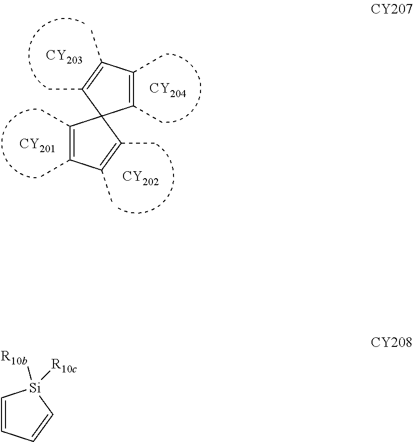

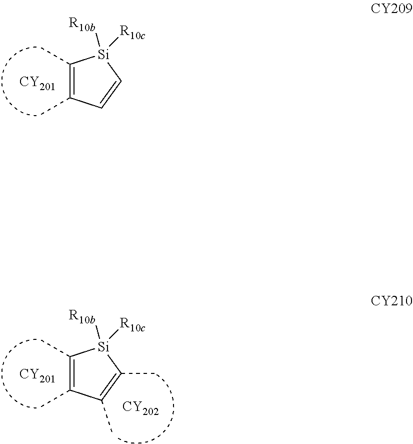

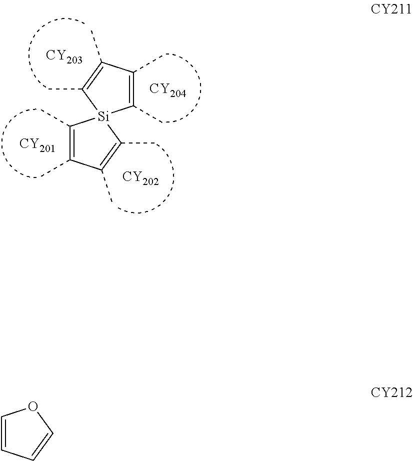

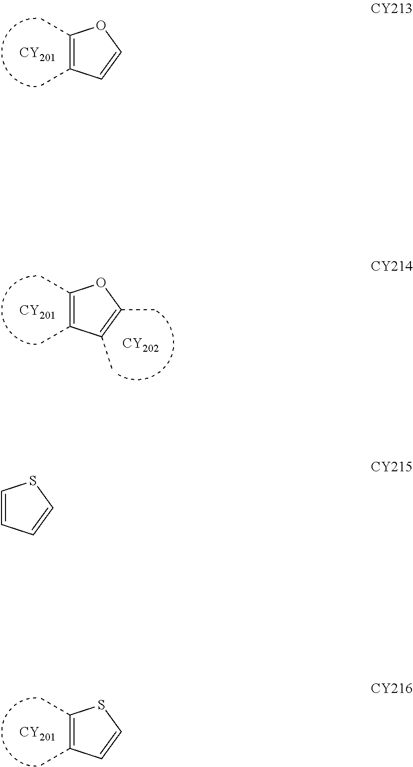

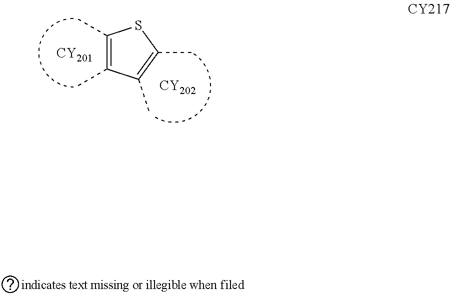

wherein, in Formulae CY201 to CY217, R_{10b} and R_{10c} are each the same as described in connection with R₁₀ₐ, ring CY201 to ring CY204 may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In an embodiment, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one of groups represented by Formulae CY201 to CY203.

In one or more embodiments, Formula 201 may include at least one of the groups represented by Formulae CY201 to CY203 and at least one of groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include a group represented by one of Formulae CY201 to CY203.

In one or more embodiments, each of Formulae 201 and 202 may not include a group represented by one of Formulae CY201 to CY203, and may include at least one of the groups represented by Formulae CY204 to CY217.

In one or more embodiments, each of Formulae 201 and 202 may not include a group represented by one of Formulae CY201 to CY217.

For example, the hole transport region may include one of Compounds HT1 to HT46, m-MTDATA, TDATA, 2-TNATA, NPB(NPD), p-NPB, TPD, Spiro-TPD, Spiro-NPB, methylated NPB, TAPC, HMTPD, 4,4′,4″-tris(N-carbazolyl)triphenylamine (TCTA), polyaniline/dodecylbenzenesulfonic acid (PANI/DBSA), poly(3,4-ethylenedioxythiophene)/poly(4-styrenesulfonate) (PEDOT/PSS), polyaniline/camphor sulfonic acid (PANI/CSA), polyaniline/poly(4-styrenesulfonate) (PANI/PSS), or any combination thereof:

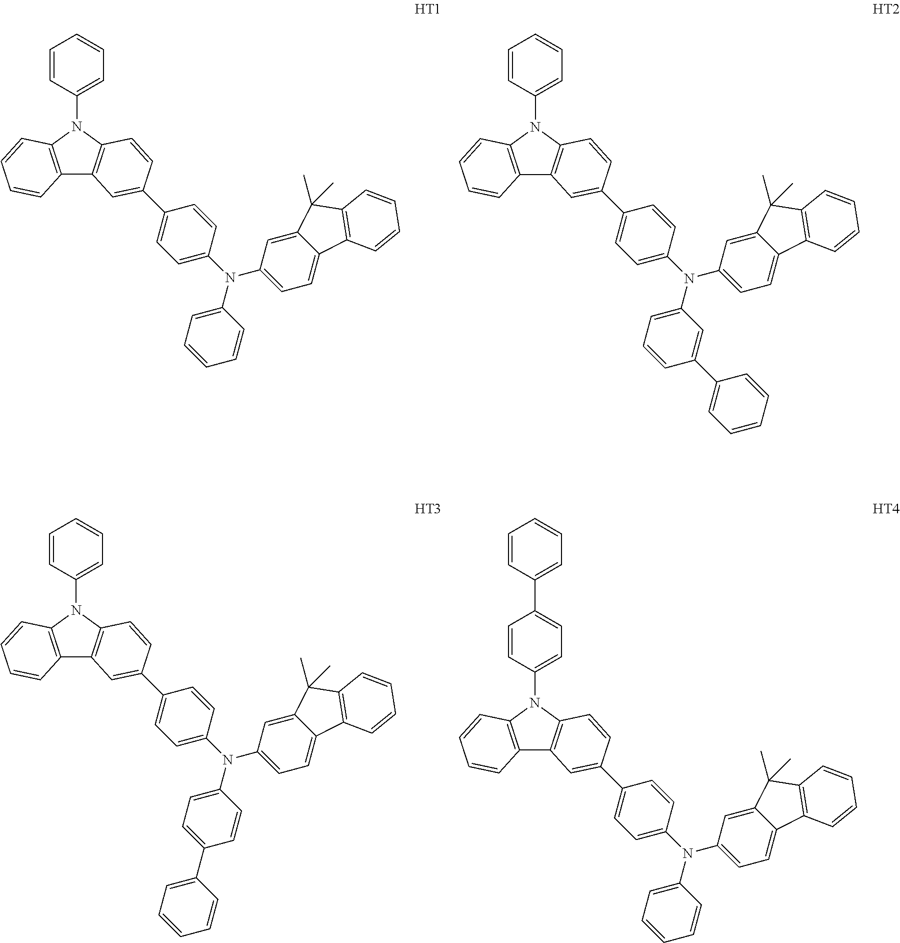 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

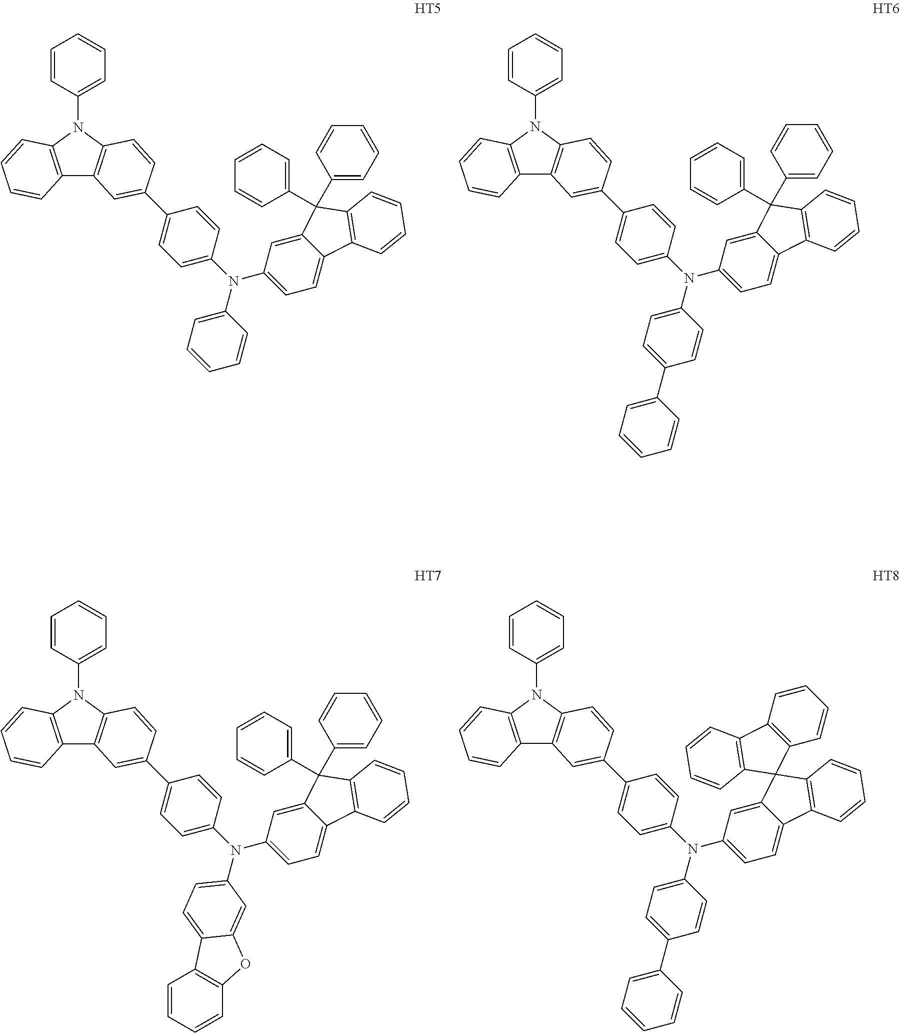 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

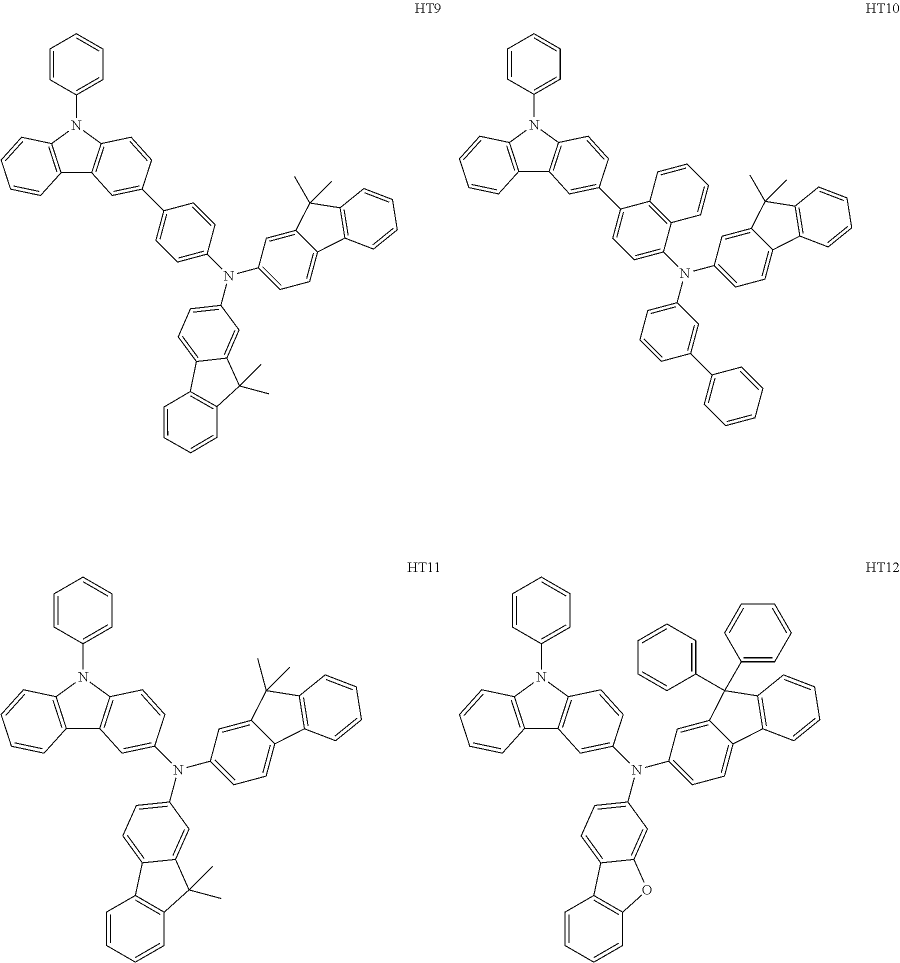 [SMILES: CC1(C)c2ccccc2-c2ccc(N(c3ccc(-c4ccc5c(c4)c4ccccc4n5-c4ccccc4)cc3)c3ccc4c(c3)C(C)(C)c3ccccc3-4)cc21 ; CC1(C)c2ccccc2-c2ccc(N(c3cccc(-c4ccccc4)c3)c3ccc(-c4ccc5c(c4)c4ccccc4n5-c4ccccc4)c4ccccc34)cc21 ; CC1(C)c2ccccc2-c2ccc(N(c3ccc4c(c3)C(C)(C)c3ccccc3-4)c3ccc4c(c3)c3ccccc3n4-c3ccccc3)cc21 ; c1ccc(-n2c3ccccc3c3cc(N(c4ccc5c(c4)C(c4ccccc4)(c4ccccc4)c4ccccc4-5)c4ccc5c(c4)oc4ccccc45)ccc32)cc1]

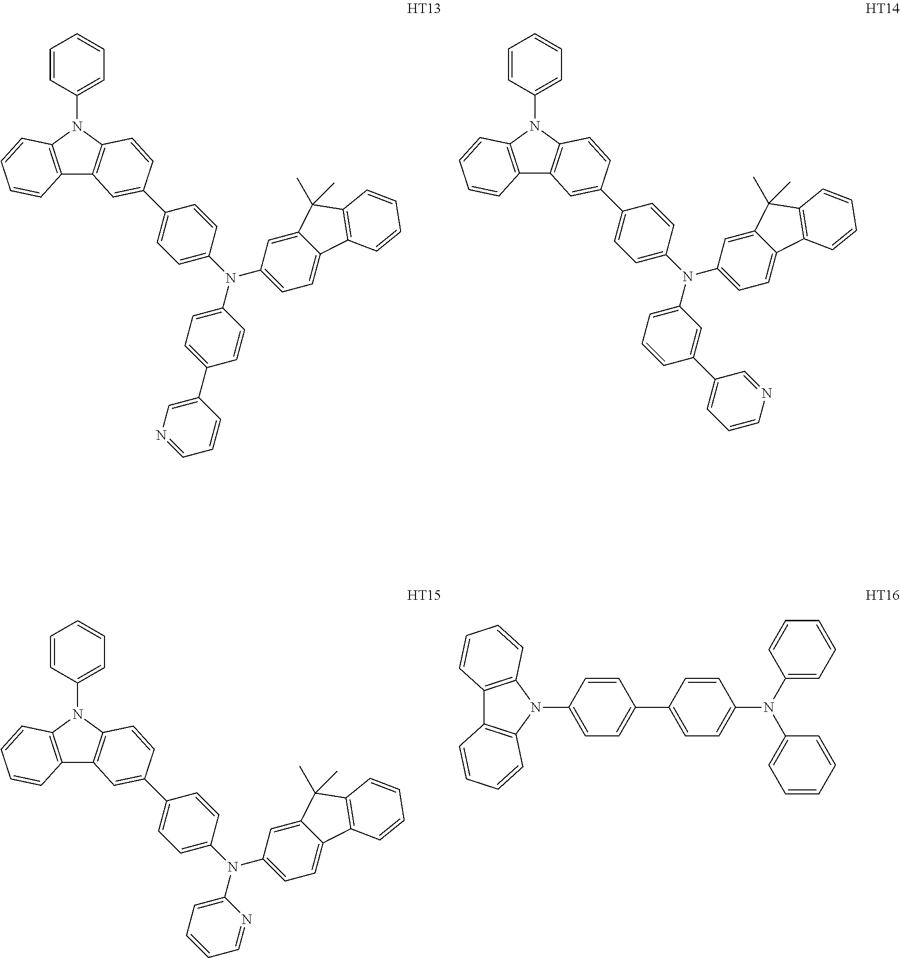 [SMILES: CC1(C)c2ccccc2-c2ccc(N(c3ccc(-c4cccnc4)cc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4cccnc4)c3)cc21 ; CC1(C)c2ccccc2-c2ccc(N(c3ccc(-c4ccc5c(c4)c4ccccc4n5-c4ccccc4)cc3)c3ccccn3)cc21 ; c1ccc(N(c2ccccc2)c2ccc(-c3ccc(-n4c5ccccc5c5ccccc54)cc3)cc2)cc1]

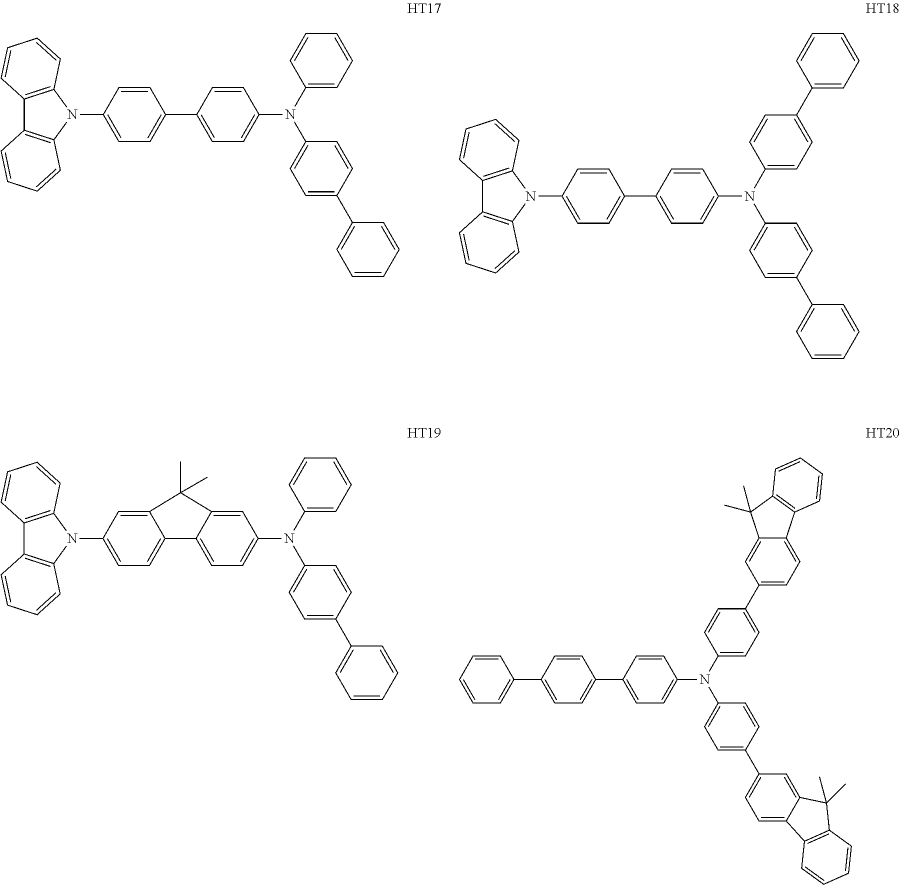 [SMILES: c1ccc(-c2ccc(N(c3ccccc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2cc(N(c3ccccc3)c3ccc(-c4ccccc4)cc3)ccc2-c2ccc(-n3c4ccccc4c4ccccc43)cc21 ; CC1(C)c2ccccc2-c2ccc(-c3ccc(N(c4ccc(-c5ccc(-c6ccccc6)cc5)cc4)c4ccc(-c5ccc6c(c5)C(C)(C)c5ccccc5-6)cc4)cc3)cc21]

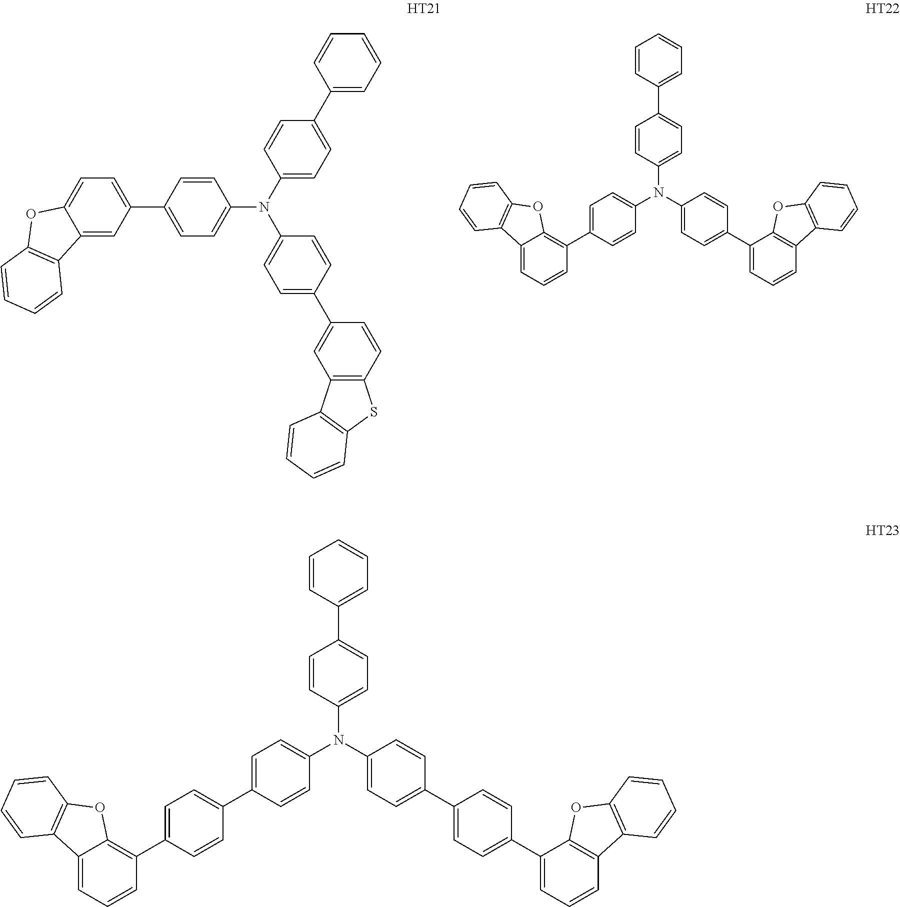 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1]

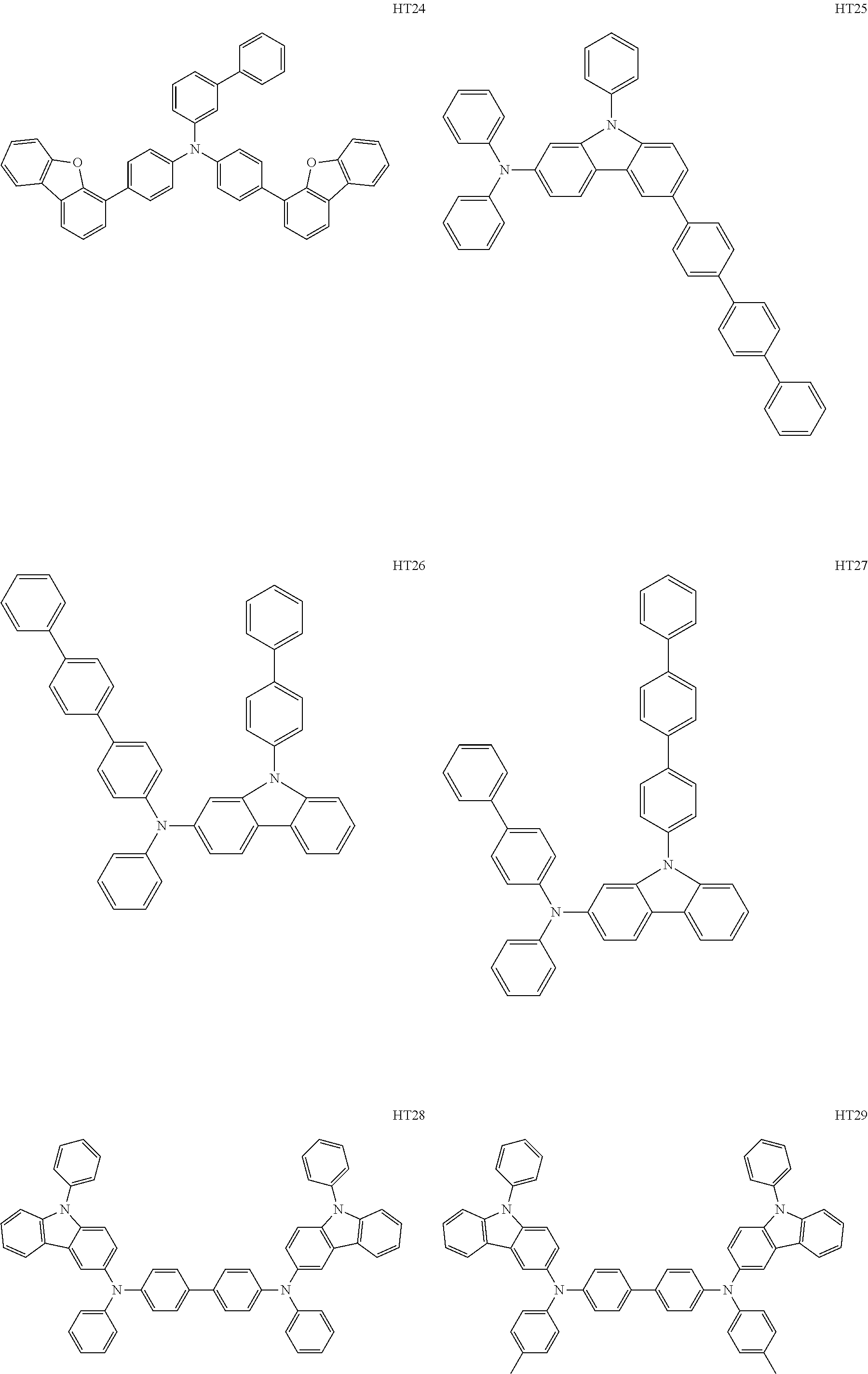

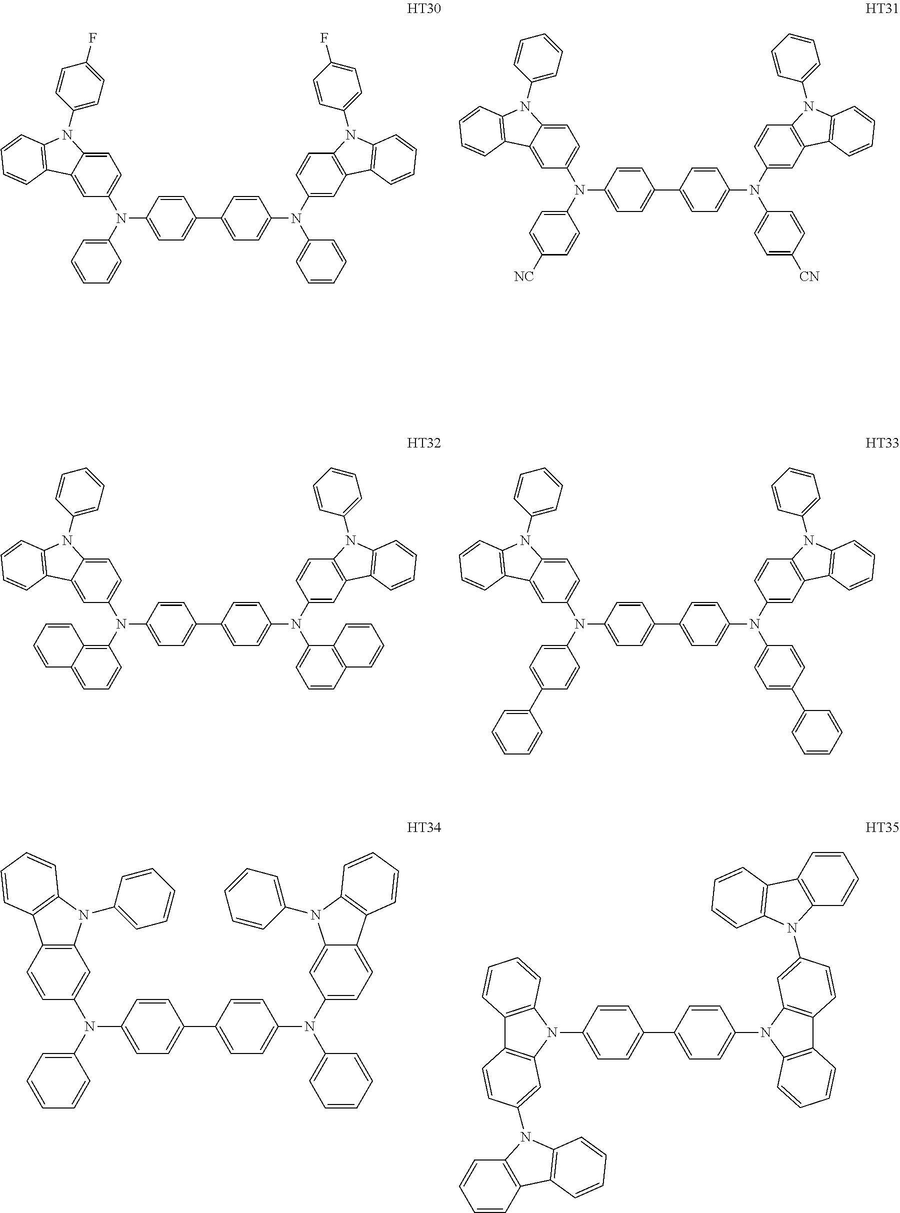

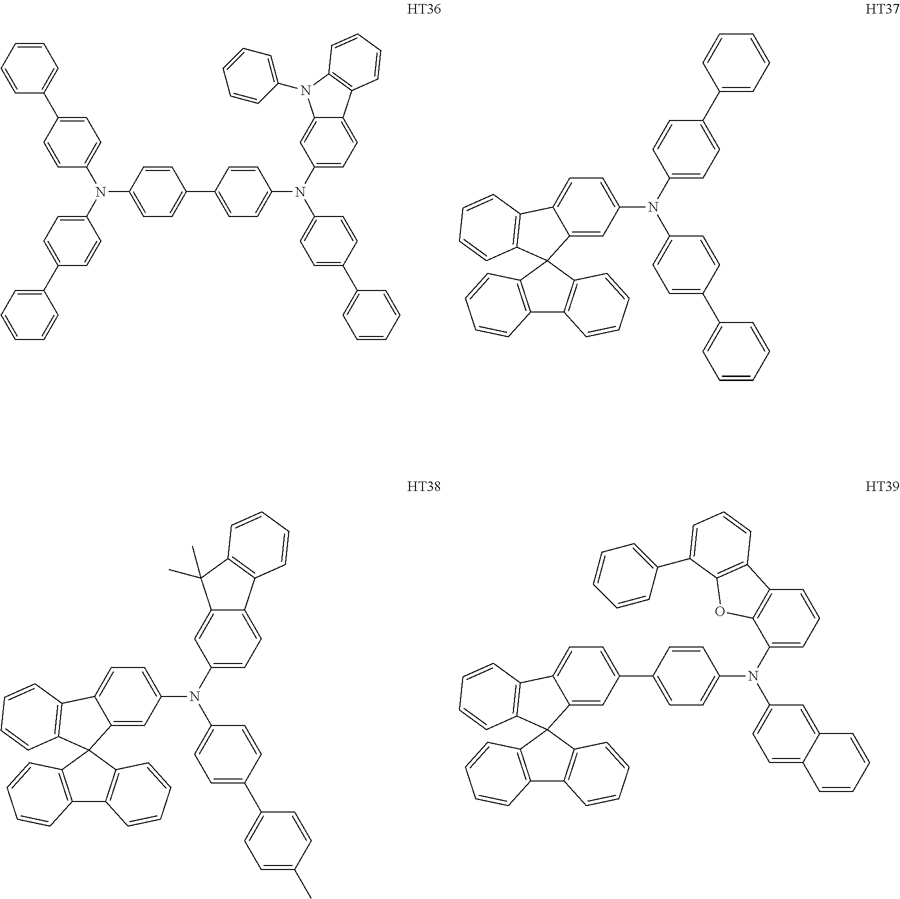 [SMILES: c1ccc(-c2ccc(N(c3ccc(-c4ccccc4)cc3)c3ccc(-c4ccc(N(c5ccc(-c6ccccc6)cc5)c5ccc6c7ccccc7n(-c7ccccc7)c6c5)cc4)cc3)cc2)cc1 ; c1ccc(-c2ccc(N(c3ccc(-c4ccccc4)cc3)c3ccc4c(c3)C3(c5ccccc5-c5ccccc53)c3ccccc3-4)cc2)cc1 ; Cc1ccc(-c2ccc(N(c3ccc4c(c3)C(C)(C)c3ccccc3-4)c3ccc4c(c3)C3(c5ccccc5-c5ccccc53)c3ccccc3-4)cc2)cc1 ; c1ccc(-c2cccc3c2oc2c(N(c4ccc(-c5ccc6c(c5)C5(c7ccccc7-c7ccccc75)c5ccccc5-6)cc4)c4ccc5ccccc5c4)cccc23)cc1]

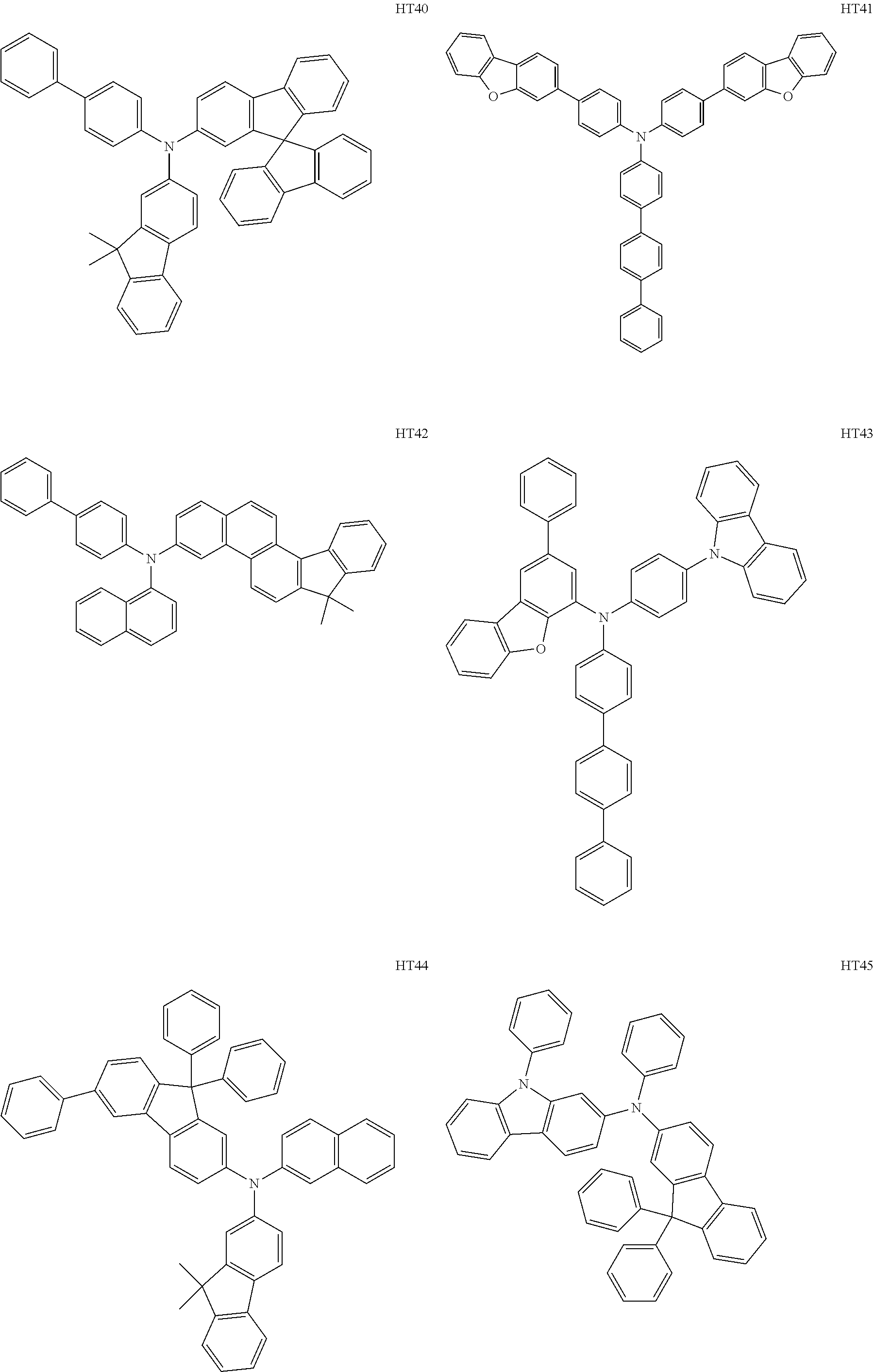

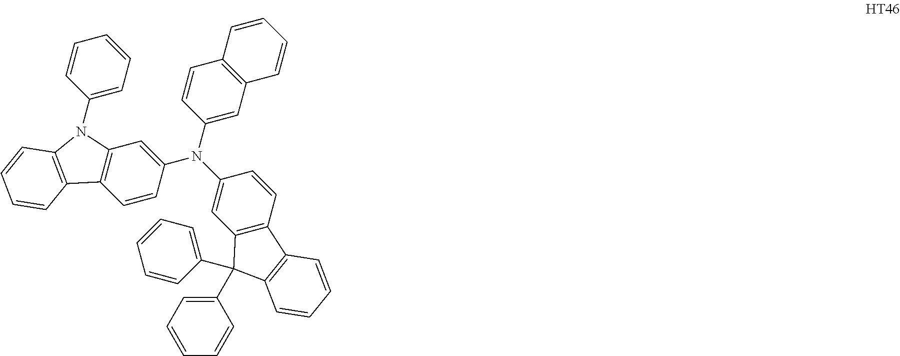 [SMILES: c1ccc(-n2c3ccccc3c3ccc(N(c4ccc5c(c4)C(c4ccccc4)(c4ccccc4)c4ccccc4-5)c4ccc5ccccc5c4)cc32)cc1]

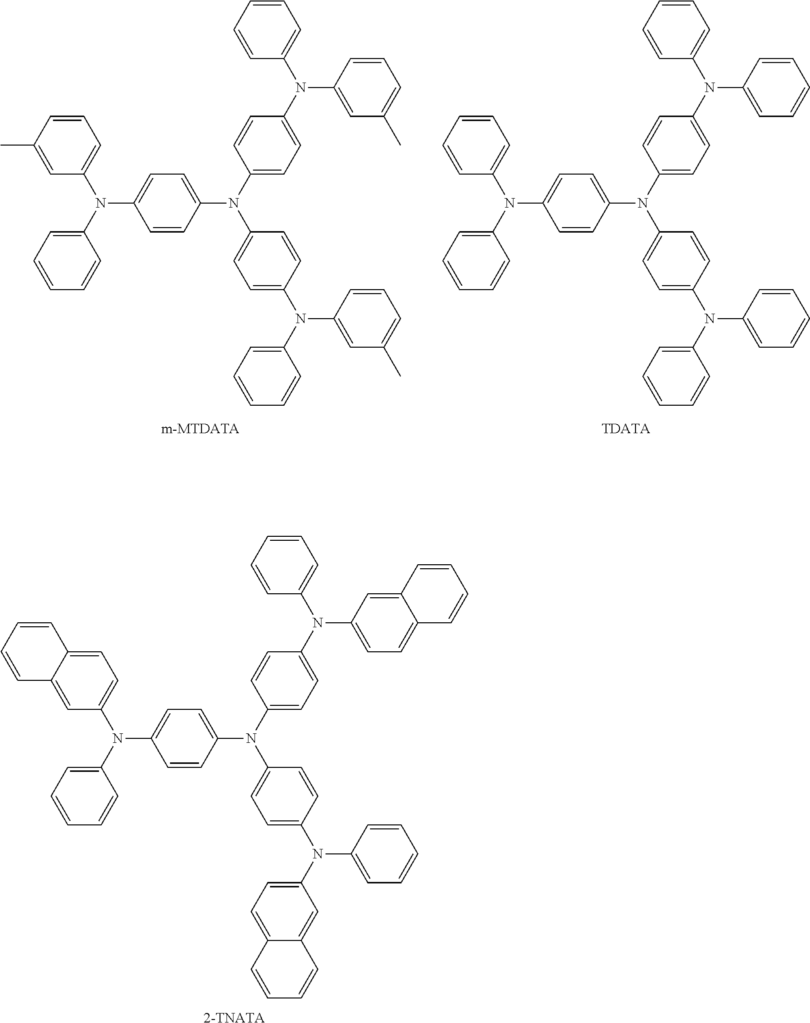

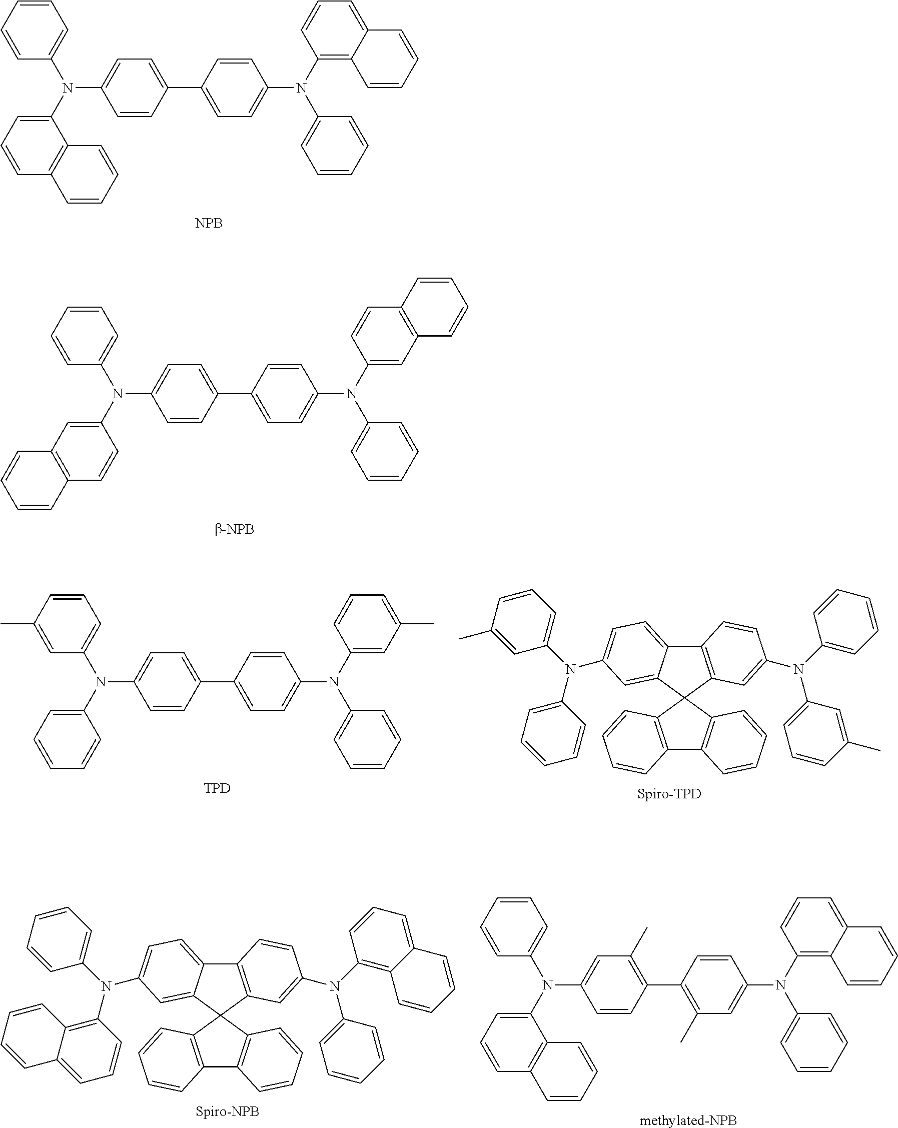

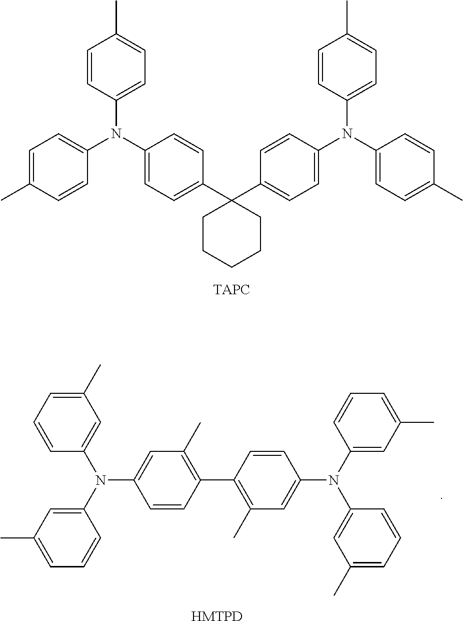

A thickness of the hole transport region may be in a range of about 50 Å to about 10,000 Å, for example, about 100 Å to about 4,000 Å. When the hole transport region includes a hole injection layer, a hole transport layer, or any combination thereof, a thickness of the hole injection layer may be in a range of about 100 Å to about 9,000 Å, for example, about 100 Å to about 1,000 Å, and a thickness of the hole transport layer may be in a range of about 50 Å to about 2,000 Å, for example, about 100 Å to about 1,500 Å. When the thicknesses of the hole transport region, the hole injection layer, and the hole transport layer are within these ranges, suitable or satisfactory hole transporting characteristics may be obtained without a substantial increase in driving voltage.

The emission auxiliary layer may compensate for an optical resonance distance according to a wavelength of light emitted from the emission layer to increase light-emission efficiency. The electron blocking layer may prevent or reduce leakage of electrons from the emission layer to the hole transport region. Materials that may be included in the hole transport region may be included in the emission auxiliary layer and the electron blocking layer.

p-Dopant

The hole transport region may further include, in addition to the materials described above, a charge generating material for improving conductive properties (e.g., electrically conductive properties). The charge generating material may be uniformly or non-uniformly dispersed in the hole transport region (e.g., in the form of a single layer consisting of a charge generating material).

The charge generating material may be, for example, a p-dopant.

For example, a lowest unoccupied molecular orbital (LUMO) energy level of the p-dopant may be about −3.5 eV or less.

In an embodiment, the p-dopant may include a quinone derivative, a cyano group-containing compound, a compound containing element EL1 and element EL2, or any combination thereof.

Examples of the quinone derivative may include TCNQ, F4-TCNQ, and the like.

Examples of the cyano group-containing compound may include HAT-CN, a compound represented by Formula 221, and the like:

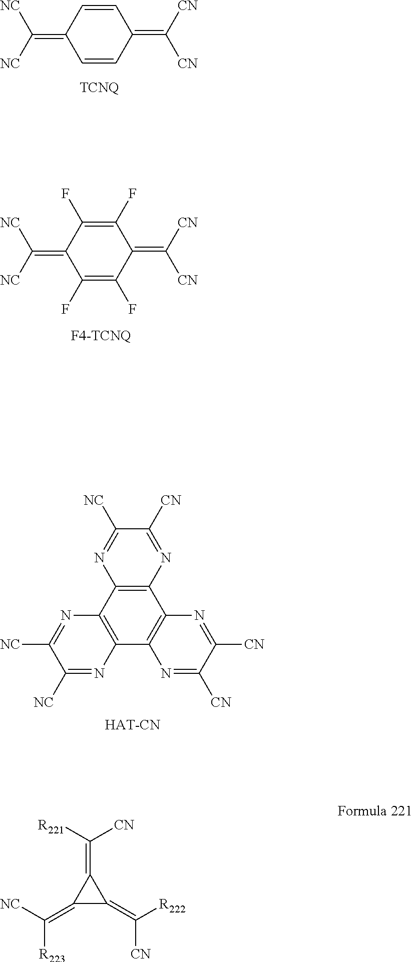

wherein, in Formula 221,
R₂₂₁ to R₂₂₃ may each independently be a C₃-C₆₀ carbocyclic group unsubstituted or substituted with at least one R₁₀ₐ or a C₁-C₆₀ heterocyclic group unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —Cl; —Br; —I; a C₁-C₂₀ alkyl group substituted with a cyano group, —F, —Cl, —Br, —I, or any combination thereof; or any combination thereof.

In the compound containing element EL1 and element EL2, element EL1 may be a metal, a metalloid, or any combination thereof, and element EL2 may be a non-metal, a metalloid, or any combination thereof.

Examples of the metal may include: an alkali metal (e.g., lithium (Li), sodium (Na), potassium (K), rubidium (Rb), cesium (Cs), etc.); an alkaline earth metal (e.g., beryllium (Be), magnesium (Mg), calcium (Ca), strontium (Sr), barium (Ba), etc.); a transition metal (e.g., titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), etc.); a post-transition metal (e.g., zinc (Zn), indium (In), tin (Sn), etc.); a lanthanide metal (e.g., lanthanum (La), cerium (Ce), praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm), ytterbium (Yb), lutetium (Lu), etc.); and the like.

Examples of the metalloid may include silicon (Si), antimony (Sb), tellurium (Te), and the like.

Examples of the non-metal may include oxygen (O), halogen (e.g., F, Cl, Br, I, etc.), and the like.

Examples of the compound containing element EL1 and element EL2 may include metal oxide, metal halide (e.g., metal fluoride, metal chloride, metal bromide, metal iodide, etc.), metalloid halide (e.g., metalloid fluoride, metalloid chloride, metalloid bromide, metalloid iodide, etc.), metal telluride, or any combination thereof.

Examples of the metal oxide may include a tungsten oxide (e.g., WO, W₂O₃, WO₂, WO₃, W₂O₅, etc.), vanadium oxide (e.g., VO, V₂O₃, VO₂, V₂O₅, etc.), molybdenum oxide (MoO, Mo₂O₃, MoO₂, MoO₃, Mo₂O₅, etc.), rhenium oxide (e.g., ReO₃, etc.), and the like.

Examples of the metal halide may include alkali metal halide, alkaline earth metal halide, transition metal halide, post-transition metal halide, lanthanide metal halide, and the like.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, CsI, and the like.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, BaI₂, and the like.

Examples of the transition metal halide may include titanium halide (e.g., TiF₄, TiCl₄, TiBr₄, TiI₄, etc.), zirconium halide (e.g., ZrF₄, ZrCl₄, ZrBr₄, ZrI₄, etc.), hafnium halide (e.g., HfF₄, HfCl₄, HfBr₄, HfI₄, etc.), vanadium halide (e.g., VF₃, VCI₃, VBr₃, VI₃, etc.), niobium halide (e.g., NbF₃, NbCl₃, NbBr₃, NbI₃, etc.), tantalum halide (e.g., TaF₃, TaCl₃, TaBr₃, TaI₃, etc.), chromium halide (e.g., CrF₃, CrC₁₃, CrBr₃, CrI₃, etc.), molybdenum halide (e.g., MoF₃, MoCl₃, MoBr₃, MoI₃, etc.), tungsten halide (e.g., WF₃, WCl₃, WBr₃, WI₃, etc.), manganese halide (e.g., MnF₂, MnCl₂, MnBr₂, MnI₂, etc.), technetium halide (e.g., TcF₂, TcCl₂, TcBr₂, TcI₂, etc.), rhenium halide (e.g., ReF₂, ReCl₂, ReBr₂, ReI₂, etc.), iron halide (e.g., FeF₂, FeCl₂, FeBr₂, FeI₂, etc.), ruthenium halide (e.g., RuF₂, RuCl₂, RuBr₂, RuI₂, etc.), osmium halide (e.g., OsF₂, OsCl₂, OsBr₂, Os₁₂, etc.), cobalt halide (e.g., CoF₂, CoCl₂, CoBr₂, COl₂, etc.), rhodium halide (e.g., RhF₂, RhCl₂, RhBr₂, RhI₂, etc.), iridium halide (e.g., IrF₂, IrCl₂, IrBr₂, IrI₂, etc.), nickel halide (e.g., NiF₂, NiCl₂, NiBr₂, NiI₂, etc.), palladium halide (e.g., PdF₂, PdCl₂, PdBr₂, PdI₂, etc.), platinum halide (e.g., PtF₂, PtCl₂, PtBr₂, PtI₂, etc.), copper halide (e.g., CuF, CuCl, CuBr, CuI, etc.), silver halide (e.g., AgF, AgCl, AgBr, AgI, etc.), gold halide (e.g., AuF, AuCl, AuBr, AuI, etc.), and the like.

Examples of the post-transition metal halide may include zinc halide (e.g., ZnF₂, ZnCl₂, ZnBr₂, ZnI₂, etc.), indium halide (e.g., InI₃, etc.), tin halide (e.g., SnI₂, etc.), and the like.

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, and the like.

Examples of the metalloid halide may include antimony halide (e.g., SbCl₅, etc.) and the like.

Examples of the metal telluride may include alkali metal telluride (e.g., Li₂Te, Na₂Te, K₂Te, Rb₂Te, Cs₂Te, etc.), alkaline earth metal telluride (e.g., BeTe, MgTe, CaTe, SrTe, BaTe, etc.), transition metal telluride (e.g., TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, etc.), post-transition metal telluride (e.g., ZnTe, etc.), lanthanide metal telluride (e.g., LaTe, CeTe, PrTe, NdTe, PmTe, EuTe, GdTe, TbTe, DyTe, HoTe, ErTe, TmTe, YbTe, LuTe, etc.), and the like.

### Emission Layer in Interlayer 130

When the light-emitting device 10 is a full-color light-emitting device, the emission layer may be patterned into a red emission layer, a green emission layer, and/or a blue emission layer, according to a sub-pixel. In an embodiment, the emission layer may have a stacked structure of two or more layers of a red emission layer, a green emission layer, and a blue emission layer, in which the two or more layers contact (e.g., physically contact) each other or are separated from each other to emit white light. In one or more embodiments, the emission layer may include two or more materials of a red light-emitting material, a green light-emitting material, and a blue light-emitting material, in which the two or more materials are mixed together with each other in a single layer to emit white light.

The emission layer may include a host and a dopant. The dopant may include a phosphorescent dopant, a fluorescent dopant, or any combination thereof.

An amount of the dopant in the emission layer may be in a range of about 0.01 part by weight to about 15 parts by weight with respect to 100 parts by weight of the host.

In an embodiment, the emission layer may include a quantum dot.

In an embodiment, the emission layer may include a delayed fluorescence material. The delayed fluorescence material may act as a host or a dopant in the emission layer.

A thickness of the emission layer may be in a range of about 100 Å to about 1,000 Å, for example, about 200 Å to about 600 Å. When the thickness of the emission layer is within this range, excellent light-emission characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21} Formula 301
wherein, in Formula 301,
Ar₃₀₁ and L₃₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xb11 may be 1, 2, or 3,
xb1 may be an integer from 0 to 5,
R₃₀₁ may be hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer from 1 to 5, and
Q₃₀₁ to Q₃₀₃ are each the same as described in connection with Q₁.

For example, when xb11 in Formula 301 is 2 or more, two or more of Ar₃₀₁(s) may be linked to each other via a single bond.

In an embodiment, the host may include a compound represented by Formula 301-1, a compound represented by Formula 301-2, or any combination thereof:

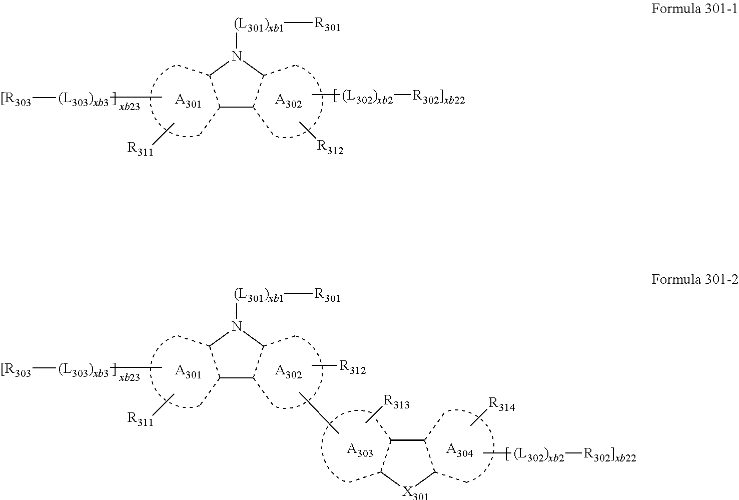

wherein, in Formulae 301-1 and 301-2,

ring A₃₀₁ to ring A₃₀₄ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
X₃₀₁ may be O, S, N-[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each the same as described herein,
L₃₀₂ to L₃₀₄ are each independently the same as described in connection with L₃₀₁,
xb2 to xb4 are each independently the same as described in connection with xb1, and
R₃₀₂ to R₃₀₅ and R₃₁₁ to R₃₁₄ are each the same as described in connection with R₃₀₁.

In an embodiment, the host may include an alkali earth metal complex, a post-transition metal complex, or any combination thereof. For example, the host may include a Be complex (e.g., Compound H55), an Mg complex, a Zn complex, or any combination thereof.

In an embodiment, the host may include one of Compounds H1 to H128, 9,10-di(2-naphthyl)anthracene (ADN), 2-methyl-9,10-bis(naphthalen-2-yl)anthracene (MADN), 9,10-di-(2-naphthyl)-2-t-butyl-anthracene (TBADN), 4,4′-bis(N-carbazolyl)-1,1′-biphenyl (CBP), 1,3-di(carbazol-9-yl)benzene (mCP), 1,3,5-tri(carbazol-9-yl)benzene (TCP), or any combination thereof:

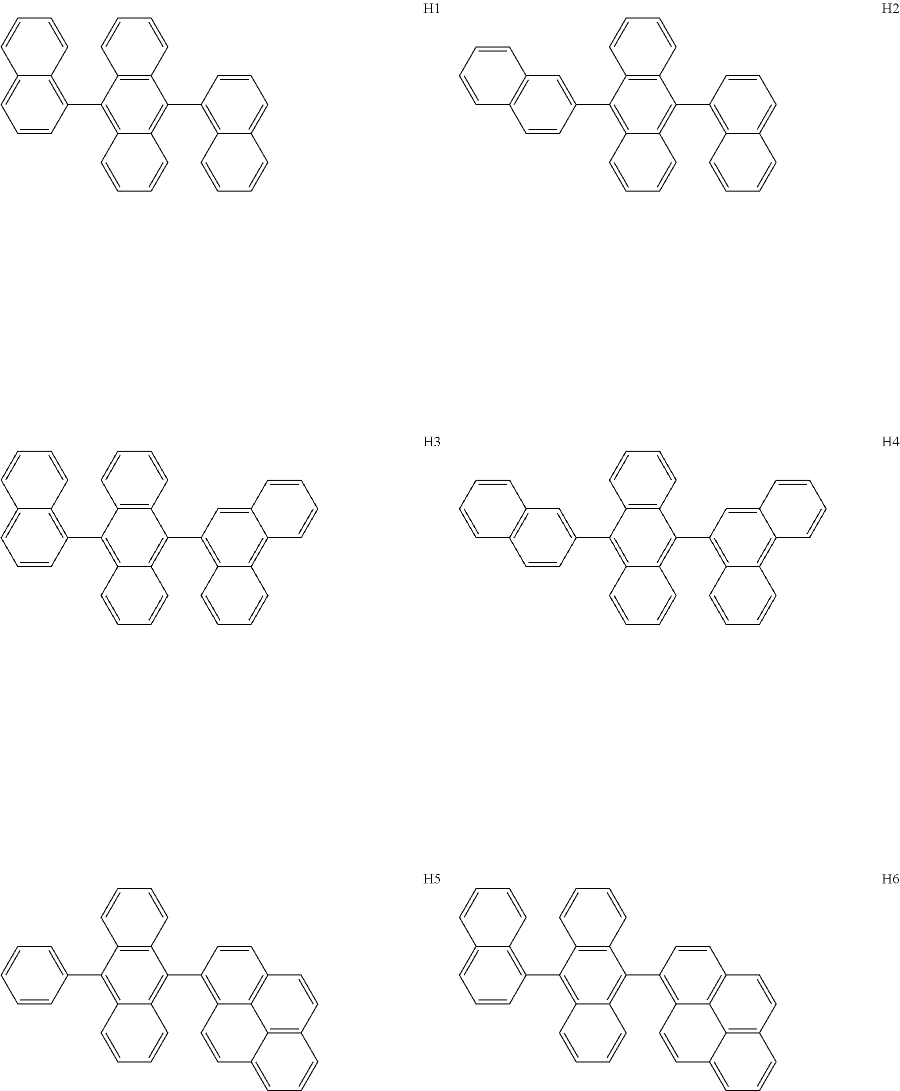

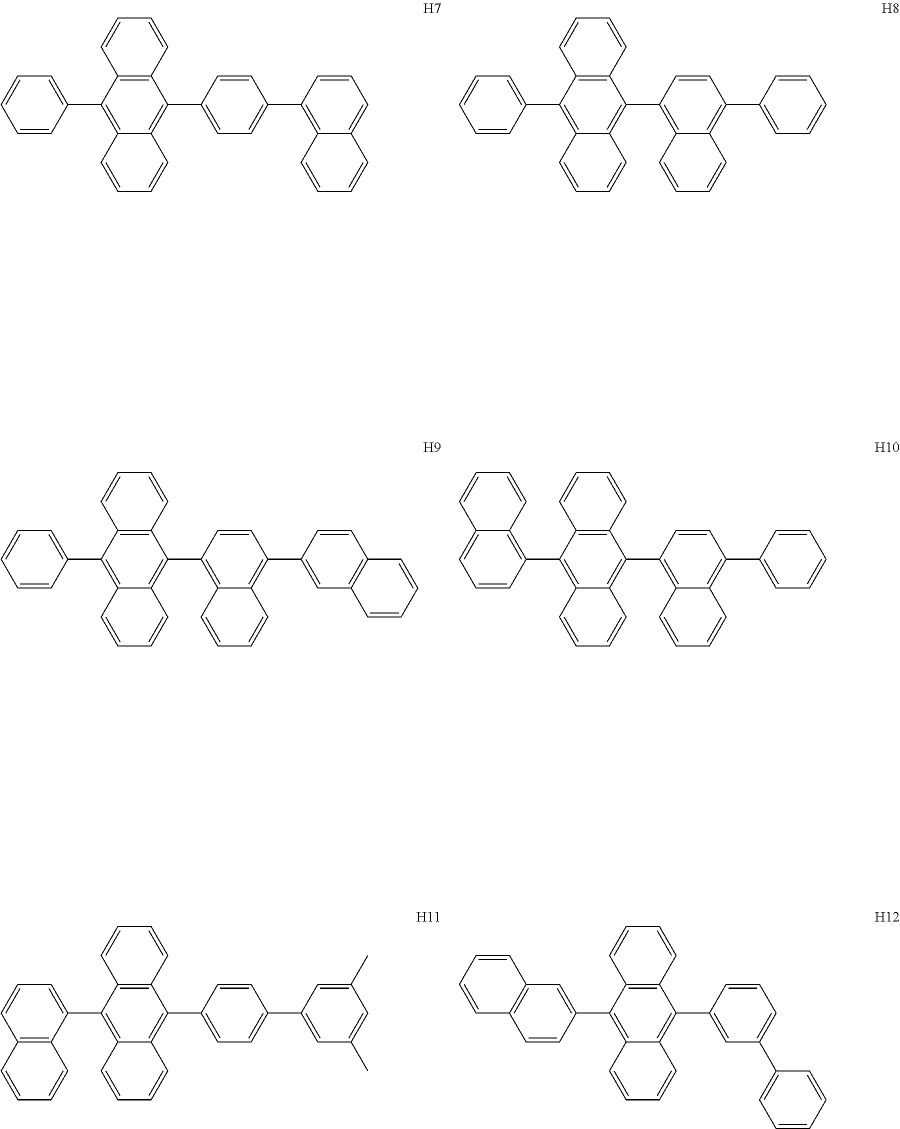

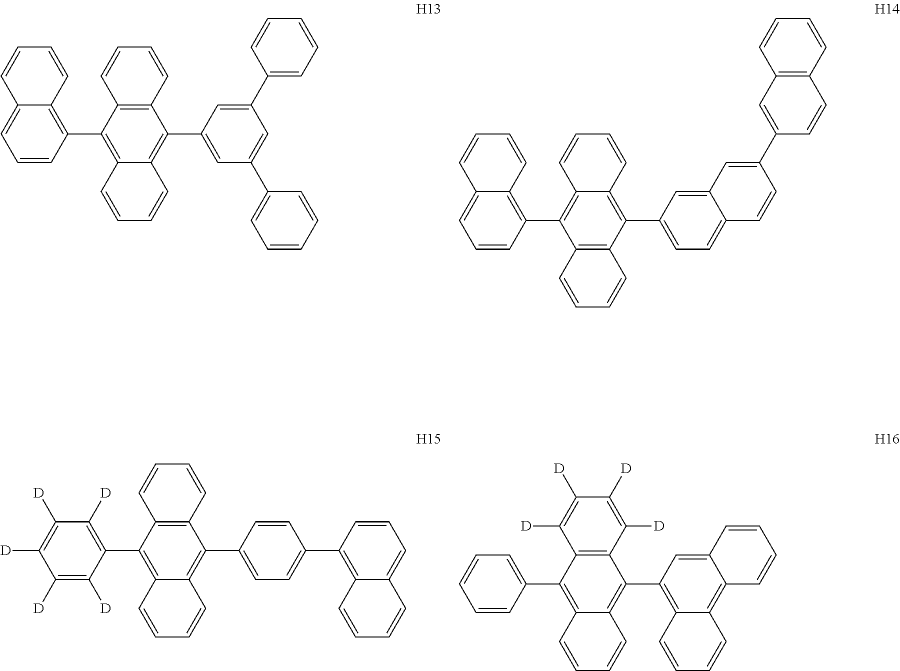

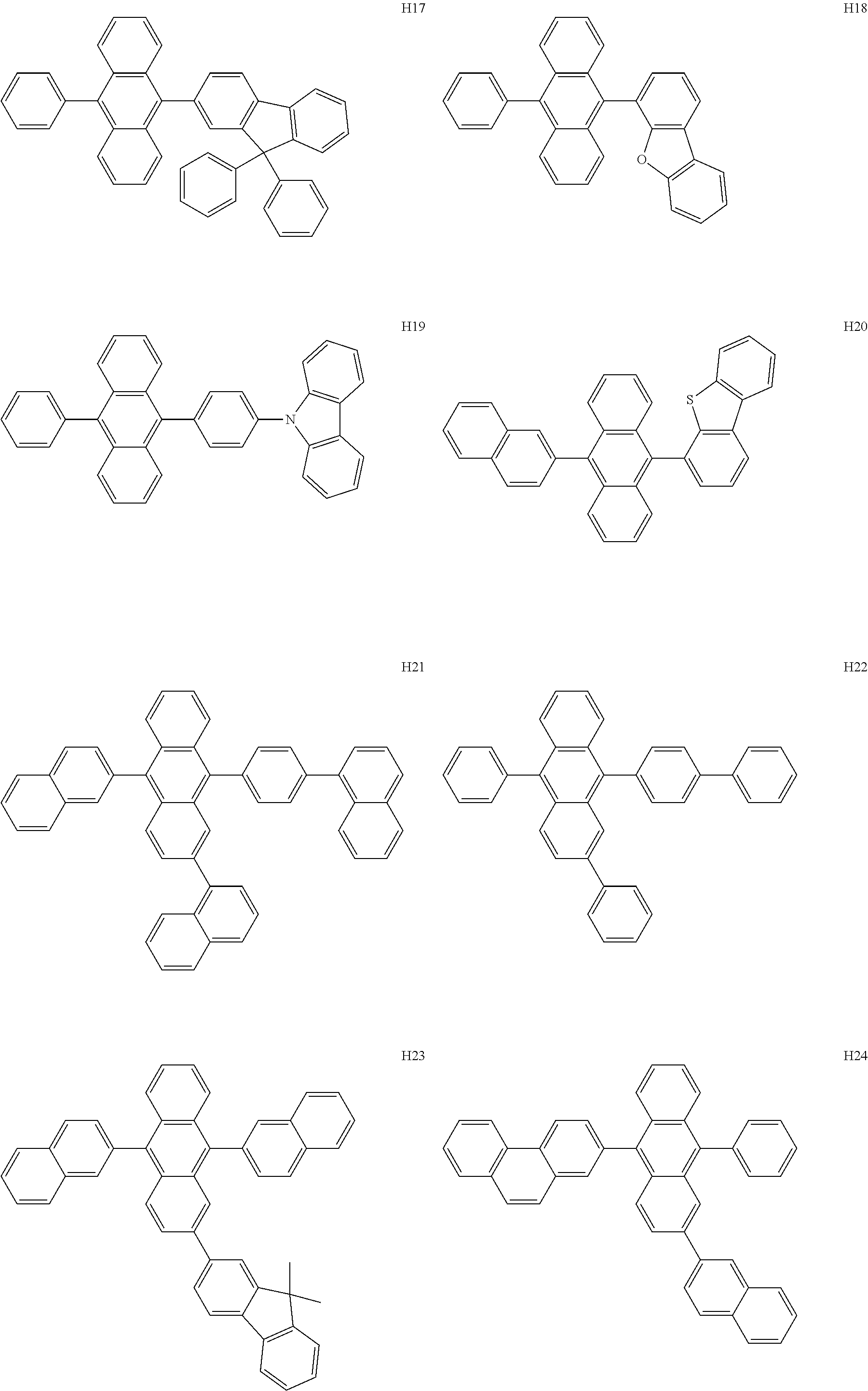

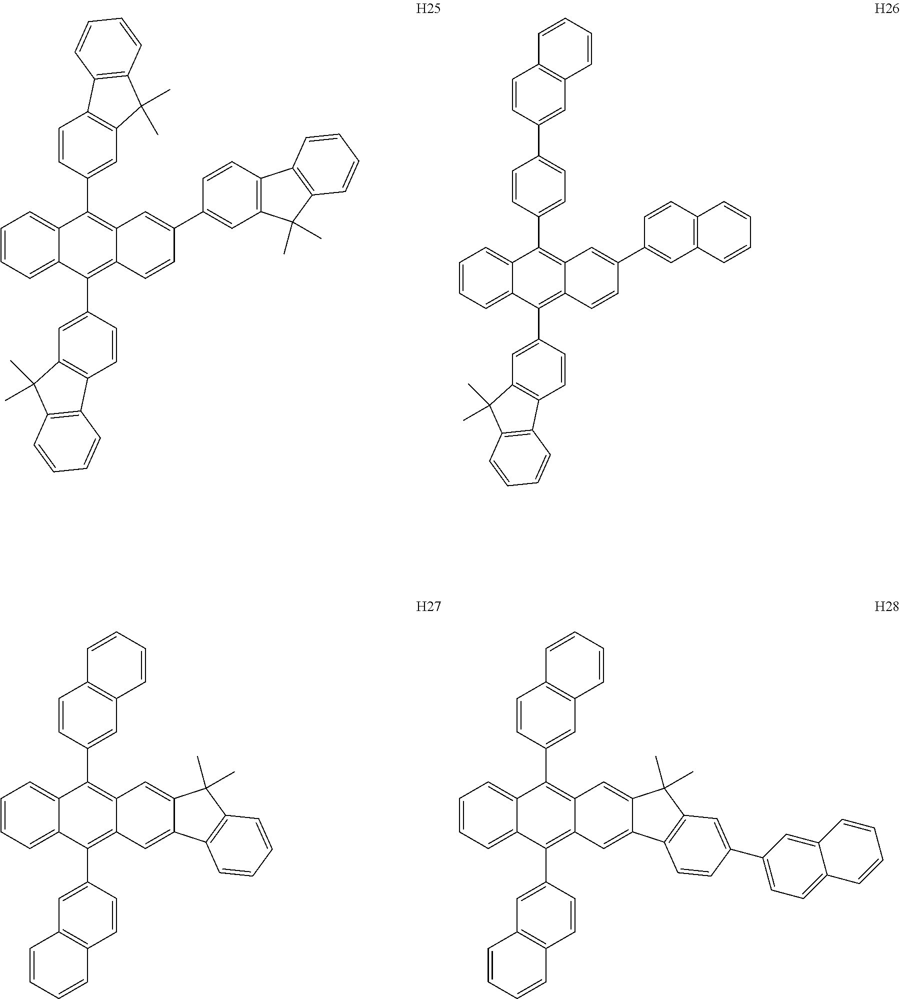

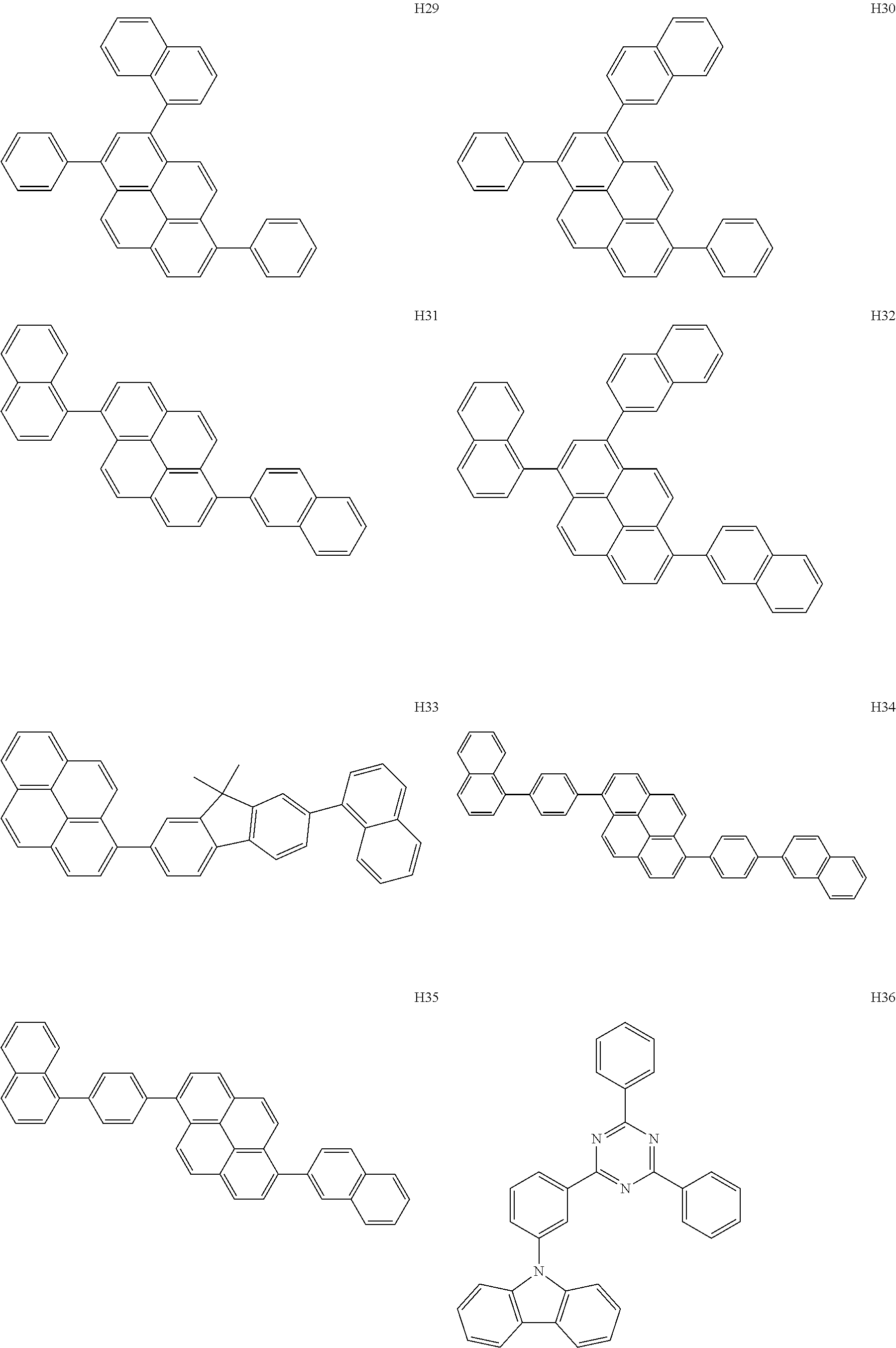

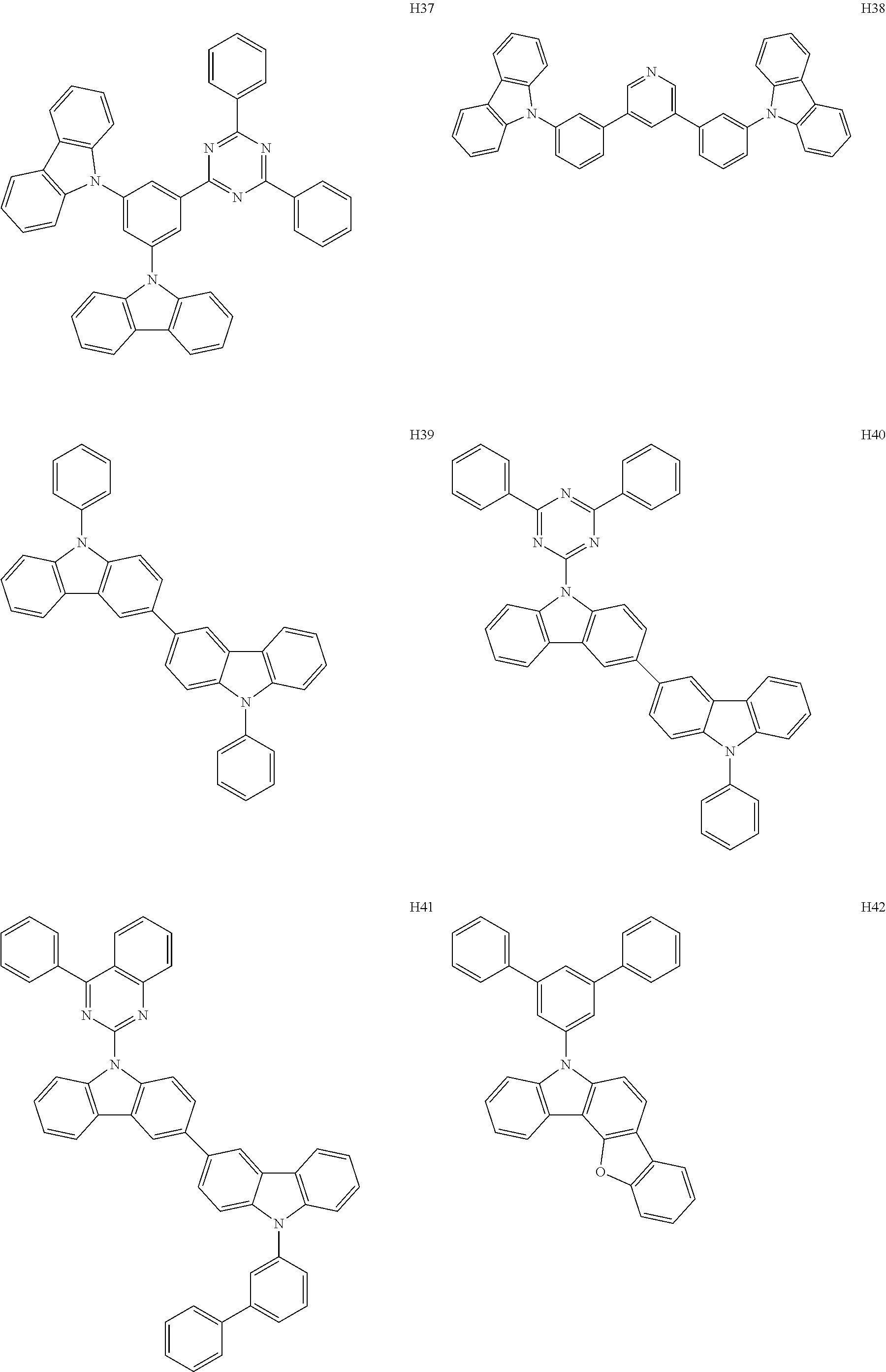 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

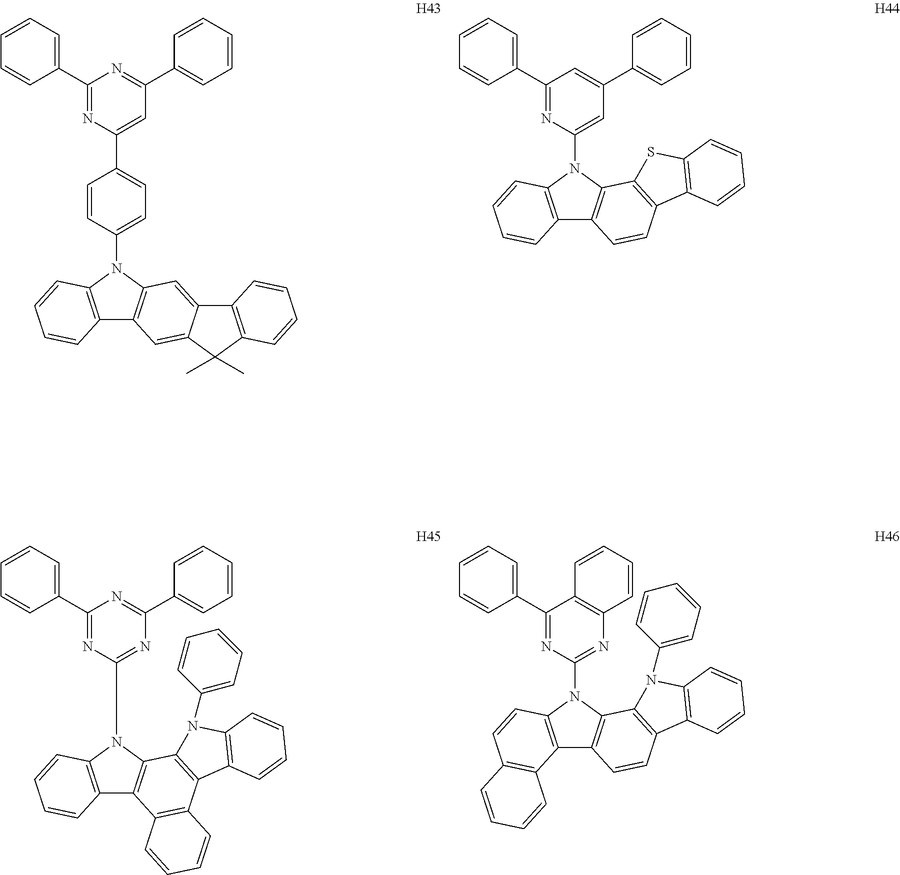 [SMILES: CC1(C)c2ccccc2-c2cc3c(cc21)c1ccccc1n3-c1ccc(-c2cc(-c3ccccc3)nc(-c3ccccc3)n2)cc1 ; c1ccc(-c2cc(-c3ccccc3)nc(-n3c4ccccc4c4ccc5c6ccccc6sc5c43)c2)cc1 ; c1ccc(-c2nc(-c3ccccc3)nc(-n3c4ccccc4c4c5ccccc5c5c6ccccc6n(-c6ccccc6)c5c43)n2)cc1 ; c1ccc(-c2nc(-n3c4ccc5ccccc5c4c4ccc5c6ccccc6n(-c6ccccc6)c5c43)nc3ccccc23)cc1]

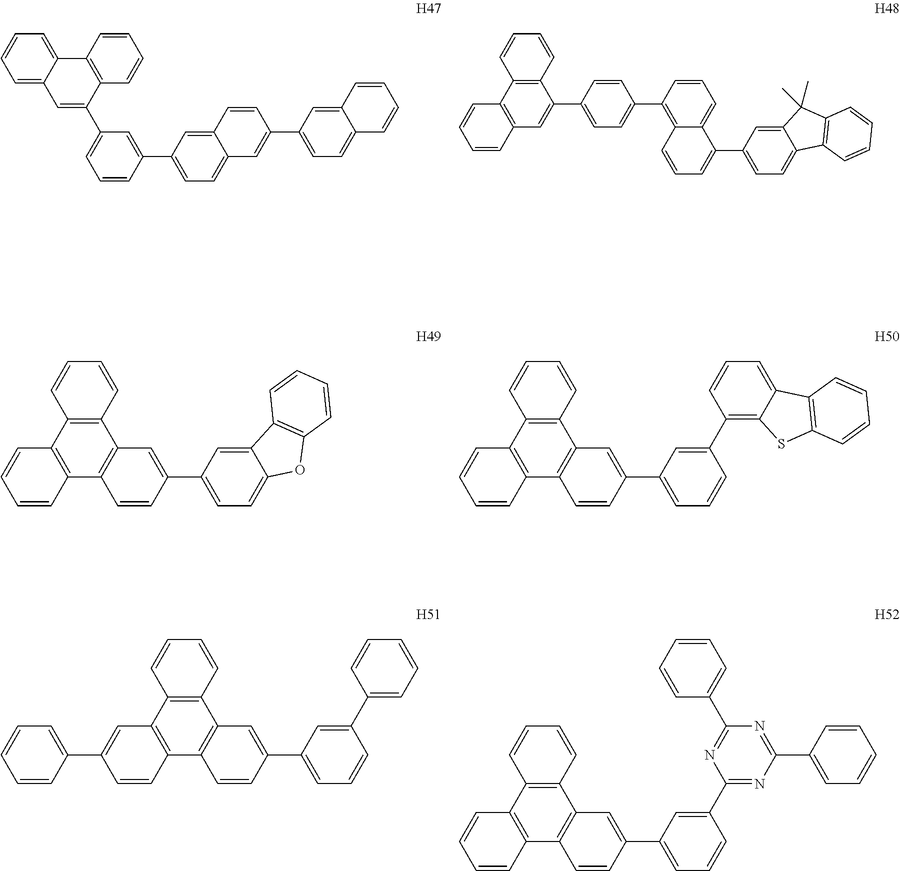

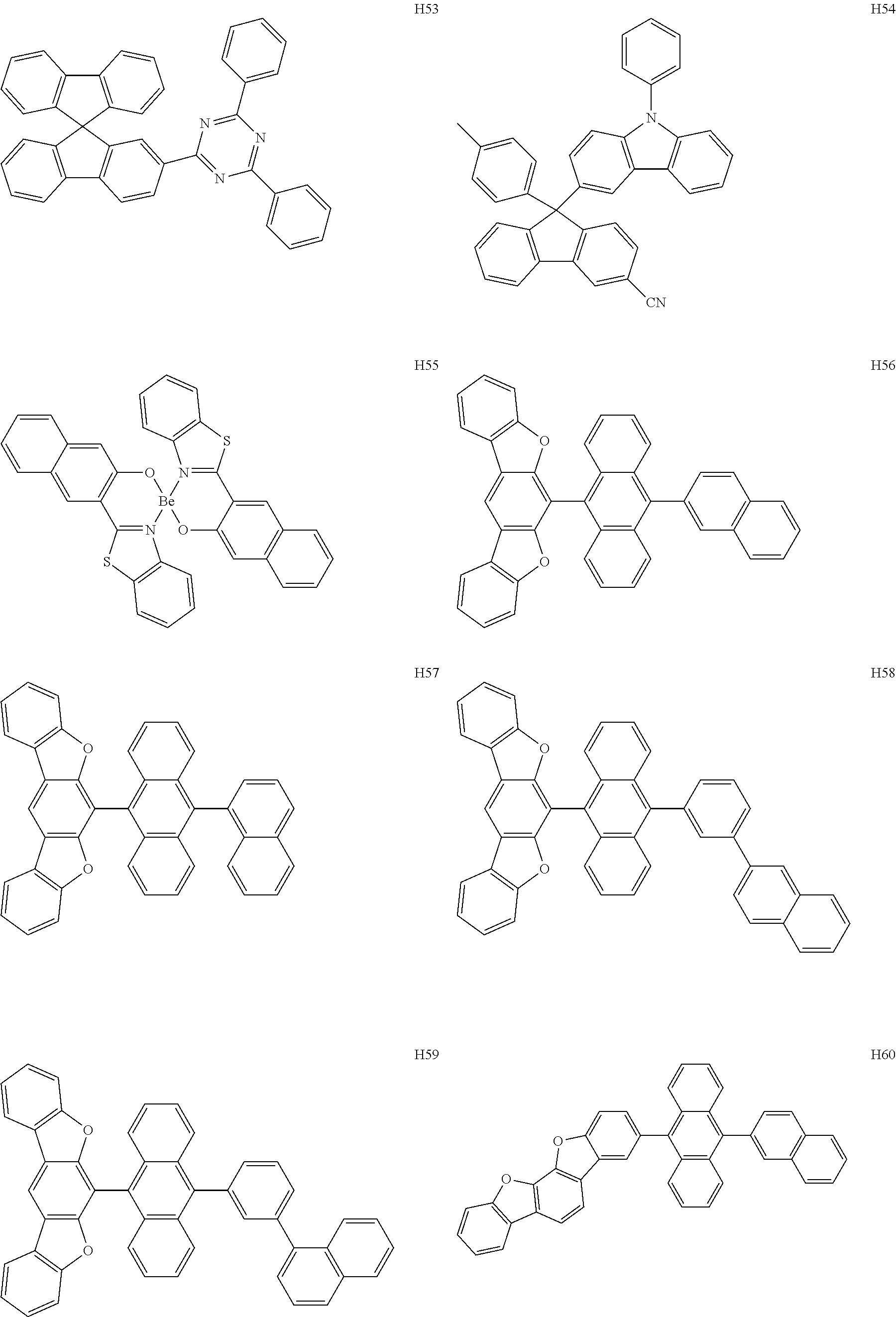

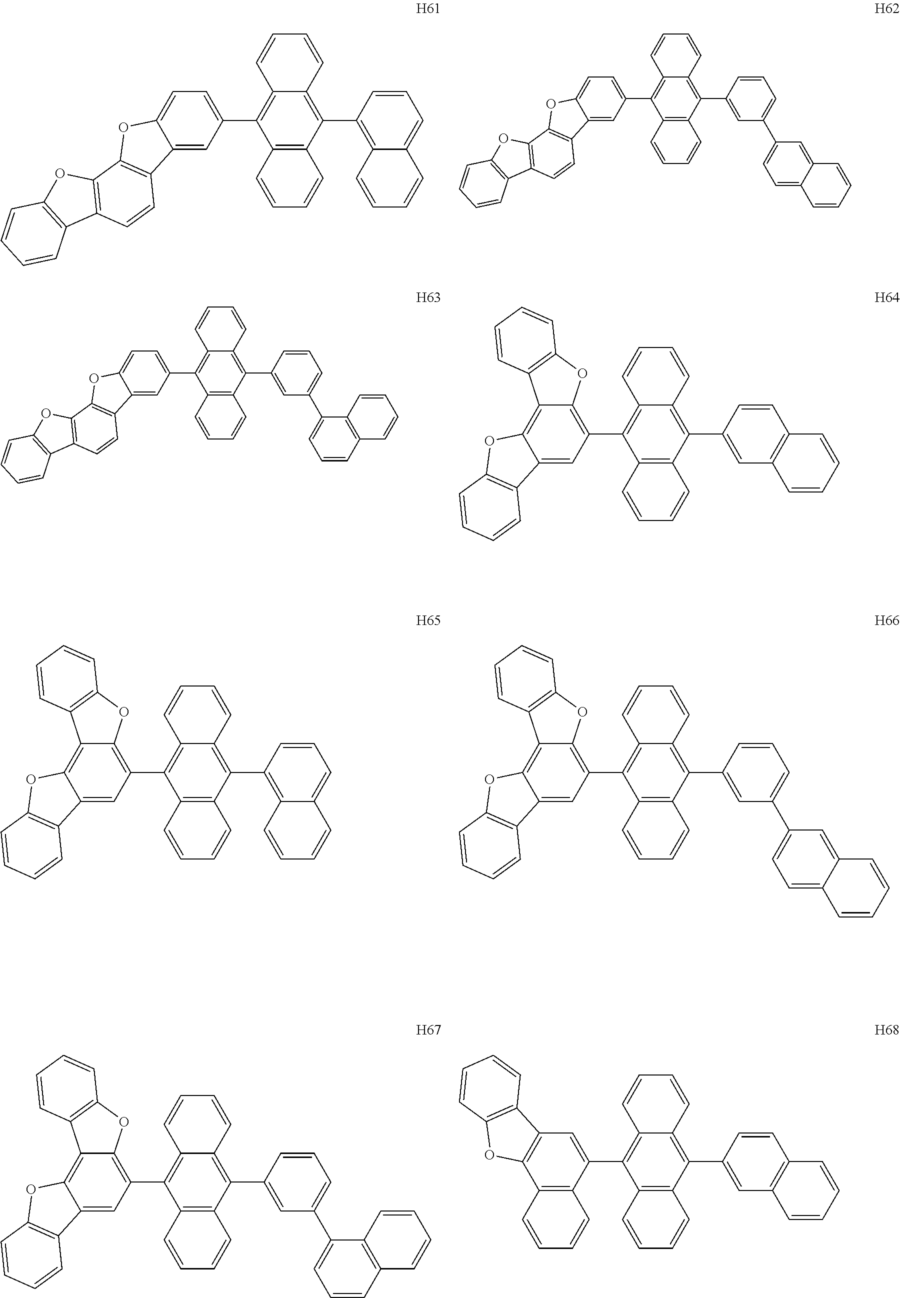

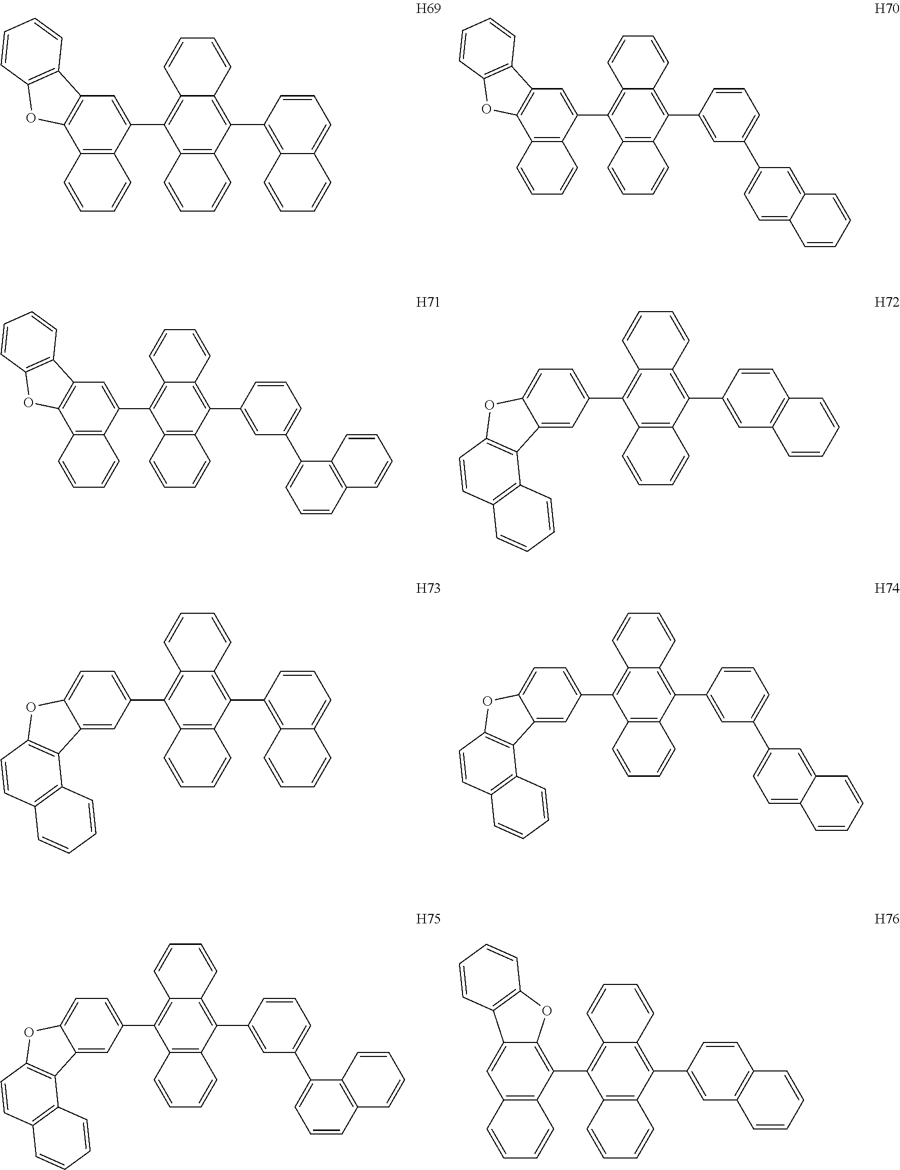

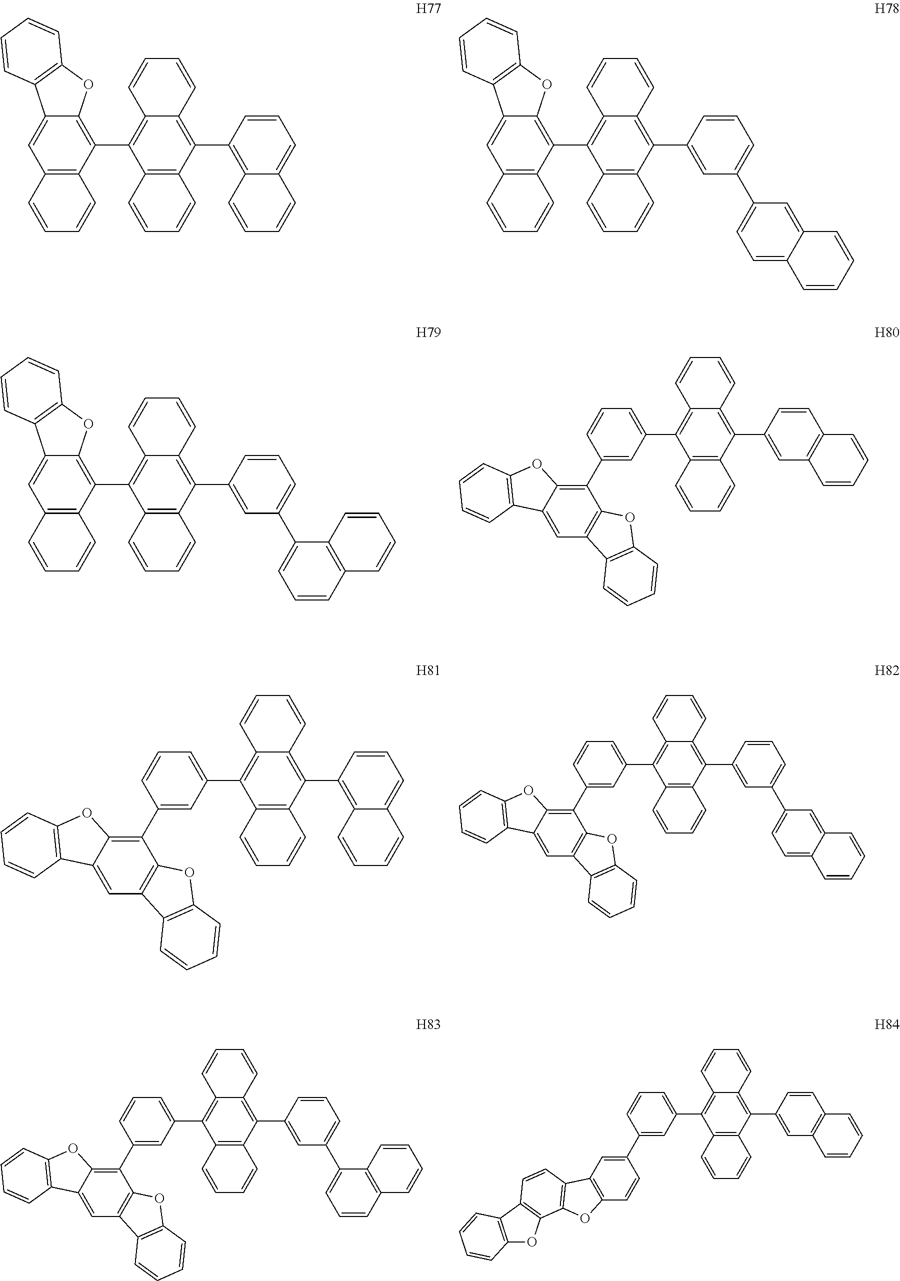

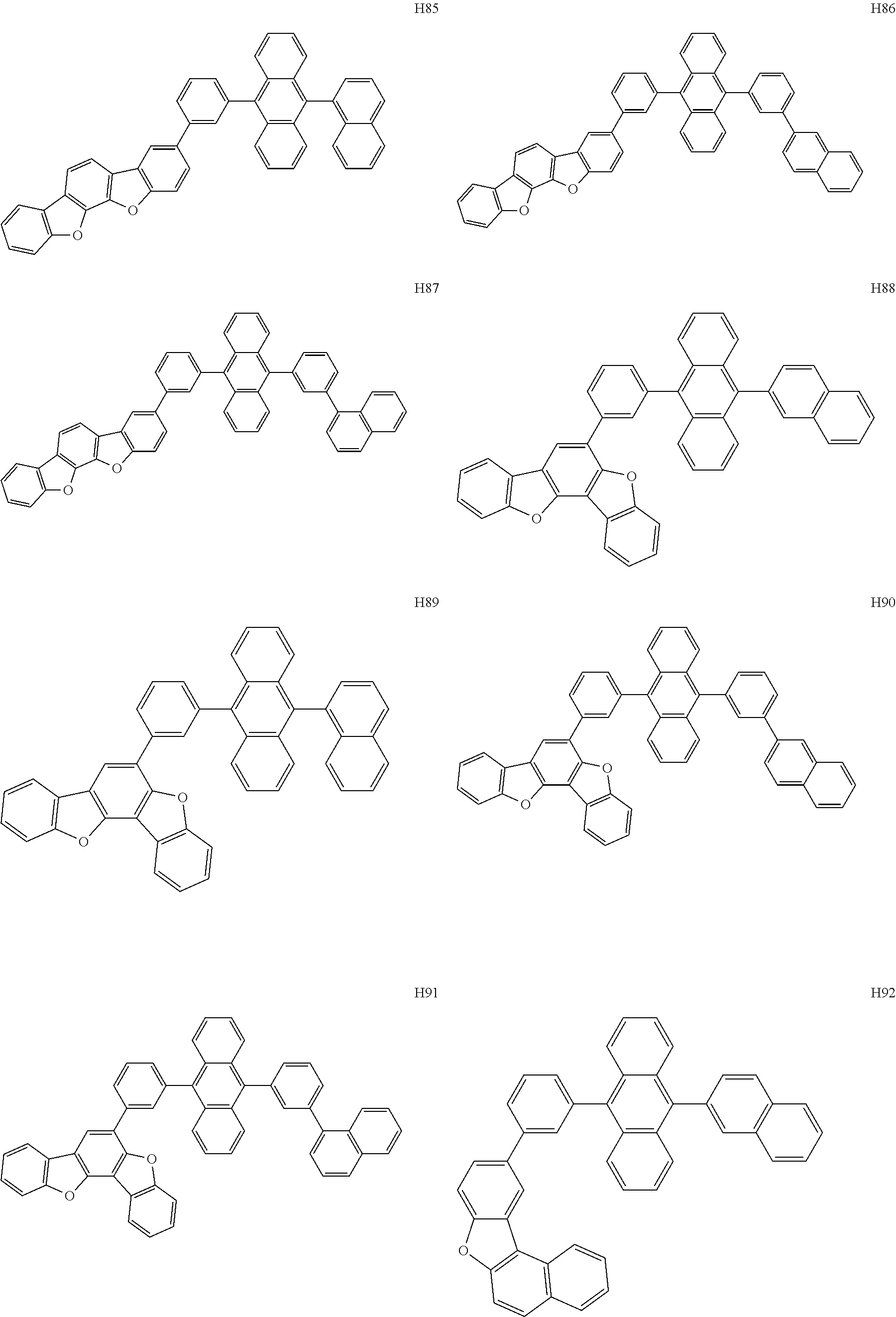

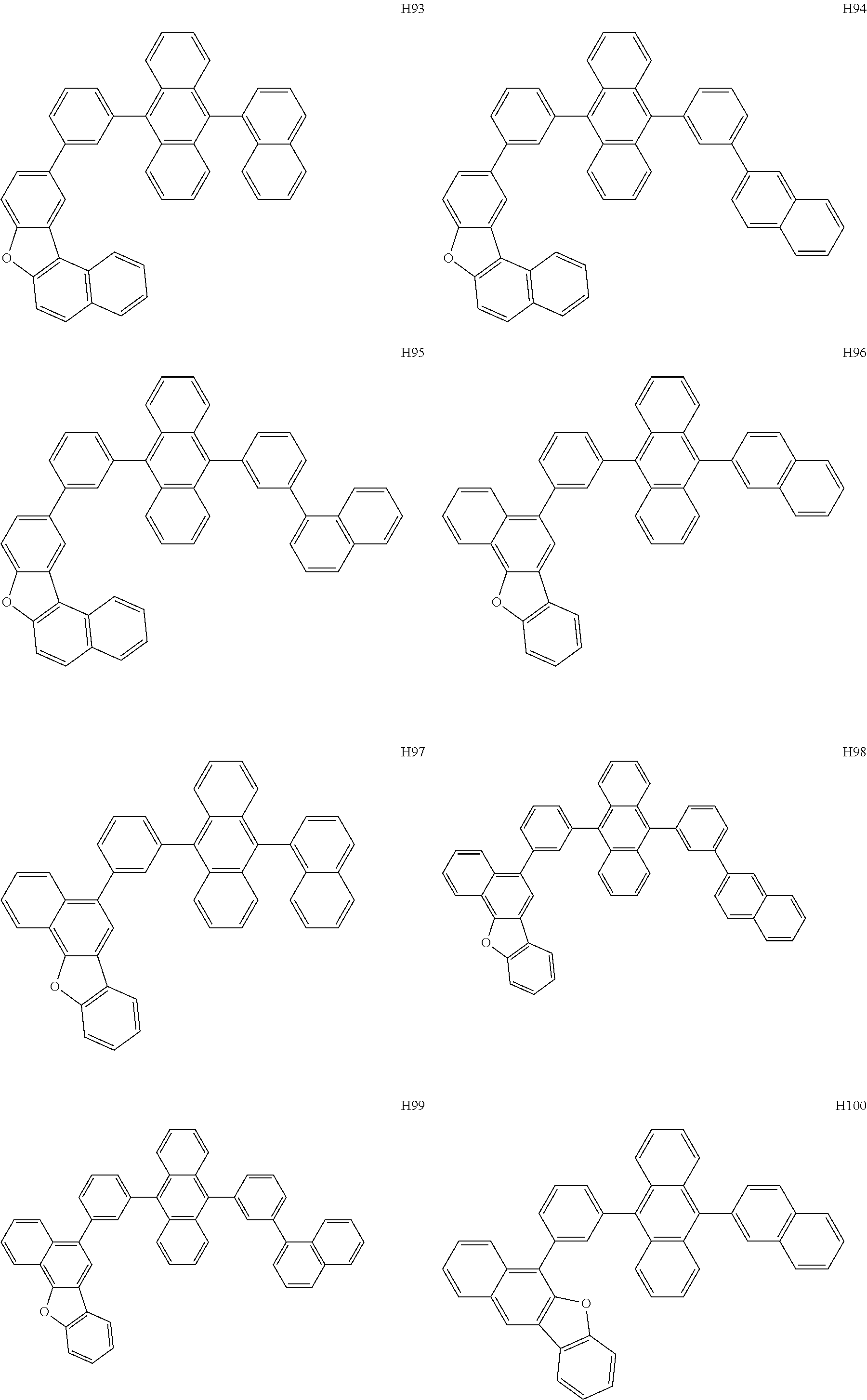

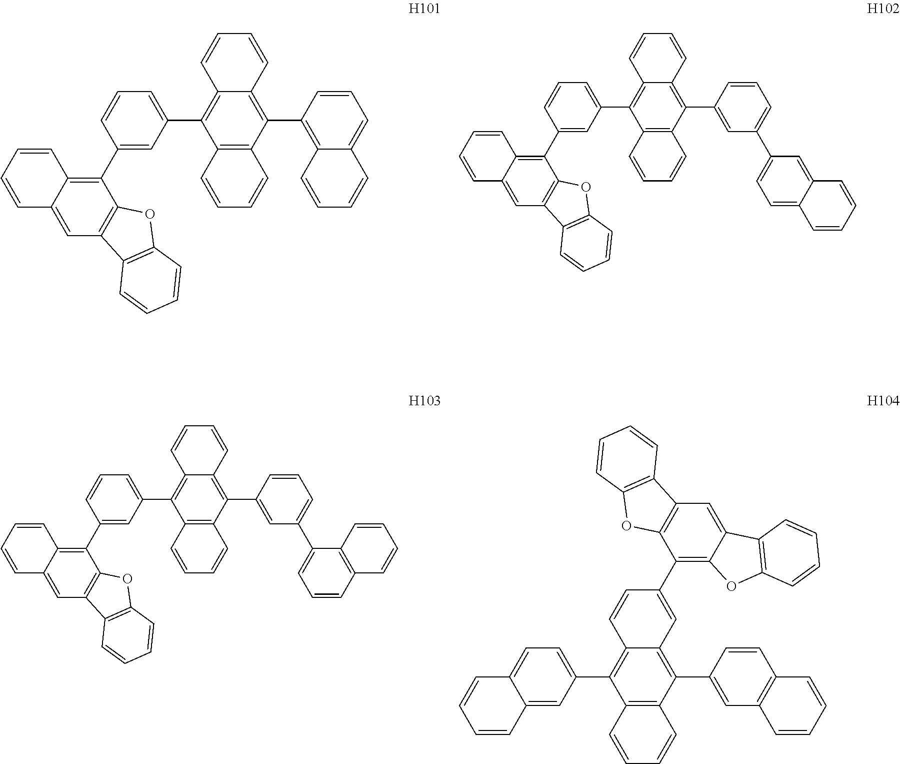 [SMILES: c1cc(-c2c3ccccc3c(-c3cccc4ccccc34)c3ccccc23)cc(-c2c3ccccc3cc3c2oc2ccccc23)c1 ; c1cc(-c2ccc3ccccc3c2)cc(-c2c3ccccc3c(-c3cccc(-c4c5ccccc5cc5c4oc4ccccc45)c3)c3ccccc23)c1 ; c1cc(-c2cccc3ccccc23)cc(-c2c3ccccc3c(-c3cccc(-c4c5ccccc5cc5c4oc4ccccc45)c3)c3ccccc23)c1 ; c1ccc2cc(-c3c4ccccc4c(-c4ccc5ccccc5c4)c4cc(-c5c6oc7ccccc7c6cc6c5oc5ccccc56)ccc34)ccc2c1]

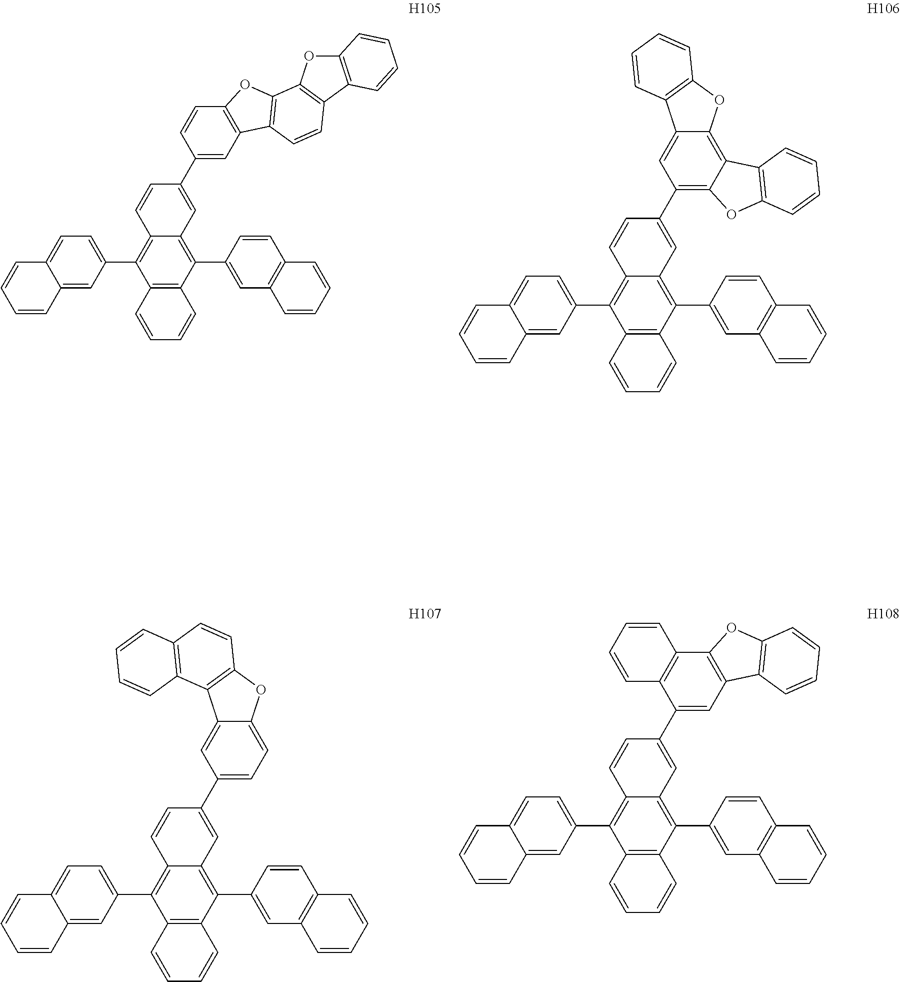 [SMILES: c1ccc2cc(-c3c4ccccc4c(-c4ccc5ccccc5c4)c4cc(-c5ccc6oc7c(ccc8c9ccccc9oc87)c6c5)ccc34)ccc2c1 ; c1ccc2cc(-c3c4ccccc4c(-c4ccc5ccccc5c4)c4cc(-c5cc6c7ccccc7oc6c6c5oc5ccccc56)ccc34)ccc2c1 ; c1ccc2cc(-c3c4ccccc4c(-c4ccc5ccccc5c4)c4cc(-c5ccc6oc7ccc8ccccc8c7c6c5)ccc34)ccc2c1 ; c1ccc2cc(-c3c4ccccc4c(-c4ccc5ccccc5c4)c4cc(-c5cc6c7ccccc7oc6c6ccccc56)ccc34)ccc2c1]

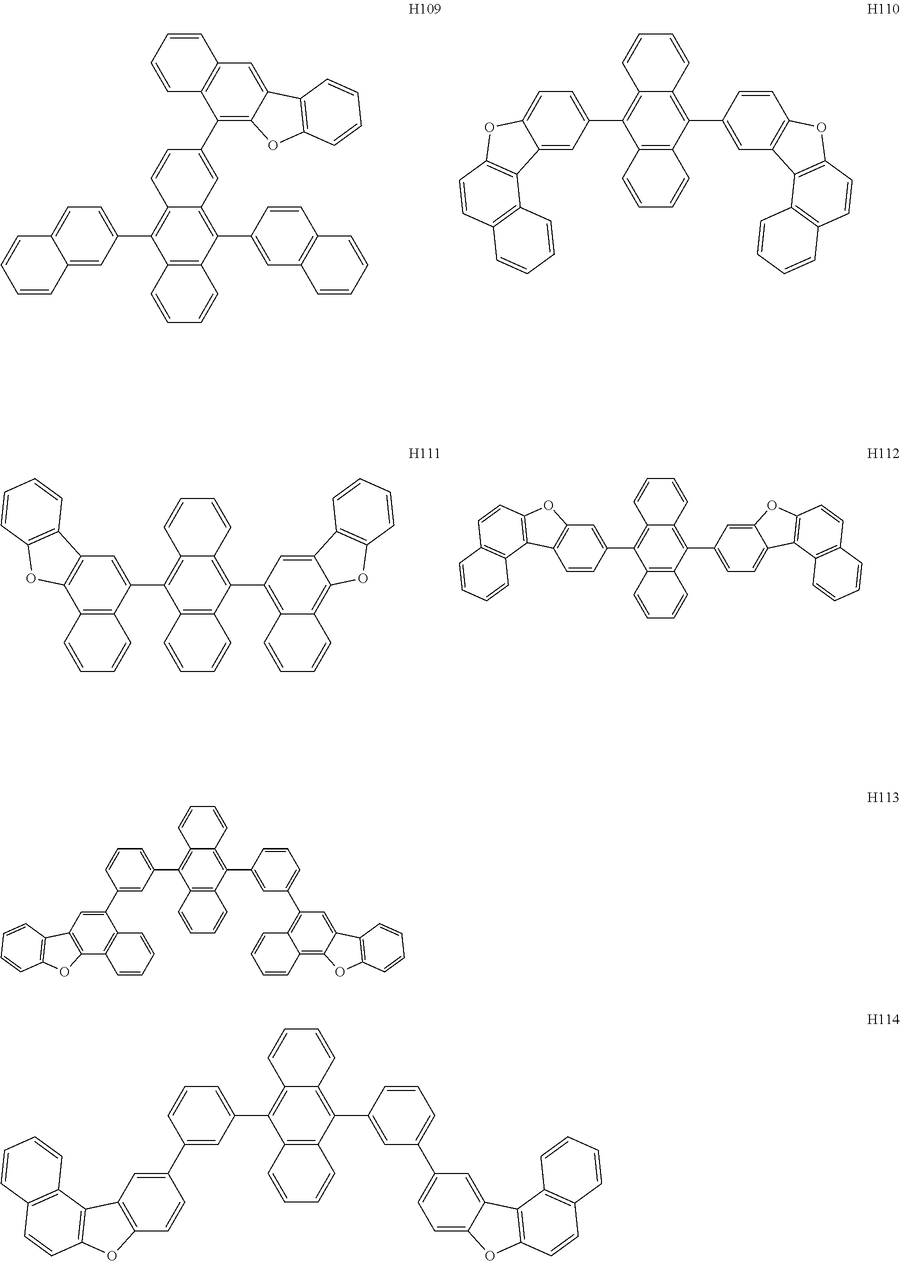

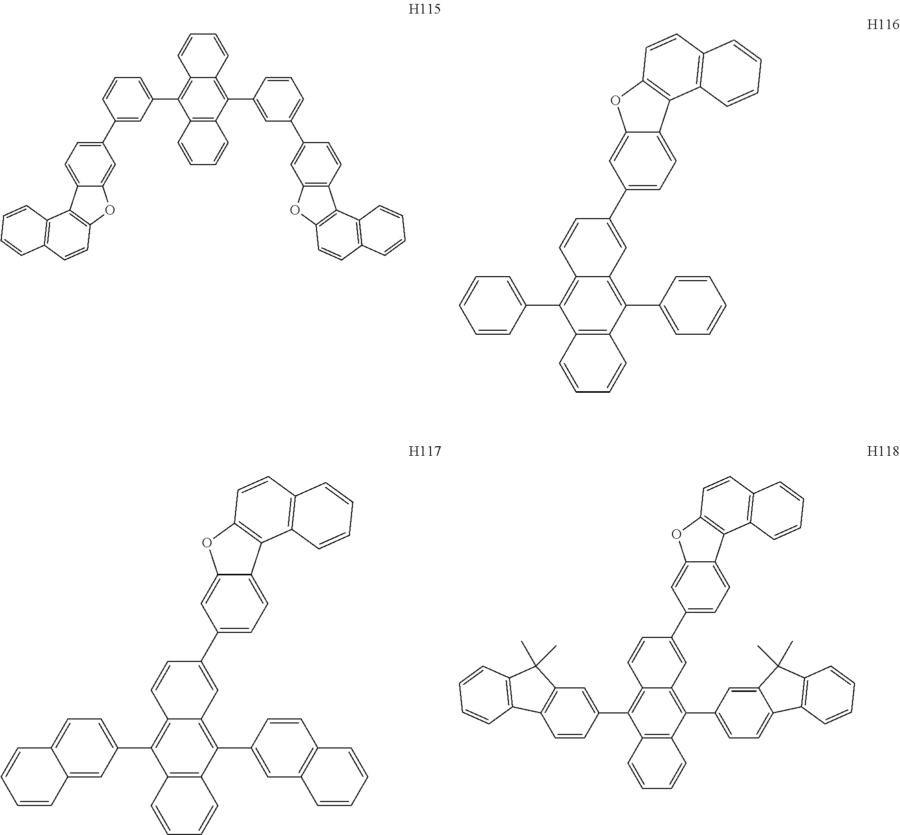 [SMILES: c1cc(-c2ccc3c(c2)oc2ccc4ccccc4c23)cc(-c2c3ccccc3c(-c3cccc(-c4ccc5c(c4)oc4ccc6ccccc6c45)c3)c3ccccc23)c1 ; c1ccc(-c2c3ccccc3c(-c3ccccc3)c3cc(-c4ccc5c(c4)oc4ccc6ccccc6c45)ccc23)cc1 ; c1ccc2cc(-c3c4ccccc4c(-c4ccc5ccccc5c4)c4cc(-c5ccc6c(c5)oc5ccc7ccccc7c56)ccc34)ccc2c1 ; CC1(C)c2ccccc2-c2ccc(-c3c4ccccc4c(-c4ccc5c(c4)C(C)(C)c4ccccc4-5)c4cc(-c5ccc6c(c5)oc5ccc7ccccc7c56)ccc34)cc21]

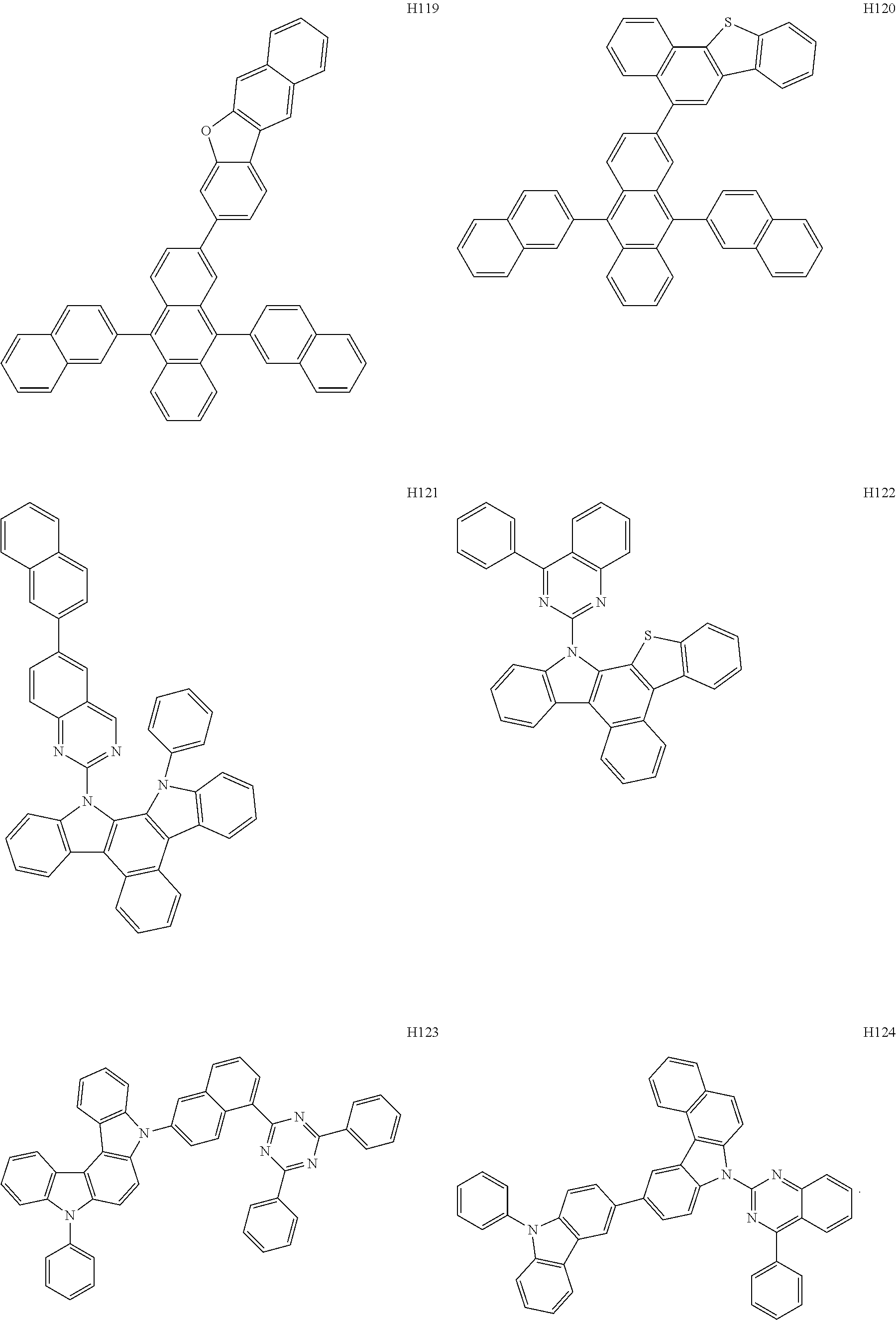

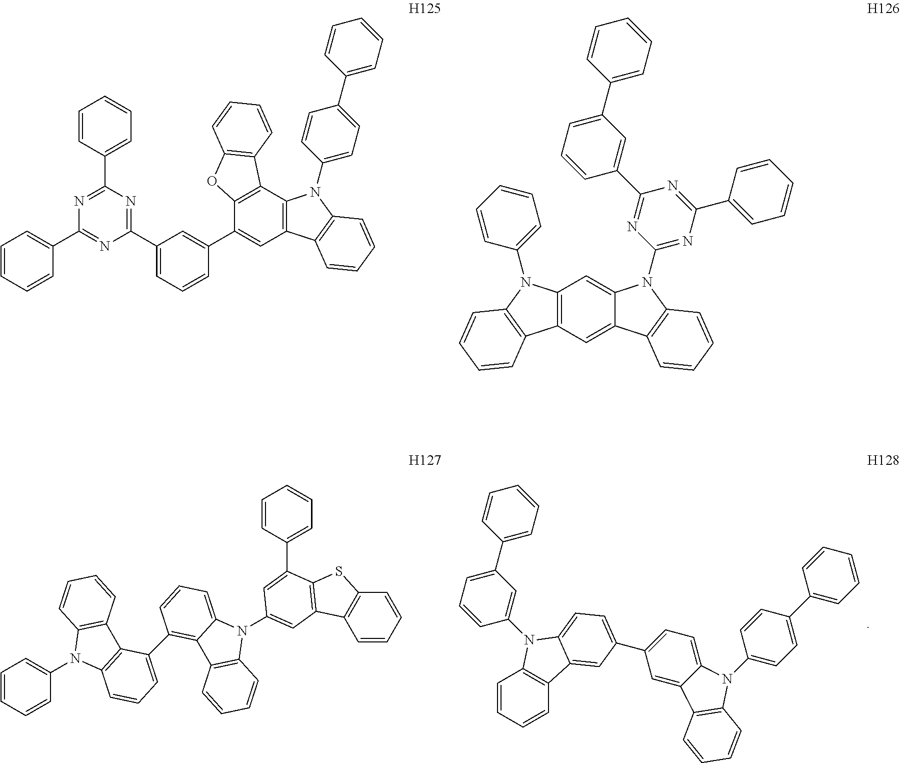 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

For example, the phosphorescent dopant may include an organometallic compound represented by Formula 401:

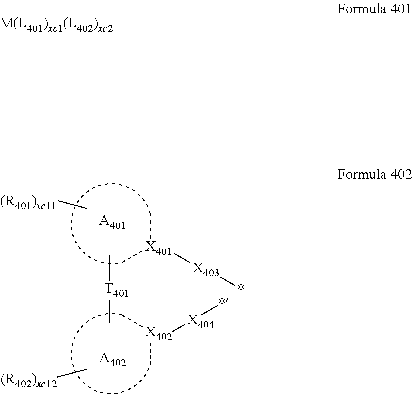

wherein, in Formulae 401 and 402,
M may be a transition metal (e.g., iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 may be 1, 2, or 3, wherein, when xc1 is 2 or more, two or more of L₄₀₁(s) may be identical to or different from each other,
L₄₀₂ may be an organic ligand, and xc2 may be 0, 1, 2, 3, or 4, wherein, when xc2 is 2 or more, two or more of L₄₀₂(s) may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*′, *—C(═O)—*′, *—N(Q₄₁₁)-*′ *—C(Q₄₁₁)(Q₄₁₂)*′, *—C(Q₄₁₁)═C(Q₄₁₂)-*, *—C(O₄₁₁)═*′, or *═C═*′,
X₄₀₃ and X₄₀₄ may each independently be a chemical bond (e.g., a covalent bond or a coordination bond, which may also be referred to as a coordinate covalent bond or a dative bond), O, S, N(Q₄₁₃), B(Q₄₁₃), P(Q₄₁₃), C(Q₄₁₃)(Q₄₁₄), or Si(Q₄₁₃)(Q₄₁₄),
Q₄₁₁ to Q₄₁₄ are each the same as described in connection with Q₁,
R₄₀₁ and R₄₀₂ may each independently be hydrogen, deuterium, —F, —Cl, —Br, —I, a hydroxyl group, a cyano group, a nitro group, a C₁-C₂₀ alkyl group unsubstituted or substituted with at least one R₁₀ₐ, a C₁-C₂₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ are each the same as described in connection with Q₁,
xc11 and xc12 may each independently be an integer from 0 to 10, and
* and *′ in Formula 402 each indicate a binding site to M in Formula 401.

For example, in Formula 402, i) X₄₀₁ may be nitrogen, and X₄₀₂ may be carbon, or ii) each of X₄₀₁ and X₄₀₂ may be nitrogen.

In an embodiment, when xc1 in Formula 401 is 2 or more, two ring A₄₀₁(s) in two or more of L₄₀₁(s) may optionally be linked to each other via T₄₀₂, which is a linking group, and two ring A₄₀₂(s) may optionally be linked to each other via T₄₀₃, which is a linking group (see Compounds PD1 to PD4 and PD7). T₄₀₂ and T₄₀₃ are each the same as described in connection with T₄₀₁.

L₄₀₂ in Formula 401 may be an organic ligand. For example, L₄₀₂ may include a halogen group, a diketone group (e.g., an acetylacetonate group), a carboxylic acid group (e.g., a picolinate group), —C(═O), an isonitrile group, a —CN group, a phosphorus group (e.g., a phosphine group, a phosphite group, etc.), or any combination thereof.

The phosphorescent dopant may include, for example, one of Compounds PD1 to PD39 or any combination thereof:

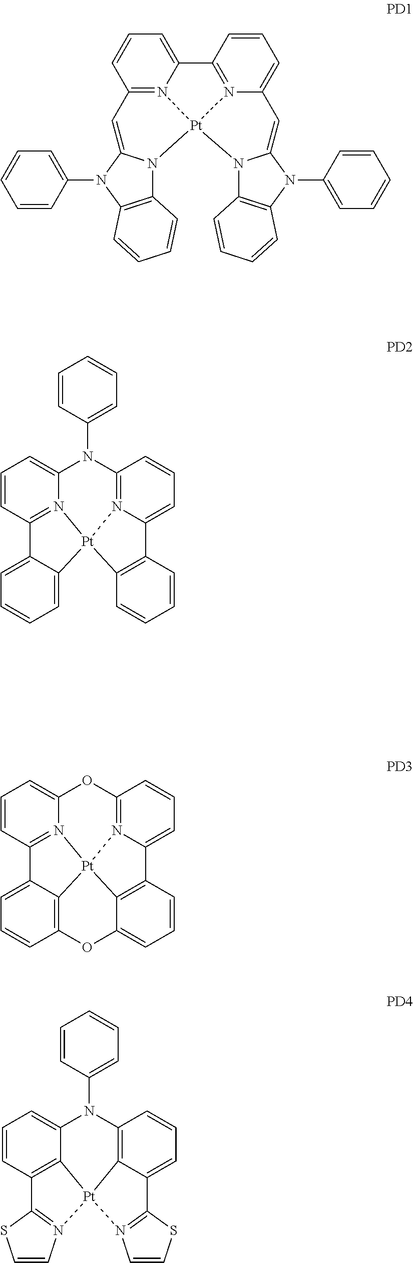

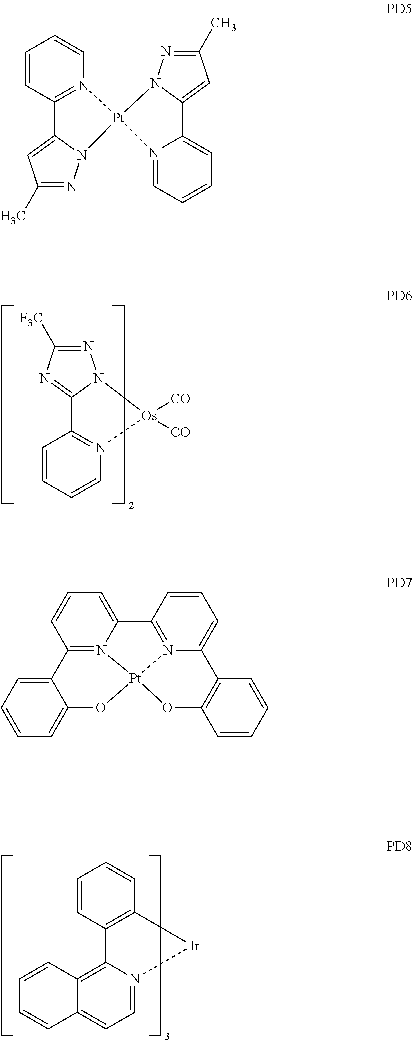

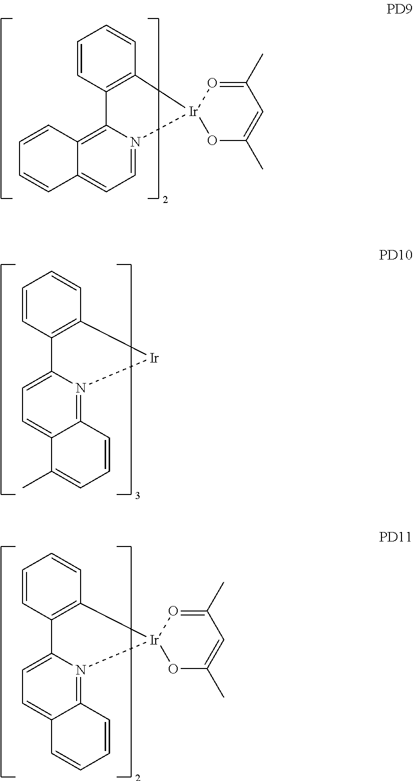

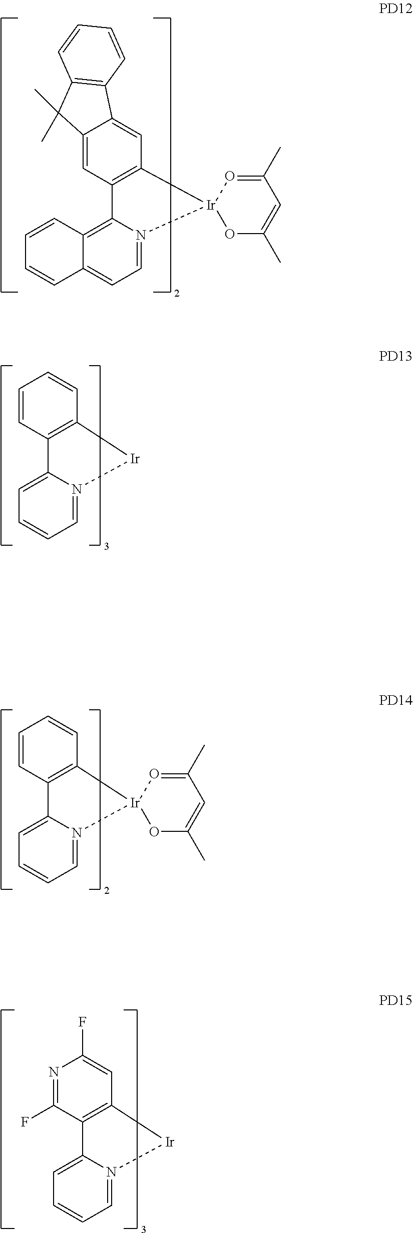

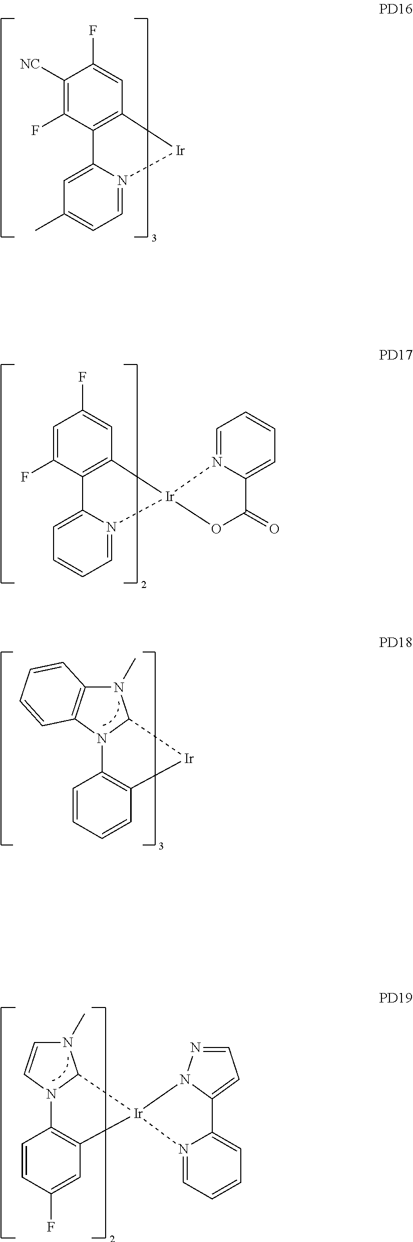

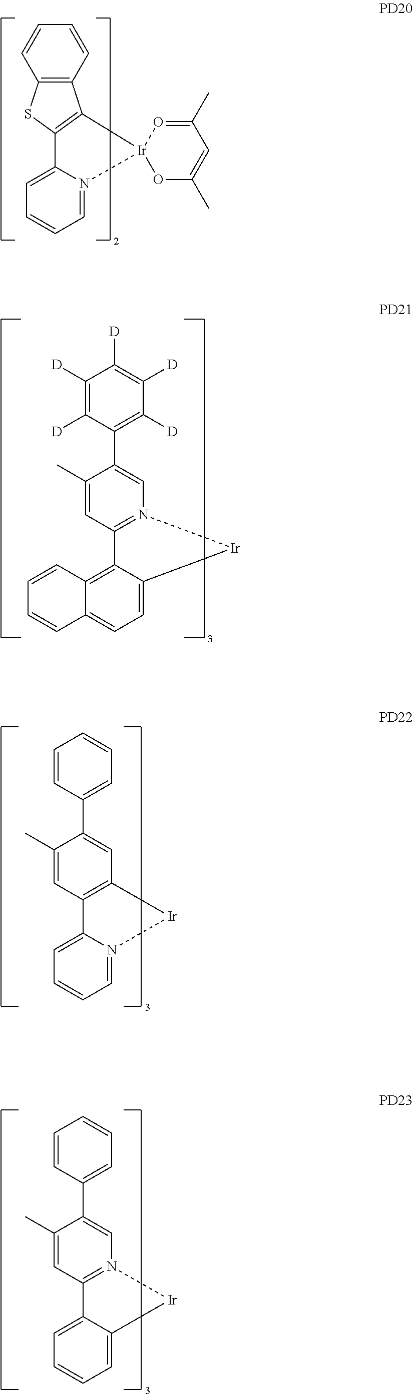

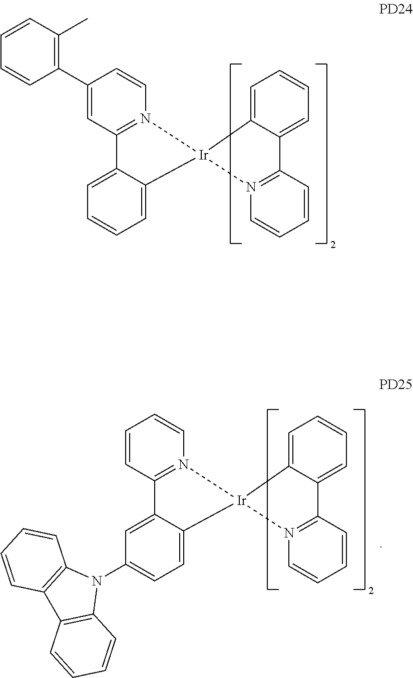

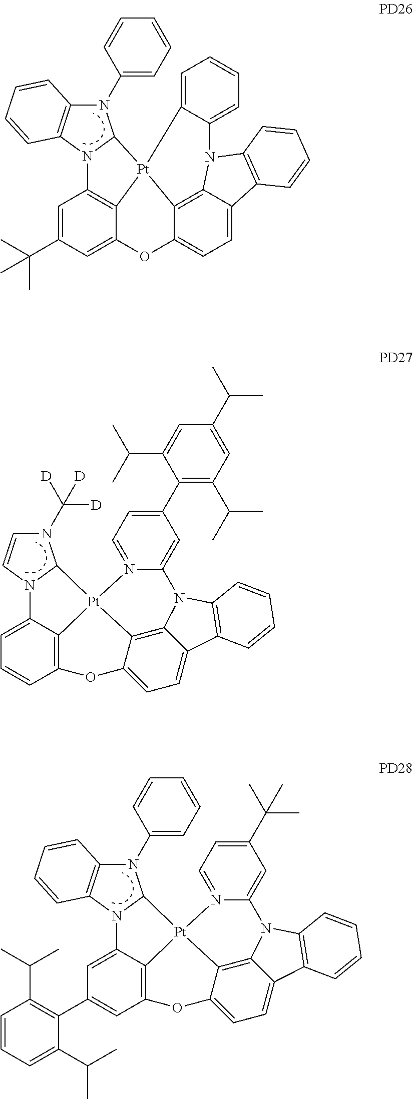

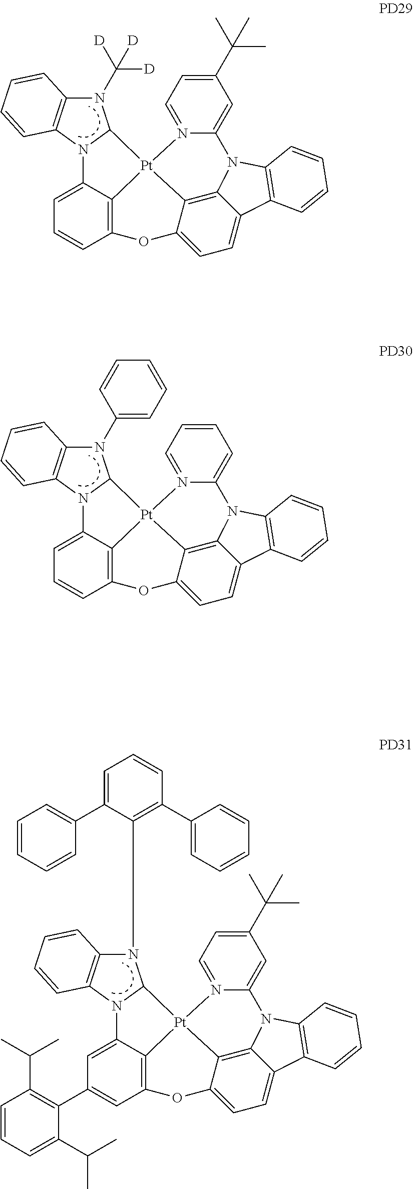

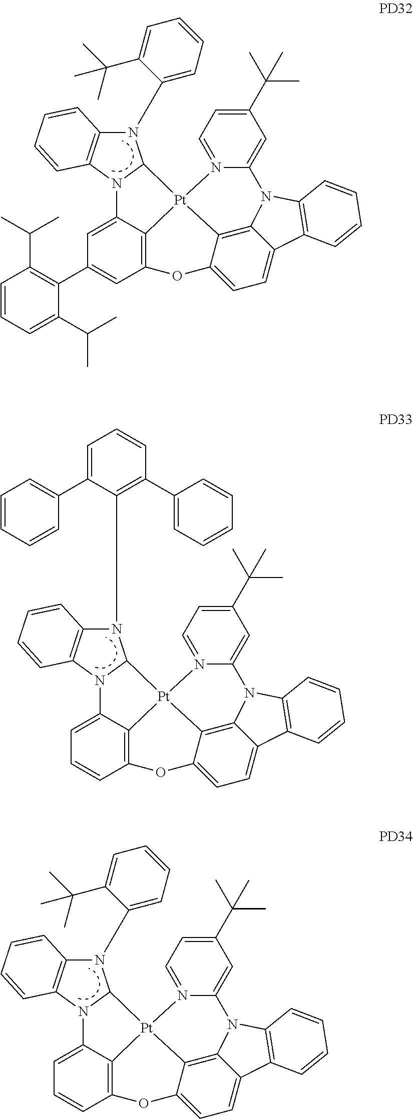

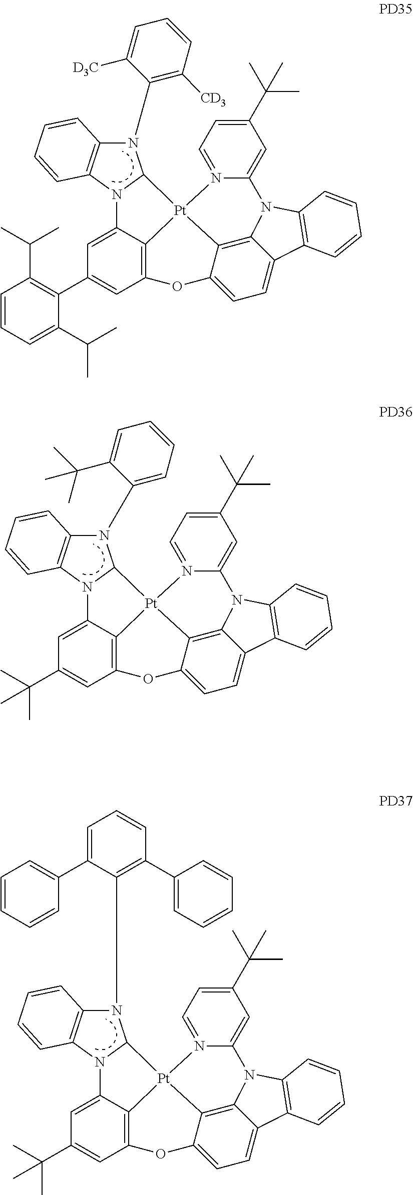

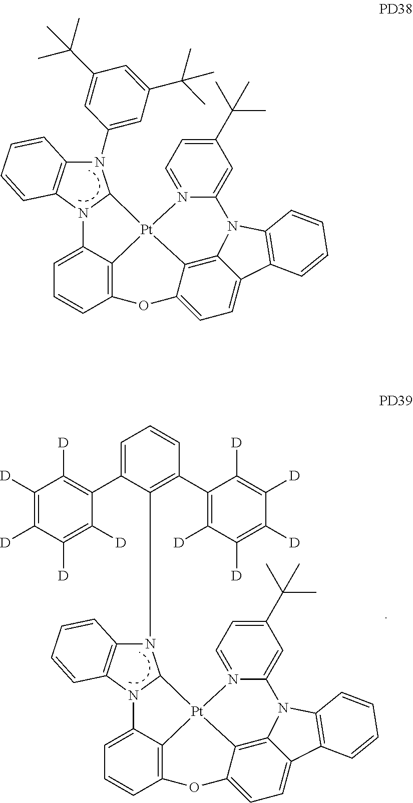

### Fluorescent Dopant

The fluorescent dopant may include an amine group-containing compound, a styryl group-containing compound, or any combination thereof.

For example, the fluorescent dopant may include a compound represented by Formula 501:

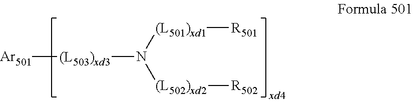

wherein, in Formula 501,
Ar₅₀₁, L₅₀₁ to L₅₀₃, R₅₀₁, and R₅₀₂ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,

xd1 to xd3 may each independently be 0, 1, 2, or 3, and

xd4 may be 1, 2, 3, 4, 5, or 6.

For example, Ar₅₀₁ in Formula 501 may be a condensed cyclic group (e.g., an anthracene group, a chrysene group, a pyrene group, etc.) in which three or more monocyclic groups are condensed together with each other.

In an embodiment, xd4 in Formula 501 may be 2.

In an embodiment, the fluorescent dopant may include: one of Compounds FD1 to FD37; DPVBi; DPAVBi; or any combination thereof:

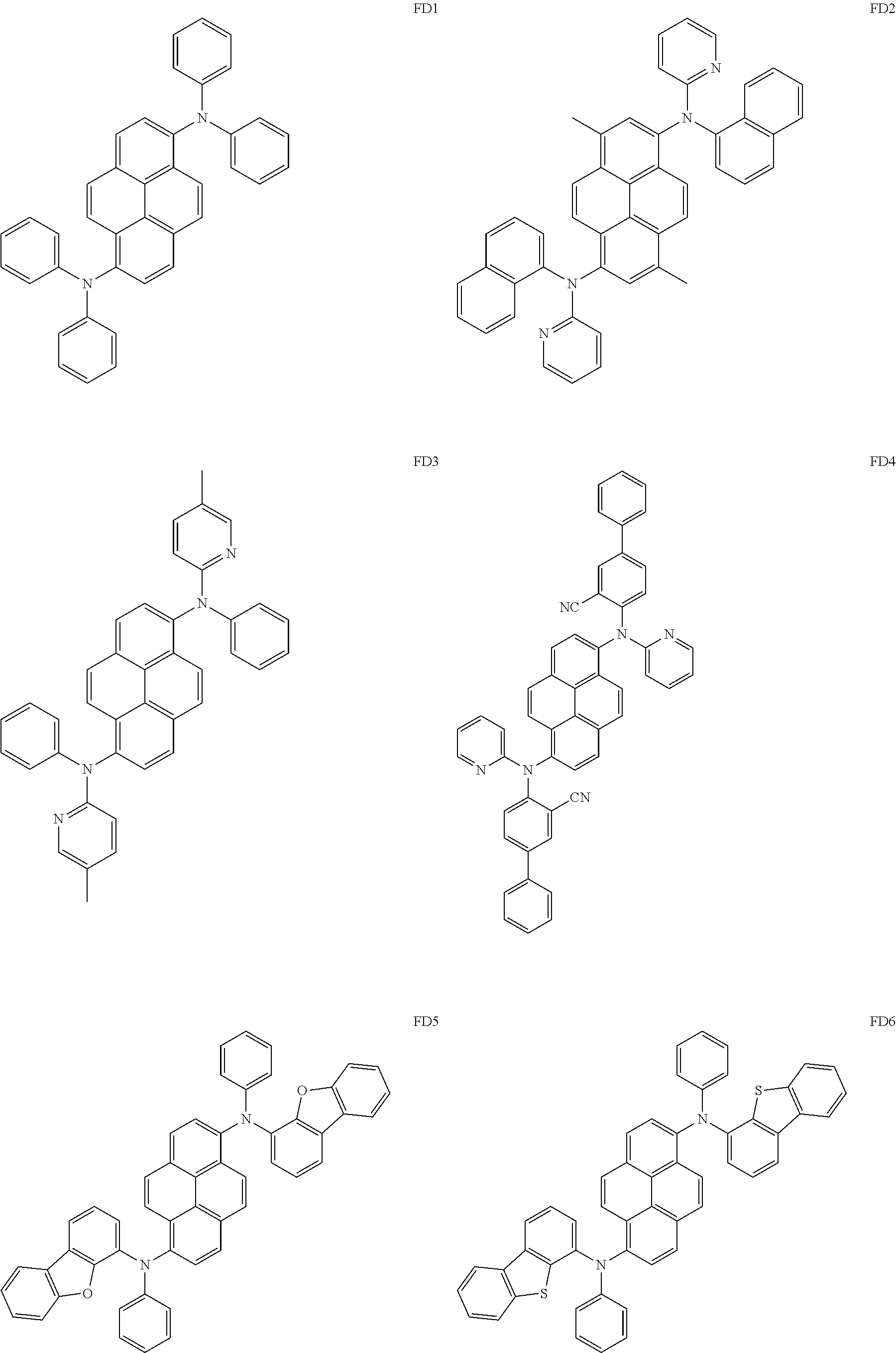

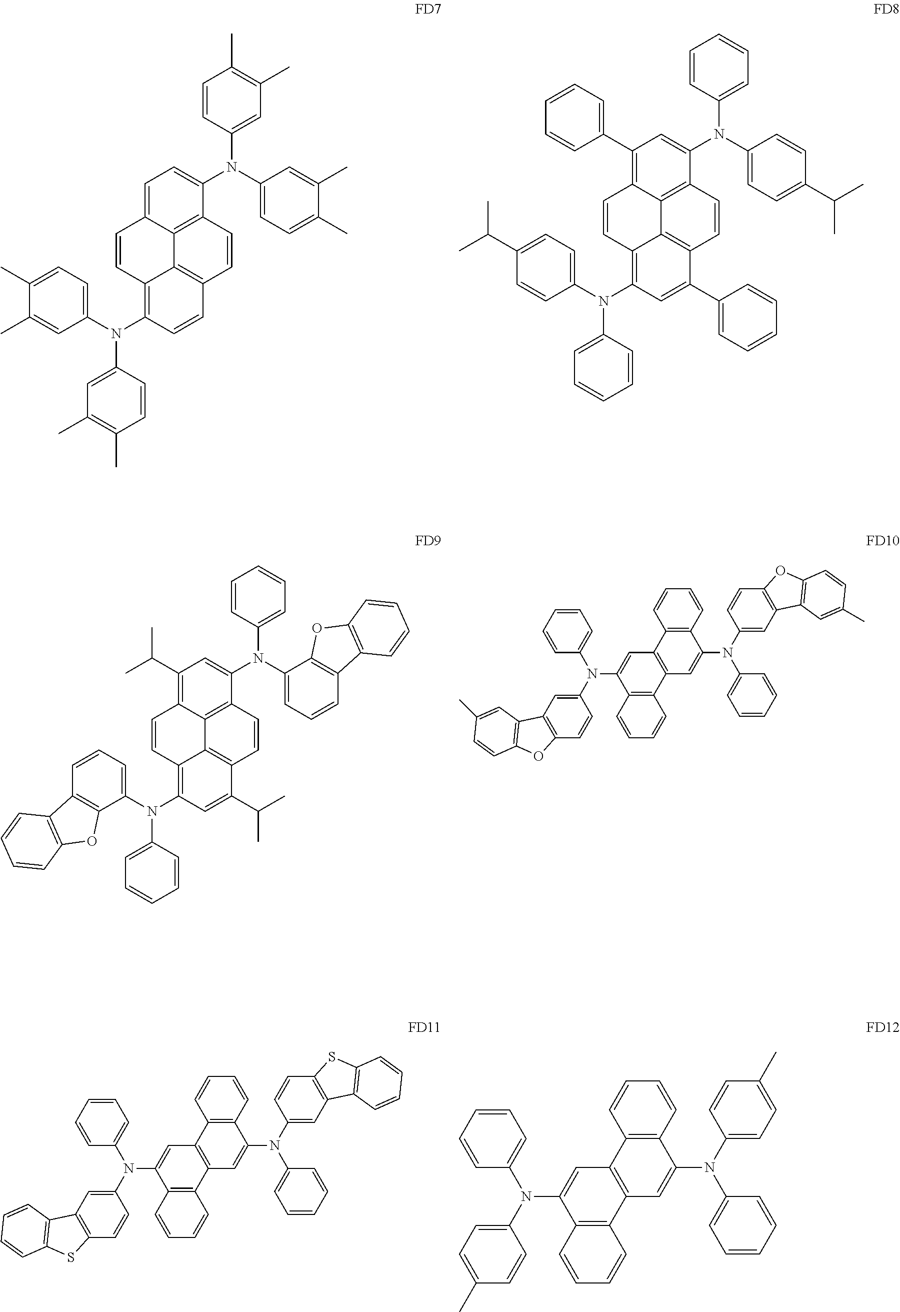

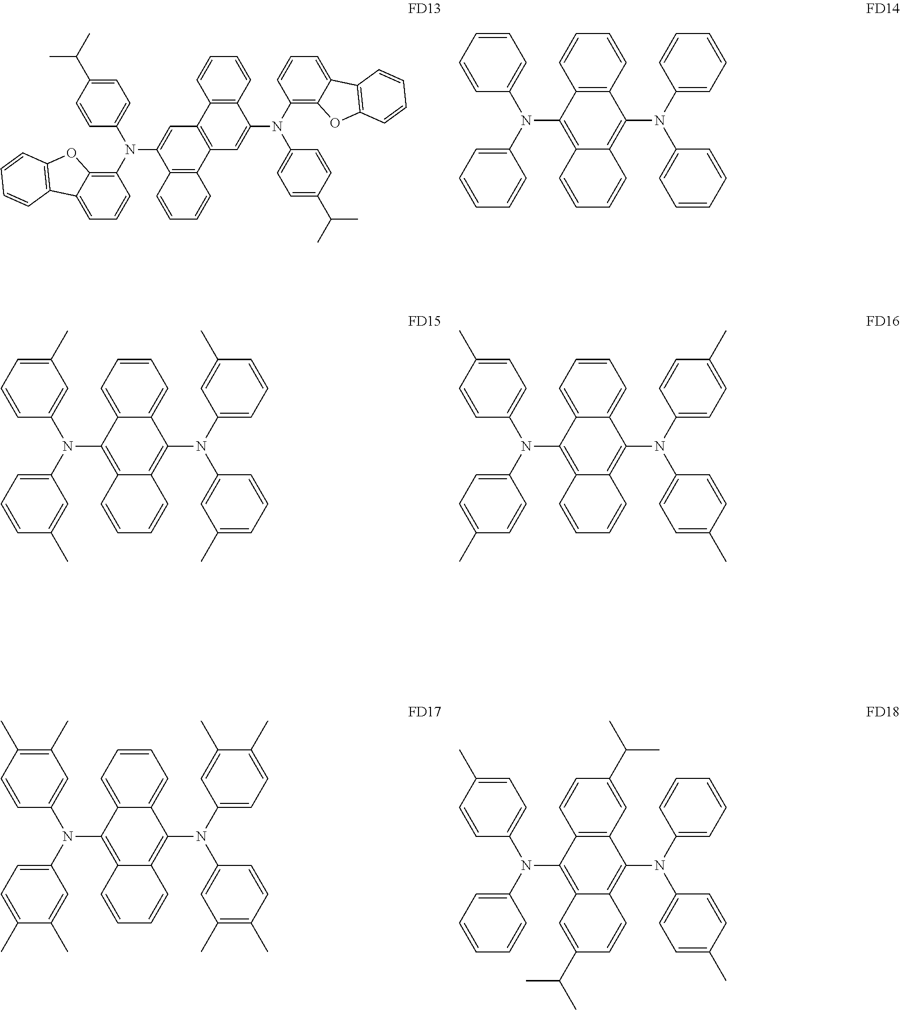 [SMILES: CC(C)c1ccc(N(c2cc3c4ccccc4c(N(c4ccc(C(C)C)cc4)c4cccc5c4oc4ccccc45)cc3c3ccccc23)c2cccc3c2oc2ccccc23)cc1 ; c1ccc(N(c2ccccc2)c2c3ccccc3c(N(c3ccccc3)c3ccccc3)c3ccccc23)cc1 ; Cc1cccc(N(c2cccc(C)c2)c2c3ccccc3c(N(c3cccc(C)c3)c3cccc(C)c3)c3ccccc23)c1 ; Cc1ccc(N(c2ccc(C)cc2)c2c3ccccc3c(N(c3ccc(C)cc3)c3ccc(C)cc3)c3ccccc23)cc1 ; Cc1ccc(N(c2ccc(C)c(C)c2)c2c3ccccc3c(N(c3ccc(C)c(C)c3)c3ccc(C)c(C)c3)c3ccccc23)cc1C ; Cc1ccc(N(c2ccccc2)c2c3ccc(C(C)C)cc3c(N(c3ccccc3)c3ccc(C)cc3)c3ccc(C(C)C)cc23)cc1]

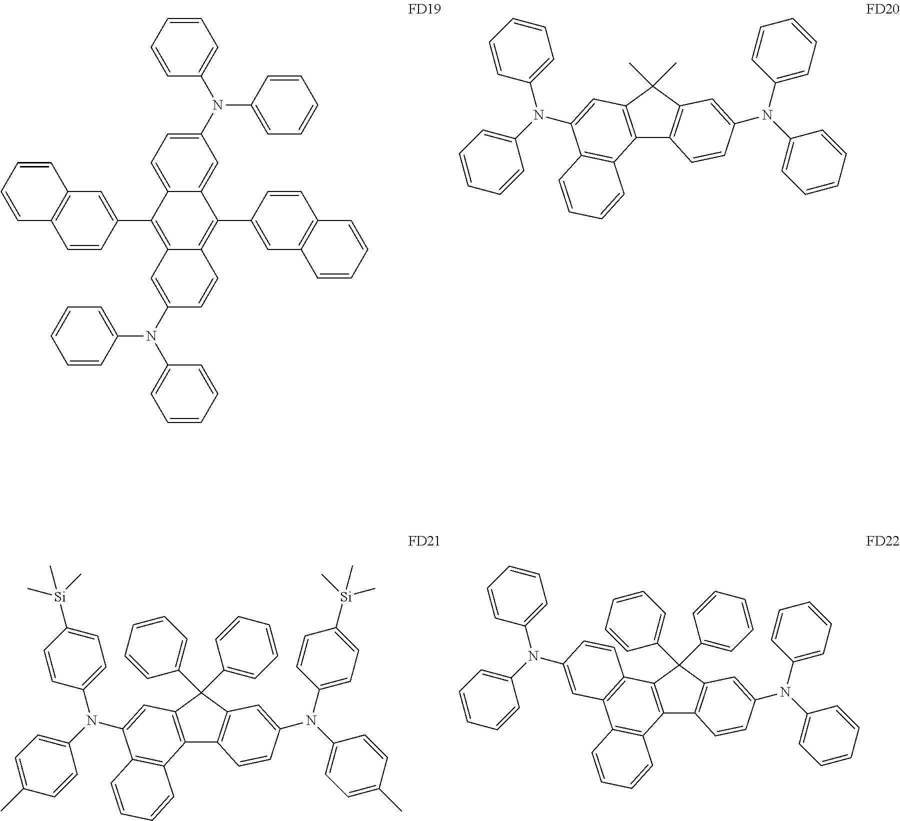

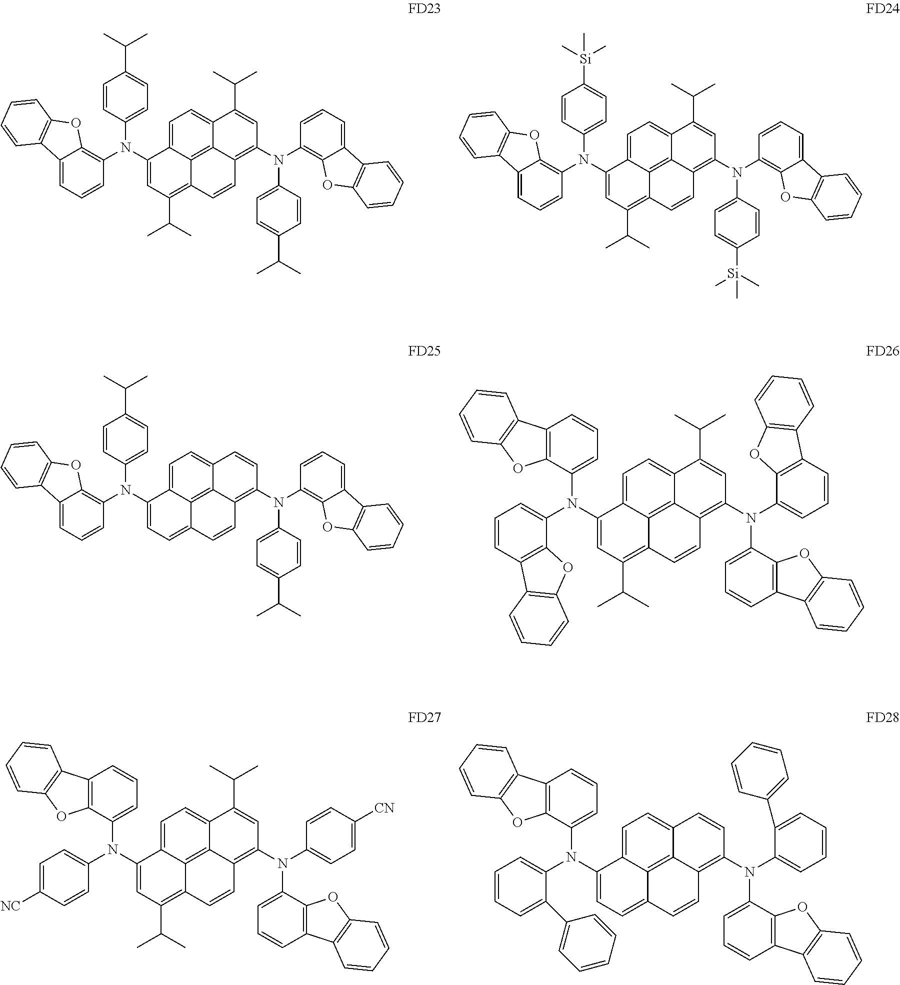

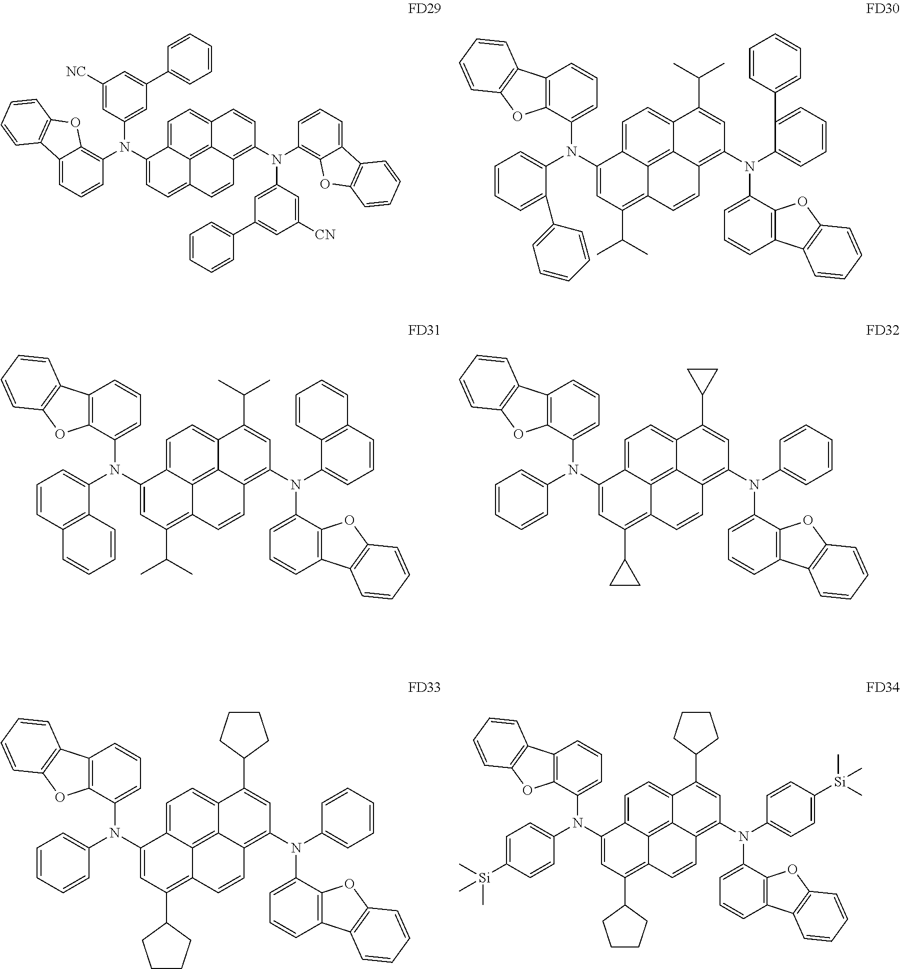

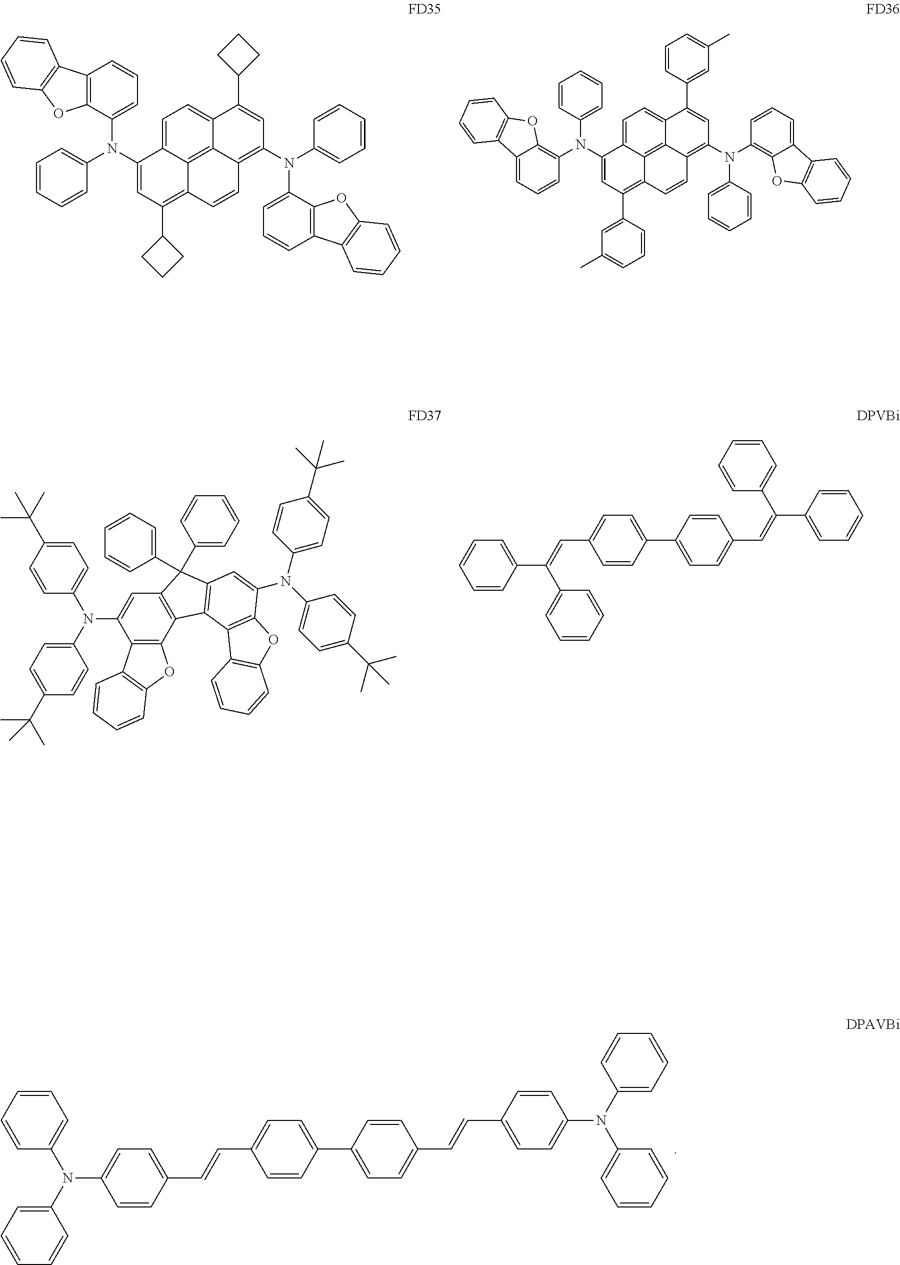

### Delayed Fluorescence Material

The emission layer may include a delayed fluorescence material.

In the present specification, the delayed fluorescence material may be selected from compounds capable of emitting delayed fluorescent light based on a delayed fluorescence emission mechanism.

The delayed fluorescence material included in the emission layer may act as a host or a dopant depending on the type or composition of other materials included in the emission layer.

In an embodiment, a difference between a triplet energy level (eV) of the delayed fluorescence material and a singlet energy level (eV) of the delayed fluorescence material may be about 0 eV or greater and about 0.5 eV or less. When the difference between the triplet energy level (eV) of the delayed fluorescence material and the singlet energy level (eV) of the delayed fluorescence material is within this range, up-conversion from the triplet state to the singlet state of the delayed fluorescence material may effectively occur, and thus, the luminescence efficiency of the light-emitting device 10 may be improved.

For example, the delayed fluorescence material may include i) a material including at least one electron donor (e.g., a π electron-rich C₃-C₆₀ cyclic group, such as a carbazole group, etc.) and at least one electron acceptor (e.g., a sulfoxide group, a cyano group, a π electron-deficient nitrogen-containing C₁-C₆₀ cyclic group, etc.), and ii) a material including a C₈-C₆₀ polycyclic group in which two or more cyclic groups are condensed together while sharing boron (B).

Examples of the delayed fluorescence material may include at least one of Compounds DF1 to DF14:

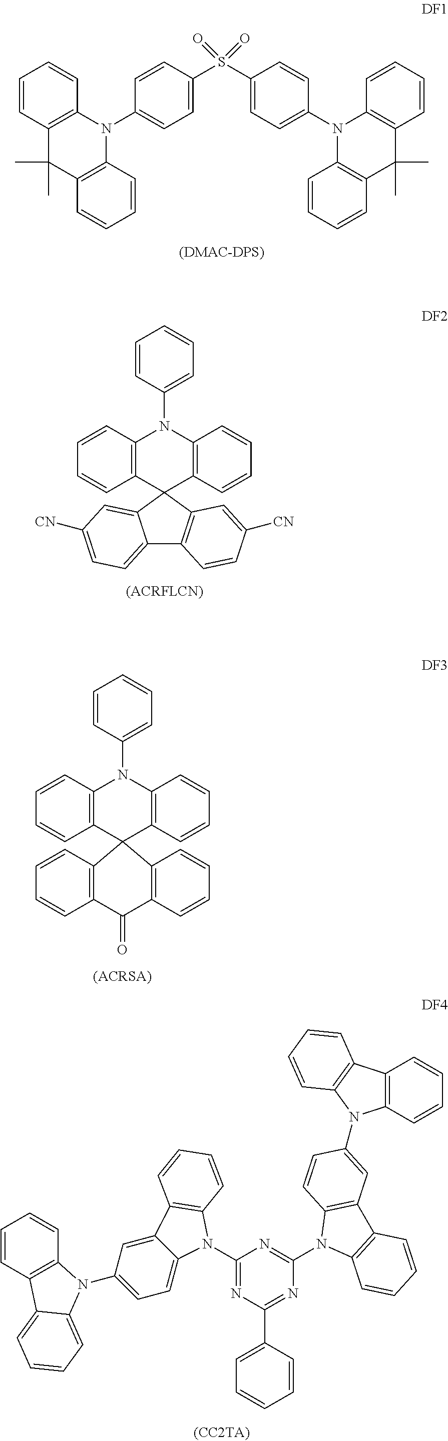

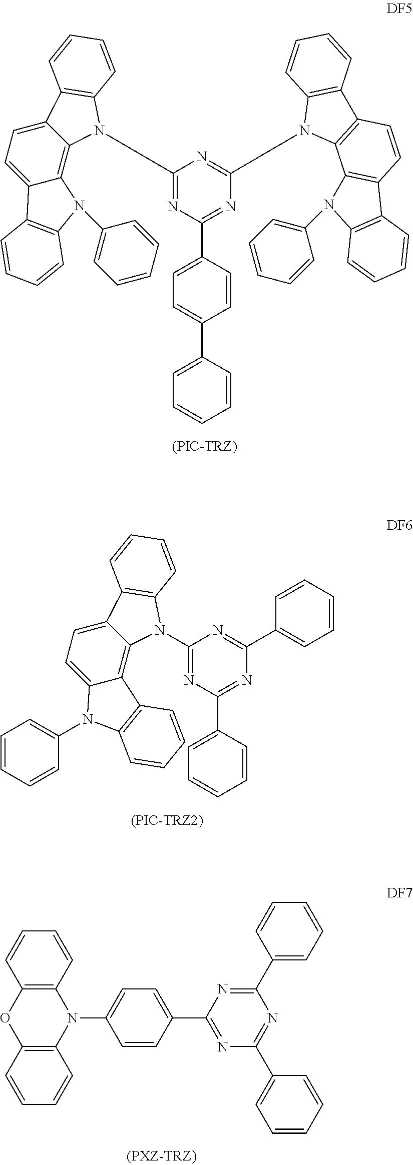 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-n3c4ccccc4c4ccc5c(c6ccccc6n5-c5ccccc5)c43)n2)cc1 ; c1ccc(-c2nc(-c3ccccc3)nc(-c3ccc(N4c5ccccc5Oc5ccccc54)cc3)n2)cc1]

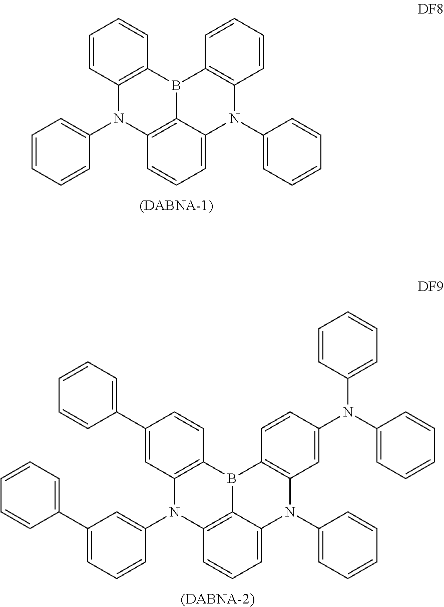

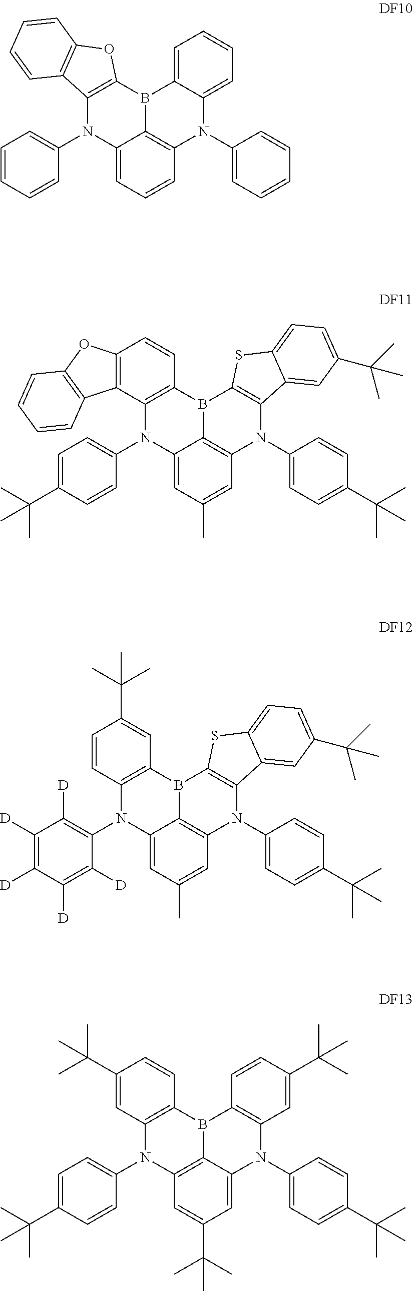

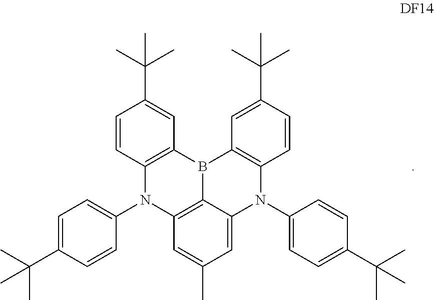 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

### Quantum Dot

The emission layer may include a quantum dot.

The term “quantum dot,” as used herein, refers to a crystal of a semiconductor compound, and may include any suitable material capable of emitting light of various suitable emission wavelengths according to the size of the crystal.

A diameter of the quantum dot may be, for example, in a range of about 1 nm to about 10 nm.

The quantum dot may be synthesized by a wet chemical process, a metal organic chemical vapor deposition process, a molecular beam epitaxy process, and/or any suitable process similar thereto.

The wet chemical process is a method including mixing a precursor material together with an organic solvent and then growing a quantum dot particle crystal. When the crystal grows, the organic solvent naturally acts as a dispersant coordinated on the surface of the quantum dot crystal and controls the growth of the crystal so that the growth of quantum dot particles can be controlled through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, and/or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, and/or MgZnS; a quaternary compound, such as CdZnSeS, CdZnSeTe, CdZnSTe, CdHgSeS, CdHgSeTe, CdHgSTe, HgZnSeS, HgZnSeTe, and/or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, and/or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, and/or InPSb; a quaternary compound, such as GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, and/or InAlPSb; or any combination thereof. In an embodiment, the Group III-V semiconductor compound may further include a Group II element. Examples of the Group III-V semiconductor compound further including a Group II element may include InZnP, InGaZnP, InAIZnP, and the like.

Examples of the Group III-VI semiconductor compound may include: a binary compound, such as GaS, GaSe, Ga2Se₃, GaTe, InS, InSe, In₂S₃, In₂Se₃, and/or InTe; a ternary compound, such as InGaS₃, and/or InGaSe₃; or any combination thereof.

Examples of the Group I-III-VI semiconductor compound may include: a ternary compound, such as AgInS, AgInS₂, CuInS, CuInS₂, CuGaO₂, AgGaO₂, and/or AgAlO₂; or any combination thereof.

Examples of the Group IV-VI semiconductor compound may include: a binary compound, such as SnS, SnSe, SnTe, PbS, PbSe, and/or PbTe; a ternary compound, such as SnSeS, SnSeTe, SnSTe, PbSeS, PbSeTe, PbSTe, SnPbS, SnPbSe, and/or SnPbTe; a quaternary compound, such as SnPbSSe, SnPbSeTe, and/or SnPbSTe; or any combination thereof.

The Group IV element or compound may include: a single element, such as Si or Ge; a binary compound, such as SiC and/or SiGe; or any combination thereof.

Each element included in a multi-element compound such as the binary compound, the ternary compound, and the quaternary compound may be present at a uniform concentration or non-uniform concentration in a particle.

In an embodiment, the quantum dot may have a single structure in which the concentration of each element in the quantum dot is uniform (e.g., substantially uniform), or a core-shell dual structure. For example, a material included in the core and a material included in the shell may be different from each other.

The shell of the quantum dot may act as a protective layer that prevents or reduces chemical degeneration of the core to maintain semiconductor characteristics, and/or as a charging layer that imparts electrophoretic characteristics to the quantum dot. The shell may be a single layer or a multi-layer. The interface between the core and the shell may have a concentration gradient in which the concentration of an element existing in the shell decreases along a direction toward the center of the core.

Examples of the shell of the quantum dot may include an oxide of metal, metalloid, and/or non-metal, a semiconductor compound, or any combination thereof. Examples of the oxide of metal, metalloid, and/or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, and/or NiO; a ternary compound, such as MgAl₂O₄, CoFe₂O₄, NiFe₂O₄, and/or CoMn₂O₄; or any combination thereof. Examples of the semiconductor compound may include, as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, or any combination thereof.

A full width at half maximum (FWHM) of an emission wavelength spectrum of the quantum dot may be about 45 nm or less, for example, about 40 nm or less, for example, about 30 nm or less, and within these ranges, color purity and/or color reproducibility may be improved. In addition, because the light emitted through the quantum dot is emitted in all (e.g., substantially all) directions, the viewing angle of light may be improved.

In addition, the quantum dot may be in the form of a spherical particle, a pyramidal particle, a multi-arm particle, a cubic nanoparticle, a nanotube particle, a nanowire particle, a nanofiber particle, and/or a nanoplate particle.

Because the energy band gap may be adjusted by controlling the size of the quantum dot, light having various suitable wavelength bands may be obtained from the quantum dot emission layer. Accordingly, by using quantum dots of different sizes, a light-emitting device that emits light of various suitable wavelengths may be implemented. In more detail, the size of the quantum dot may be selected to emit red, green and/or blue light. In addition, the size of the quantum dot may be configured to emit white light by combination of light of various suitable colors.

### Electron Transport Region in Interlayer 130

The electron transport region may have i) a single-layered structure consisting of a single layer consisting of a single material, ii) a single-layered structure consisting of a single layer including a plurality of different materials, or iii) a multi-layered structure including a plurality of layers including different materials.

The electron transport region may include a buffer layer, a hole blocking layer, an electron control layer, an electron transport layer, an electron injection layer, or any combination thereof.

For example, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, the layers of each structure being sequentially stacked from the emission layer.

The electron transport region (e.g., the buffer layer, the hole blocking layer, the electron control layer, and/or the electron transport layer in the electron transport region) may include a metal-free compound including at least one π electron-deficient nitrogen-containing C₁-C₆₀ cyclic group.

For example, the electron transport region may include a compound represented by Formula 601:

[Ar₆₀₁]ₓₑ₁₁-[(L₆₀₁)ₓₑ₁-R₆₀₁]ₓₑ₂₁ Formula 601

wherein, in Formula 601,
Ar₆₀₁ and L₆₀₁ may each independently be a C₃-C₆₀ carbocyclic group unsubstituted or substituted with at least one R₁₀ₐ or a C₁-C₆₀ heterocyclic group unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group unsubstituted or substituted with at least one R₁₀ₐ, a C₁-C₆₀ heterocyclic group unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ are each the same as described in connection with Q₁,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group unsubstituted or substituted with at least one R₁₀ₐ.

For example, when xe11 in Formula 601 is 2 or more, two or more of Ar₆₀₁(s) may be linked to each other via a single bond.

In an embodiment, Ar₆₀₁ in Formula 601 may be a substituted or unsubstituted anthracene group.

In an embodiment, the electron transport region may include a compound represented by Formula 601-1:

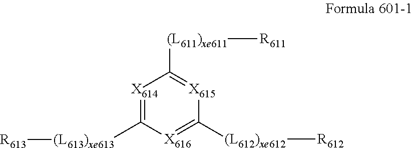

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ are each the same as described in connection with L₆₀₁,
xe611 to xe613 are each the same as described in connection with xe1,
R₆₁₁ to R₆₁₃ are each the same as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group unsubstituted or substituted with at least one R₁₀ₐ, or a C₁-C₆₀ heterocyclic group unsubstituted or substituted with at least one R₁₀ₐ.

For example, xe1 and xe611 to xe613 in Formulae 601 and 601-1 may each independently be 0, 1, or 2.

The electron transport region may include one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

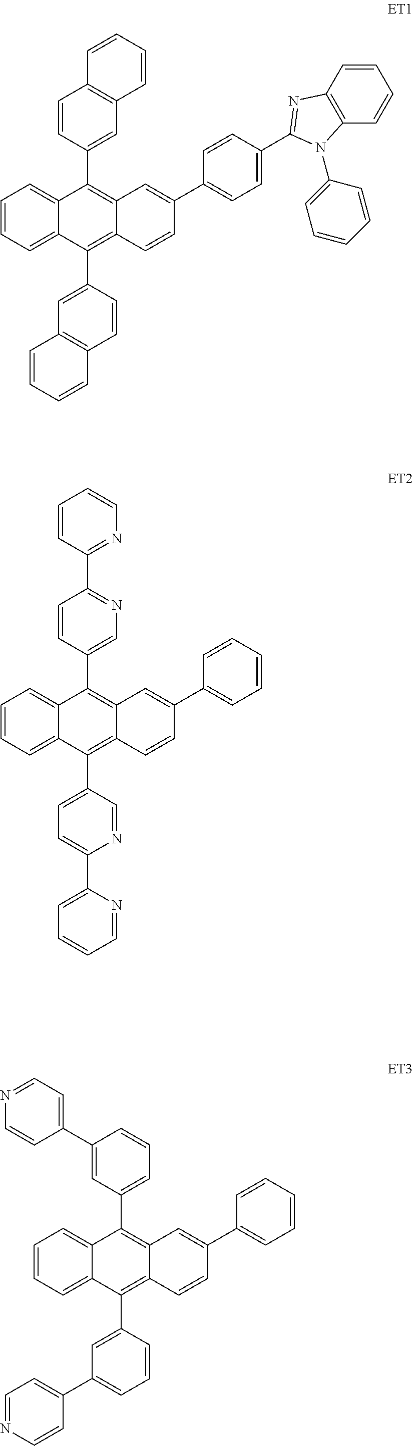 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

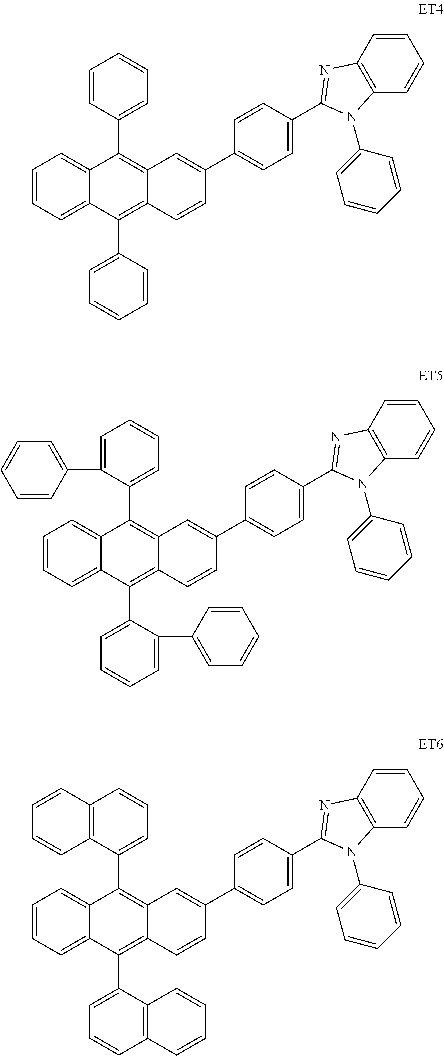 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

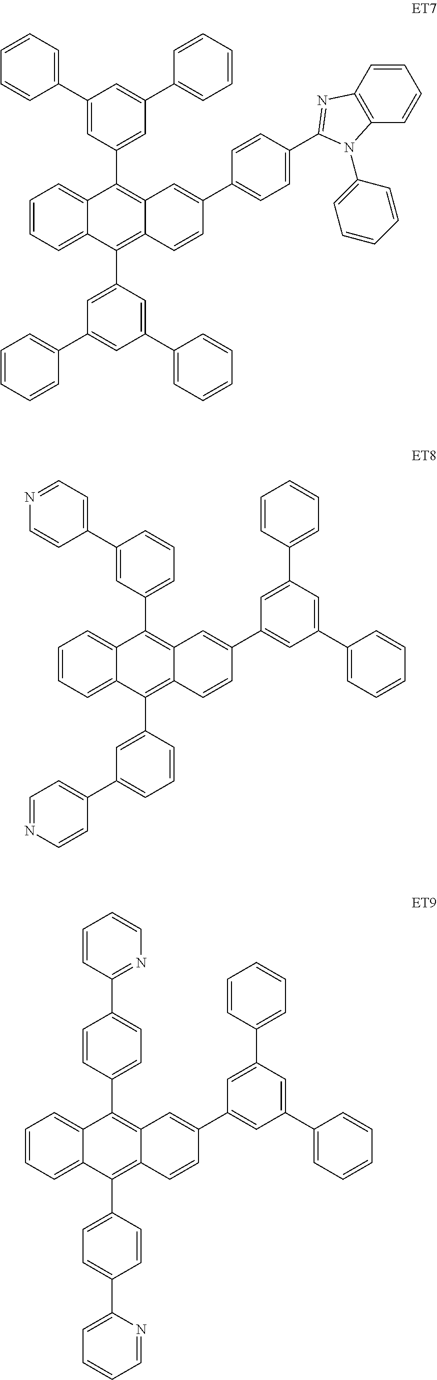 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccncc6)c5)c5ccccc5c(-c5cccc(-c6ccncc6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

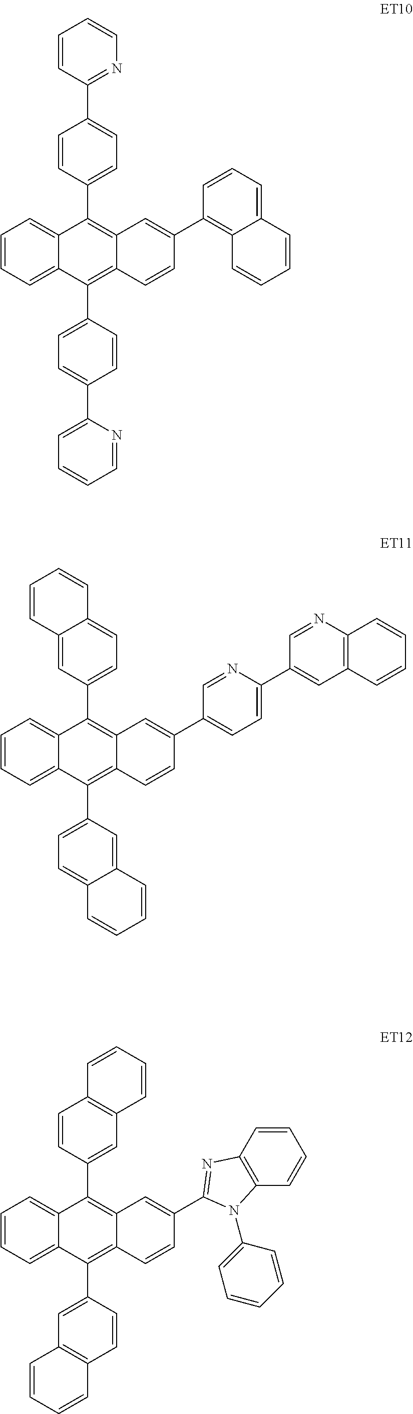 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

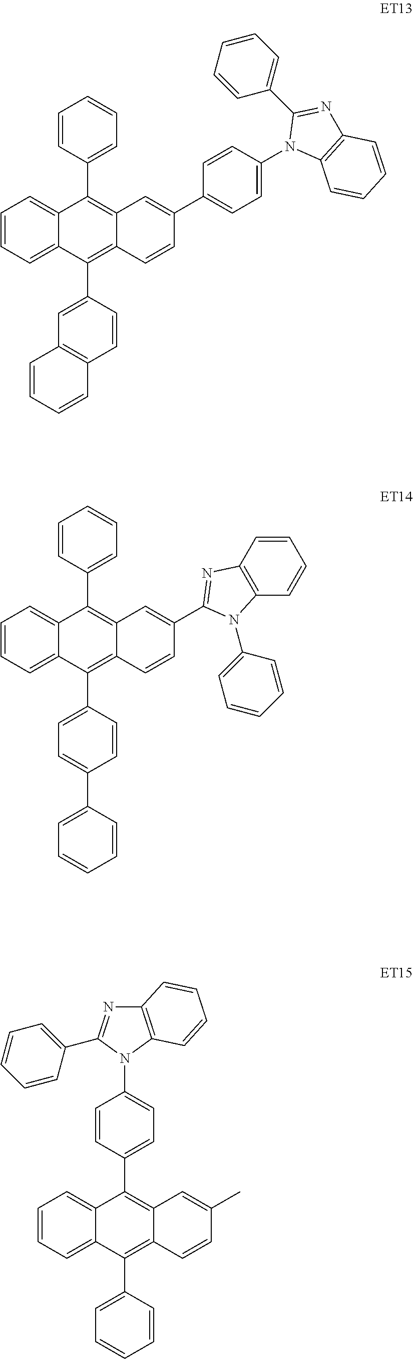 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

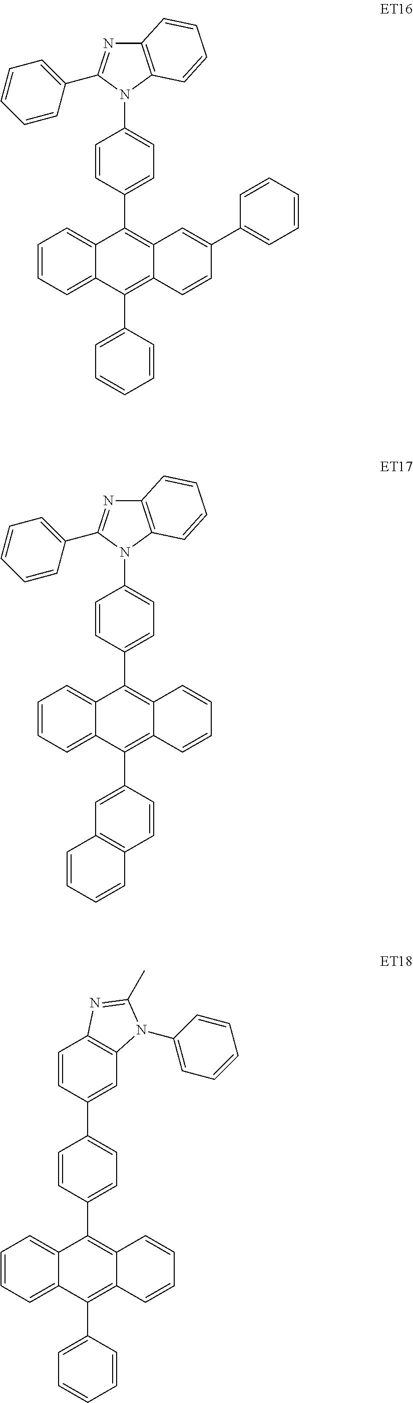 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

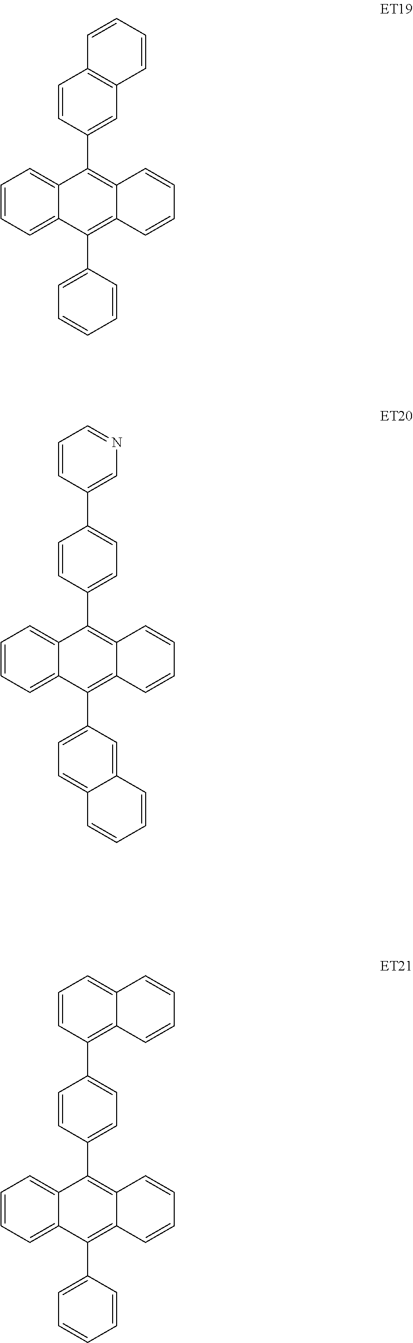

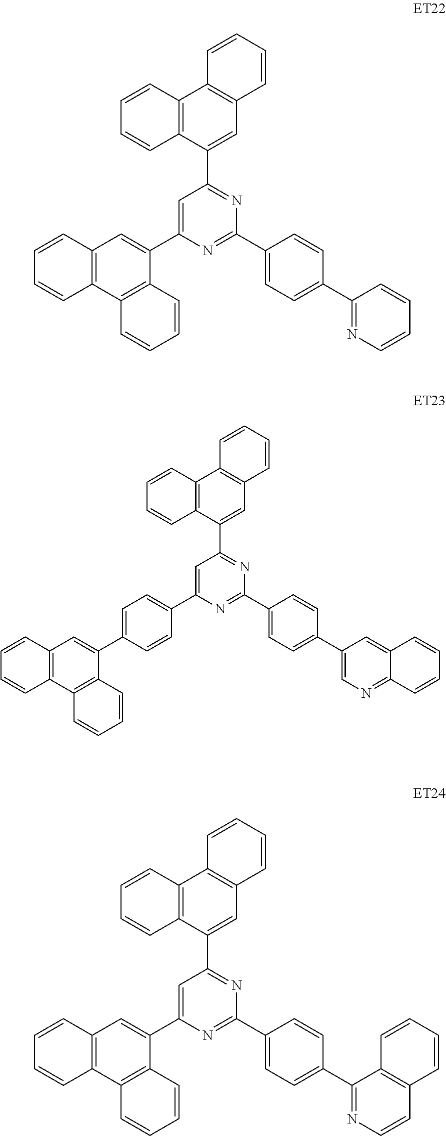 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

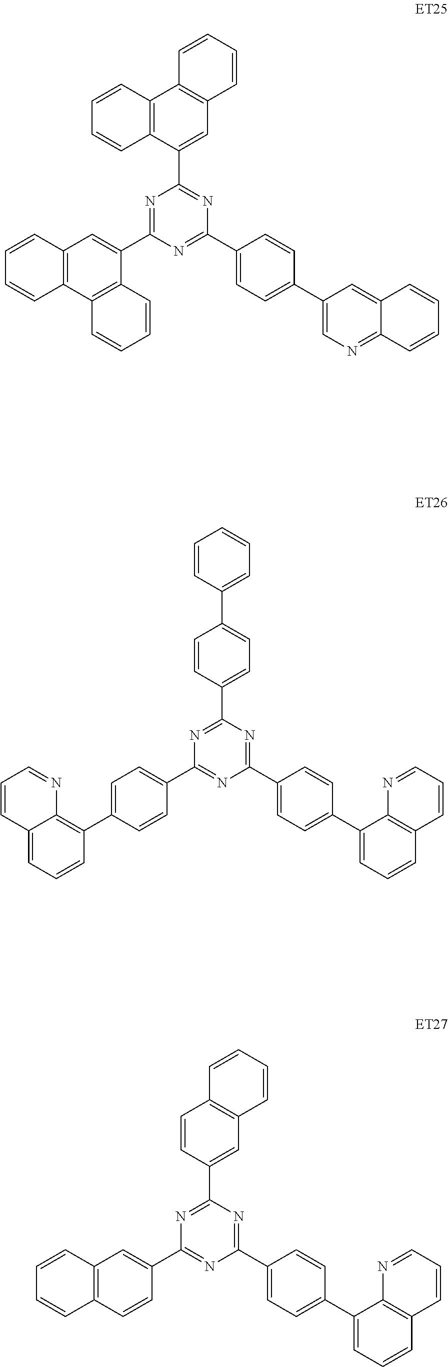 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

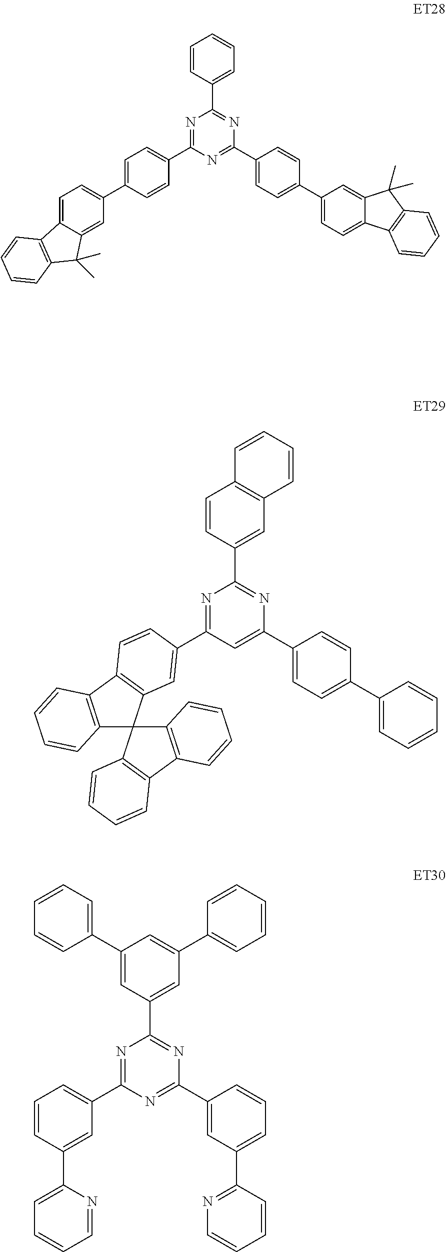 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

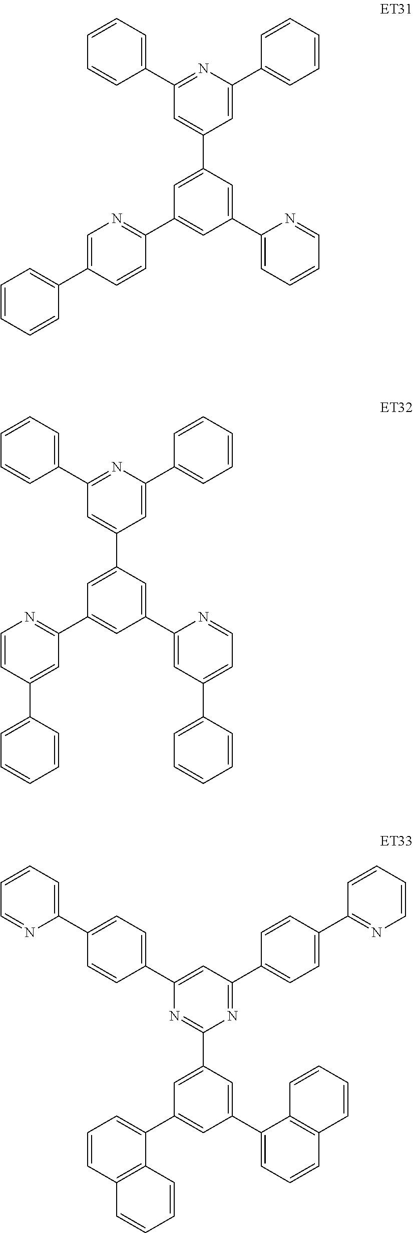 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

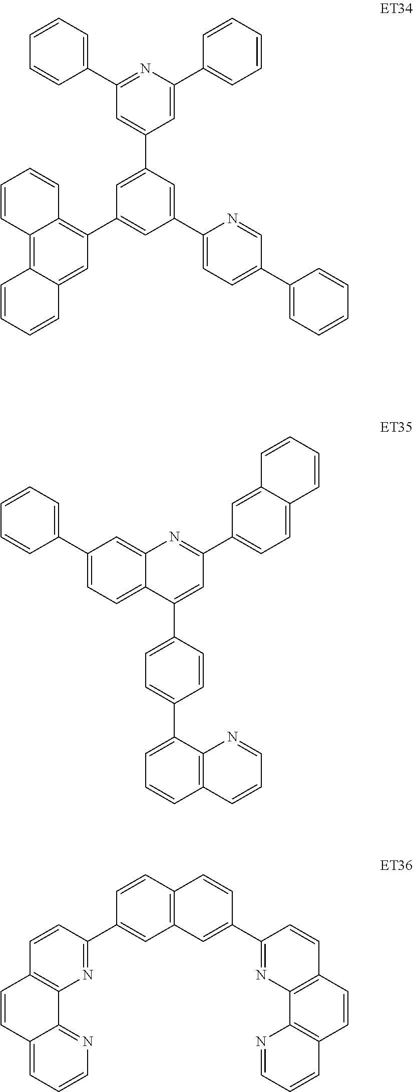 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

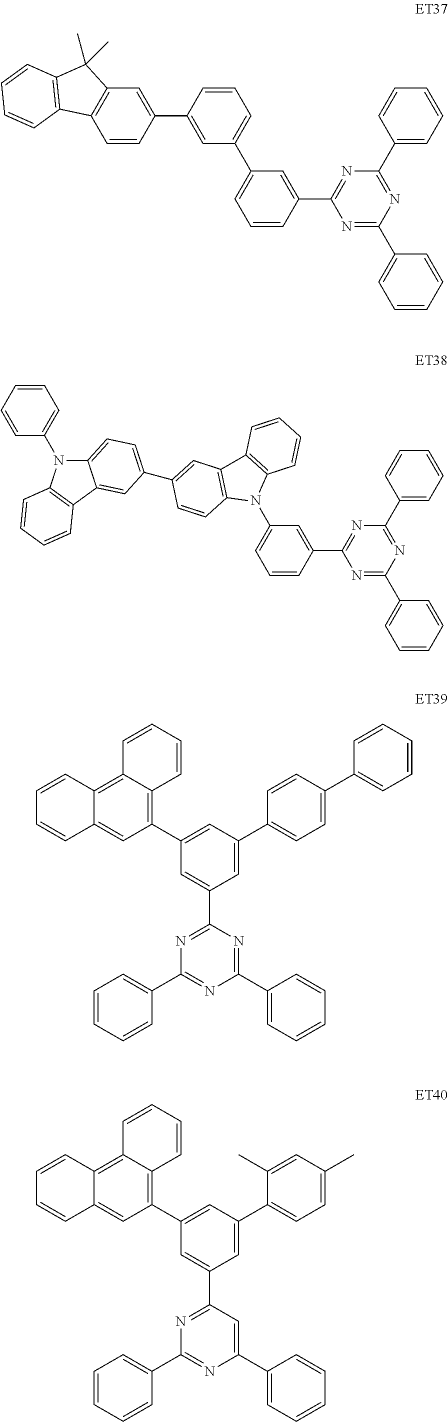 [SMILES: CC1(C)c2ccccc2-c2ccc(-c3cccc(-c4cccc(-c5nc(-c6ccccc6)nc(-c6ccccc6)n5)c4)c3)cc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2ccc(-c3cc(-c4nc(-c5ccccc5)nc(-c5ccccc5)n4)cc(-c4cc5ccccc5c5ccccc45)c3)cc2)cc1 ; Cc1ccc(-c2cc(-c3cc(-c4ccccc4)nc(-c4ccccc4)n3)cc(-c3cc4ccccc4c4ccccc34)c2)c(C)c1]

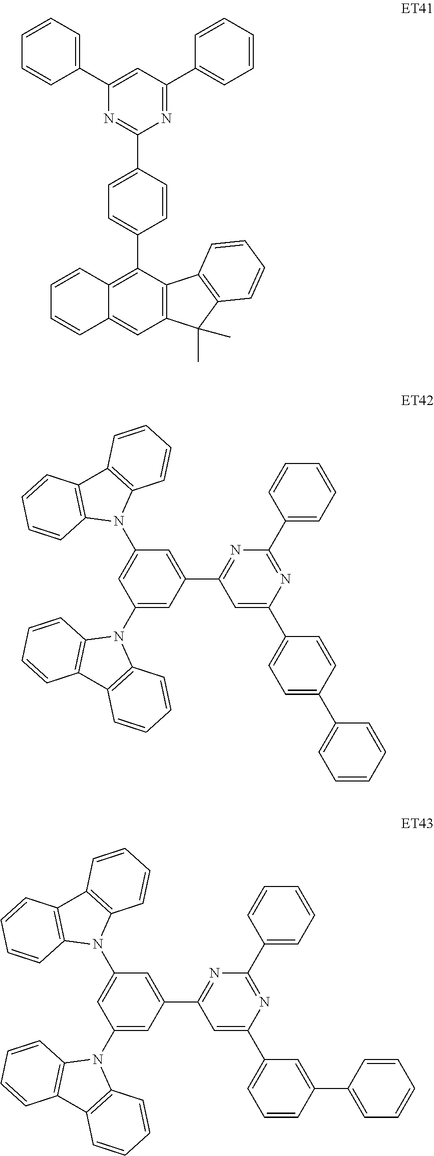 [SMILES: CC1(C)c2ccccc2-c2c1cc1ccccc1c2-c1ccc(-c2nc(-c3ccccc3)cc(-c3ccccc3)n2)cc1 ; c1ccc(-c2ccc(-c3cc(-c4cc(-n5c6ccccc6c6ccccc65)cc(-n5c6ccccc6c6ccccc65)c4)nc(-c4ccccc4)n3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

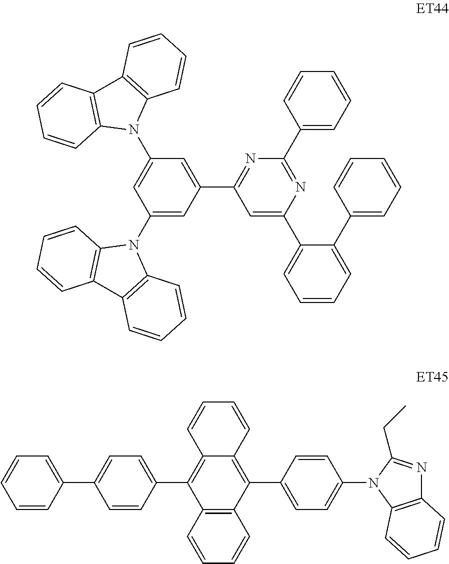 [SMILES: c1ccc(-c2nc(-c3cc(-n4c5ccccc5c5ccccc54)cc(-n4c5ccccc5c5ccccc54)c3)cc(-c3ccccc3-c3ccccc3)n2)cc1 ; CCc1nc2ccccc2n1-c1ccc(-c2c3ccccc3c(-c3ccc(-c4ccccc4)cc3)c3ccccc23)cc1]

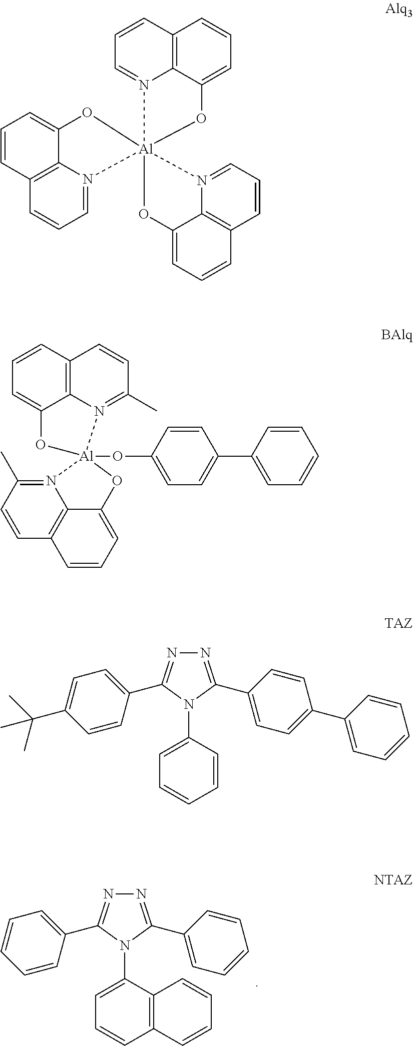

A thickness of the electron transport region may be in a range of about 100 Å to about 5,000 Å, for example, about 160 Å to about 4,000 Å. When the electron transport region includes a buffer layer, a hole blocking layer, an electron control layer, an electron transport layer, or any combination thereof, a thickness of the buffer layer, the hole blocking layer, or the electron control layer may each independently be in a range of about 20 Å to about 1,000 Å, for example, about 30 Å to about 300 Å, and a thickness of the electron transport layer may be in a range of about 100 Å to about 1,000 Å, for example, about 150 Å to about 500 Å. When the thicknesses of the buffer layer, the hole blocking layer, the electron control layer, the electron transport layer, and/or the electron transport region are within these ranges, suitable or satisfactory electron transporting characteristics may be obtained without a substantial increase in driving voltage.

The electron transport region (e.g., the electron transport layer in the electron transport region) may further include, in addition to the materials described above, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenylbenzimidazole, hydroxyphenylbenzothiazole, bipyridine, phenanthroline, cyclopentadiene, or any combination thereof.

For example, the metal-containing material may include a Li complex. The Li complex may include, for example, Compound ET-D1 (LiQ) and/or ET-D2:

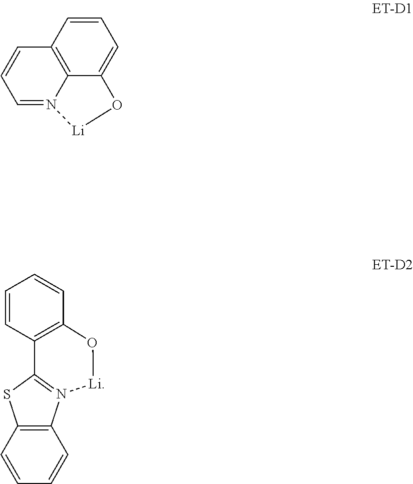

The electron transport region may include an electron injection layer that facilitates injection of electrons from the second electrode 150. The electron injection layer may be in direct contact (e.g., physical contact) with the second electrode 150.

The electron injection layer may have i) a single-layered structure consisting of a single layer consisting of a single material, ii) a single-layered structure consisting of a single layer including a plurality of different materials, or iii) a multi-layered structure including a plurality of layers including different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may be oxides, halides (e.g., fluorides, chlorides, bromides, iodides, etc.), and/or tellurides of the alkali metal, the alkaline earth metal, and the rare earth metal, or any combination thereof.

The alkali metal-containing compound may include: alkali metal oxide, such as Li₂O, Cs₂O, and/or K₂O; alkali metal halide, such as LiF, NaF, CsF, KF, LiI, NaI, CsI, and/or KI; or any combination thereof. The alkaline earth metal-containing compound may include an alkaline earth metal compound, such as BaO, SrO, CaO, BaxSr₁₋ₓO (wherein x is a real number satisfying the condition of 0<x<1), and/or BaxCa₁₋ₓO (wherein x is a real number satisfying the condition of 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In an embodiment, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, Lu₂Te₃, and the like.

The alkali metal complex, the alkaline earth-metal complex, and the rare earth metal complex may include i) one of ions of the alkali metal, the alkaline earth metal, and the rare earth metal and ii), as a ligand linked to the metal ion, for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described above. In an embodiment, the electron injection layer may further include an organic material (e.g., a compound represented by Formula 601).

In an embodiment, the electron injection layer may include (or consist of) i) an alkali metal-containing compound (e.g., alkali metal halide), or ii) a) an alkali metal-containing compound (e.g., alkali metal halide); and b) an alkali metal, an alkaline earth metal, a rare earth metal, or any combination thereof. For example, the electron injection layer may be a KI:Yb co-deposited layer, a RbI:Yb co-deposited layer, a LiF:Yb co-deposited layer, and/or the like.

When the electron injection layer further includes an organic material, an alkali metal, an alkaline earth metal, a rare earth metal, an alkali metal-containing compound, an alkaline earth metal-containing compound, a rare earth metal-containing compound, an alkali metal complex, an alkaline earth-metal complex, a rare earth metal complex, or any combination thereof, which may be uniformly or non-uniformly dispersed in a matrix including the organic material.

A thickness of the electron injection layer may be in a range of about 1 Å to about 100 Å, for example, about 3 Å to about 90 Å. When the thickness of the electron injection layer is within this range, suitable or satisfactory electron injection characteristics may be obtained without a substantial increase in driving voltage.

### Second Electrode 150

The second electrode 150 may be on the interlayer 130. The second electrode 150 may be a cathode that is an electron injection electrode. A material for forming the second electrode 150 may include a metal, an alloy, an electrically conductive compound, or a combination thereof, each of which has a low work function.

The second electrode 150 may include lithium (Li), silver (Ag), magnesium (Mg), aluminum (Al), aluminum-lithium (Al—Li), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layered structure or a multi-layered structure including a plurality of layers.

### Capping Layer

A first capping layer may be outside the first electrode 110, and/or a second capping layer may be outside the second electrode 150. In more detail, the light-emitting device 10 may have a structure in which the first capping layer, the first electrode 110, the interlayer 130, and the second electrode 150 are sequentially stacked in the stated order, a structure in which the first electrode 110, the interlayer 130, the second electrode 150, and the second capping layer are sequentially stacked in the stated order, or a structure in which the first capping layer, the first electrode 110, the interlayer 130, the second electrode 150, and the second capping layer are sequentially stacked in the stated order.

Light generated by the emission layer in the interlayer 130 of the light-emitting device 10 may be extracted to the outside through the first electrode 110, which is a semi-transmissive electrode or a transmissive electrode, and the first capping layer. The light generated by the emission layer in the interlayer 130 of the light-emitting device 10 may be extracted to the outside through the second electrode 150, which is a semi-transmissive electrode or a transmissive electrode, and the second capping layer.

The first capping layer and the second capping layer may increase external luminescence efficiency according to the principle of constructive interference. Accordingly, the light extraction efficiency of the light-emitting device 10 may be increased, so that the luminescence efficiency of the light-emitting device 10 may be improved.

The first capping layer and the second capping layer may each include a material having a refractive index of about 1.6 or more (at a wavelength of 589 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may optionally be substituted with a substituent including O, N, S, Se, Si, F, Cl, Br, I, or any combination thereof. In an embodiment, at least one of the first capping layer and the second capping layer may each independently include an amine group-containing compound.

For example, at least one of the first capping layer and the second capping layer may each independently include the compound represented by Formula 201, the compound represented by Formula 202, or any combination thereof.

In an embodiment, at least one of the first capping layer and the second capping layer may each independently include one of Compounds HT28 to HT33, one of Compounds CP1 to CP6, p-NPB, or any combination thereof:

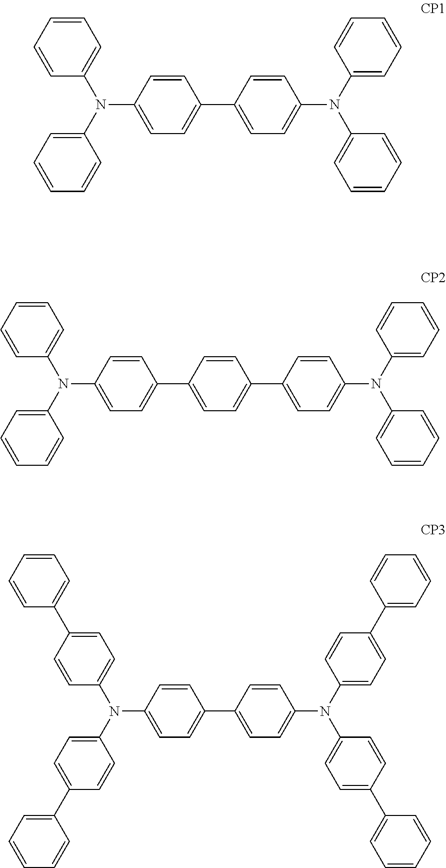 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

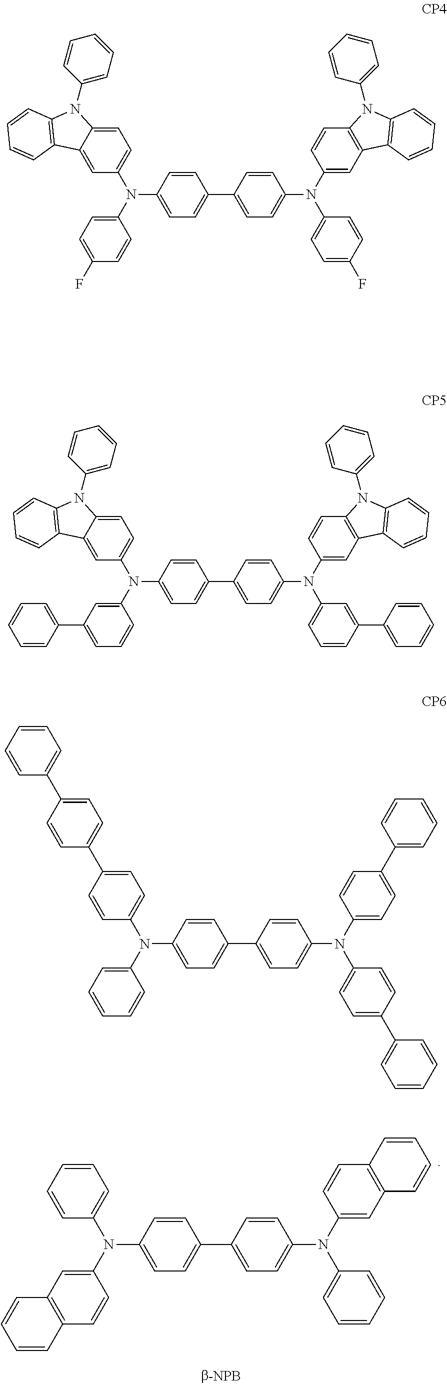 [SMILES: Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1 ; c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

A film may be an optical member (or a light control means) (e.g., a color filter, a color conversion member, a capping layer, a light extraction efficiency improvement layer, a selective light-absorbing layer, a polarizing layer, a quantum dot-containing layer, etc.), a light-blocking member (e.g., a light reflection layer, a light-absorbing layer, etc.), a protection member (e.g., an insulating layer, a dielectric material layer, etc.), and/or the like.

### Electronic Apparatus

The light-emitting device may be included in various suitable electronic apparatuses. For example, the electronic apparatus including the light-emitting device may be a light-emitting apparatus, an authentication apparatus, and/or the like.

The electronic apparatus (e.g., a light-emitting apparatus) may further include, in addition to the light-emitting device, i) a color filter, ii) a color conversion layer, or iii) a color filter and a color conversion layer. The color filter and/or the color conversion layer may be arranged in at least one direction in which light emitted from the light-emitting device travels. For example, the light emitted from the light-emitting device may be blue light and/or white light. The light-emitting device is the same as described above. In an embodiment, the color conversion layer may include a quantum dot. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel defining film may be arranged among the subpixel areas to define each of the subpixel areas.

The color filter may further include a plurality of color filter areas and light-shielding patterns arranged among the color filter areas, and the color conversion layer may include a plurality of color conversion areas and light-shielding patterns arranged among the color conversion areas.

The color filter areas (or the color conversion areas) may include: a first area that emits a first color light; a second area that emits a second color light; and/or a third area that emits a third color light. The first color light, the second color light, and/or the third color light may have different maximum emission wavelengths. For example, the first color light may be red light, the second color light may be green light, and the third color light may be blue light. For example, the color filter areas (or the color conversion areas) may include quantum dots. In more detail, the first area may include red quantum dots, the second area may include green quantum dots, and the third area may not include quantum dots. The quantum dots are the same as described herein. The first area, the second area, and/or the third area may each further include a scatterer (e.g., a light scatterer).

For example, the light-emitting device may emit a first light, the first area may absorb the first light to emit a first-first color light, the second area may absorb the first light to emit a second-first color light, and the third area may absorb the first light to emit a third-first color light. The first-first color light, the second-first color light, and the third-first color light may have different maximum emission wavelengths. In more detail, the first light may be blue light, the first-first color light may be red light, the second-first color light may be green light, and the third-first color light may be blue light.

The electronic apparatus may further include a thin-film transistor, in addition to the light-emitting device described above. The thin-film transistor may include a source electrode, a drain electrode, and an activation layer, wherein one of the source electrode and the drain electrode may be electrically connected to one of the first electrode and the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be between the color filter and/or the color conversion layer and the light-emitting device. The sealing portion allows light from the light-emitting device to be extracted to the outside, and concurrently (e.g., simultaneously) prevents or reduces penetration of ambient air and moisture into the light-emitting device. The sealing portion may be a sealing substrate including a transparent glass substrate and/or a plastic substrate. The sealing portion may be a thin-film encapsulation layer including at least one of an organic layer and/or an inorganic layer. When the sealing portion is a thin-film encapsulation layer, the electronic apparatus may be flexible.

Various suitable functional layers may be additionally on the sealing portion, in addition to the color filter and/or the color conversion layer, according to the use of the electronic apparatus. The functional layers may include a touch screen layer, a polarizing layer, and/or the like. The touch screen layer may be a pressure-sensitive touch screen layer, a capacitive touch screen layer, and/or an infrared touch screen layer. The authentication apparatus may be, for example, a biometric authentication apparatus that authenticates an individual by using biometric information of a living body (e.g., fingertips, pupils, etc.).

The authentication apparatus may further include, in addition to the light-emitting device described above, a biometric information collector.

The electronic apparatus may be applied to various suitable displays, light sources, lighting, personal computers (e.g., a mobile personal computer), mobile phones, digital cameras, electronic organizers, electronic dictionaries, electronic game machines, medical instruments (e.g., electronic thermometers, sphygmomanometers, blood glucose meters, pulse measurement devices, pulse wave measurement devices, electrocardiogram displays, ultrasonic diagnostic devices, and/or endoscope displays), fish finders, various suitable measuring instruments, meters (e.g., meters for a vehicle, an aircraft, and/or a vessel), projectors, and/or the like.

### Electronic Device

The light-emitting device may be included in various suitable electronic devices.

For example, the electronic device including the light-emitting device may be one of a flat panel display, a curved display, a computer monitor, a medical monitor, a television, a billboard, an indoor and/or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater and/or stadium screen, a phototherapy device, and/or a signboard.

The light-emitting device may have excellent effects in terms of luminescence efficiency and long lifespan, and thus, the electronic device including the light-emitting device may have characteristics, such as high luminance, high resolution, and low power consumption.

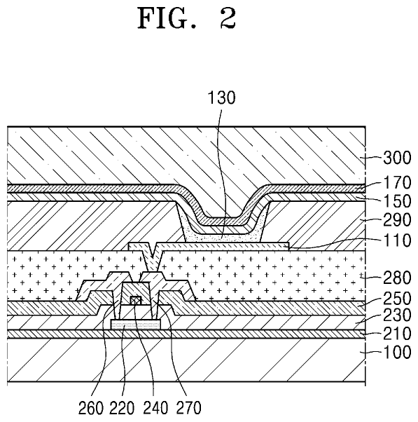

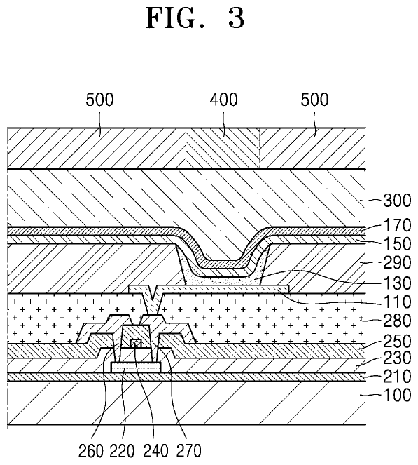

### Description of FIGS. 2 and 3

FIG. 2 is a schematic view of an electronic apparatus according to an embodiment.

The electronic apparatus (e.g., a light-emitting apparatus) of FIG. 2 may include a substrate 100, a thin-film transistor (TFT), a light-emitting device, and an encapsulation portion 300 that seals the light-emitting device.

The substrate 100 may be a flexible substrate, a glass substrate, and/or a metal substrate. A buffer layer 210 may be on the substrate 100. The buffer layer 210 may prevent or reduce penetration of impurities through the substrate 100. The buffer layer 210 may provide a flat surface on the substrate 100.

The TFT may be on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor. The activation layer 220 may include a source area, a drain area, and a channel area.

A gate insulating film 230 may be on the activation layer 220. The gate insulating film 230 may electrically insulate between the activation layer 220 and the gate electrode 240.

The gate electrode 240 may be on the gate insulating film 230.

An interlayer insulating film 250 may be on the gate electrode 240. The interlayer insulating film 250 may be between the gate electrode 240 and the source electrode 260 and between the gate electrode 240 and the drain electrode 270 to provide insulation therebetween.

The source electrode 260 and the drain electrode 270 may be on the interlayer insulating film 250. The interlayer insulating film 250 and the gate insulating film 230 may be formed to expose the source area and the drain area of the activation layer 220. The source electrode 260 and the drain electrode 270 may be in contact (e.g., physical contact) with the exposed portions of the source area and drain area of the activation layer 220.

The TFT may be electrically connected to the light-emitting device to drive the light-emitting device. The TFT may be covered and protected by a passivation layer 280. The passivation layer 280 may include an inorganic insulating film, an organic insulating film, or any combination thereof. The light-emitting device may be provided on the passivation layer 280. The light-emitting device may include the first electrode 110, the interlayer 130, and the second electrode 150.

The first electrode 110 may be on the passivation layer 280. The passivation layer 280 may not entirely cover the drain electrode 270. The passivation layer 280 may expose a portion of the drain electrode 270. The first electrode 110 may be connected to the exposed portion of the drain electrode 270.

A pixel defining layer 290 including an insulating material may be on the first electrode 110. The pixel defining layer 290 may expose a portion of the first electrode 110. The interlayer 130 may be formed in the exposed portion. The pixel defining layer 290 may be a polyimide-based organic film and/or a polyacrylic-based organic film. In some embodiments, at least some layers of the interlayer 130 may extend beyond the upper portion of the pixel defining layer 290 in the form of a common layer.

The second electrode 150 may be on the interlayer 130, and a capping layer 170 may be additionally on the second electrode 150. The capping layer 170 may cover the second electrode 150.

The encapsulation portion 300 may be on the capping layer 170. The encapsulation portion 300 may be on the light-emitting device to protect the light-emitting device from moisture and/or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxy methylene, polyarylate, hexamethyl disiloxane, an acrylic resin (e.g., polymethyl methacrylate, polyacrylic acid, etc.), an epoxy resin (e.g., aliphatic glycidyl ether (AGE), etc.), or any combination thereof; or a combination of the inorganic film and the organic film.

FIG. 3 is a schematic view of an electronic apparatus according to another embodiment.

The electronic apparatus (e.g., a light-emitting apparatus) of FIG. 3 is identical to the electronic apparatus of FIG. 2, except that a light-shielding pattern 500 and a functional region 400 are additionally on the encapsulation portion 300. The functional region 400 may be i) a color filter area, ii) a color conversion area, or iii) a combination of the color filter area and the color conversion area. In an embodiment, the light-emitting device included in the electronic apparatus of FIG. 3 may be a tandem light-emitting device.

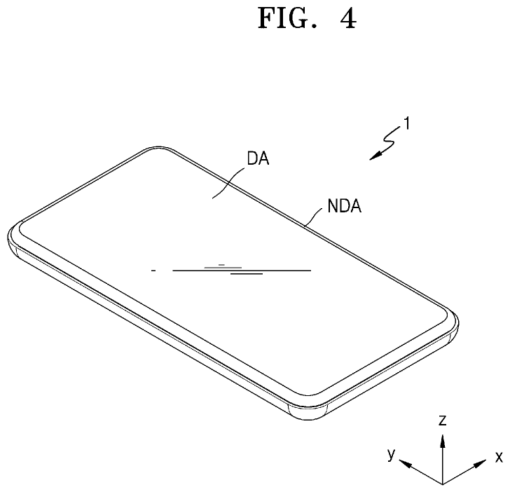

### Description of FIG. 4

FIG. 4 is a schematic perspective view of an electronic device 1 including a light-emitting device, according to an embodiment.

The electronic device 1 is an apparatus that displays a moving image or a still image, and may include a portable electronic device, such as a mobile phone, a smart phone, a tablet personal computer (PC), a mobile communication terminal, an electronic notebook, an electronic book, a portable multimedia player (PMP), a navigation device, and/or an ultra mobile PC (UMPC), as well as various suitable products, such as a television, a laptop, a monitor, a billboard, and/or an internet of things (IOT) device, and/or a part thereof. In addition, the electronic device 1 may be a wearable device, such as a smart watch, a watch phone, a glasses-type display (e.g., a display included in eyeglasses), and/or a head mounted display (HMD), and/or a part thereof. However, embodiments of the disclosure are not limited thereto. For example, the electronic device 1 may be an instrument panel of a vehicle, a center information display (CID) on a center fascia and/or a dashboard of a vehicle, a room mirror display that replaces a side mirror of a vehicle, an entertainment display for a rear seat of a vehicle or a display on a rear surface of a front seat, a head up display (HUD) installed at a front of a vehicle and/or projected on a front window glass, and/or a computer generated hologram augmented reality head up display (CGH AR HUD). For convenience of description, FIG. 4 shows a case in which the electronic device 1 is a smart phone.

The electronic device 1 may include a display area DA and a non-display area NDA outside the display area DA. A display apparatus may implement an image through an array of a plurality of pixels that are two-dimensionally arranged in the display area DA.

The non-display area NDA is an area in which an image is not displayed, and may entirely surround the display area DA. A driver for providing electrical signals or power to display elements in the display area DA may be in the non-display area NDA. A pad, which is an area to which an electronic element or a printed circuit board may be electrically connected, may be in the non-display area NDA.

The electronic device 1 may have different lengths in the x-axis direction and in the y-axis direction. For example, as shown in FIG. 4, the length in the x-axis direction may be shorter than the length in the y-axis direction. In an embodiment, the length in the x-axis direction may be the same as the length in the y-axis direction. In one or more embodiments, the length in the x-axis direction may be longer than the length in the y-axis direction.

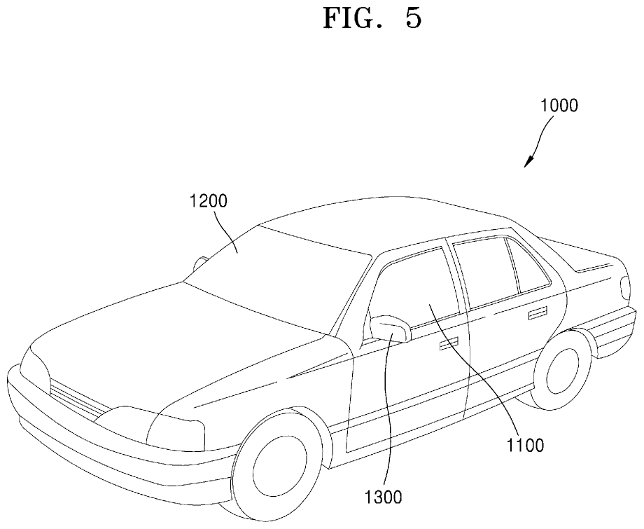

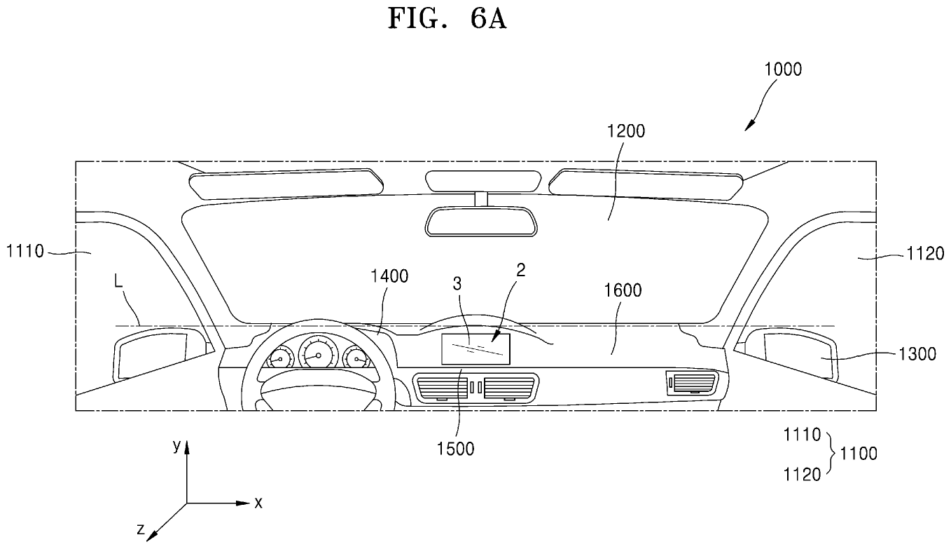

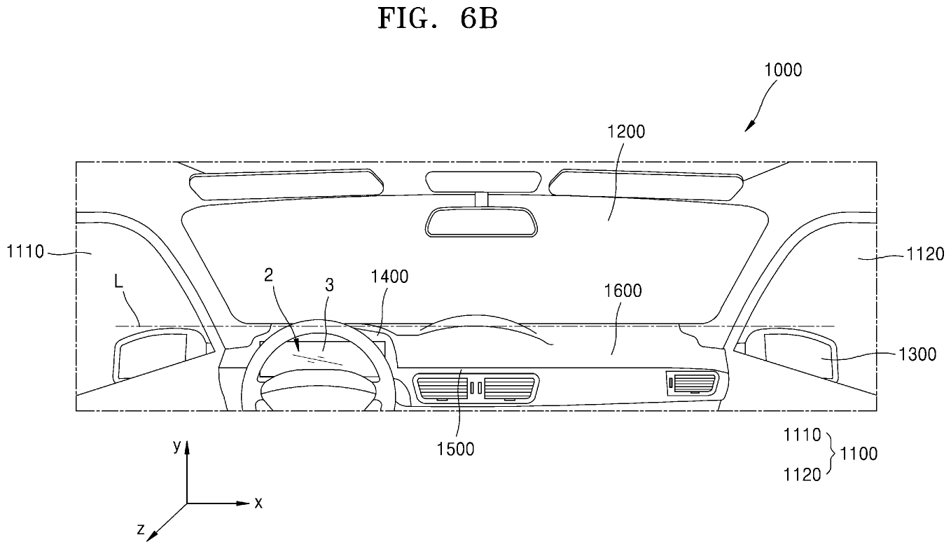

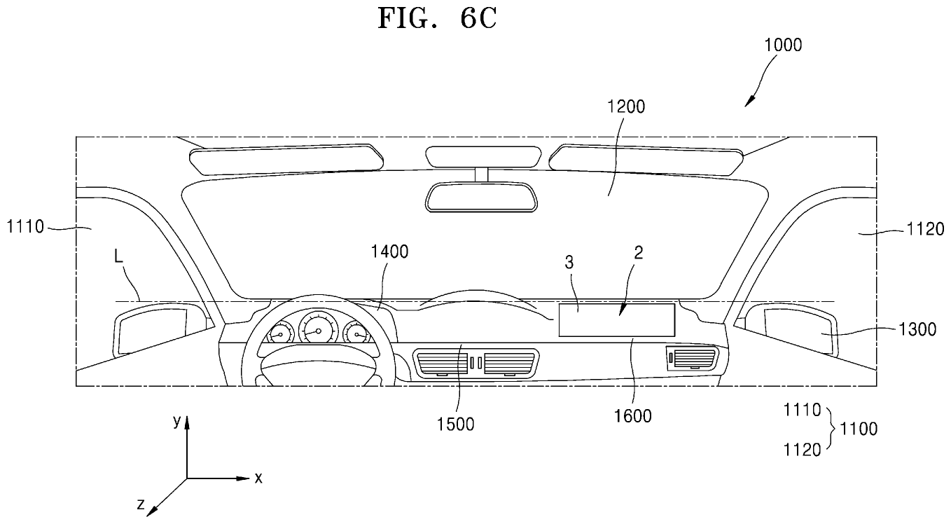

### Descriptions of FIGS. 5 and 6A to 6C

FIG. 5 is a schematic view of the exterior of a vehicle 1000 as an electronic device including a light-emitting device, according to an embodiment. FIGS. 6A to 6C are schematic views of the interior of the vehicle 1000 according to various embodiments.

Referring to FIGS. 5, 6A, 6B, and 6C, the vehicle 1000 may refer to various suitable apparatuses for moving a subject to be transported, such as a human, an object, and/or an animal, from a departure point to a destination. The vehicle 1000 may include a vehicle traveling on a road and/or a track, a vessel moving over the sea and/or a river, and an airplane flying in the sky by using the action of air.

The vehicle 1000 may travel on a road and/or a track. The vehicle 1000 may move in a certain direction according to rotation of at least one wheel. For example, the vehicle 1000 may include a three-wheeled or four-wheeled vehicle, a construction machine, a two-wheeled vehicle, a motorbike, a bicycle, and a train running on a track.

The vehicle 1000 may include a body having an interior and an exterior, and a chassis in which mechanical apparatuses necessary for driving are installed as the remaining parts except for the body. The exterior of the body may include a front panel, a bonnet, a roof panel, a rear panel, a trunk, and a pillar provided at a boundary between doors. The chassis of the vehicle 1000 may include a power generating apparatus, a power transmitting apparatus, a driving apparatus, a steering apparatus, a braking apparatus, a suspension apparatus, a transmission apparatus, a fuel apparatus, front and rear wheels, left and right wheels, and the like.

The vehicle 1000 may include a side window glass 1100, a front window glass 1200, a side mirror 1300, a cluster 1400, a center fascia 1500, a passenger seat dashboard 1600, and a display apparatus 2.

The side window glass 1100 and the front window glass 1200 may be partitioned by a pillar between the side window glass 1100 and the front window glass 1200.

The side window glass 1100 may be installed on a side surface of the vehicle 1000. In an embodiment, the side window glass 1100 may be installed on a door of the vehicle 1000. A plurality of side window glasses 1100 may be provided and may face each other. In an embodiment, the side window glass 1100 may include a first side window glass 1110 and a second side window glass 1120. In an embodiment, the first side window glass 1110 may be adjacent to the cluster 1400. The second side window glass 1120 may be adjacent to the passenger seat dashboard 1600.

In an embodiment, the side window glasses 1100 may be apart from each other in the x direction or the −x direction. For example, the first side window glass 1110 and the second side window glass 1120 may be apart from each other in the x direction or the −x direction. In other words, an imaginary straight line L connecting the side window glasses 1100 may extend in the x direction or the −x direction. For example, the imaginary straight line L connecting the first side window glass 1110 to the second side window glass 1120 may extend in the x direction or the −x direction.

The front window glass 1200 may be installed at the front of the vehicle 1000. The front window glass 1200 may be between the side window glasses 1100 facing each other.

The side mirror 1300 may provide a rear view of the vehicle 1000. The side mirror 1300 may be installed on the exterior of the body. In an embodiment, a plurality of side mirrors 1300 may be provided. One of the plurality of side mirrors 1300 may be outside the first side window glass 1110. Another one of the plurality of side mirrors 1300 may be outside the second side window glass 1120.

The cluster 1400 may be at the front of the steering wheel. The cluster 1400 may include a tachometer, a speedometer, a coolant thermometer, a fuel gauge, a direction change indicator light, a high beam indicator light, a warning light, a seat belt warning light, an odometer, a hodometer, an automatic transmission selection lever indicator light, a door open warning light, an engine oil warning light, and/or a low fuel warning light.

The center fascia 1500 may include a control panel on which a plurality of buttons for adjusting an audio apparatus, an air conditioning apparatus, and a heater of a seat are arranged. The center fascia 1500 may be on one side of the cluster 1400.

The passenger seat dashboard 1600 may be apart from the cluster 1400 with the center fascia 1500 therebetween. In an embodiment, the cluster 1400 may correspond to a driver seat, and the passenger seat dashboard 1600 may correspond to a passenger seat. In an embodiment, the cluster 1400 may be adjacent to the first side window glass 1110, and the passenger seat dashboard 1600 may be adjacent to the second side window glass 1120.

In an embodiment, the display apparatus 2 may include a display panel 3, and the display panel 3 may display an image. The display apparatus 2 may be inside the vehicle 1000. In an embodiment, the display apparatus 2 may be between the side window glasses 1100 facing each other. The display apparatus 2 may be on at least one of the cluster 1400, the center fascia 1500, and the passenger seat dashboard 1600.

The display apparatus 2 may include an organic light-emitting display apparatus, an inorganic light-emitting display apparatus, a quantum dot display apparatus, and the like. Hereinafter, an organic light-emitting display apparatus including the light-emitting device according to the disclosure will be described as an example of the display apparatus 2. However, various suitable types or kinds of display apparatuses as described above may be used in embodiments of the disclosure.

Referring to FIG. 6A, the display apparatus 2 may be on the center fascia 1500. In an embodiment, the display apparatus 2 may display navigation information. In an embodiment, the display apparatus 2 may display audio, video, or information regarding vehicle settings.

Referring to FIG. 6B, the display apparatus 2 may be on the cluster 1400. In this case, the cluster 1400 may display driving information and the like through the display apparatus 2. For example, the cluster 1400 may be digitally implemented. The digital cluster 1400 may display vehicle information and driving information as images. For example, a needle and a gauge of a tachometer and various suitable warning light icons may be displayed by digital signals.

Referring to FIG. 6C, the display apparatus 2 may be on the passenger seat dashboard 1600. The display apparatus 2 may be embedded in the passenger seat dashboard 1600 or on the passenger seat dashboard 1600. In an embodiment, the display apparatus 2 on the passenger seat dashboard 1600 may display an image related to information displayed on the cluster 1400 and/or information displayed on the center fascia 1500. In an embodiment, the display apparatus 2 on the passenger seat dashboard 1600 may display information different from information displayed on the cluster 1400 and/or information displayed on the center fascia 1500.

### Manufacturing Method

Respective layers included in the hole transport region, the emission layer, and respective layers included in the electron transport region may be formed in a certain region by using one or more suitable methods, such as vacuum deposition, spin coating, casting, Langmuir-Blodgett (LB) deposition, ink-jet printing, laser-printing, and/or laser-induced thermal imaging (LITI).

When layers constituting the hole transport region, the emission layer, and layers constituting the electron transport region are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group,” as used herein, refers to a cyclic group having 3 to 60 carbon atoms and consisting of only carbon atoms as ring-forming atoms. The term “C₁-C₆₀ heterocyclic group,” as used herein, refers to a cyclic group having 1 to 60 carbon atoms and further including, in addition to carbon atoms, a heteroatom as a ring-forming atom. The C₃-C₆₀ carbocyclic group and the C₁-C₆₀ heterocyclic group may each be a monocyclic group consisting of one ring or a polycyclic group in which two or more rings are condensed together with each other. For example, the C₁-C₆₀ heterocyclic group may have 3 to 61 ring-forming atoms.

The term “cyclic group,” as used herein, may include both the C₃-C₆₀ carbocyclic group and the C₁-C₆₀ heterocyclic group.

The term “π electron-rich C₃-C₆₀ cyclic group,” as used herein, refers to a cyclic group that has 3 to 60 carbon atoms and does not include *—N═*′ as a ring-forming moiety. The term “π electron-deficient nitrogen-containing C₁-C₆₀ cyclic group,” as used herein, refers to a heterocyclic group having 1 to 60 carbon atoms and including *—N═*′ as a ring-forming moiety.

For example,

the C₃-C₆₀ carbocyclic group may be i) a T1 group or ii) a group in which at least two T1 groups are condensed together with each other (e.g., a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group).

The C₁-C₆ heterocyclic group may be i) a T2 group, ii) a condensed cyclic group in which at least two T2 groups are condensed together with each other, or iii) a condensed cyclic group in which at least one T2 group is condensed together with at least one T1 group (e.g., a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.).

The π electron-rich C₃-C₆₀ cyclic group may be i) a T1 group, ii) a condensed cyclic group in which at least two T1 groups are condensed together with each other, iii) a T3 group, iv) a condensed cyclic group in which at least two T3 groups are condensed together with each other, or v) a condensed cyclic group in which at least one T3 group is condensed together with at least one T1 group (e.g., the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, etc.).

The π electron-deficient nitrogen-containing C₁-C₆₀ cyclic group may be i) a T4 group, ii) a group in which at least two T4 groups are condensed together with each other, iii) a group in which at least one T4 group is condensed together with at least one T1 group, iv) a group in which at least one T4 group is condensed with at least one T3 group, or v) a group in which at least one T4 group, at least one T1 group, and at least one T3 group are condensed together with each other (e.g., a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, etc.).

The T1 group may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or a bicyclo[2.2.1]heptane) group, a norbornene group, a bicyclo[1.1.1]pentane group, a bicyclo[2.1.1]hexane group, a bicyclo[2.2.2]octane group, or a benzene group.

The T2 group may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

The T3 group may be a furan group, a thiophene group, a 1H-pyrrole group, a silole group, or a borole group.

The T4 group may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group,” “the C₃-C₆₀ carbocyclic group,” “the C₁-C₆₀ heterocyclic group,” “the π electron-rich C₃-C₆₀ cyclic group,” or “the π electron-deficient nitrogen-containing C₁-C₆₀ cyclic group,” as used herein, refer to a group condensed to any cyclic group, a monovalent group, or a polyvalent group (e.g., a divalent group, a trivalent group, a tetravalent group, etc.) according to the structure of a formula for which the corresponding term is used. For example, a “benzene group” may be a benzo group, a phenyl group, a phenylene group, or the like, which may be easily understand by one of ordinary skill in the art according to the structure of a formula including the “benzene group.”

Examples of the monovalent C₃-C₆₀ carbocyclic group and the monovalent C₁-C₆ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆₀ carbocyclic group and the divalent C₁-C₆₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group,” as used herein, refers to a linear or branched aliphatic hydrocarbon monovalent group having 1 to 60 carbon atoms. For example, the C₁-C₆₀ alkyl group may include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, a tert-decyl group, and the like.

The term “C₁-C₆₀ alkylene group,” as used herein, refers to a divalent group having substantially the same structure as the C₁-C₆₀ alkyl group.

The term “C₂-C₆₀ alkenyl group,” as used herein, refers to a monovalent hydrocarbon group having at least one carbon-carbon double bond at a main chain (e.g., in the middle) or at a terminal end (e.g., the terminus) of the C₂-C₆₀ alkyl group. For example, the C₂-C₆₀ alkenyl group may include an ethenyl group, a propenyl group, a butenyl group, and the like.

The term “C₂-C₆₀ alkenylene group,” as used herein, refers to a divalent group having substantially the same structure as the C₂-C₆₀ alkenyl group.

The term “C₂-C₆₀ alkynyl group,” as used herein, refers to a monovalent hydrocarbon group having at least one carbon-carbon triple bond at a main chain (e.g., in the middle) or at a terminal end (e.g., the terminus) of the C₂-C₆₀ alkyl group. For example, the C₂-C₆₀ alkynyl group may include an ethynyl group, a propynyl group, and the like.

The term “C₂-C₆₀ alkynylene group,” as used herein, refers to a divalent group having substantially the same structure as the C₂-C₆₀ alkynyl group.

The term “C₁-C₆₀ alkoxy group,” as used herein, refers to a monovalent group having the formula of—OA₁₀₁ (wherein A₁₀₁ is the C₁-C₆₀ alkyl group). For example, the C₁-C₆₀ alkoxy group may include a methoxy group, an ethoxy group, an isopropyloxy group, and the like.

The term “C₃-C₁₀ cycloalkyl group,” as used herein, refers to a monovalent saturated hydrocarbon cyclic group having 3 to 10 carbon atoms. For example, the C₃-C₁₀ cycloalkyl group may include a cyclopropyl group, a cyclobutyl group, a cyclopentyl, cyclohexyl group, a cycloheptyl group, a cyclooctyl group, an adamantanyl group, a norbornanyl group (or a bicyclo[2.2.1]heptyl group), a bicyclo[1.1.1]pentyl group, a bicyclo[2.1.1]hexyl group, a bicyclo[2.2.2]octyl group, and the like.

The term “C₃-C₁₀ cycloalkylene group,” as used herein, refers to a divalent group having substantially the same structure as the C₃-C₁₀ cycloalkyl group.

The term “C₁-C₁₀ heterocycloalkyl group,” as used herein, refers to a monovalent cyclic group having 1 to 10 carbon atoms and further including, in addition to carbon atoms, at least one heteroatom as a ring-forming atom. For example, the C₁-C₁₀ heterocycloalkyl group may include a 1,2,3,4-oxatriazolidinyl group, a tetrahydrofuranyl group, a tetrahydrothiophenyl group, and the like.

The term “C₁-C₁₀ heterocycloalkylene group,” as used herein, refers to a divalent group having substantially the same structure as the C₁-C₁₀ heterocycloalkyl group.

The term “C₃-C₁₀ cycloalkenyl group,” as used herein, refers to a monovalent cyclic group of 3 to 10 carbon atoms that has at least one carbon-carbon double bond in its ring and no aromaticity (e.g., is not aromatic). For example, the C₃-C₁₀ cycloalkenyl group may include a cyclopentenyl group, a cyclohexenyl group, a cycloheptenyl group, and the like.

The term “C₃-C₁₀ cycloalkenylene group,” as used herein, refers to a divalent group having substantially the same structure as the C₃-C₁₀ cycloalkenyl group.

The term “C₁-C₁₀ heterocycloalkenyl group,” as used herein, refers to a monovalent cyclic group of 1 to 10 carbon atoms that further includes, in addition to carbon atoms, at least one heteroatom as a ring-forming atom and has at least one double bond in its ring. For example, the C₁-C₁₀ heterocycloalkenyl group may include a 4,5-dihydro-1,2,3,4-oxatriazolyl group, a 2,3-dihydrofuranyl group, a 2,3-dihydrothiophenyl group, and the like.

The term “C₁-C₁₀ heterocycloalkenylene group,” as used herein, refers to a divalent group having substantially the same structure as the C₁-C₁₀ heterocycloalkenyl group.

The term “C₆-C₆₀ aryl group,” as used herein, refers to a monovalent group having a carbocyclic aromatic system of 6 to 60 carbon atoms. For example, the C₆-C₆₀ aryl group may include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, an ovalenyl group, and the like.

The term “C₆-C₆₀ arylene group,” as used herein, refers to a divalent group having a carbocyclic aromatic system of 6 to 60 carbon atoms.

When the C₆-C₆₀ aryl group and the C₆-C₆₀ arylene group each include two or more rings, the two or more rings may be condensed together with each other.

The term “C₁-C₆₀ heteroaryl group,” as used herein, refers to a monovalent group that further includes, in addition to carbon atoms, at least one heteroatom as a ring-forming atom and has a heterocyclic aromatic system of 1 to 60 carbon atoms. For example, the C₁-C₆₀ heteroaryl group may include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, a naphthyridinyl group, and the like.

The term “C₁-C₆₀ heteroarylene group,” as used herein, refers to a divalent group that further includes, in addition to carbon atoms, at least one heteroatom as a ring-forming atom and has a heterocyclic aromatic system of 1 to 60 carbon atoms.

When the C₁-C₆ heteroaryl group and the C₁-C₆₀ heteroarylene group each include two or more rings, the two or more rings may be condensed together with each other.

The term “monovalent non-aromatic condensed polycyclic group,” as used herein, refers to a monovalent group (e.g., having 8 to 60 carbon atoms) having two or more rings condensed together with each other, only carbon atoms as ring-forming atoms, and no aromaticity in its entire molecular structure (e.g., is not aromatic when considered as a whole). For example, the monovalent non-aromatic condensed polycyclic group may include an indenyl group, a fluorenyl group, a spiro-bifluorenyl group, a benzofluorenyl group, an indenophenanthrenyl group, an indenoanthracenyl group, and the like.

The term “divalent non-aromatic condensed polycyclic group,” as used herein, refers to a divalent group having substantially the same structure as the monovalent non-aromatic condensed polycyclic group.

The term “monovalent non-aromatic condensed heteropolycyclic group,” as used herein, refers to a monovalent group (e.g., having 1 to 60 carbon atoms) having two or more rings condensed together with each other, at least one heteroatom other than carbon atoms, as a ring-forming atom, and no aromaticity in its entire molecular structure (e.g., is not aromatic when considered as a whole). For example, the monovalent non-aromatic condensed heteropolycyclic group may include a pyrrolyl group, a thiophenyl group, a furanyl group, an indolyl group, a benzoindolyl group, a naphthon indolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indenocarbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphtho silolyl group, a benzofurodibenzofuranyl group, a benzofurodibenzothiophenyl group, a benzothienodibenzothiophenyl group, and the like.

The term “divalent non-aromatic condensed heteropolycyclic group,” as used herein, refers to a divalent group having substantially the same structure as the monovalent non-aromatic condensed heteropolycyclic group described above.

The term “C₆-C₆₀ aryloxy group,” as used herein, refers to —OA₁₀₂ (wherein A₁₀₂ is the C₆-C₆₀ aryl group).

The term “C₆-C₆₀ arylthio group,” as used herein, refers to —SA₁₀₃ (wherein A₁₀₃ is the C₆-C₆₀ aryl group).

The term “C₇-C₆₀ arylalkyl group,” as used herein, refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group,” as used herein, refers to -A₁₀₆A₁₀₇ (wherein A₁₀₆ is a C₁-C₅₉ alkylene group, and A₁₀₇ is a C₁-C₅₉ heteroaryl group).

The term “R₁₀ₐ,” as used herein, refers to:

deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or

—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Q₁ to Q₃, Q₁₁ to Q₁₃, Q₂₁ to Q₂₃, and Q₃₁ to Q₃₃ used herein may each independently be: hydrogen; deuterium; —F; —Cl; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; a C₃-C₆₀ carbocyclic group or a C₁-C₆₀ heterocyclic group, each unsubstituted or substituted with deuterium, —F, a cyano group, a C₁-C₆₀ alkyl group, a C₁-C₆₀ alkoxy group, a phenyl group, a biphenyl group, or any combination thereof; a C₇-C₆₀ arylalkyl group; or a C₂-C₆₀ heteroarylalkyl group.

The term “heteroatom,” as used herein, refers to any atom other than a carbon atom. Examples of the heteroatom may include O, S, N, P, Si, B, Ge, Se, or any combination thereof.

The term “the third-row transition metal,” as used herein, may include hafnium (Hf), tantalum (Ta), tungsten (W), rhenium (Re), osmium (Os), iridium (Ir), platinum (Pt), gold (Au), and the like.

The term “Ph,” as used herein, refers to a phenyl group, the term “Me,” as used herein, refers to a methyl group, the term “Et,” as used herein, refers to an ethyl group, the term “tert-Bu” or “Bu^{t},” as used herein, refers to a tert-butyl group, and the term “OMe,” as used herein, refers to a methoxy group.

The term “biphenyl group,” as used herein, refers to “a phenyl group substituted with a phenyl group.” In other words, the “biphenyl group” is a substituted phenyl group having a C₆-C₆₀ aryl group as a substituent.

The term “terphenyl group,” as used herein, refers to “a phenyl group substituted with a biphenyl group.” In other words, the “terphenyl group” is a substituted phenyl group having, as a substituent, a C₆-C₆₀ aryl group substituted with a C₆-C₆₀ aryl group.

* and *′, as used herein, unless defined otherwise, each refer to a binding site to a neighboring atom in a corresponding formula or moiety.

The x-axis, y-axis, and z-axis, as used herein, are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in more detail with reference to Synthesis Examples and Examples. The wording “B was used instead of A” used in describing Synthesis Examples means that an identical molar equivalent of B was used in place of A.

### Synthesis Example 1 (Synthesis of Compound 10)

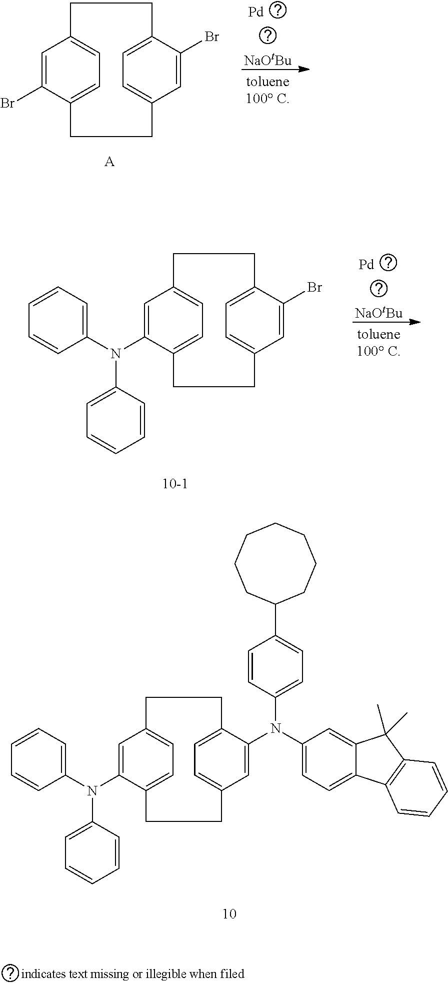

### Synthesis of Intermediate 10-1

3.66 g of Intermediate A, 1.69 g of diphenylamine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 10-1 (3.22 g, yield: 71%).

### Synthesis of Compound 10

4.54 g of Intermediate 10-1, 3.95 g of N-(4-cyclooctylphenyl)-9,9-dimethyl-9H-fluoren-2-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 10 (4.99 g, yield: 65%).

### Synthesis Example 2 (Synthesis of Compound 15)

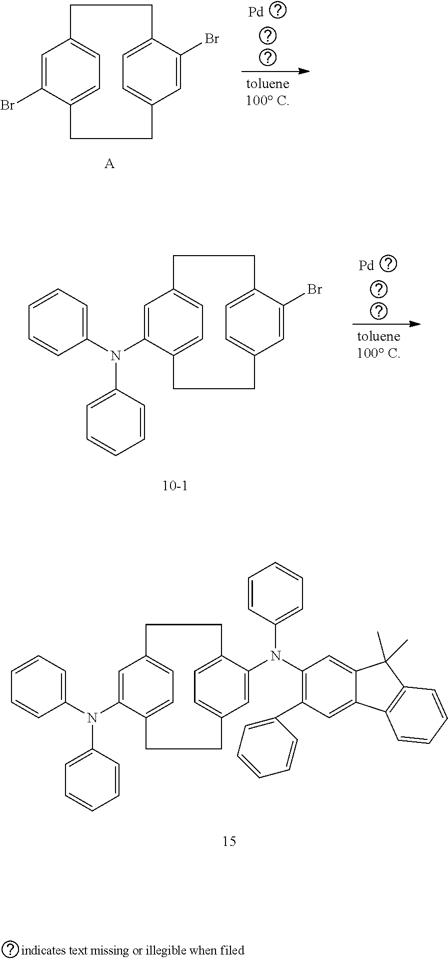

### Synthesis of Compound 15

4.54 g of Intermediate 10-1, 3.61 g of 9,9-dimethyl-N,3-diphenyl-9H-fluoren-2-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 15 (5.06 g, yield: 69%).

### Synthesis Example 3 (Synthesis of Compound 25)

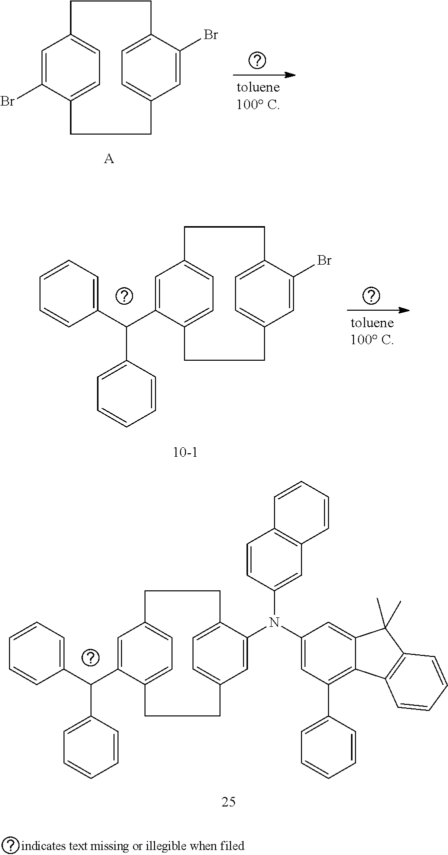

### Synthesis of Compound 25

4.54 g of Intermediate 10-1, 4.11 g of 9,9-dimethyl-N-(naphthalen-2-yl)-4-phenyl-9H-fluoren-2-amine, 0.46 g of Pd₂(dba)₃, 0.21 g, of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 25 (5.57 g, yield: 71%).

### Synthesis Example 4 (Synthesis of Compound 67)

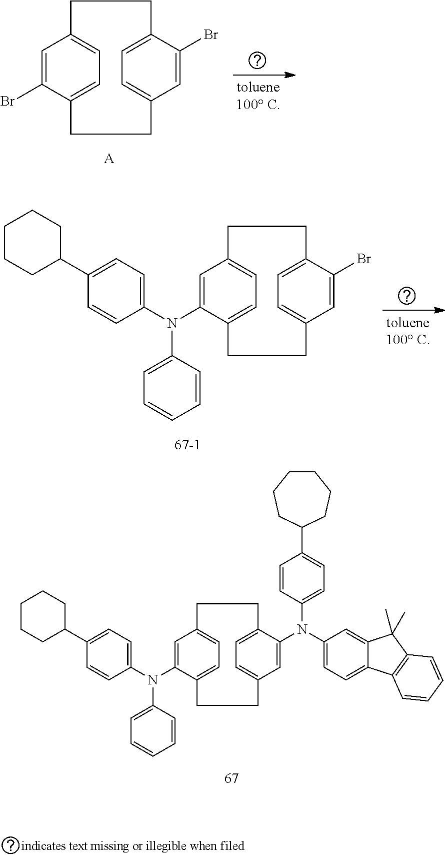

### Synthesis of Intermediate 67-1

3.66 g of Intermediate A, 2.51 g of 4-cyclohexyl-N-phenylaniline, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 67-1 (3.26 g, yield: 61%).

### Synthesis of Compound 67

5.36 g of Intermediate 67-1, 3.81 g of N-(4-cycloheptylphenyl)-9,9-dimethyl-9H-fluoren-2-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 67 (6.44 g, yield: 77%).

### Synthesis Example 5 (Synthesis of Compound 90)

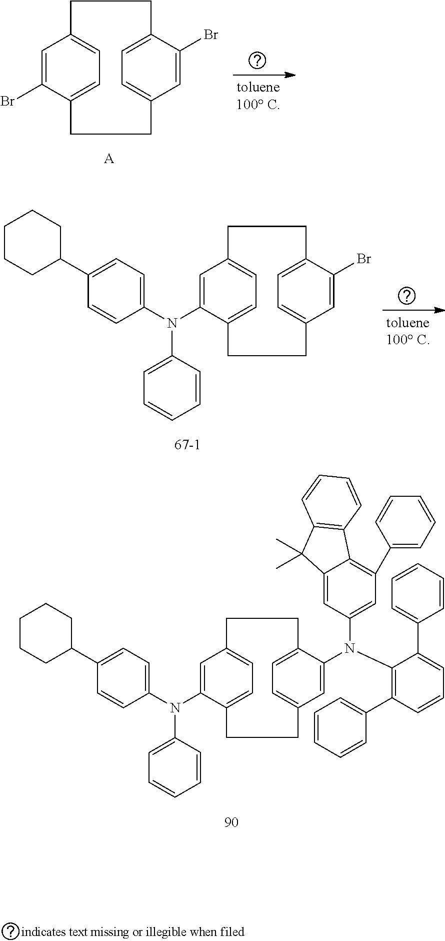

### Synthesis of Compound 90

5.36 g of Intermediate 67-1, 5.13 g of N-([1,1′:3′,1″-terphenyl]-2′-yl)-9,9-dimethyl-4-phenyl-9H-fluoren-2-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 90 (7.84 g, yield: 81%).

### Synthesis Example 6 (Synthesis of Compound 99)

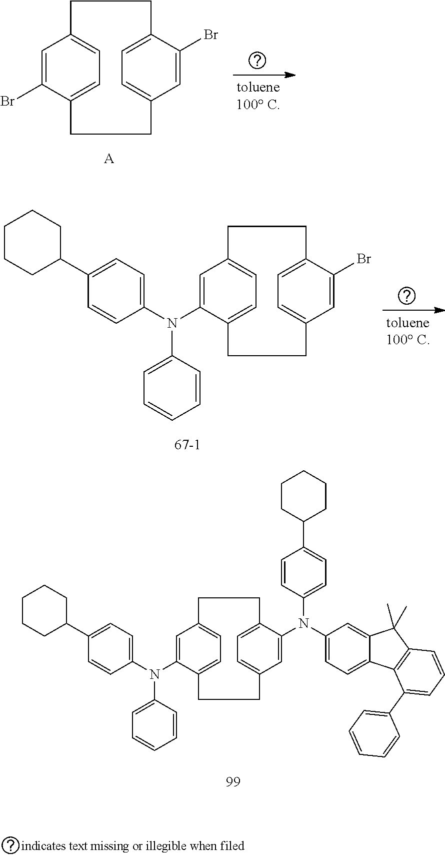

### Synthesis of Compound 99

5.36 g of Intermediate 67-1, 4.43 g of N-(4-cyclohexylphenyl)-9,9-dimethyl-5-phenyl-9H-fluoren-2-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 99 (6.74 g, yield: 75%).

### Synthesis Example 7 (Synthesis of Compound 132)

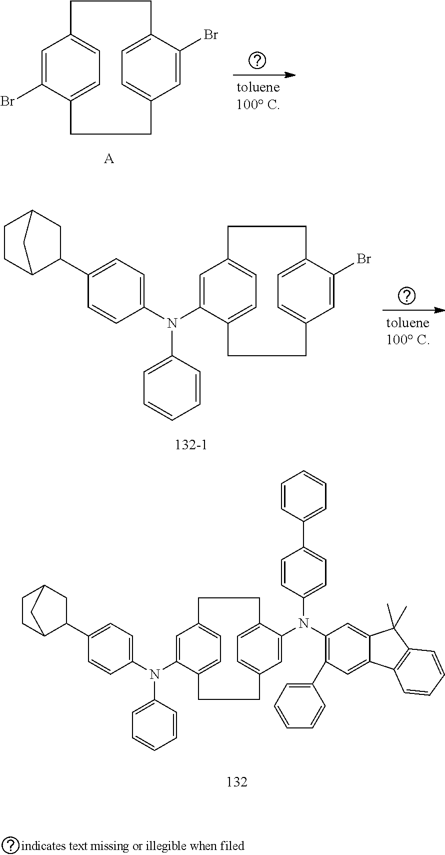

### Synthesis of Intermediate 132-1

3.66 g of Intermediate A, 2.63 g of 4-(bicyclo[2.2.1]heptan-2-yl)-N-phenylaniline, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 132-1 (4.06 g, yield: 74%).

### Synthesis of Compound 132

5.48 g of Intermediate 132-1, 4.37 g of N-([1,1′-biphenyl]-4-yl)-9,9-dimethyl-3-phenyl-9H-fluoren-2-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 132 (6.24 g, yield: 69%).

### Synthesis Example 8 (Synthesis of Compound 145)

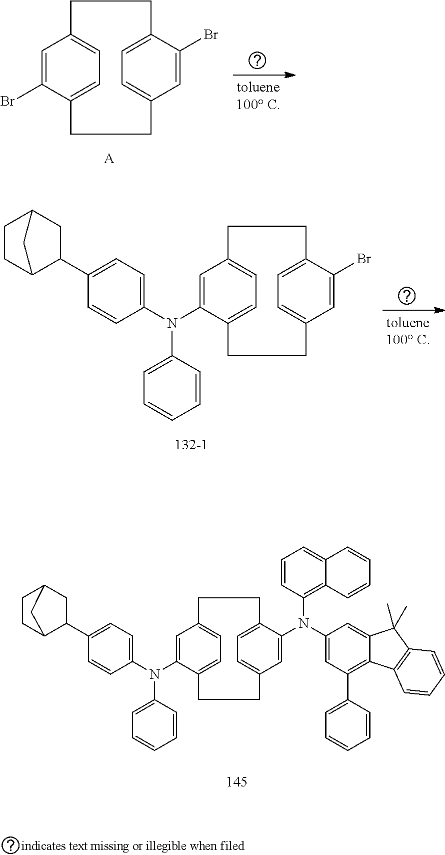

### Synthesis of Compound 145

5.48 g of Intermediate 132-1, 4.11 g of 9,9-dimethyl-N-(naphthalen-1-yl)-4-phenyl-9H-fluoren-2-amine, 0.46 g of Pd₂(dba)₃, 0.21 g, of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 145 (6.50 g, yield: 74%).

### Synthesis Example 9 (Synthesis of Compound 169)

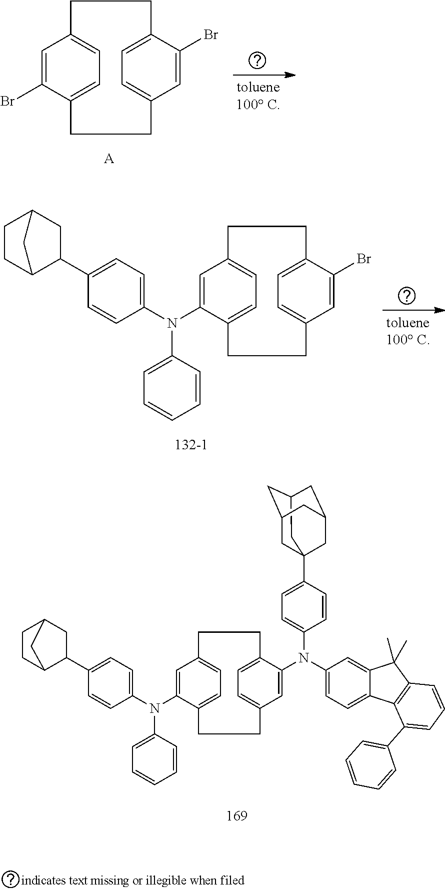

### Synthesis of Compound 169

5.48 g of Intermediate 132-1, 4.95 g of N-(4-((3r,5r,7r)-adamantan-1-yl)phenyl)-9,9-dimethyl-5-phenyl-9H-fluoren-2-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 169 (6.54 g, yield: 68%).

### Synthesis Example 10 (Synthesis of Compound 206)

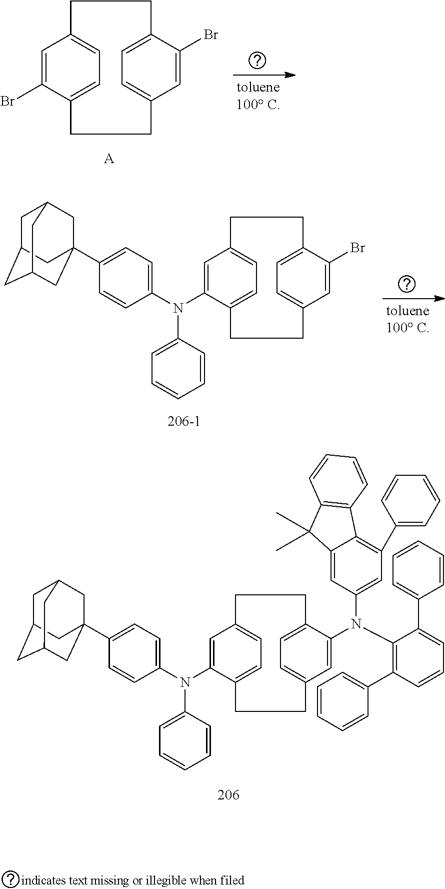

### Synthesis of Intermediate 206-1

3.66 g of Intermediate A, 3.03 g of 4-((3r,5r,7r)-adamantan-1-yl)-N-phenylaniline, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 206-1 (4.52 g, yield: 77%).

### Synthesis of Compound 206

5.88 g of Intermediate 206-1, 5.13 g of N-([1,1′:3′,1″-terphenyl]-2′-yl)-9,9-dimethyl-4-phenyl-9H-fluoren-2-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 206 (7.41 g, yield: 74%).

### Synthesis Example 11 (Synthesis of Compound 233)

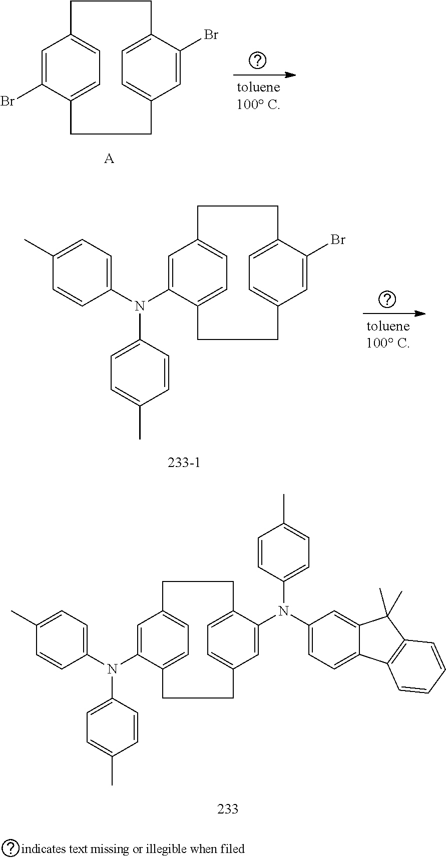

### Synthesis of Intermediate 233-1

3.66 g of Intermediate A, 1.97 g of di-p-tolylamine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 233-1 (3.71 g, yield: 77%).

### Synthesis of Compound 233

4.82 g of Intermediate 233-1, 2.99 g of 9,9-dimethyl-N-(p-tolyl)-9H-fluoren-2-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 233 (4.90 g, yield: 70%).

### Synthesis Example 12 (Synthesis of Compound 234)

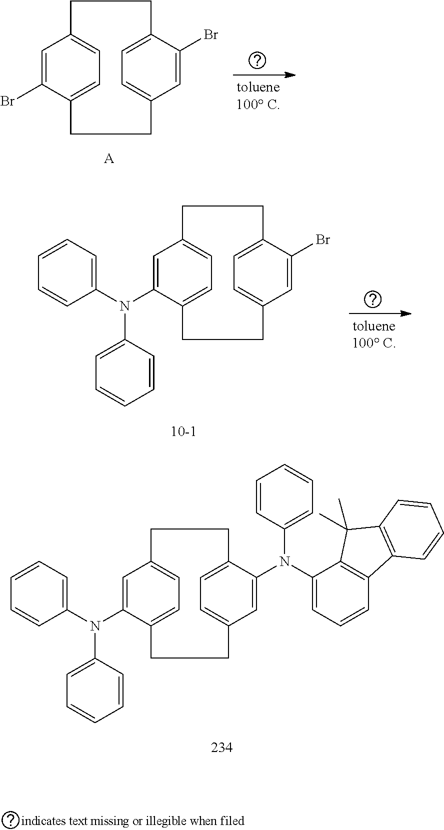

### Synthesis of Compound 234

4.54 g of Intermediate 10-1, 2.85 g of 9,9-dimethyl-N-phenyl-9H-fluoren-1-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 234 (4.40 g, yield: 67%).

### Synthesis Example 13 (Synthesis of Compound 235)

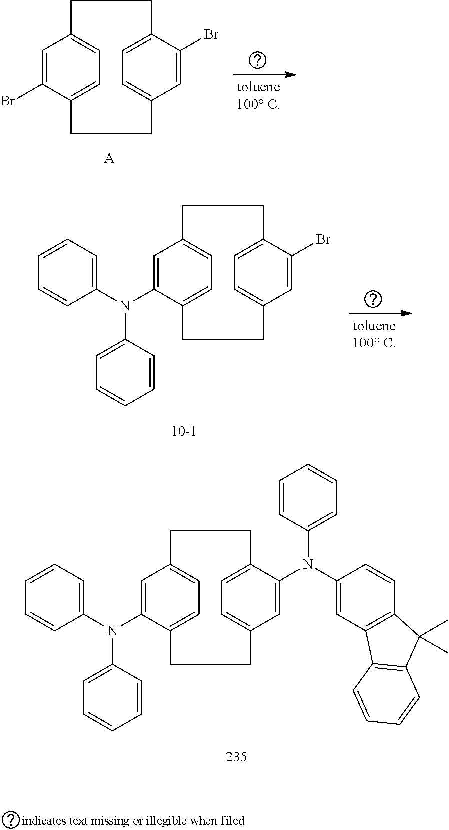

### Synthesis of Compound 235

4.54 g of Intermediate 10-1, 2.85 g of 9,9-dimethyl-N-phenyl-9H-fluoren-3-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 235 (4.67 g, yield: 71%).

### Synthesis Example 14 (Synthesis of Compound 236)

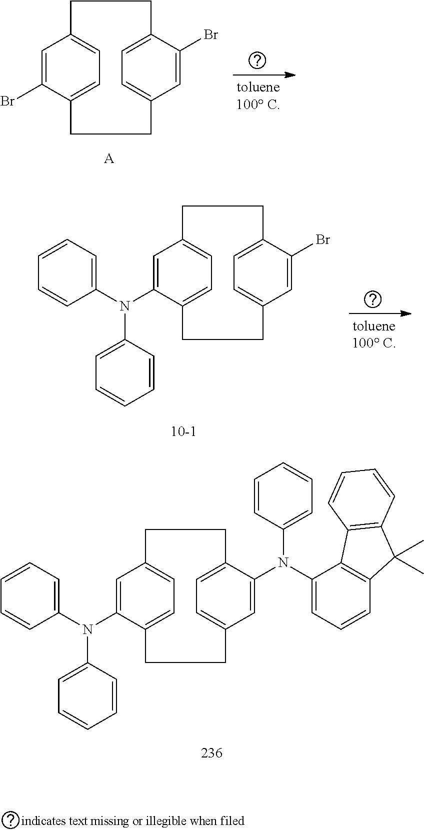

### Synthesis of Compound 236

4.54 g of Intermediate 10-1, 2.85 g of 9,9-dimethyl-N-phenyl-9H-fluoren-4-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 236 (4.93 g, yield: 75%).

### Synthesis Example 15 (Synthesis of Compound 237)

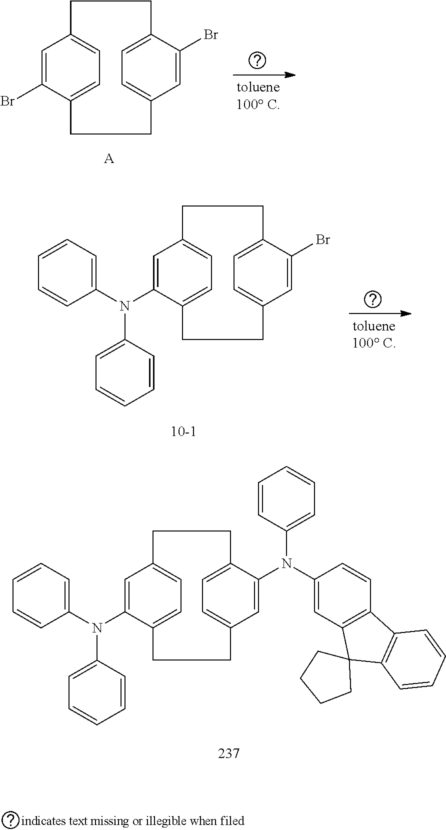

### Synthesis of Compound 237

4.54 g of Intermediate 10-1, 3.11 g of N-phenylspiro[cyclopentane-1,9′-fluoren]-2′-amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 237 (5.47 g, yield: 80%).

### Synthesis Example 16 (Synthesis of Compound 238)

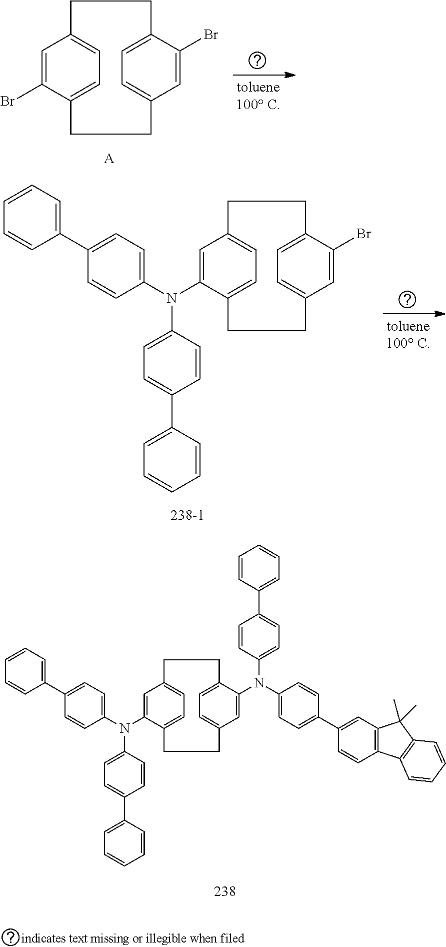

### Synthesis of Intermediate 238-1

3.66 g of Intermediate A, 3.21 g of di([1,1′-biphenyl]-4-yl)amine, 0.46 g of Pd₂(dba)₃, 0.21 g of P(t-Bu)₃, and 2.44 g of NaOtBu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 238-1 (3.71 g, yield: 77%).

### Synthesis of Compound 238

6.06 g of Intermediate 238-1, 4.37 g of N-(4-(9,9-dimethyl-9H-fluoren-2-yl)phenyl)-[1,1′-biphenyl]-4-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 238 (6.64 g, yield: 69%).

### Synthesis Example 17 (Synthesis of Compound 239)

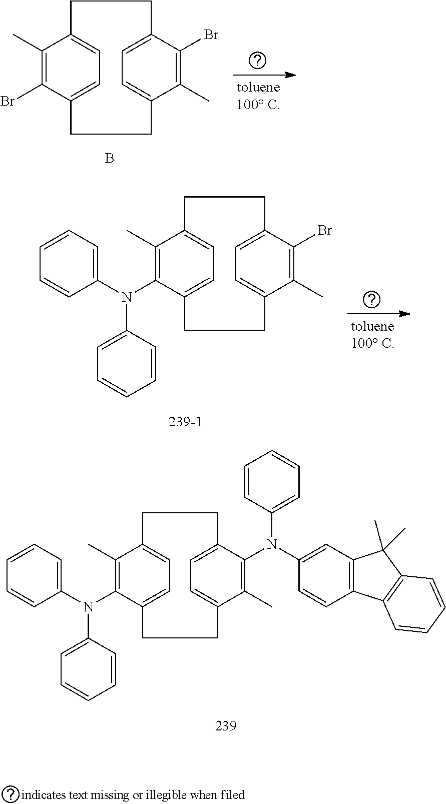

### Synthesis of Intermediate 239-1

3.97 g of Intermediate B, 1.69 g of diphenylamine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Intermediate 239-1 (2.94 g, yield: 61%).

### Synthesis of Compound 239

4.82 g of Intermediate 239-1, 4.37 g of N-(4-(9,9-dimethyl-9H-fluoren-2-yl)phenyl)-[1,1′-biphenyl]-4-amine, 0.46 g of Pd₂(dba)₃, 0.21 g of P(t-Bu)₃, and 2.44 g of NaO^{t}Bu were dissolved in 50 ml of toluene, and then stirred at 100° C. for 1 hour. After the resultant reaction solution was cooled to room temperature, the reaction was terminated by using water. Then, an extraction process was performed thereon three times by using ethyl ether. An organic layer extracted therefrom was dried by using anhydrous magnesium sulfate and distilled under reduced pressure, and a residue thus obtained was separated and purified by column chromatography, thereby obtaining Compound 239 (5.55 g, yield: 81%).

### Comparative Example 1

As an anode, a glass substrate with a 15 Ω/cm² (1,200 Å) ITO formed thereon (available from Corning Co., Ltd) was cut to a size of 50 mm×50 mm×0.7 mm, sonicated by using isopropyl alcohol and pure water for 5 minutes in each solvent, cleaned by irradiation of ultraviolet rays and exposure of ozone thereto for 30 minutes, and then mounted on a vacuum deposition apparatus.

2-TNATA was vacuum-deposited on the anode to form a hole injection layer having a thickness of 600 Å. 4,4′-bis[N-(1-naphthyl)-N-phenylamino]biphenyl (NPB) was vacuum-deposited on the hole injection layer to form a hole transport layer having a thickness of 300 Å.

9,10-di(naphthalen-2-yl)anthracene (DNA) and 4,4′-bis[2-(4-(N,N-diphenylamino)phenyl)vinyl]biphenyl (DPAVBi) were vacuum-deposited on the hole transport layer at a weight ratio of 98:2 to form an emission layer having a thickness of 300 Å.

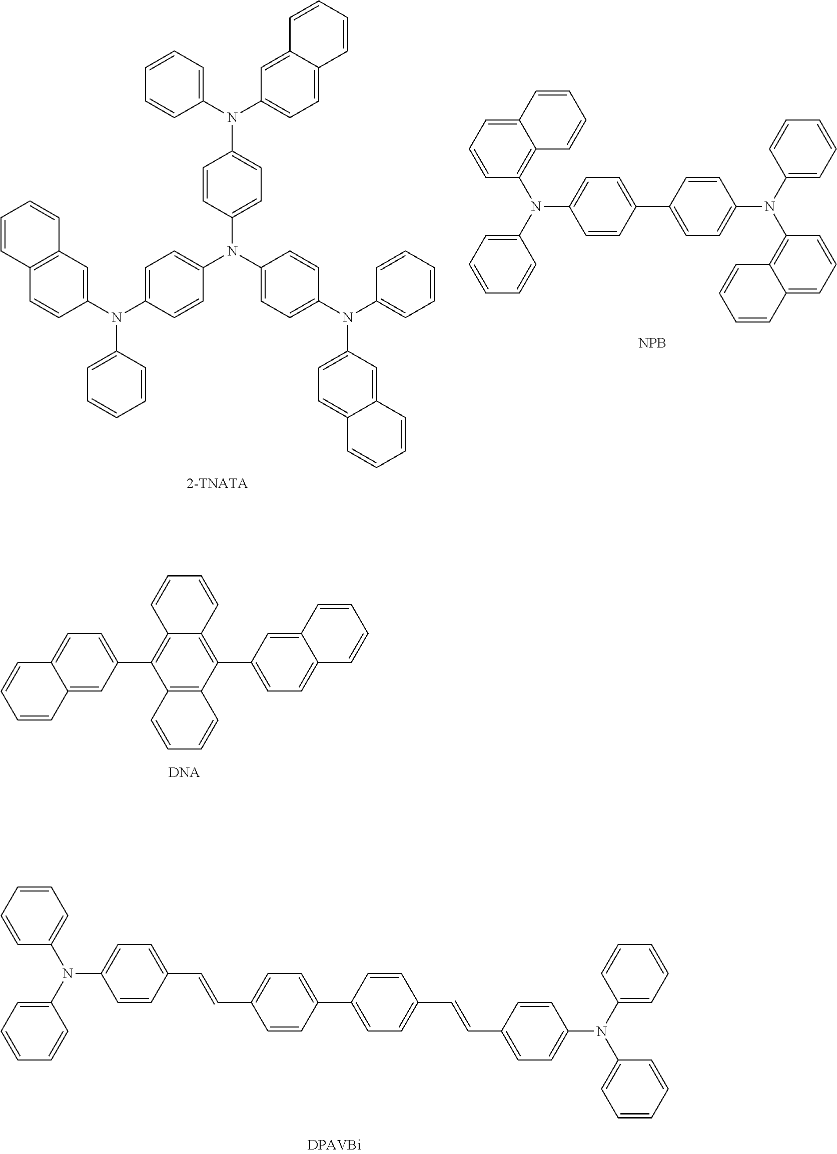

Alq₃ was vacuum-deposited on the emission layer to form an electron transport layer having a thickness of 300 Å. LiF was vacuum-deposited on the electron transport layer to form an electron injection layer having a thickness of 10 Å. Al was vacuum-deposited on the electron injection layer to form a cathode having a thickness of 3,000 Å, thereby completing the manufacture of a light-emitting device.

### Comparative Examples 2 to 8 and Examples 1 to 17

Light-emitting devices were manufactured in substantially the same manner as in Comparative Example 1, except that compounds shown in Table 1 were each used instead of NPB in the formation of the hole transport layer.

### Evaluation Example 1

To evaluate characteristics of each of the light-emitting devices manufactured according to Comparative Examples 1 to 8 and Examples 1 to 17, the driving voltage, luminance, luminescence efficiency, and lifespan thereof were measured, and the results are shown in Table 1.

The driving voltage was measured at a current density of 50 mA/cm² by using a source meter (Keithley Instrument, 2400 series).

The luminance and the luminescence efficiency were measured using a luminance meter PR650 while power was supplied from a current-voltmeter (Keithley SMU 236).

TABLE 1

| | | Driving | Current | | Luminescence | | Half lifespan |
|---|---|---|---|---|---|---|---|
| | | voltage | density | Luminance | efficiency | Emission | (hr @ |
| | Compound | (V) | (mA/cm²) | (cd/m²) | (cd/A) | color | 100 mA/cm²) |
| Comparative | NPB | 7.01 | 50 | 2645 | 5.29 | Blue | 258 |
| Example 1 | | | | | | | |
| Comparative | A | 4.81 | 50 | 3565 | 7.13 | Blue | 400 |
| Example 2 | | | | | | | |
| Comparative | B | 4.78 | 50 | 3620 | 7.24 | Blue | 392 |
| Example 3 | | | | | | | |
| Comparative | C | 6.21 | 50 | 3835 | 7.67 | Blue | 311 |
| Example 4 | | | | | | | |
| Comparative | D | 5.78 | 50 | 3220 | 6.44 | Blue | 287 |
| Example 5 | | | | | | | |
| Comparative | E | 5.88 | 50 | 3560 | 7.12 | Blue | 312 |
| Example 6 | | | | | | | |
| Comparative | F | 6.41 | 50 | 3160 | 6.32 | Blue | 334 |
| Example 7 | | | | | | | |
| Comparative | G | 5.91 | 50 | 3370 | 6.74 | Blue | 354 |
| Example 8 | | | | | | | |
| Example 1 | Compound | 4.32 | 50 | 4115 | 8.23 | Blue | 499 |
| | 10 | | | | | | |
| Example 2 | Compound | 4.43 | 50 | 4265 | 8.53 | Blue | 496 |
| | 15 | | | | | | |
| Example 3 | Compound | 4.44 | 50 | 4205 | 8.41 | Blue | 416 |
| | 25 | | | | | | |
| Example 4 | Compound | 4.19 | 50 | 4155 | 8.31 | Blue | 454 |
| | 67 | | | | | | |
| Example 5 | Compound | 4.32 | 50 | 4275 | 8.55 | Blue | 495 |
| | 90 | | | | | | |
| Example 6 | Compound | 4.95 | 50 | 4370 | 8.74 | Blue | 493 |
| | 99 | | | | | | |
| Example 7 | Compound | 4.67 | 50 | 4205 | 8.41 | Blue | 476 |
| | 132 | | | | | | |
| Example 8 | Compound | 4.57 | 50 | 4255 | 8.51 | Blue | 484 |
| | 145 | | | | | | |
| Example 9 | Compound | 4.47 | 50 | 4280 | 8.56 | Blue | 444 |
| | 169 | | | | | | |
| Example 10 | Compound | 4.36 | 50 | 4300 | 8.60 | Blue | 455 |
| | 206 | | | | | | |
| Example 11 | Compound | 4.37 | 50 | 3990 | 7.98 | Blue | 405 |
| | 233 | | | | | | |
| Example 12 | Compound | 4.51 | 50 | 4160 | 8.32 | Blue | 432 |
| | 234 | | | | | | |
| Example 13 | Compound | 4.33 | 50 | 4075 | 8.15 | Blue | 487 |
| | 235 | | | | | | |
| Example 14 | Compound | 4.41 | 50 | 4220 | 8.44 | Blue | 463 |
| | 236 | | | | | | |
| Example 15 | Compound | 4.44 | 50 | 4180 | 8.36 | Blue | 499 |
| | 237 | | | | | | |
| Example 16 | Compound | 4.32 | 50 | 4355 | 8.71 | Blue | 488 |
| | 238 | | | | | | |
| Example 17 | Compound | 4.27 | 50 | 4085 | 8.17 | Blue | 489 |
| | 239 | | | | | | |

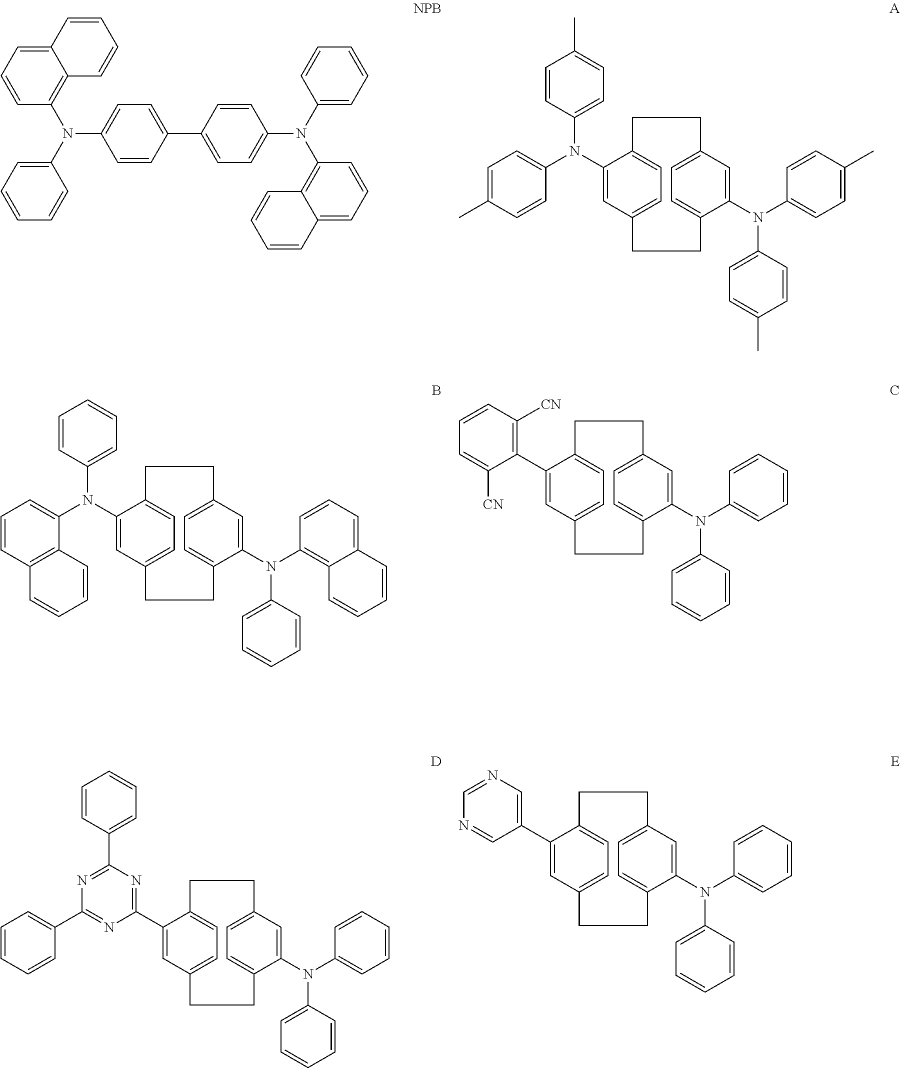

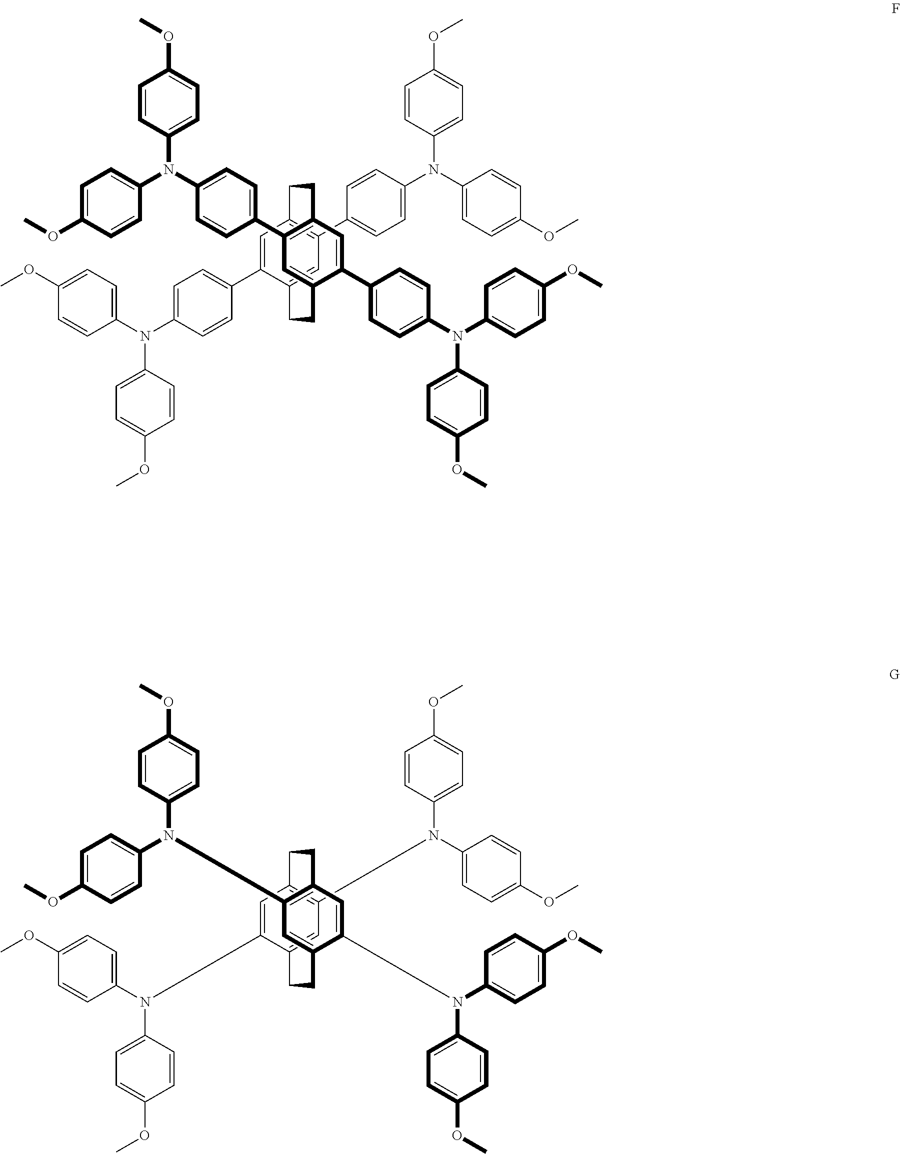

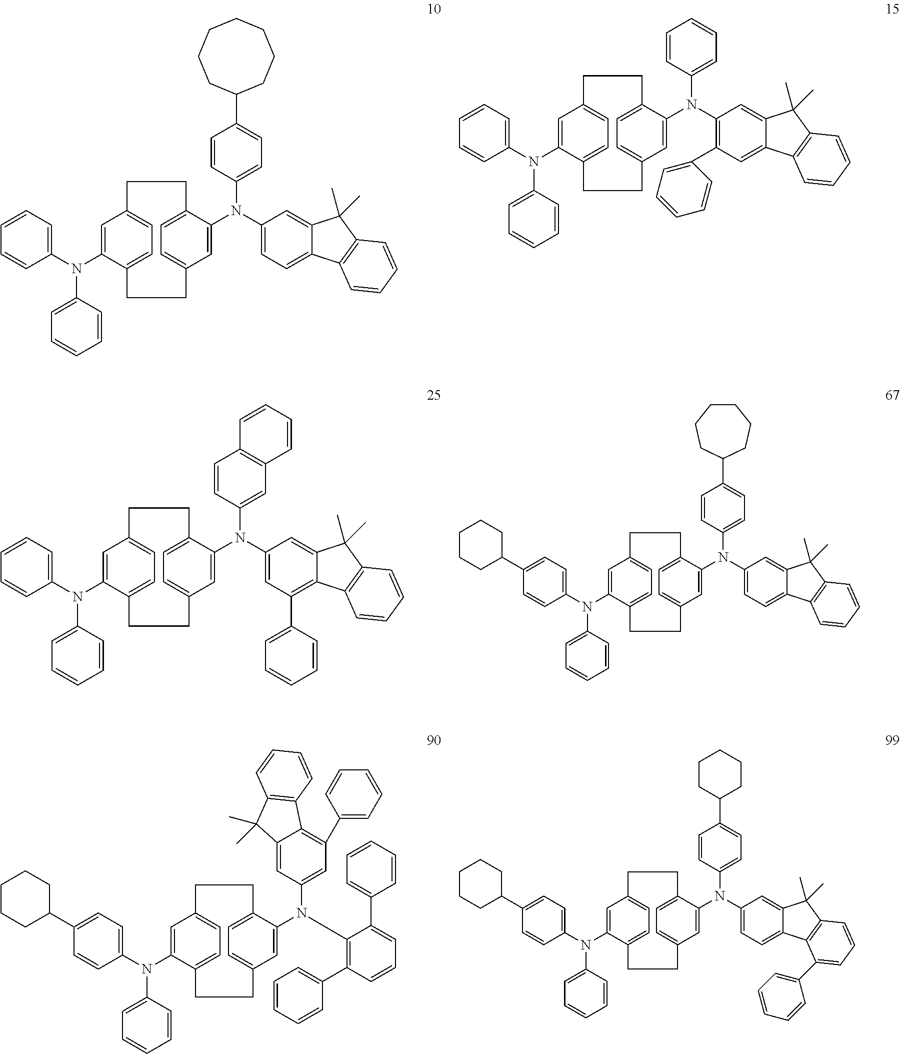

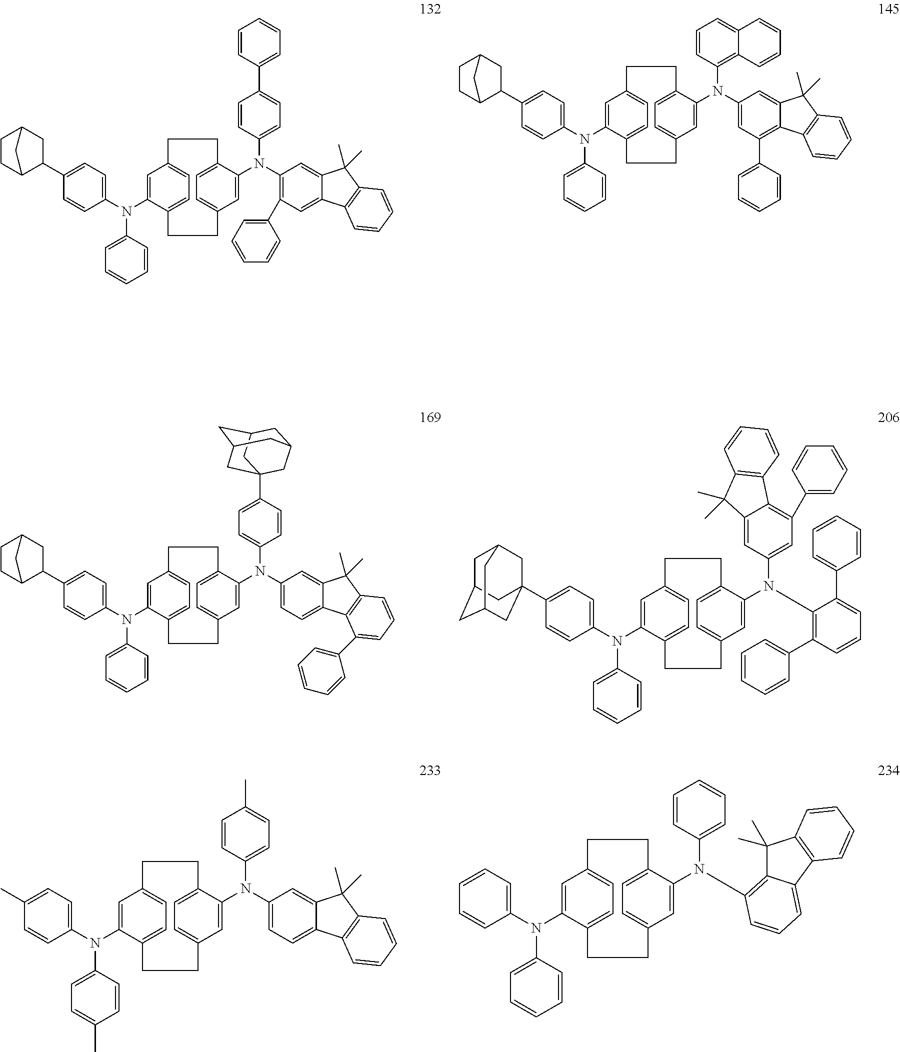

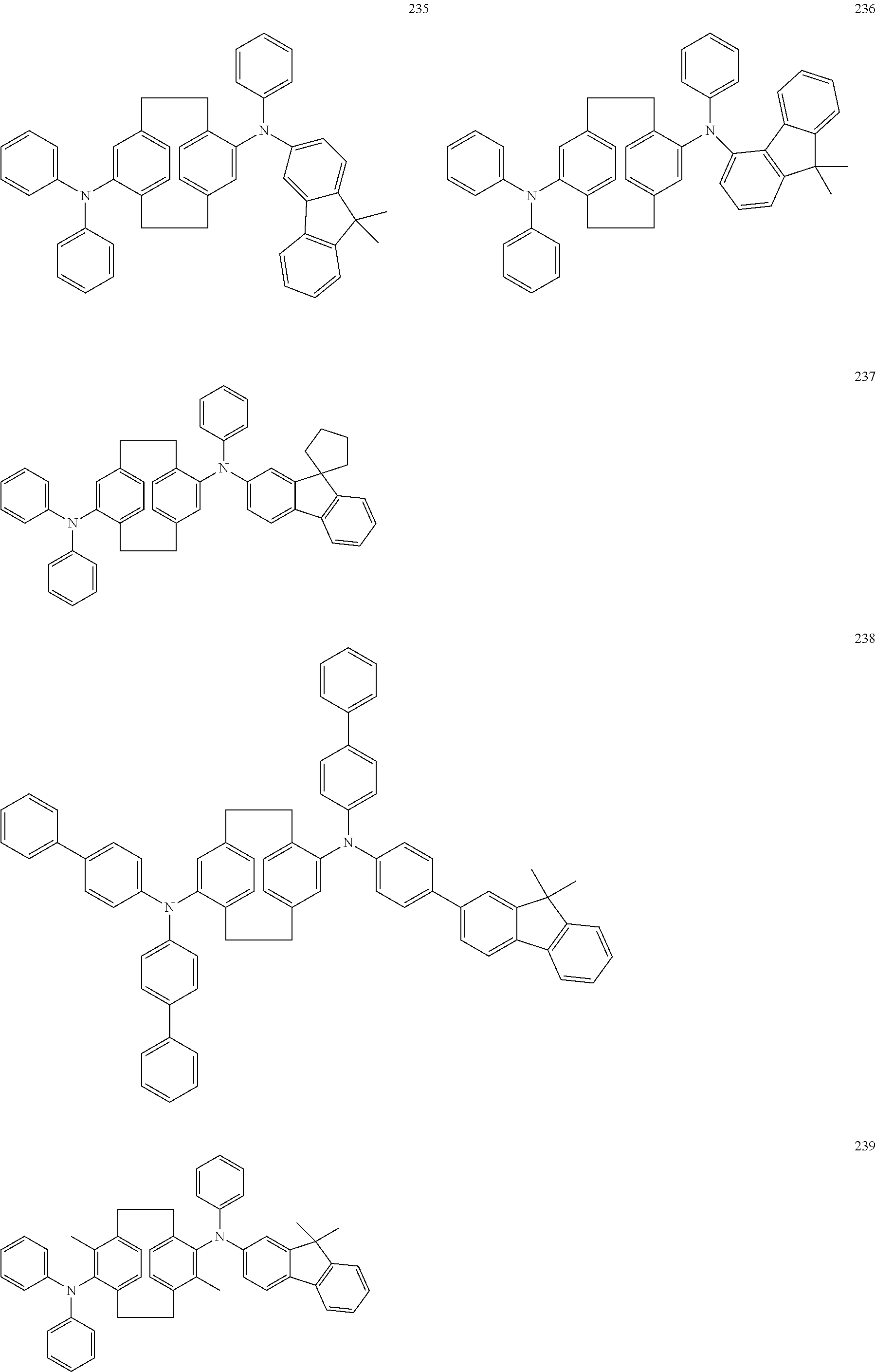 [SMILES: CC1(C)c2ccccc2-c2cc(N(c3ccccc3)c3cc4ccc3CCc3ccc(c(N(c5ccccc5)c5ccccc5)c3)CC4)ccc21 ; CC1(C)c2ccccc2-c2c(N(c3ccccc3)c3cc4ccc3CCc3ccc(c(N(c5ccccc5)c5ccccc5)c3)CC4)cccc21 ; c1ccc(N(c2ccccc2)c2cc3ccc2CCc2ccc(c(N(c4ccccc4)c4ccc5c(c4)C4(CCCC4)c4ccccc4-5)c2)CC3)cc1 ; CC1(C)c2ccccc2-c2ccc(-c3ccc(N(c4ccc(-c5ccccc5)cc4)c4cc5ccc4CCc4ccc(c(N(c6ccc(-c7ccccc7)cc6)c6ccc(-c7ccccc7)cc6)c4)CC5)cc3)cc21 ; Cc1c2ccc(c1N(c1ccccc1)c1ccccc1)CCc1ccc(c(N(c3ccccc3)c3ccc4c(c3)C(C)(C)c3ccccc3-4)c1C)CC2]

Referring to Table 1, the light-emitting devices according to Examples 1 to 17 were found to have a relatively equal or lower driving voltage, relatively higher luminance, relatively higher luminescence efficiency, and/or a relatively longer lifespan than the light-emitting devices according to Comparative Examples 1 to 8.

According to the one or more embodiments, an amine-containing compound represented by Formula 1 may have excellent hole transporting characteristics. A light-emitting device including the amine-containing compound may have a low driving voltage, high luminance, high efficiency, and/or a long lifespan. An electronic apparatus including the light-emitting device and an electronic device using the electronic apparatus may have improved display quality.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims, and equivalents thereof.

What is claimed is:

1. A light-emitting device comprising:
a first electrode;
a second electrode facing the first electrode;
an interlayer between the first electrode and the second electrode and comprising an emission layer; and
an amine-containing compound represented by Formula 1: 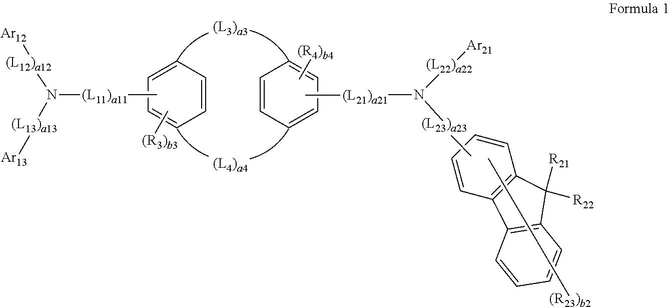
wherein, in Formula 1,
L₁₁ to L₁₃, L₂₁ to L₂₃, Ar₁₂, Ar₁₃, and Ar₂₁ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a11 to a13 and a21 to a23 are each independently an integer from 0 to 5, wherein, i) when a11 is 0, a group represented by *-(L₁₁)ₐ₁₁-*′ is a single bond, ii) when a12 is 0, a group represented by *-(L₁₂)ₐ₁₂-*′ is a single bond, iii) when a13 is 0, a group represented by *-(L₁₃)ₐ₁₃-*′ is a single bond, iv) when a21 is 0, a group represented by *-(L₂₁)ₐ₂₁-*′ is a single bond, v) when a22 is 0, a group represented by *-(L₂₂)ₐ₂₂-*′ is a single bond, vi) when a23 is 0, a group represented by *-(L₂₃)ₐ₂₃-*′ is a single bond, vii) when a11 is one of 2 to 5, a plurality of L₁₁(s) are identical to or different from each other, viii) when a12 is one of 2 to 5, a plurality of L₁₂(s) are identical to or different from each other, ix) when a13 is one of 2 to 5, a plurality of L₁₃(s) are identical to or different from each other, x) when a21 is one of 2 to 5, a plurality of L₂₁(s) are identical to or different from each other, xi) when a22 is one of 2 to 5, a plurality of L₂₂(s) are identical to or different from each other, and xii) when a23 is one of 2 to 5, a plurality of L₂₃(s) are identical to or different from each other,
L₃ and L₄ are each independently *—O—*′, *—S*′, *—N(Q₁)-*′, or *—C(Q₁)(Q₂)-*′,
a3 and a4 are each independently an integer from 0 to 4, wherein, i) when a3 is 0, a group represented by *-(L₃)ₐ₃*′ is a single bond, ii) when a4 is 0, a group represented by *-(L₄)ₐ₄-*′ is a single bond, iii) when a3 is one of 2 to 4, a plurality of L₃(s) are identical to or different from each other, and iv) when a4 is one of 2 to 4, a plurality of L₄(s) are identical to or different from each other,
R₃, R₄, R₂₁, R₂₂, and R₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
R₂₁ and R₂₂ are optionally bonded to each other to form a C₃-C₆₀ carbocyclic group unsubstituted or substituted with at least one R₁₀ₐ or a C₁-C₆₀ heterocyclic group unsubstituted or substituted with at least one R₁₀ₐ,
b2 is an integer from 0 to 7, wherein, when b2 is one of 2 to 7, a plurality of R₂₃(s) are identical to or different from each other,
b3 is an integer from 0 to 3, wherein, when b3 is one of 2 and 3, a plurality of R₃(s) are identical to or different from each other,
b4 is an integer from 0 to 3, wherein, when b4 is one of 2 and 3, a plurality of R₄(s) are identical to or different from each other,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆ alkoxy group, a phenyl group, a biphenyl group, or any combination thereof, and
* and *′ each indicate a binding site to a neighboring atom.

2. The light-emitting device of claim 1, wherein the interlayer further comprises a hole transport region between the first electrode and the emission layer and an electron transport region between the emission layer and the second electrode layer,
the hole transport region comprises a hole injection layer, a hole transport layer, an emission auxiliary layer, an electron blocking layer, or any combination thereof, and
the electron transport region comprises a buffer layer, a hole blocking layer, an electron control layer, an electron transport layer, an electron injection layer, or any combination thereof.

3. The light-emitting device of claim 1, wherein the amine-containing compound is comprised in the interlayer.

4. The light-emitting device of claim 2, wherein the amine-containing compound is comprised in the hole transport region.

5. The light-emitting device of claim 2, wherein the amine-containing compound is comprised in the hole transport layer, and
the hole transport layer is in direct contact with the emission layer.

6. The light-emitting device of claim 1, further comprising a capping layer outside the first electrode,
wherein the amine-containing compound is comprised in the capping layer.

7. The light-emitting device of claim 1, further comprising:
a first capping layer outside the first electrode; and
a second capping layer outside the second electrode,
wherein the amine-containing compound is comprised in the first capping layer or the second capping layer.

8. An electronic apparatus comprising the light-emitting device of claim 1.

9. The electronic apparatus of claim 8, further comprising:
a thin-film transistor electrically connected to the light-emitting device; and
a color filter, a color conversion layer, a touch screen layer, a polarizing layer, or any combination thereof.

10. An electronic device comprising the electronic apparatus of claim 8, wherein the electronic device is one of a flat panel display, a curved display, a computer monitor, a medical monitor, a television, a billboard, an indoor or outdoor lighting and/or signal light, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a microdisplay, a three-dimensional (3D) display, a virtual or augmented-reality display, a vehicle, a video wall including multiple displays tiled together, a theater or stadium screen, a phototherapy device, and a signboard.

11. An amine-containing compound represented by Formula 1: 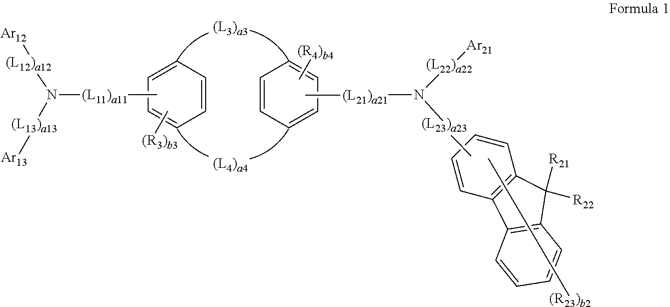
wherein, in Formula 1,
L₁₁ to L₁₃, L₂₁ to L₂₃, Ar₁₂, Ar₁₃, and Ar₂₁ are each independently a C₃-C₆₀ carbocyclic group unsubstituted or substituted with at least one R₁₀ₐ or a C₁-C₆₀ heterocyclic group unsubstituted or substituted with at least one R₁₀ₐ,
a11 to a13 and a21 to a23 are each independently an integer from 0 to 5, wherein, i) when a11 is 0, a group represented by *-(L₁₁)ₐ₁₁-*′ is a single bond, ii) when a12 is 0, a group represented by *-(L₁₂)ₐ₁₂-*′ is a single bond, iii) when a13 is 0, a group represented by *-(L₁₃)ₐ₁₃-*′ is a single bond, iv) when a21 is 0, a group represented by *-(L₂₁)ₐ₂₁-*′ is a single bond, v) when a22 is 0, a group represented by *-(L₂₂)ₐ₂₂-*′ is a single bond, vi) when a23 is 0, a group represented by *-(L₂₃)ₐ₂₃-*′ is a single bond, vii) when a11 is one of 2 to 5, a plurality of L₁₁(s) are identical to or different from each other, viii) when a12 is one of 2 to 5, a plurality of L₁₂(s) are identical to or different from each other, ix) when a13 is one of 2 to 5, a plurality of L₁₃(s) are identical to or different from each other, x) when a21 is one of 2 to 5, a plurality of L₂₁(s) are identical to or different from each other, xi) when a22 is one of 2 to 5, a plurality of L₂₂(s) are identical to or different from each other, and xii) when a23 is one of 2 to 5, a plurality of L₂₃(s) are identical to or different from each other,
L₃ and L₄ are each independently *--*′, *—S*′, *—N(Q₁)-*′, or *—C(Q₁)(Q₂)*′,
a3 and a4 are each independently an integer from 0 to 4, wherein, i) when a3 is 0, a group represented by *-(L₃)ₐ₃-*′ is a single bond, ii) when a4 is 0, a group represented by *-(L₄)ₐ₄-*′ is a single bond, iii) when a3 is one of 2 to 4, a plurality of L₃(s) are identical to or different from each other, and iv) when a4 is one of 2 to 4, a plurality of L₄(s) are identical to or different from each other,
R₃, R₄, R₂₁, R₂₂, and R₂₃ are each independently hydrogen, deuterium, —F, —Cl, —Br, —I, a hydroxyl group, a cyano group, a nitro group, a C₁-C₆₀ alkyl group unsubstituted or substituted with at least one R₁₀ₐ, a C₂-C₆₀ alkenyl group unsubstituted or substituted with at least one R₁₀ₐ, a C₂-C₆₀ alkynyl group unsubstituted or substituted with at least one R₁₀ₐ, a C₁-C₆₀ alkoxy group unsubstituted or substituted with at least one R₁₀ₐ, a C₃-C₆₀ carbocyclic group unsubstituted or substituted with at least one R₁₀ₐ, a C₁-C₆₀ heterocyclic group unsubstituted or substituted with at least one R₁₀ₐ, a C₆-C₆₀ aryloxy group unsubstituted or substituted with at least one R₁₀ₐ, a C₆-C₆₀ arylthio group unsubstituted or substituted with at least one R₁₀ₐ, a C₇-C₆₀ arylalkyl group unsubstituted or substituted with at least one R₁₀ₐ, a C₂-C₆₀ heteroarylalkyl group unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b2 is an integer from 0 to 7, wherein, when b2 is one of 2 to 7, a plurality of R₂₃(s) are identical to or different from each other,
b3 is an integer from 0 to 3, wherein, when b3 is one of 2 and 3, a plurality of R₃(s) are identical to or different from each other,
b4 is an integer from 0 to 3, wherein, when b4 is one of 2 and 3, a plurality of R₄(s) are identical to or different from each other,
R₁₀ₐ is:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂),
Q₁ to Q₃, Q₁₁ to Q₁₃, Q₂₁ to Q₂₃, and Q₃₁ to Q₃₃ are each independently:
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, or a C₁-C₆₀ alkoxy group; or
a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆ alkoxy group, a phenyl group, a biphenyl group, or any combination thereof, and
* and *′ each indicate a binding site to a neighboring atom.

12. The amine-containing compound of claim 11, wherein R₃ and R₄ are each not —N(Q₁)(Q₂).

13. The amine-containing compound of claim 11, wherein Ar₂₁ is a cyclohexane group, a cycloheptane group, a cyclooctane group, an adamantane group, a norbornane group, a benzene group, a biphenyl group, a naphthalene group, a phenanthrene group, a fluorene group, a spiro-bifluorene group, a dibenzofuran group, or a dibenzothiophene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a biphenyl group, a naphthyl group, a phenanthrenyl group, a fluorenyl group, a spiro-bifluorenyl group, a dibenzofuranyl group, a dibenzothiophenyl group, or any combination thereof.

14. The amine-containing compound of claim 11, wherein L₁₁ to L₁₃ and L₂₁ to L₂₃ are each independently a group represented by one of Formulae 2-1 to 2-28: 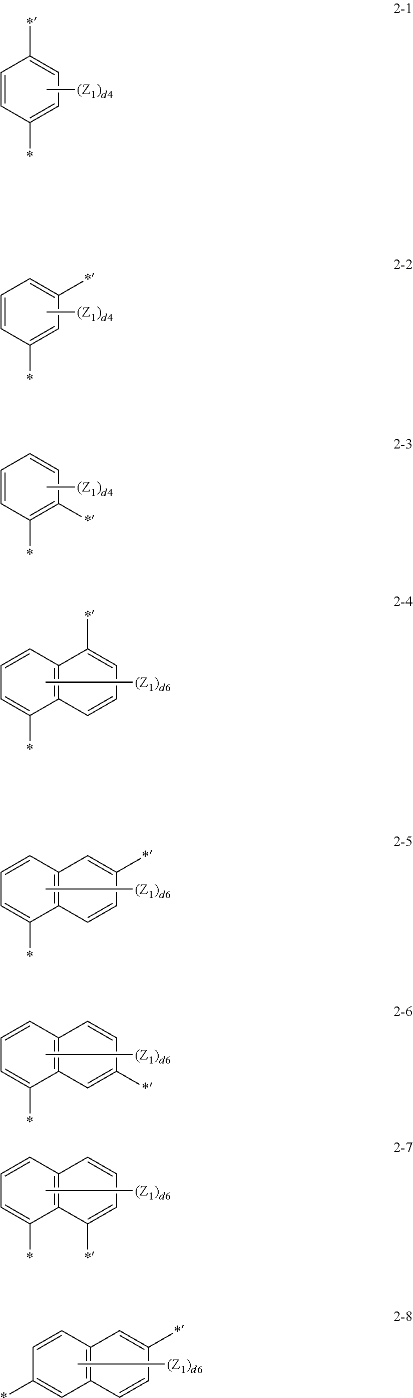 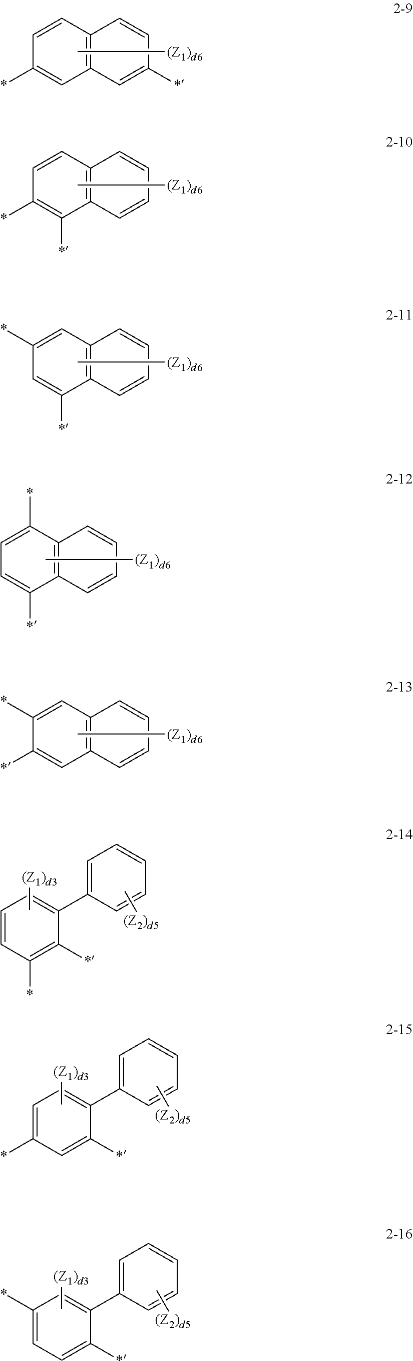 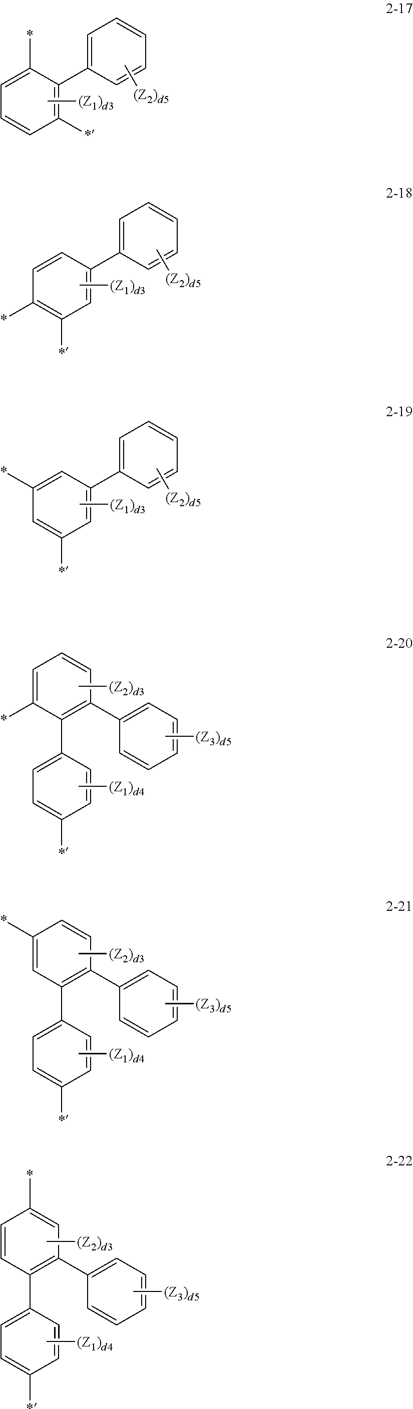 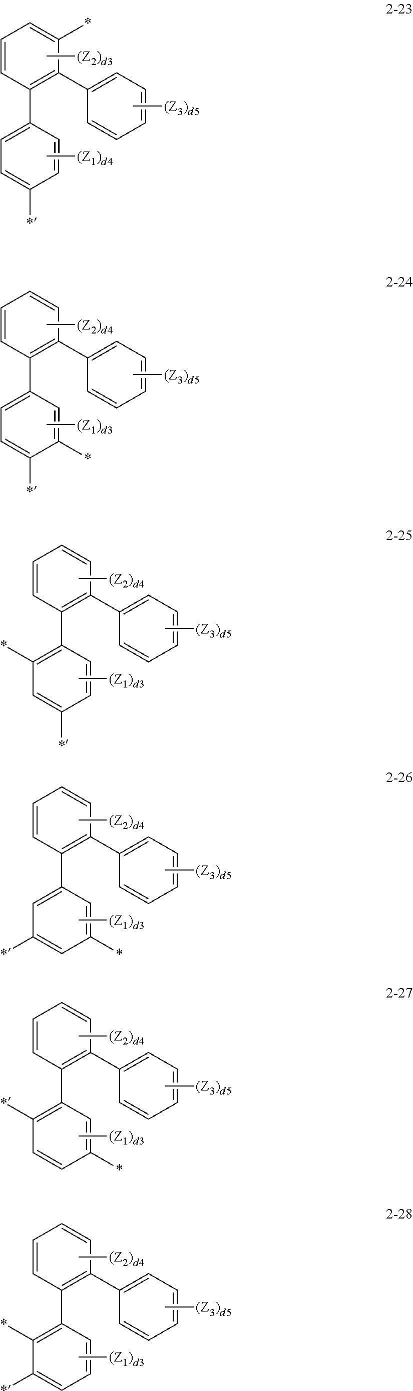
wherein, in Formulae 2-1 to 2-28,
Z₁ to Z₃ are each as described in connection with R₁₀ₐ in claim 11,
d3 is 0, 1, 2, or 3,
d4 is 0, 1, 2, 3, or 4,
d5 is 0, 1, 2, 3, 4, or 5, and
d6 is 0, 1, 2, 3, 4, 5, or 6.

15. The amine-containing compound of claim 11, wherein Ar₁₂ and Ar₁₃ are each independently a cyclohexane group, a cycloheptane group, a cyclooctane group, an adamantane group, a norbornane group, a benzene group, a naphthalene group, or a phenanthrene group, each unsubstituted or substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, or any combination thereof.

16. The amine-containing compound of claim 11, wherein at least one of Ar₂₁, Ar₁₂, and Ar₁₃ is a benzene group, a naphthalene group, or a phenanthrene group, each substituted with deuterium, a C₁-C₂₀ alkyl group, a cyclohexyl group, a cycloheptyl group, a cyclooctyl group, an adamantanyl group, a norbornanyl group, a phenyl group, a naphthyl group, a phenanthrenyl group, or any combination thereof.

17. The amine-containing compound of claim 11, wherein b2 is 0 or 1, and
when b2 is 1, R₂₃ is a phenyl group or a naphthyl group, each unsubstituted or substituted with at least one R₁₀ₐ.

18. The amine-containing compound of claim 11, wherein R₂₁ and R₂₂ are each independently hydrogen, deuterium, or a C₁-C₆₀ alkyl group, and
R₂₁ and R₂₂ are optionally bonded to each other to form a cyclopentane group or a cyclohexane group, each unsubstituted or substituted with at least one R₁₀ₐ.

19. The amine-containing compound of claim 11, wherein at least one of *-(L₃)-*′ and *-(L₄)-*′ is *—CH₂CH₂—*′.

20. The amine-containing compound of claim 11, wherein the amine-containing compound is one of Compounds 1 to 20, 22 to 60, and 62 to 239: 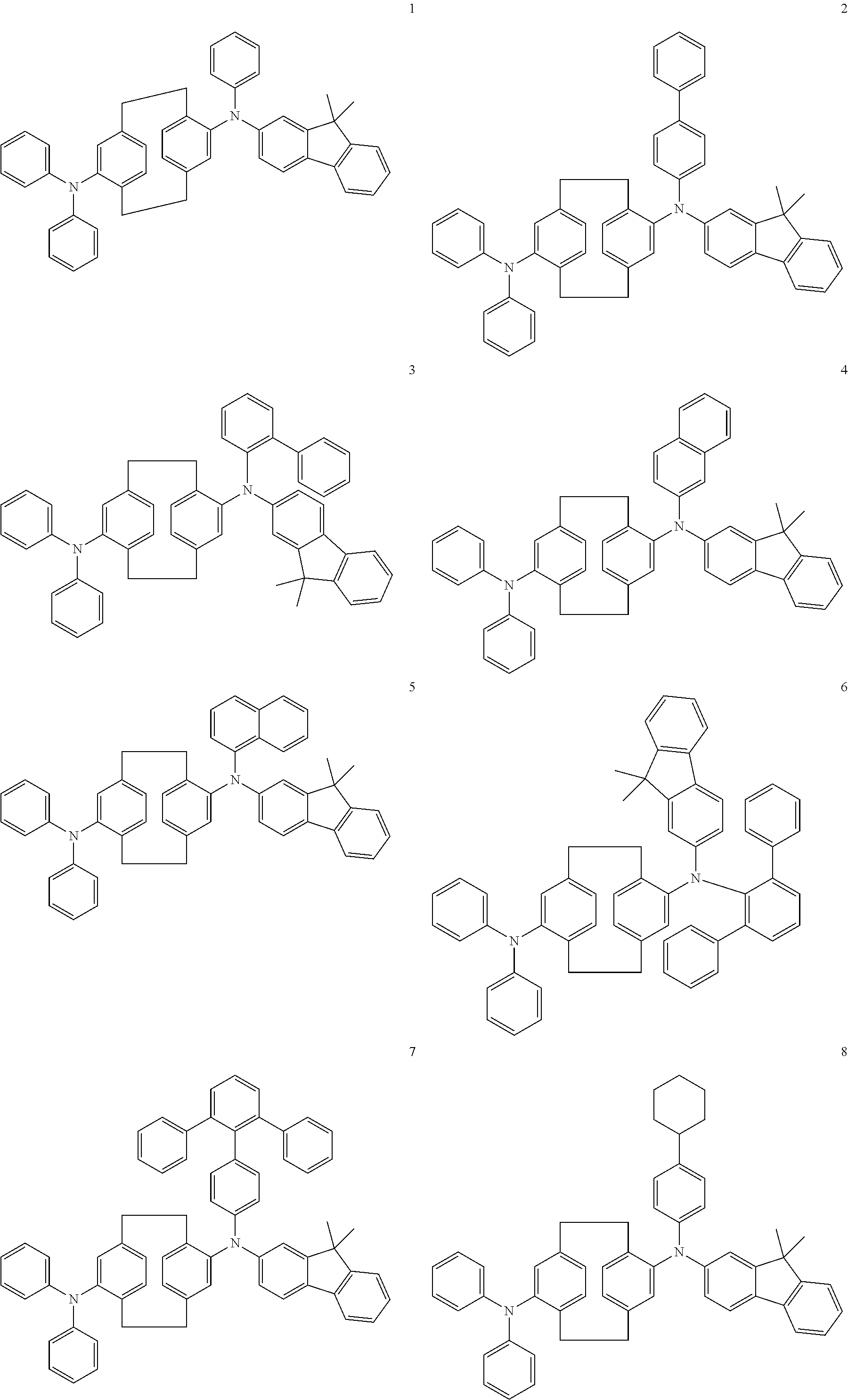 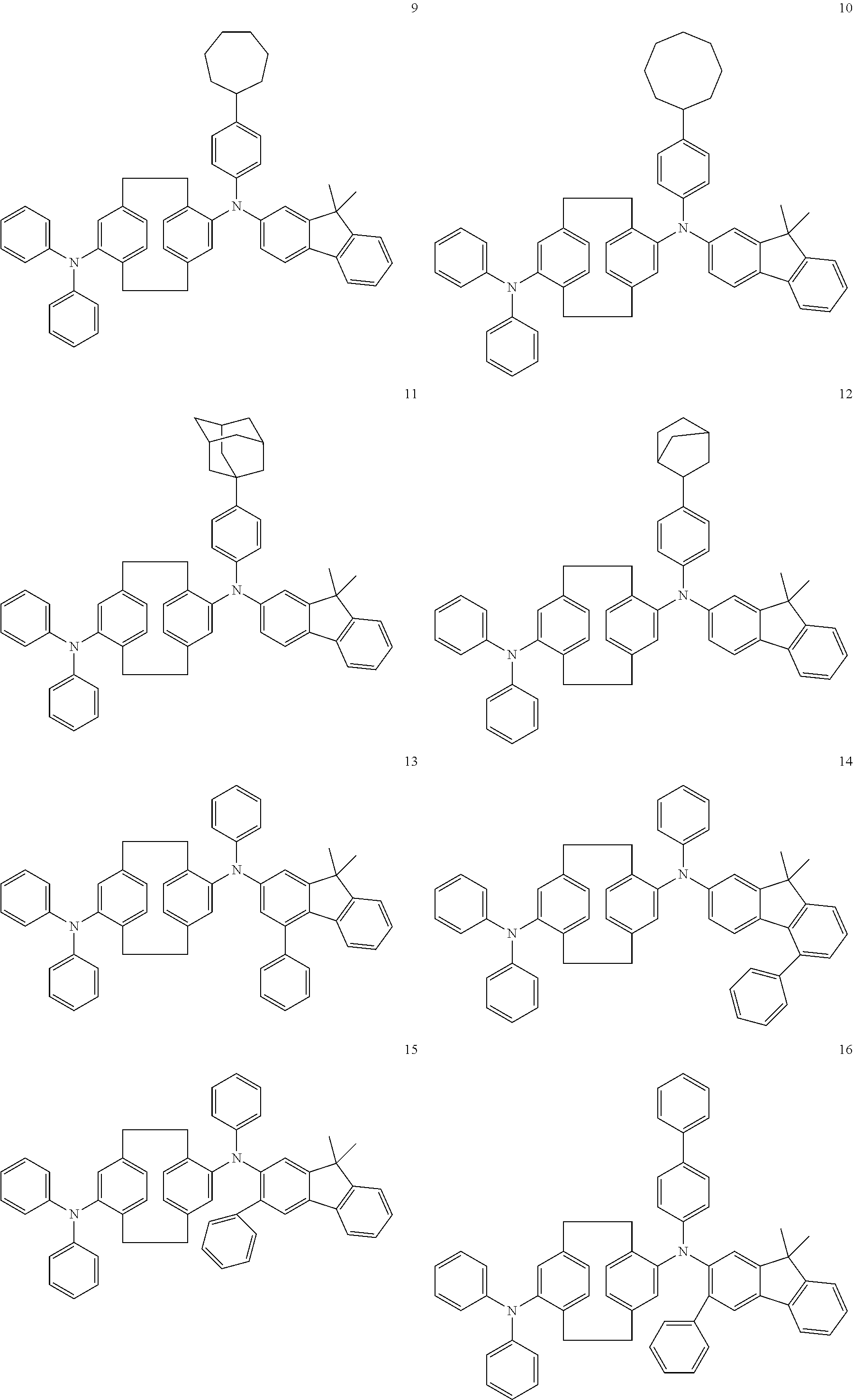 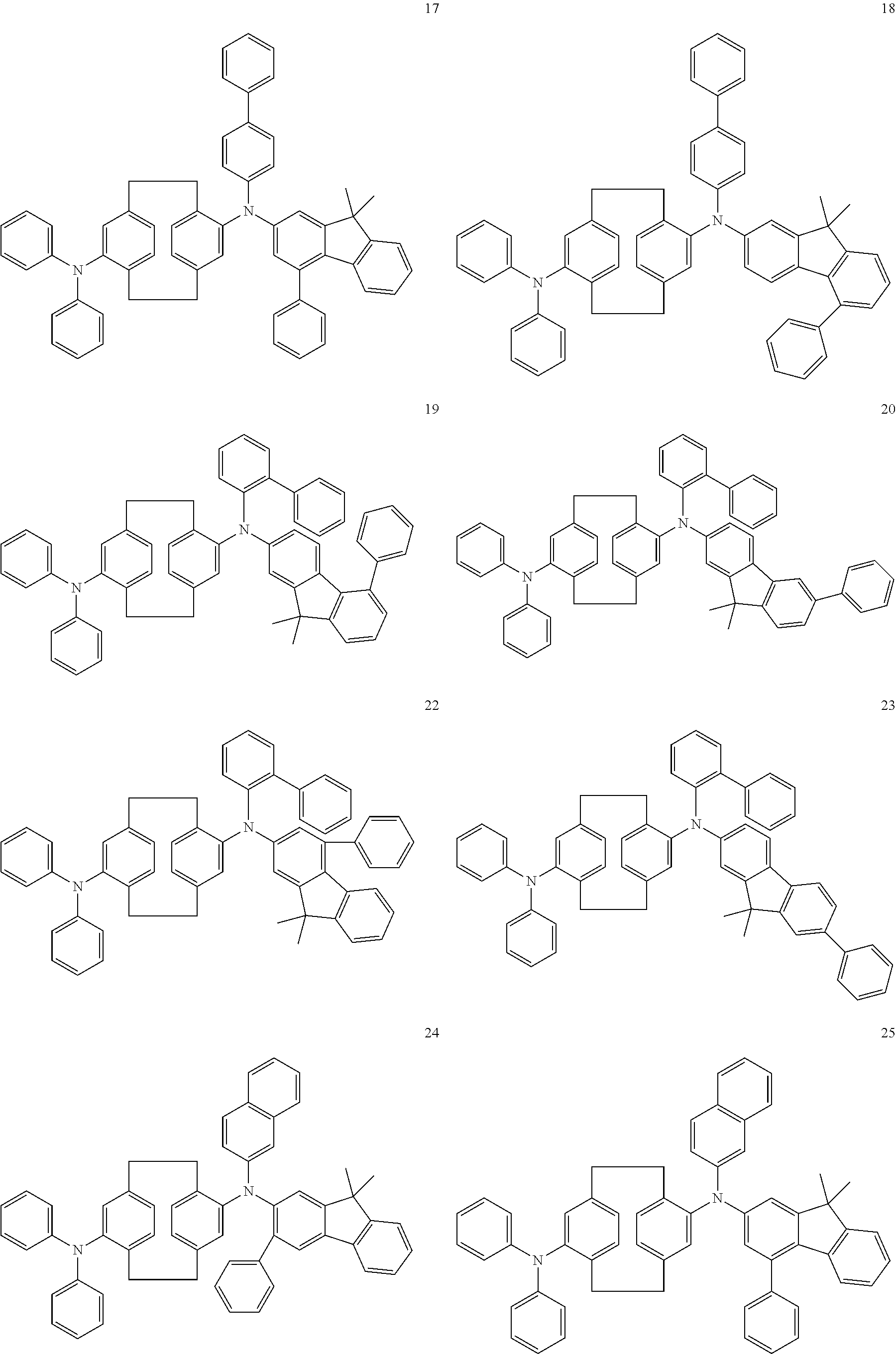 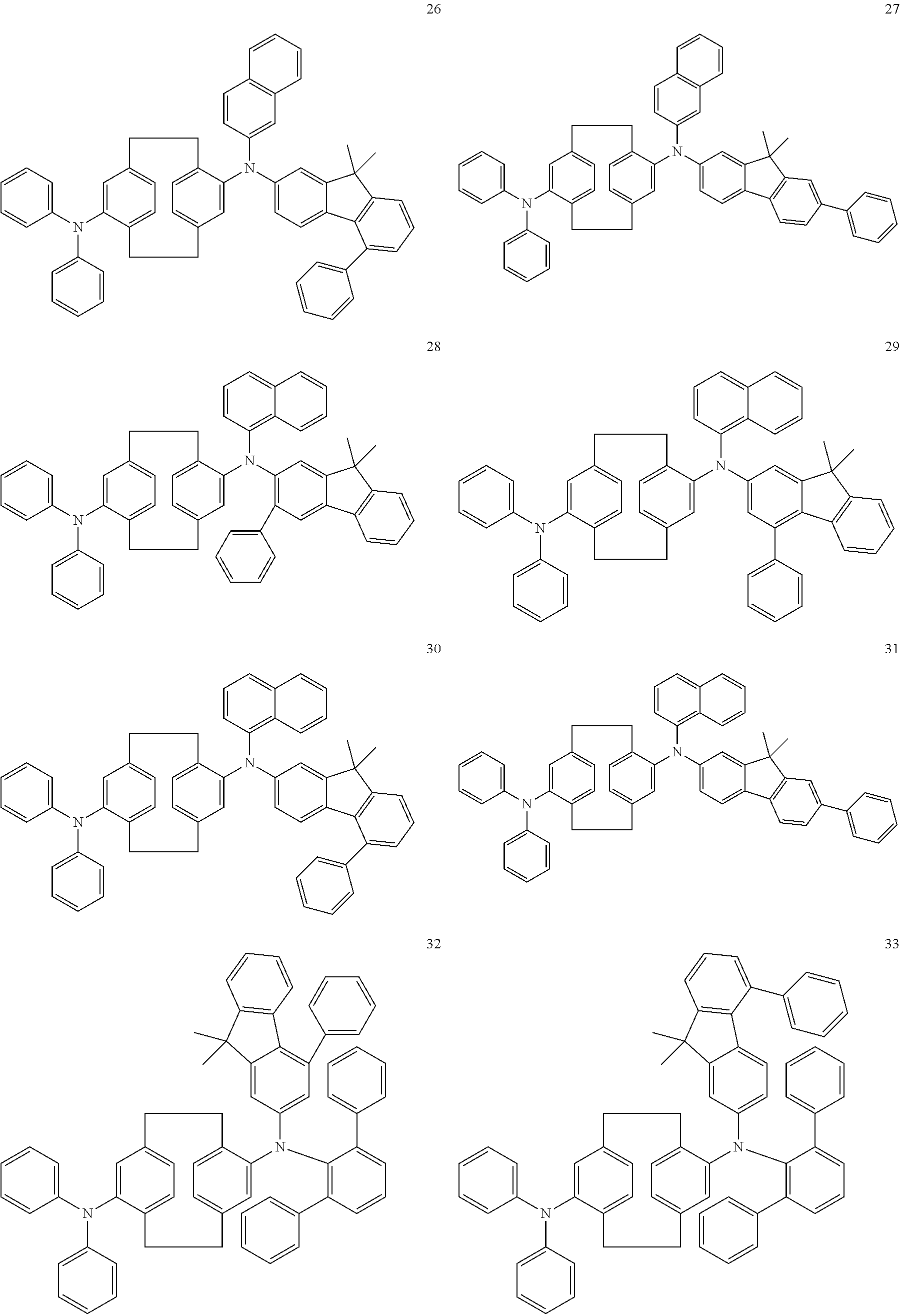 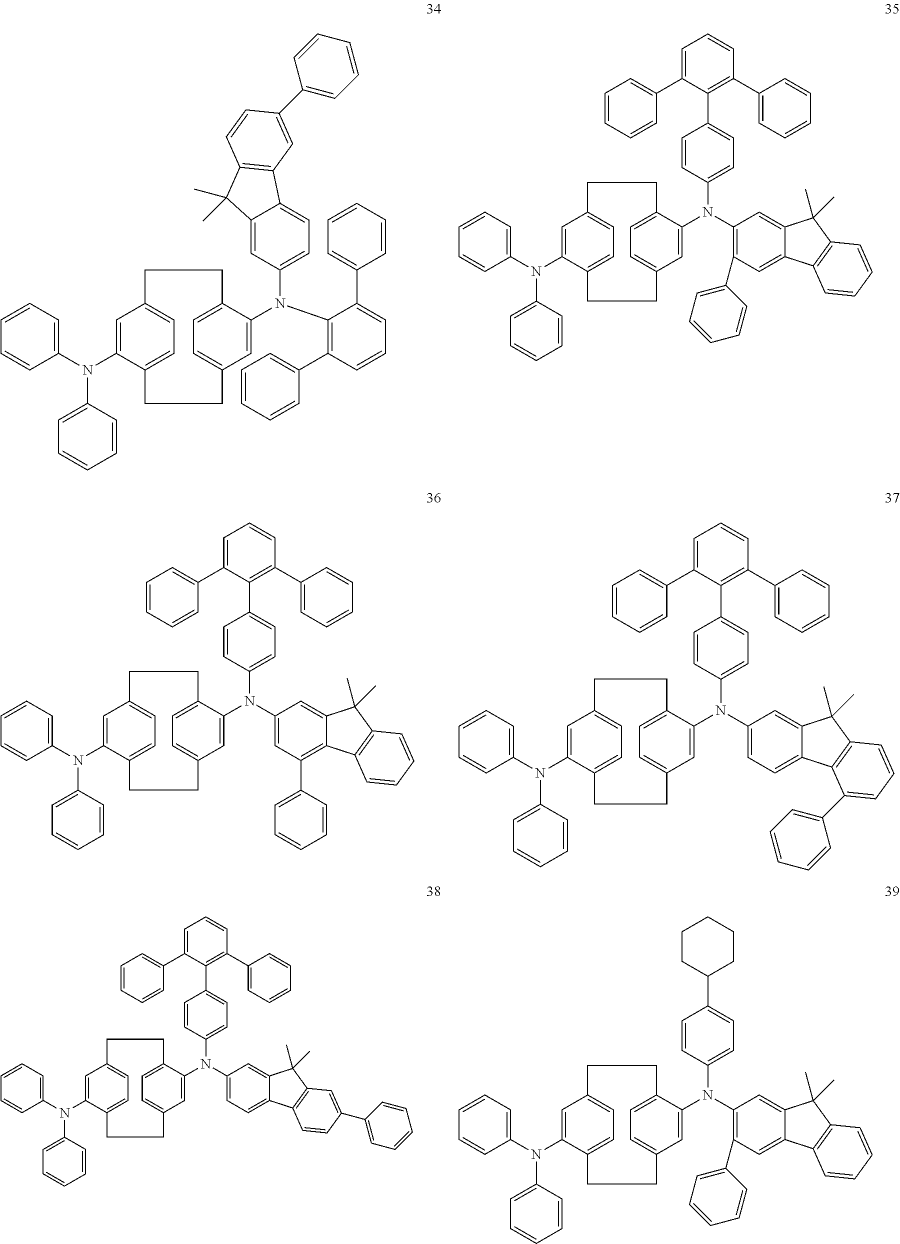 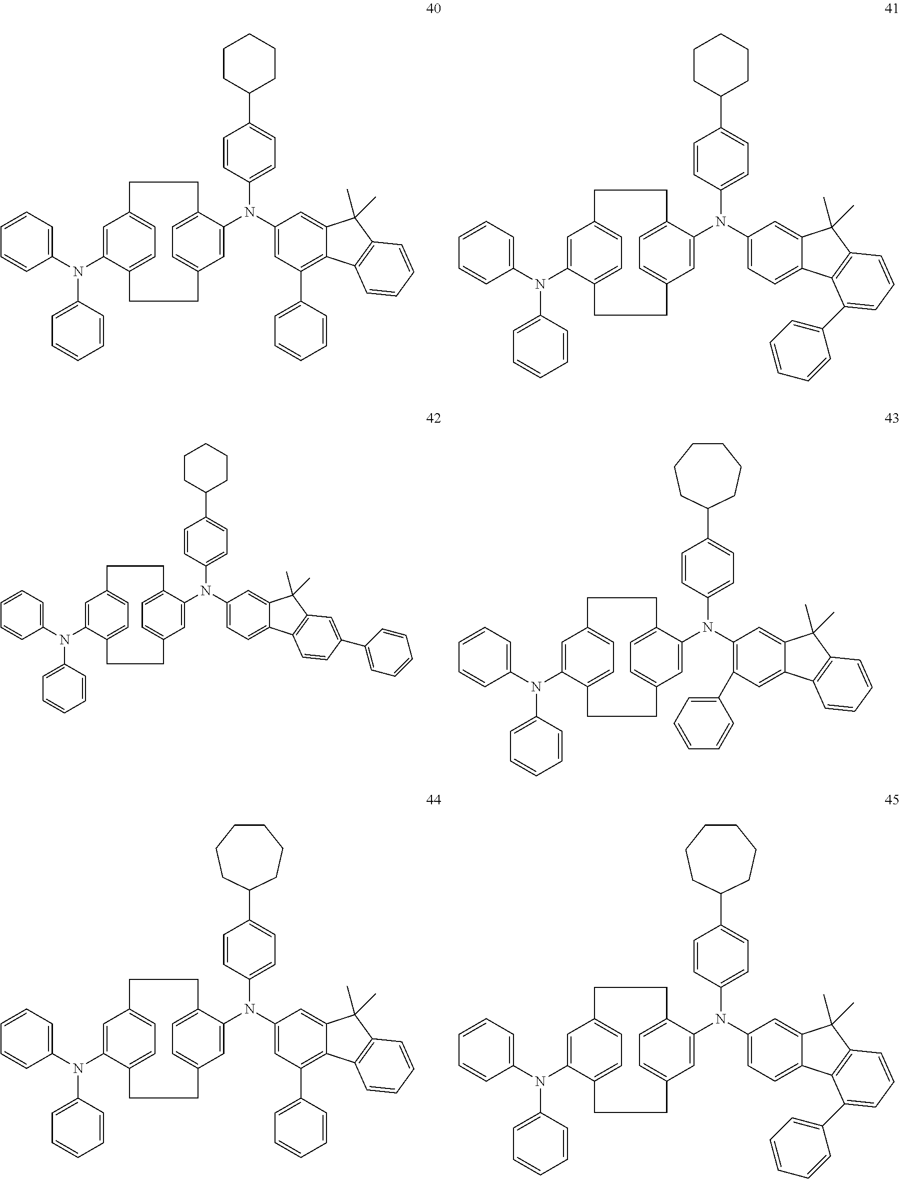 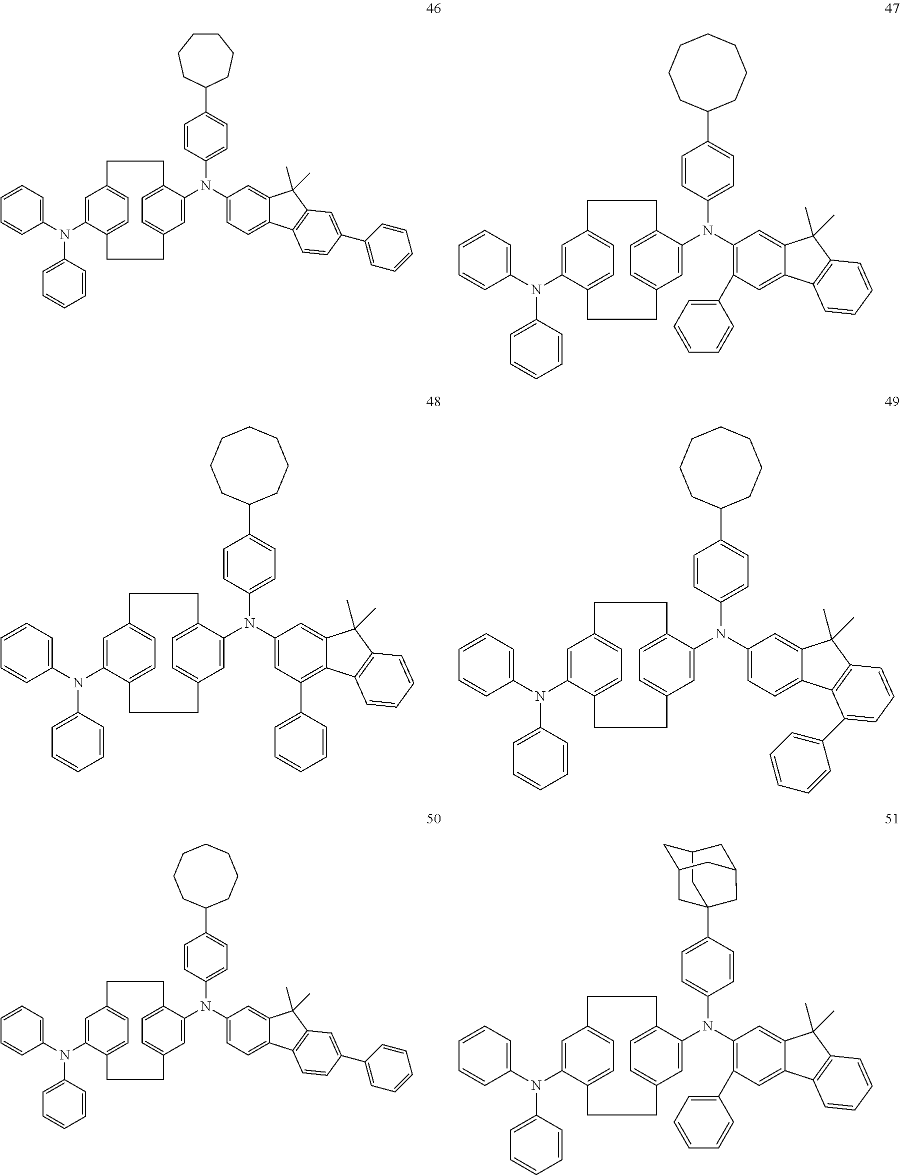 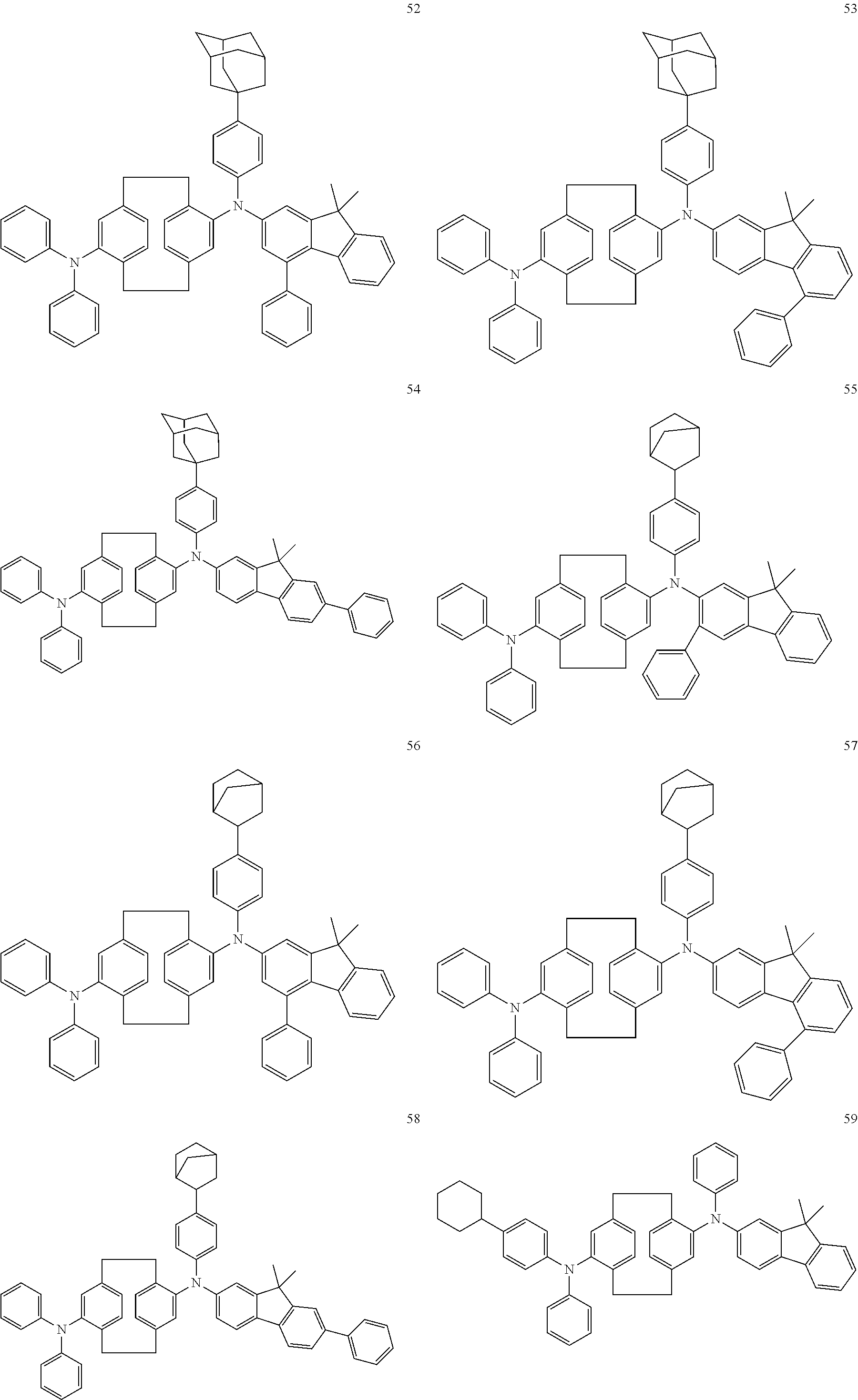 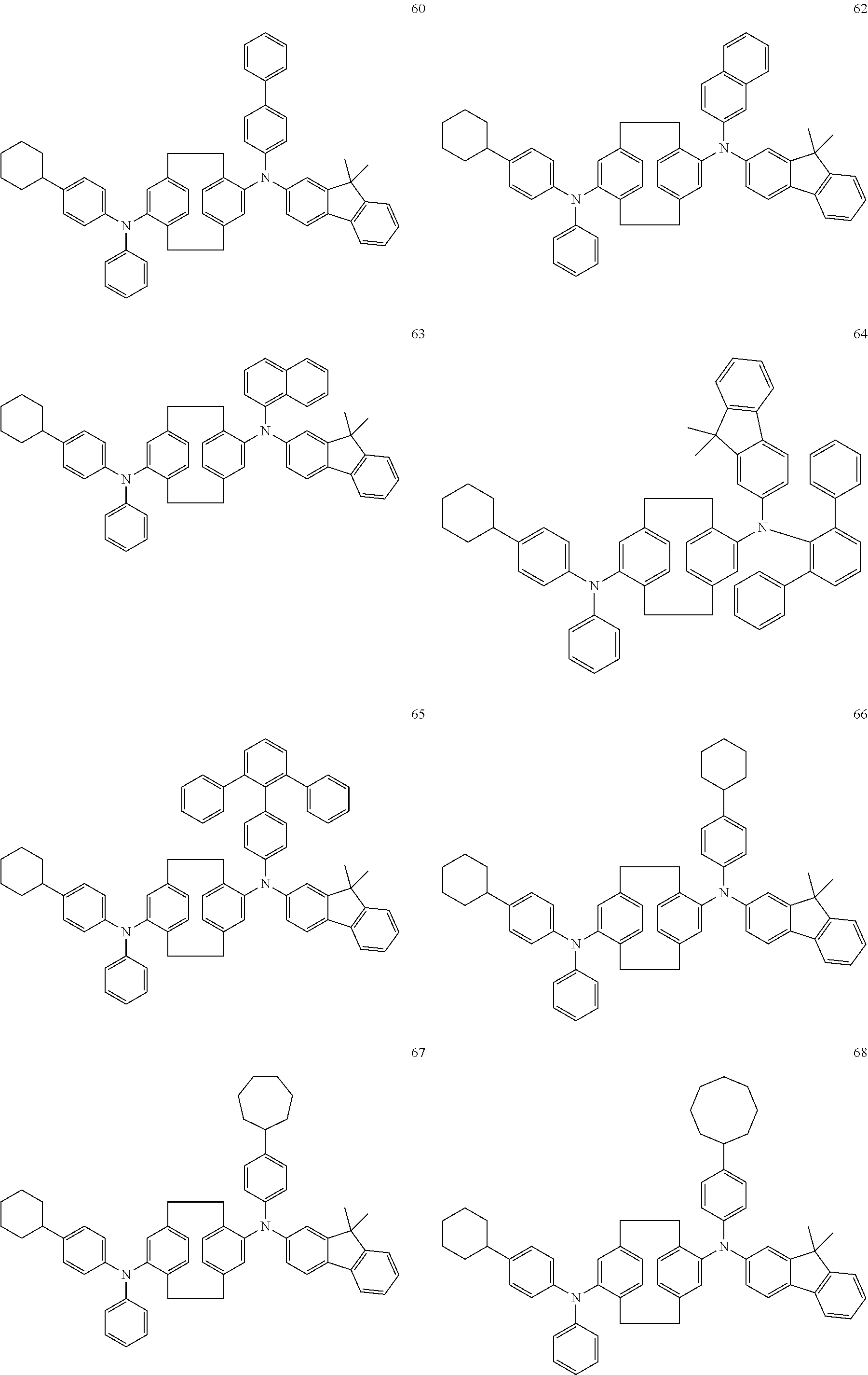 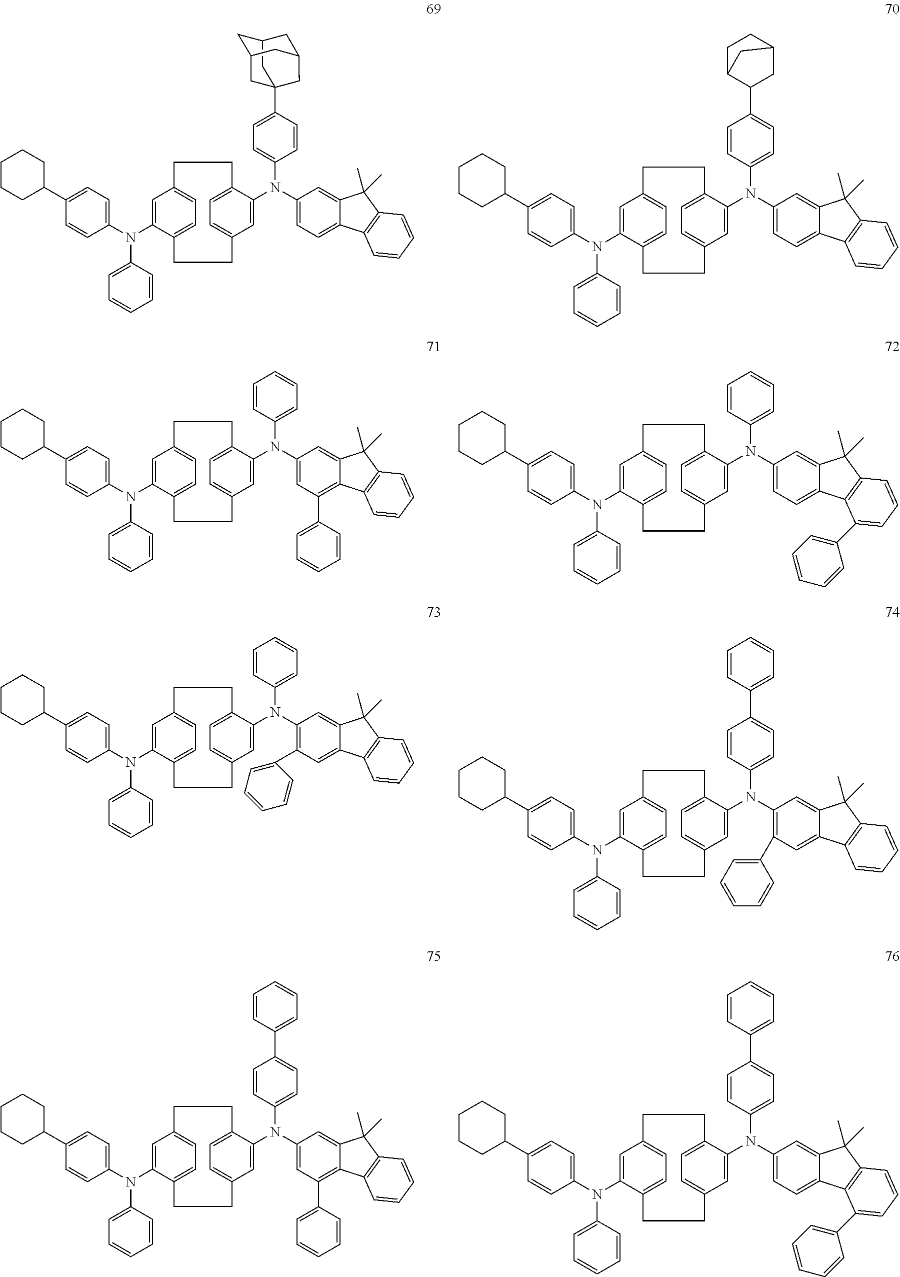 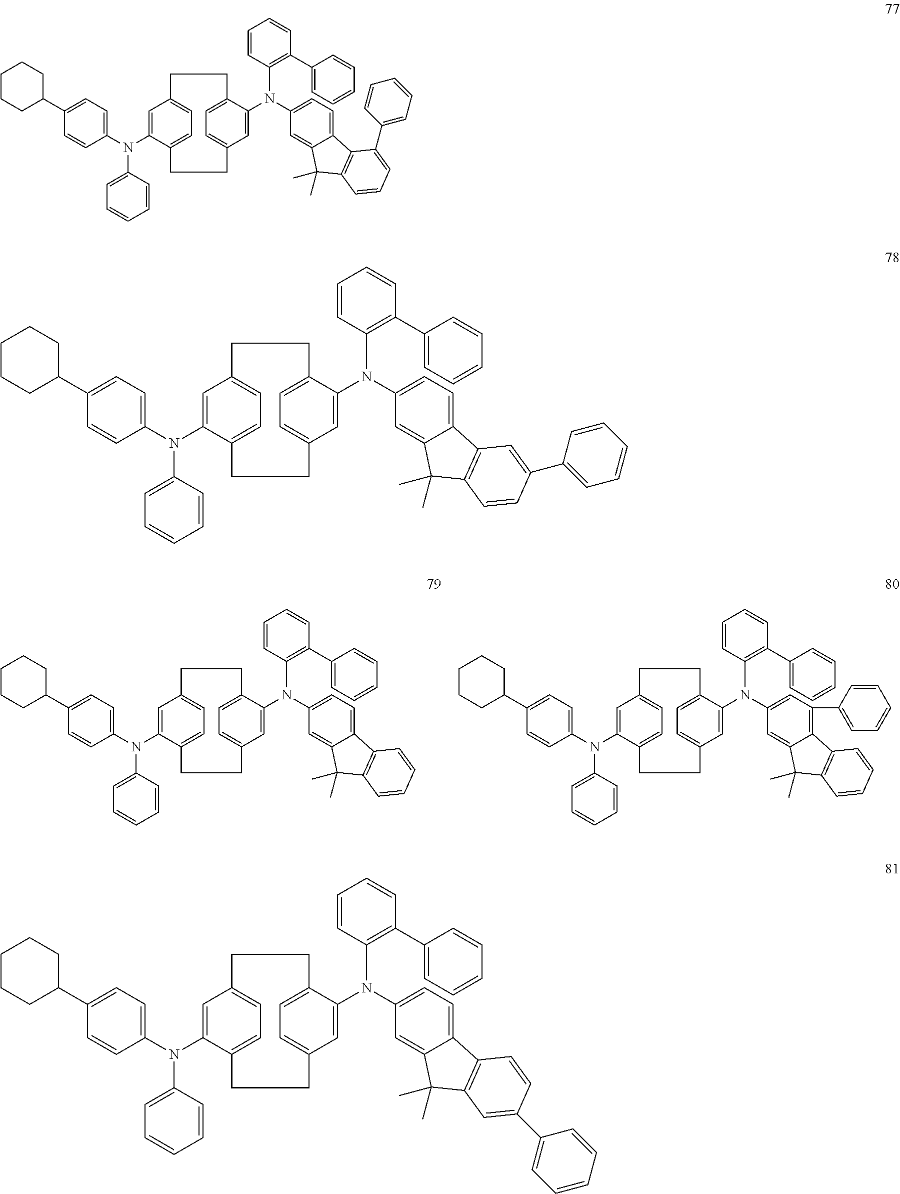 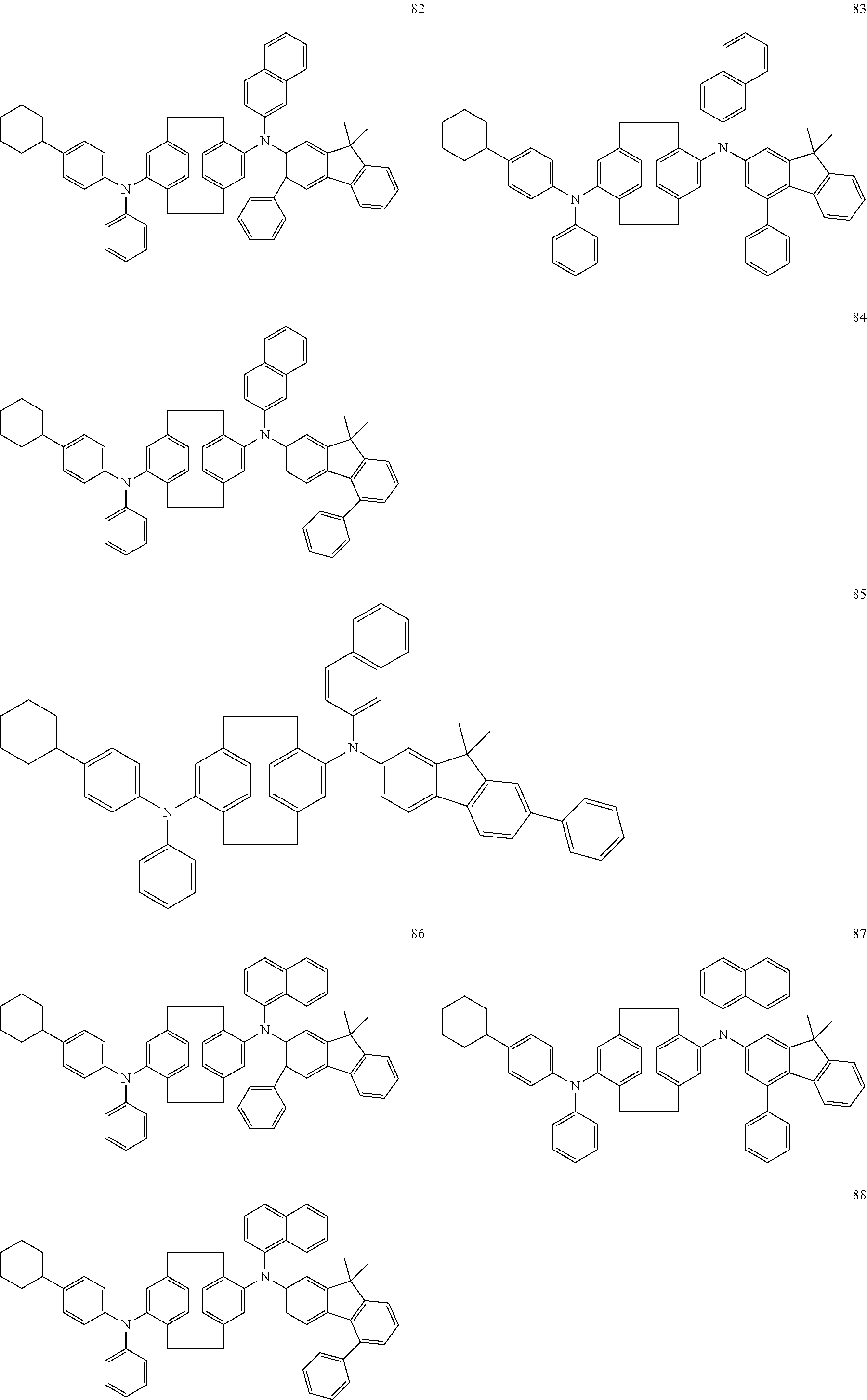 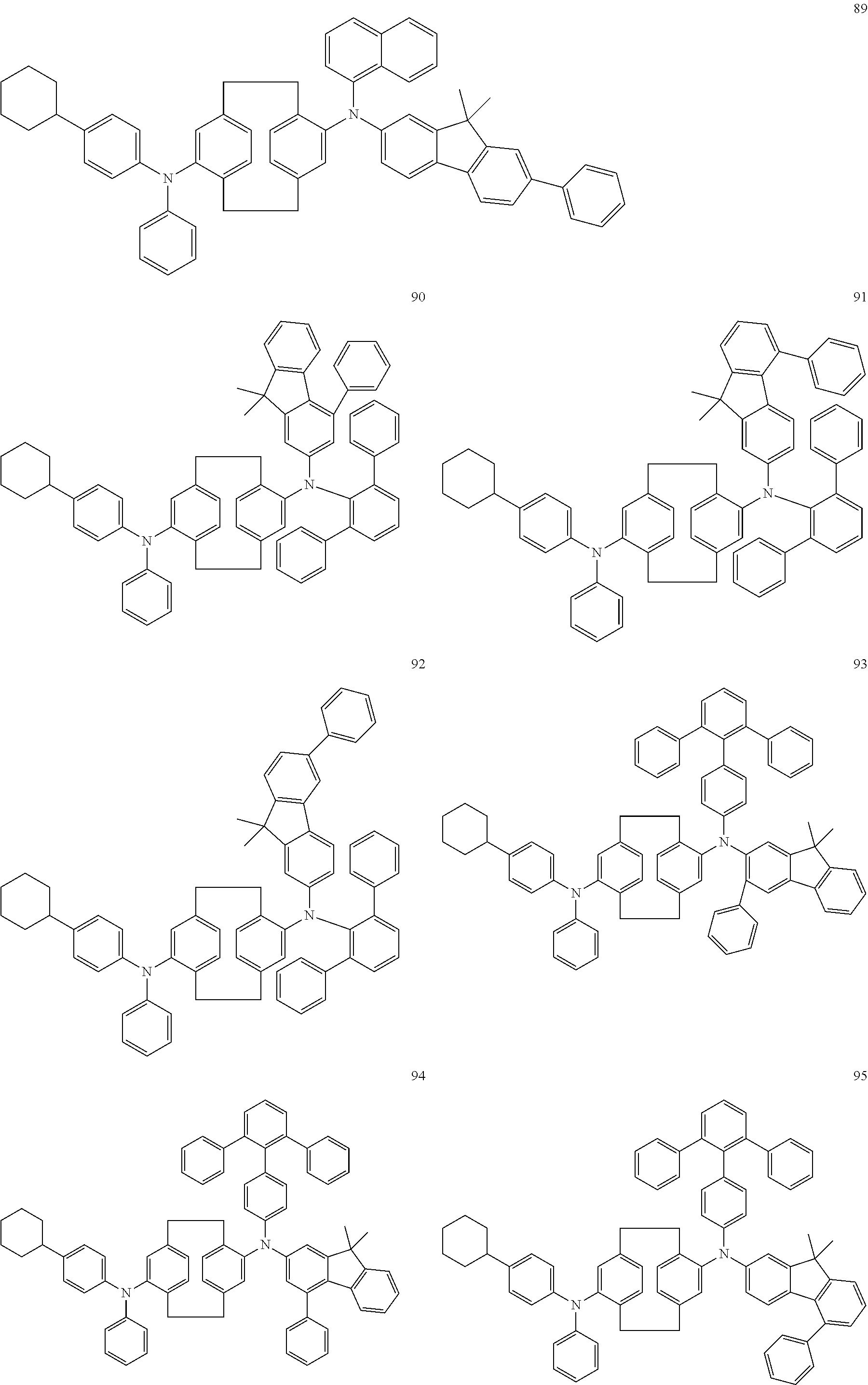 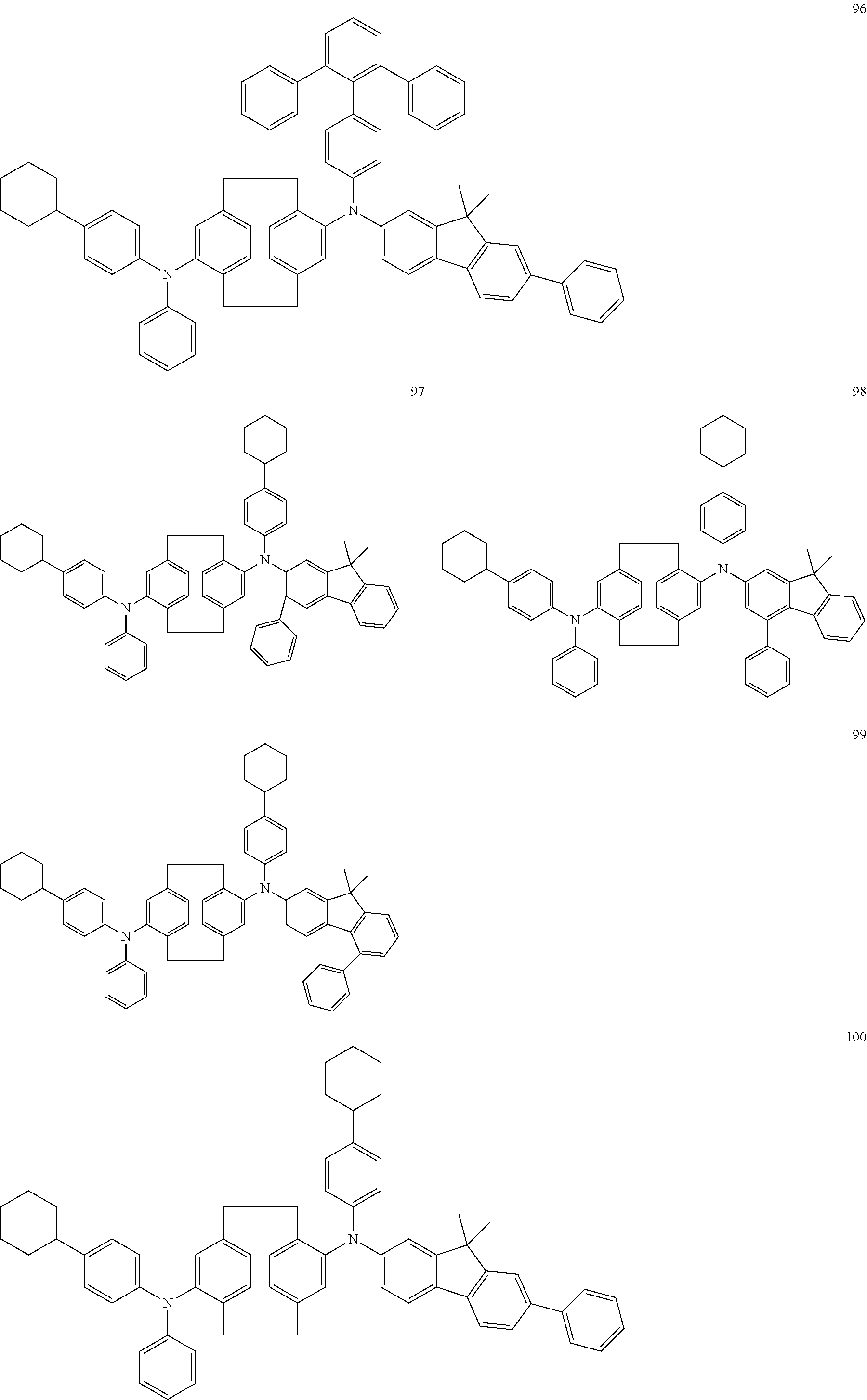 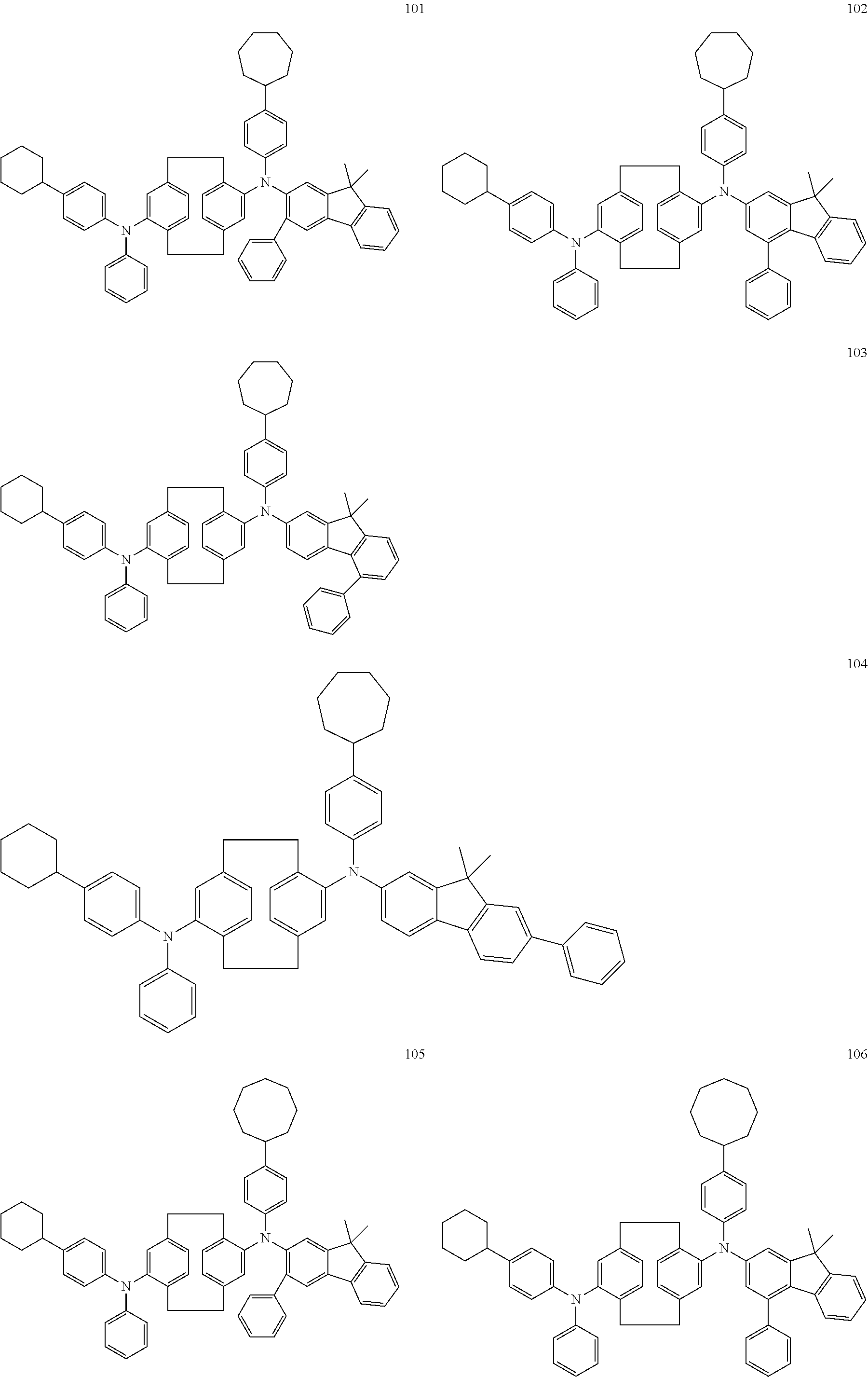 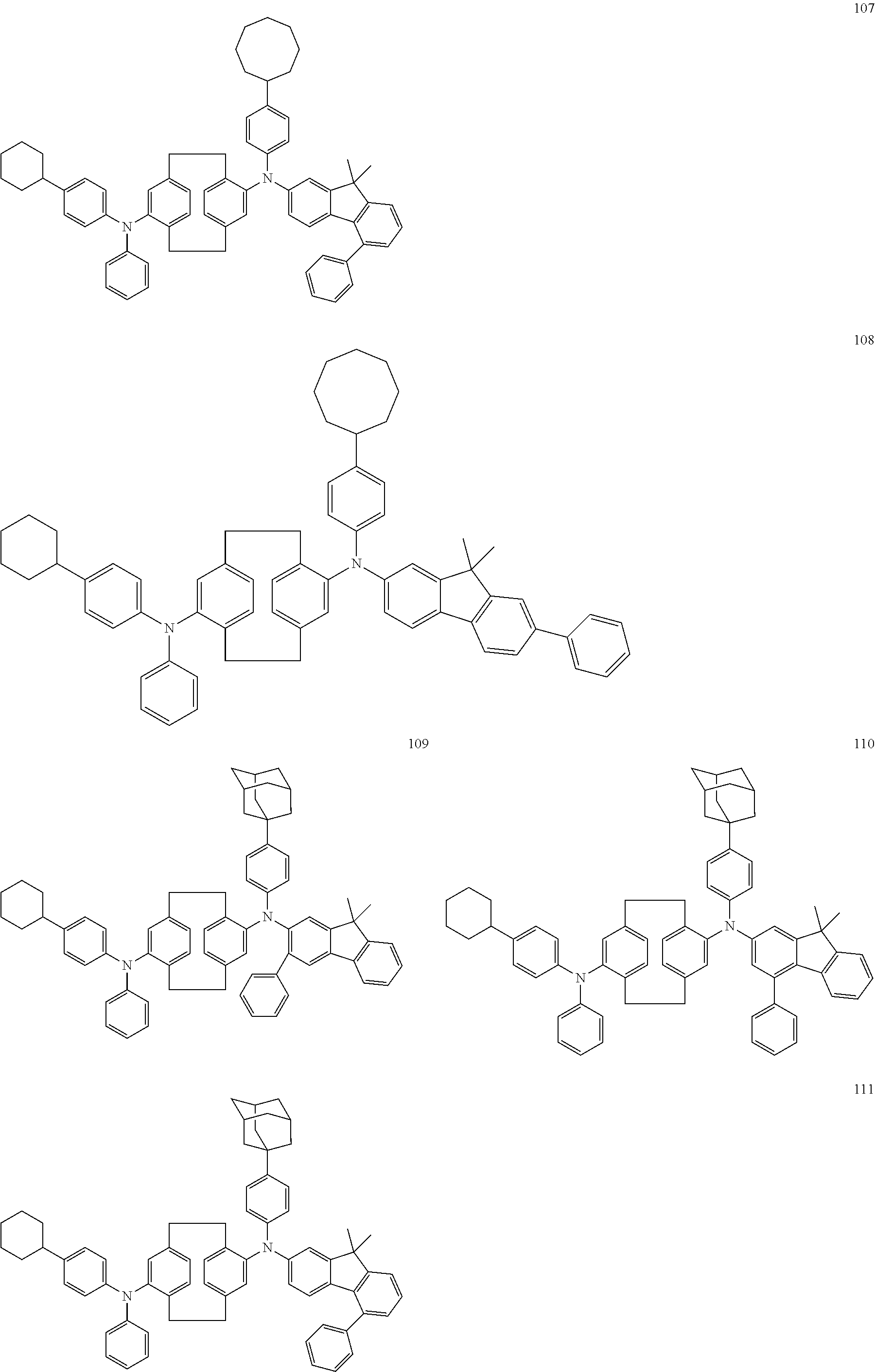 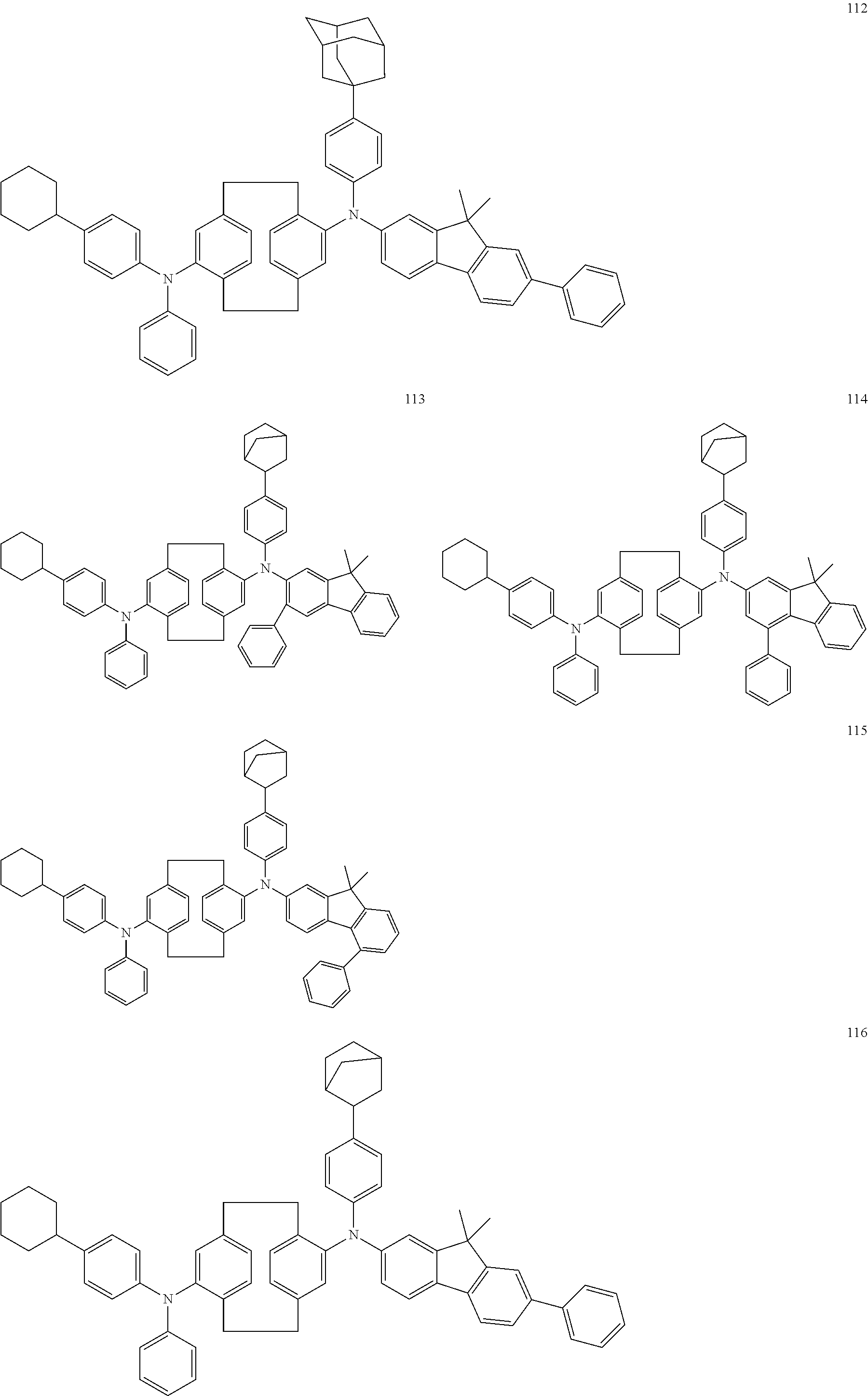 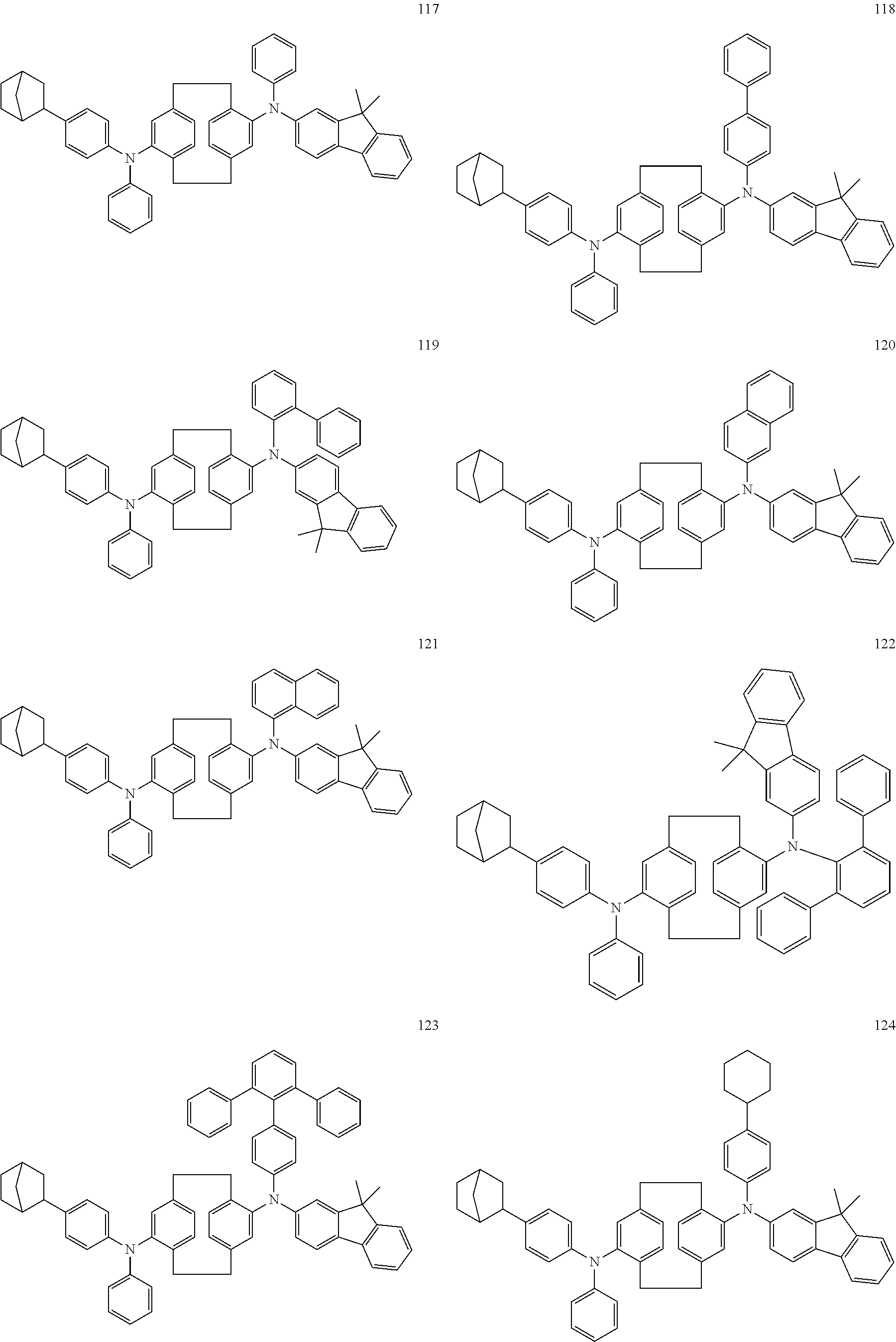 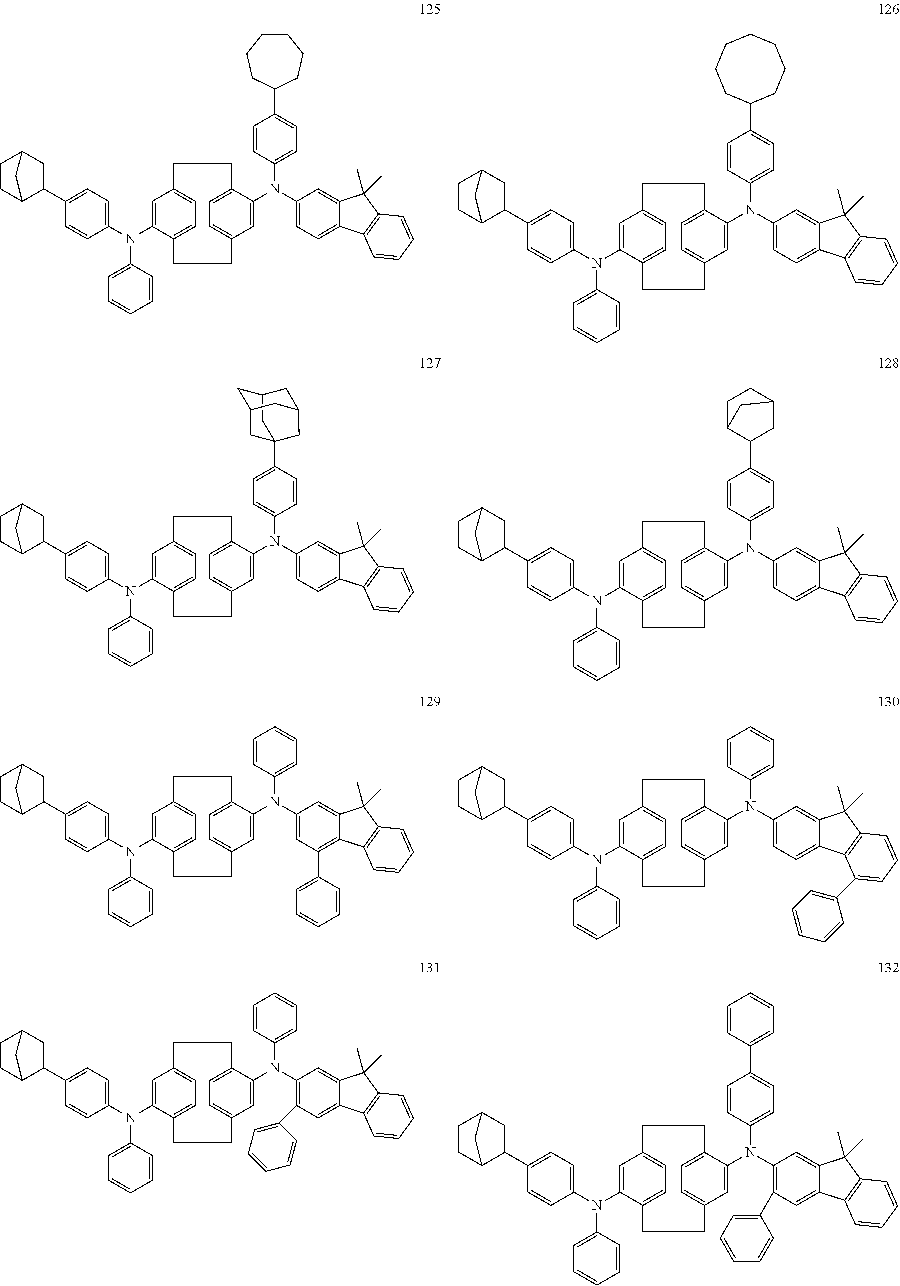 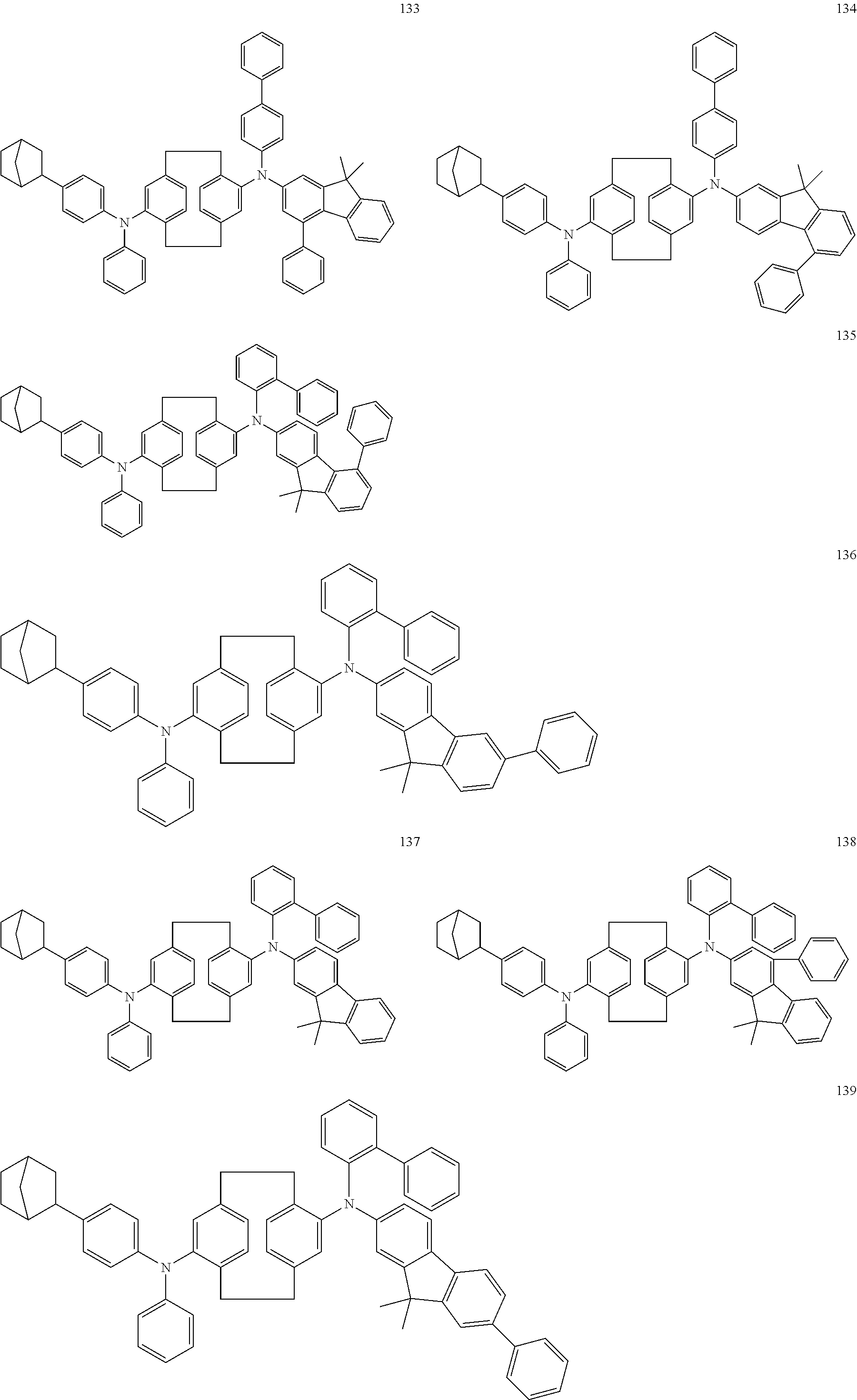 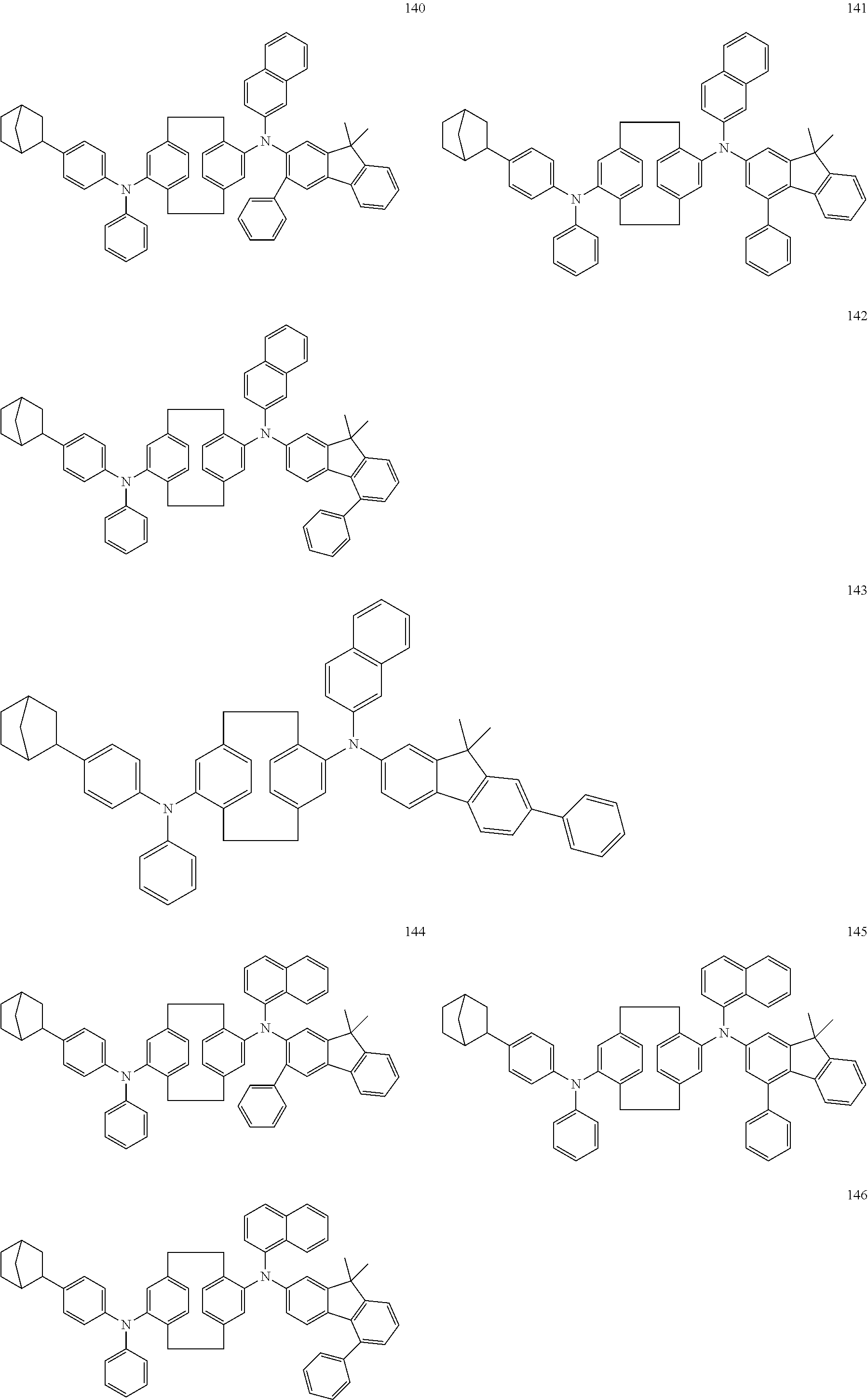 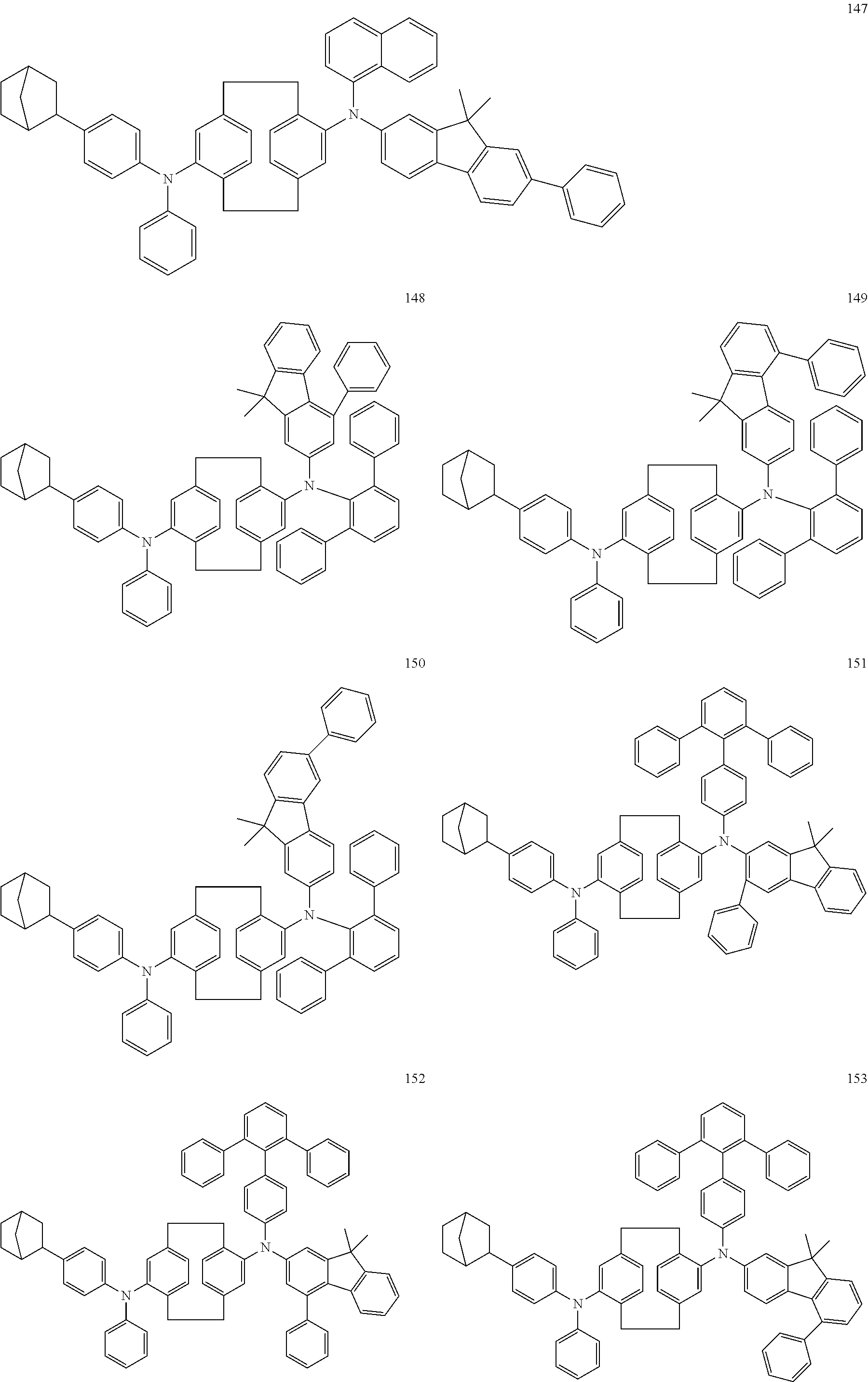 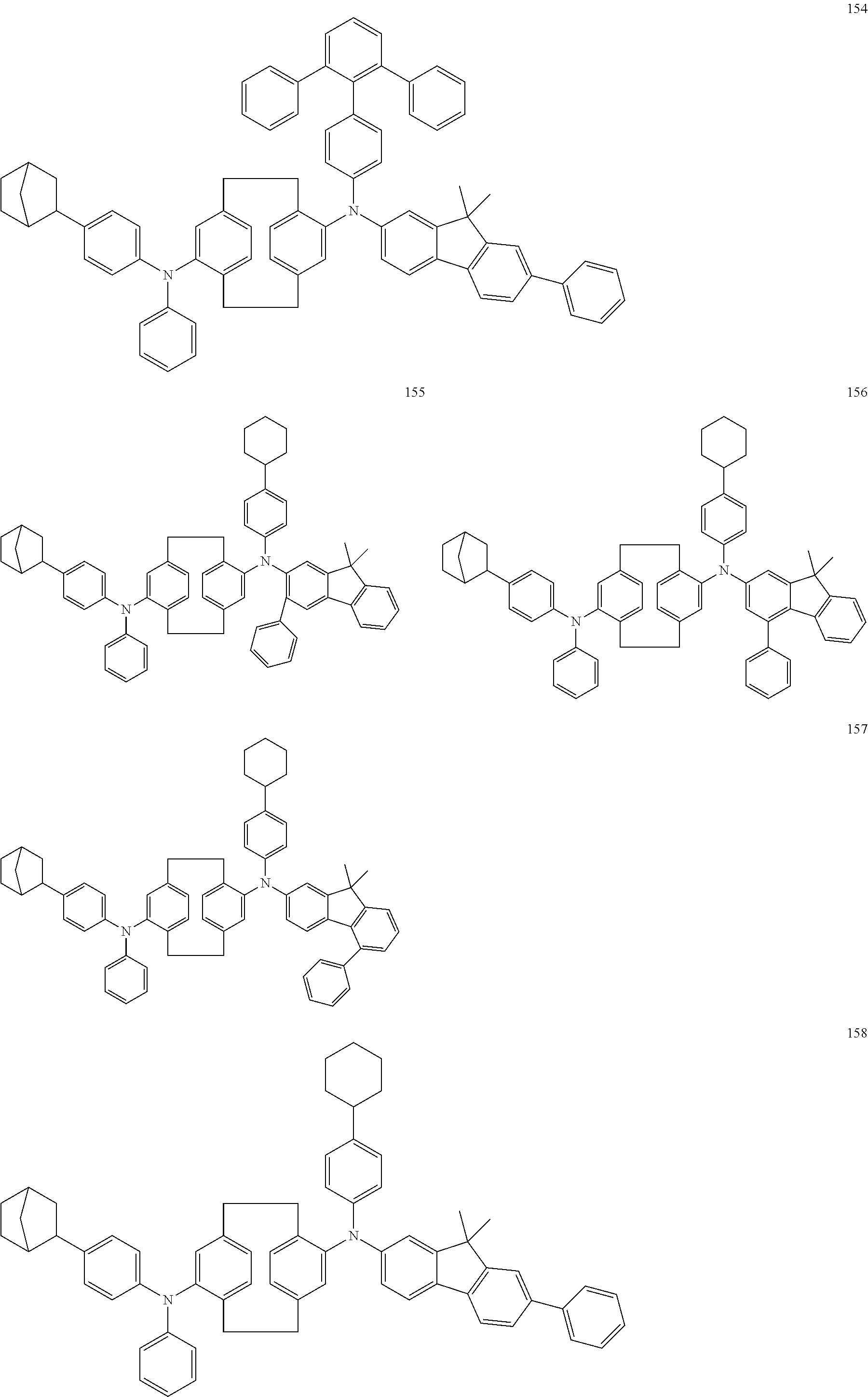 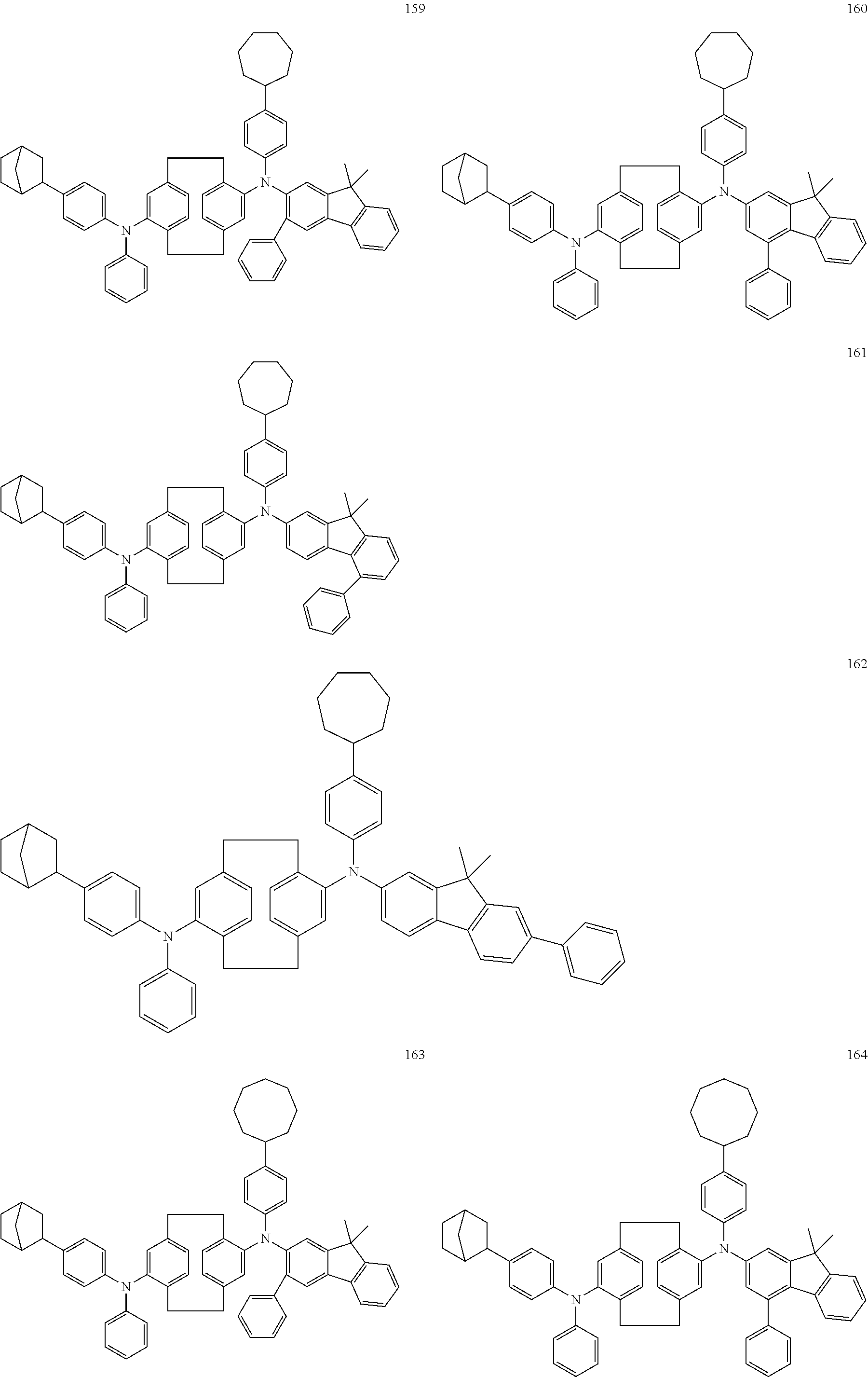 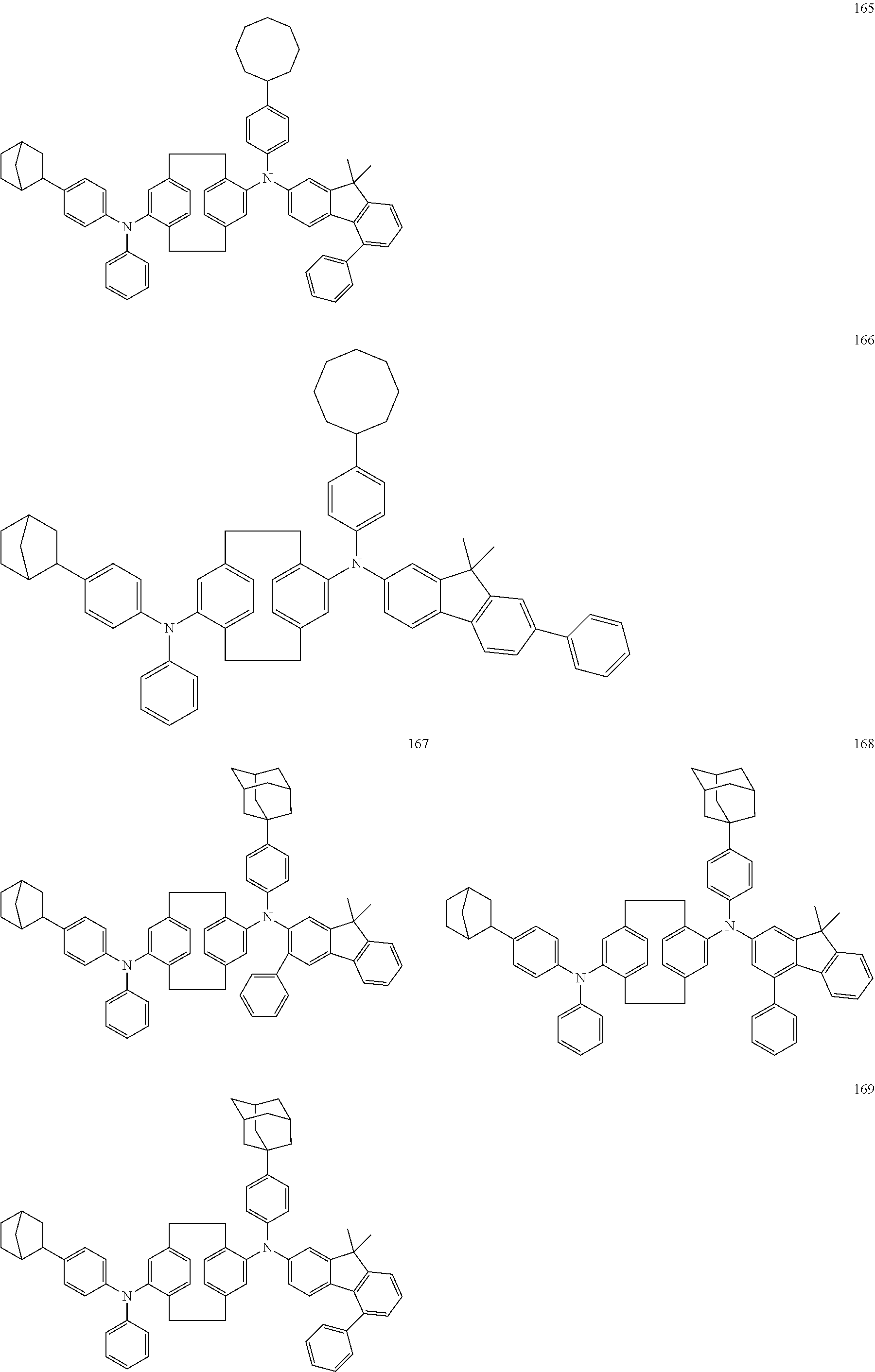 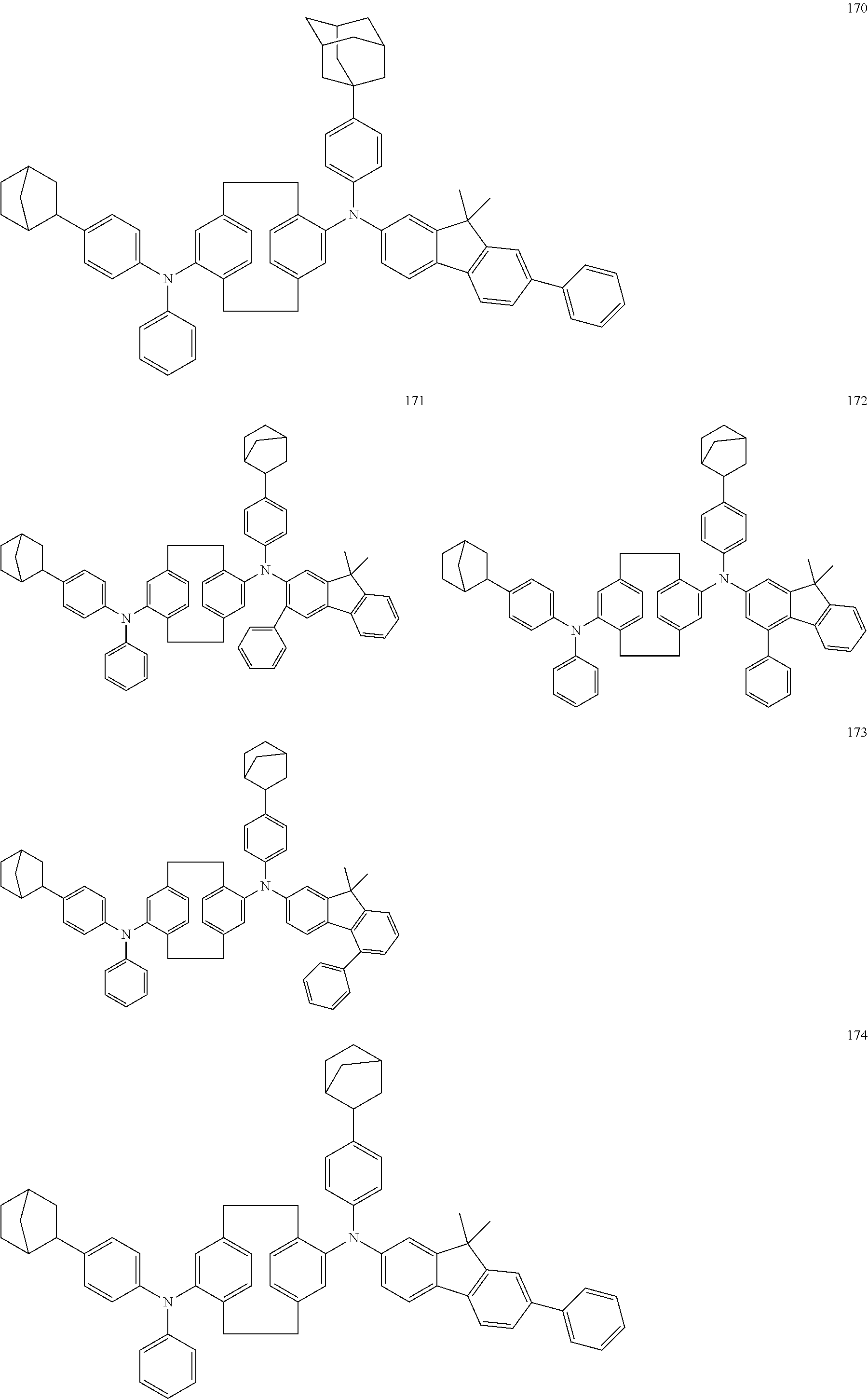 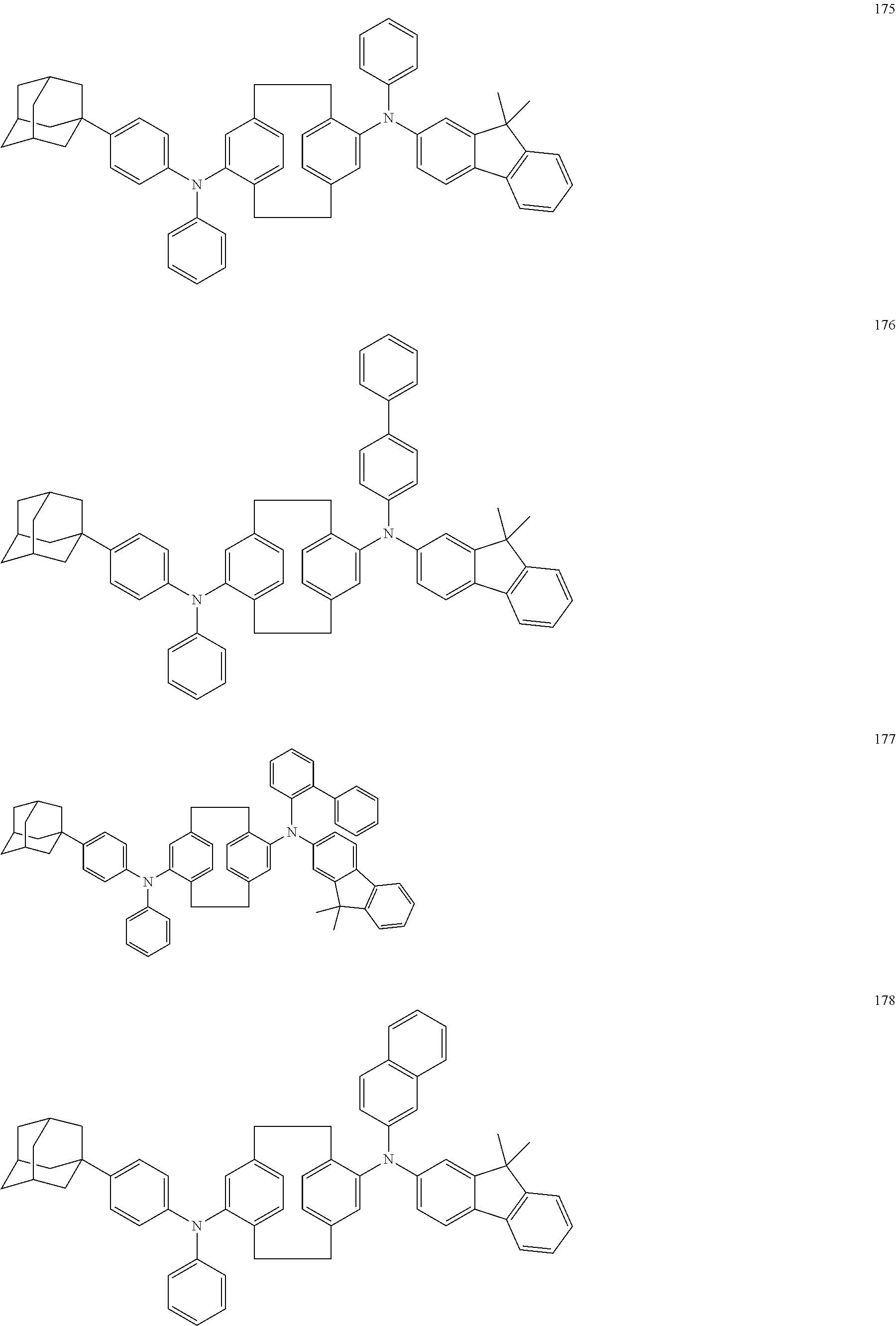 [SMILES: CC1(C)c2ccccc2-c2ccc(N(c3ccccc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2ccc(N(c3ccc(-c4ccccc4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2ccc(N(c3cc4ccc3CCc3ccc(c(N(c5ccccc5)c5ccc(C67CC8CC(CC(C8)C6)C7)cc5)c3)CC4)c3ccccc3-c3ccccc3)cc21 ; CC1(C)c2ccccc2-c2ccc(N(c3ccc4ccccc4c3)c3cc4ccc3CCc3ccc(c(N(c5ccccc5)c5ccc(C67CC8CC(CC(C8)C6)C7)cc5)c3)CC4)cc21] 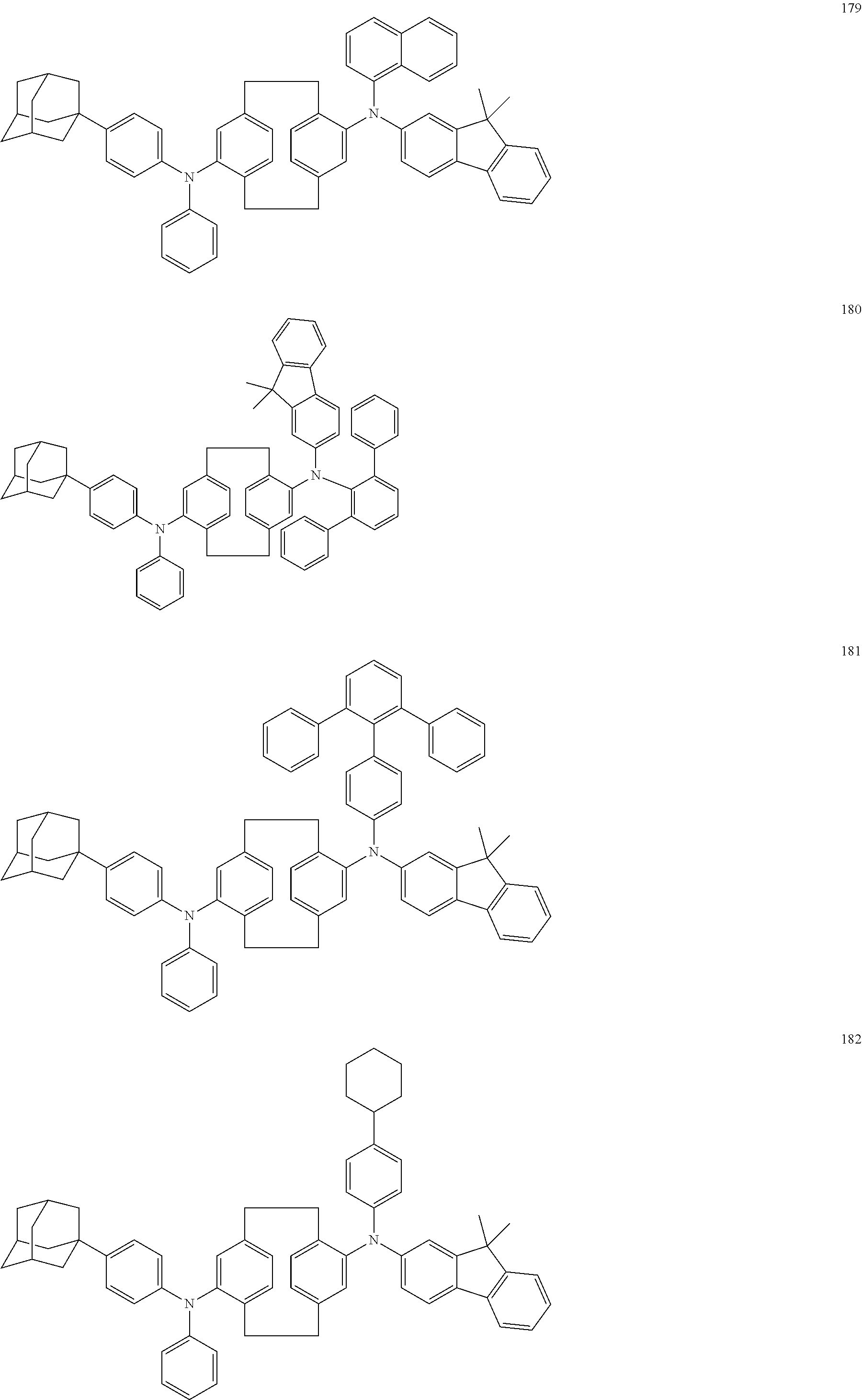 [SMILES: CC1(C)c2ccccc2-c2ccc(N(c3cc4ccc3CCc3ccc(c(N(c5ccccc5)c5ccc(C67CC8CC(CC(C8)C6)C7)cc5)c3)CC4)c3cccc4ccccc34)cc21 ; CC1(C)c2ccccc2-c2ccc(N(c3cc4ccc3CCc3ccc(c(N(c5ccccc5)c5ccc(C67CC8CC(CC(C8)C6)C7)cc5)c3)CC4)c3c(-c4ccccc4)cccc3-c3ccccc3)cc21 ; CC1(C)c2ccccc2-c2ccc(N(c3ccc(-c4c(-c5ccccc5)cccc4-c4ccccc4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2ccc(N(c3ccc(C4CCCCC4)cc3)c3cc4ccc3CCc3ccc(c(N(c5ccccc5)c5ccc(C67CC8CC(CC(C8)C6)C7)cc5)c3)CC4)cc21] 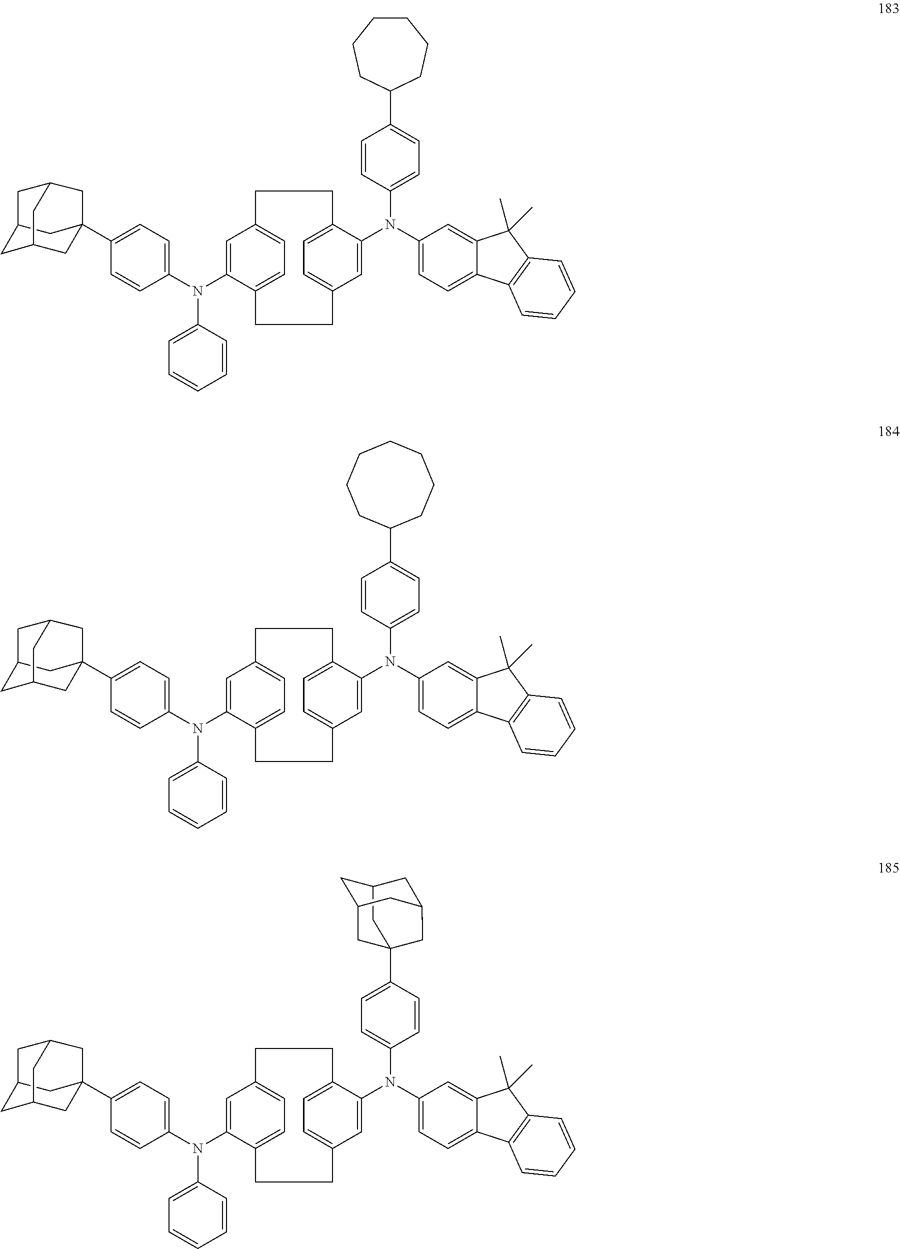 [SMILES: CC1(C)c2ccccc2-c2ccc(N(c3ccc(C4CCCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2ccc(N(c3ccc(C4CCCCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2ccc(N(c3ccc(C45CC6CC(CC(C6)C4)C5)cc3)c3cc4ccc3CCc3ccc(c(N(c5ccccc5)c5ccc(C67CC8CC(CC(C8)C6)C7)cc5)c3)CC4)cc21] 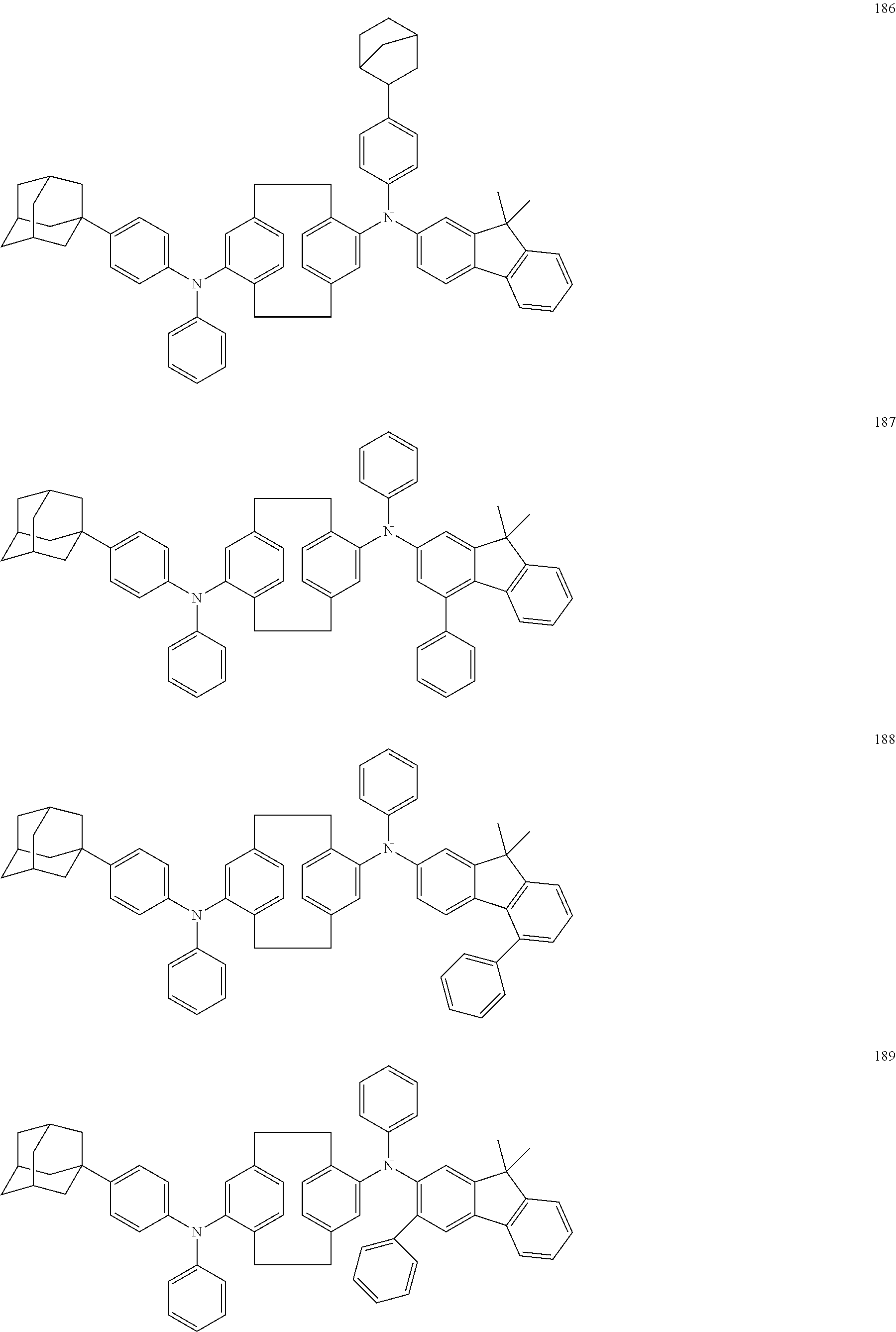 [SMILES: CC1(C)c2ccccc2-c2ccc(N(c3ccc(C4CC5CCC4C5)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccccc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccccc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21 ; CC1(C)c2ccccc2-c2cc(-c3ccccc3)c(N(c3ccccc3)c3cc4ccc3CCc3ccc(c(N(c5ccccc5)c5ccc(C67CC8CC(CC(C8)C6)C7)cc5)c3)CC4)cc21] 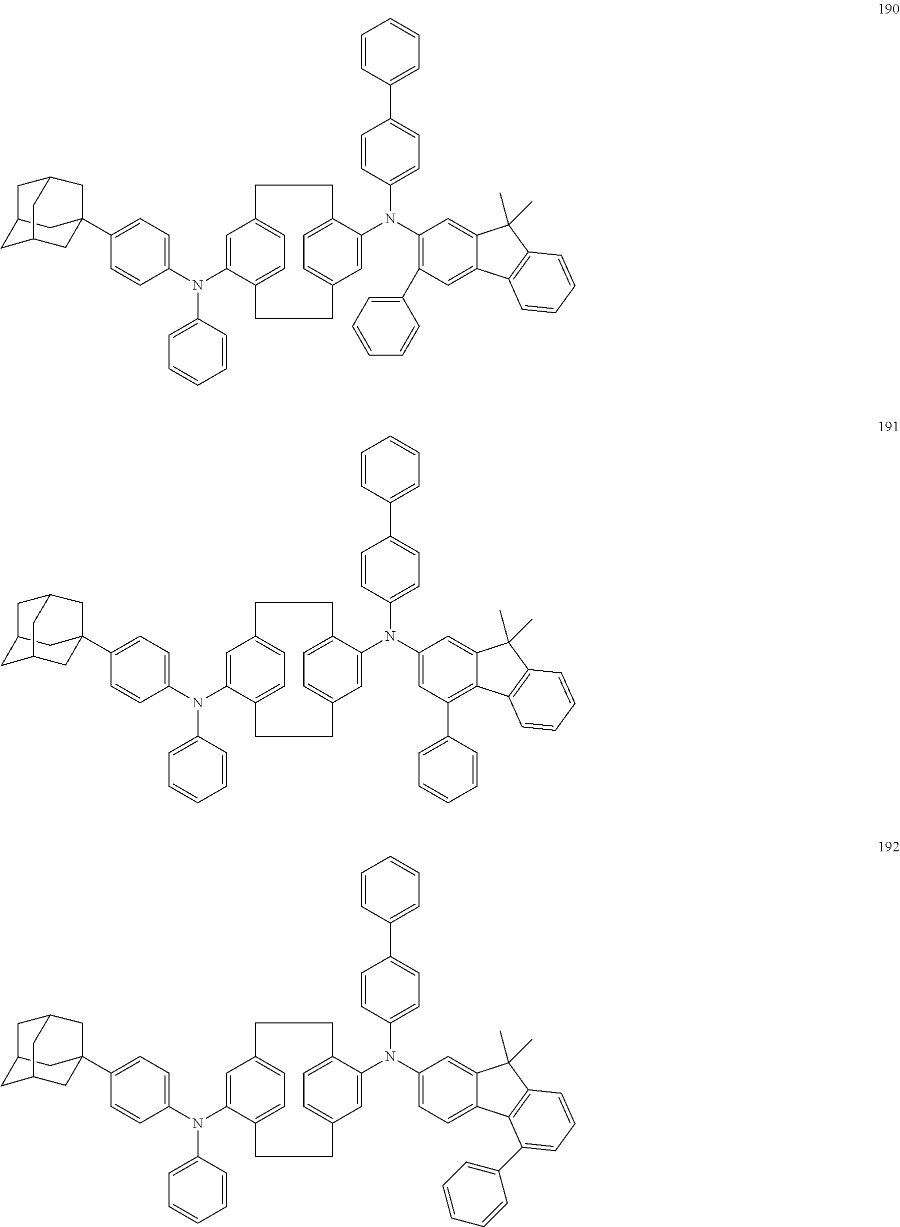 [SMILES: CC1(C)c2ccccc2-c2cc(-c3ccccc3)c(N(c3ccc(-c4ccccc4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc(-c4ccccc4)cc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc(-c4ccccc4)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21] 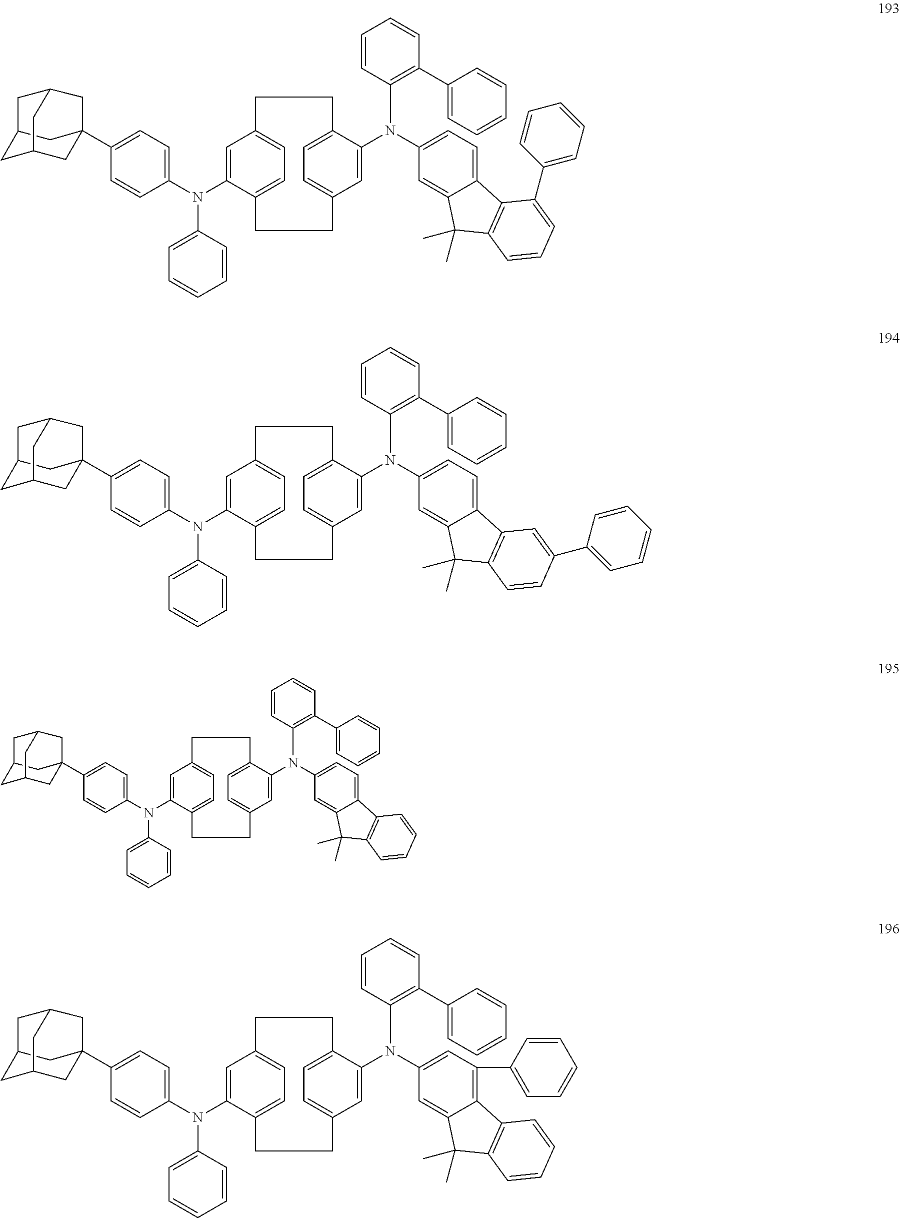 [SMILES: CC1(C)c2cc(N(c3cc4ccc3CCc3ccc(c(N(c5ccccc5)c5ccc(C67CC8CC(CC(C8)C6)C7)cc5)c3)CC4)c3ccccc3-c3ccccc3)ccc2-c2c(-c3ccccc3)cccc21 ; CC1(C)c2ccc(-c3ccccc3)cc2-c2ccc(N(c3cc4ccc3CCc3ccc(c(N(c5ccccc5)c5ccc(C67CC8CC(CC(C8)C6)C7)cc5)c3)CC4)c3ccccc3-c3ccccc3)cc21 ; CC1(C)c2ccccc2-c2ccc(N(c3cc4ccc3CCc3ccc(c(N(c5ccccc5)c5ccc(C67CC8CC(CC(C8)C6)C7)cc5)c3)CC4)c3ccccc3-c3ccccc3)cc21 ; CC1(C)c2ccccc2-c2c(-c3ccccc3)cc(N(c3cc4ccc3CCc3ccc(c(N(c5ccccc5)c5ccc(C67CC8CC(CC(C8)C6)C7)cc5)c3)CC4)c3ccccc3-c3ccccc3)cc21] 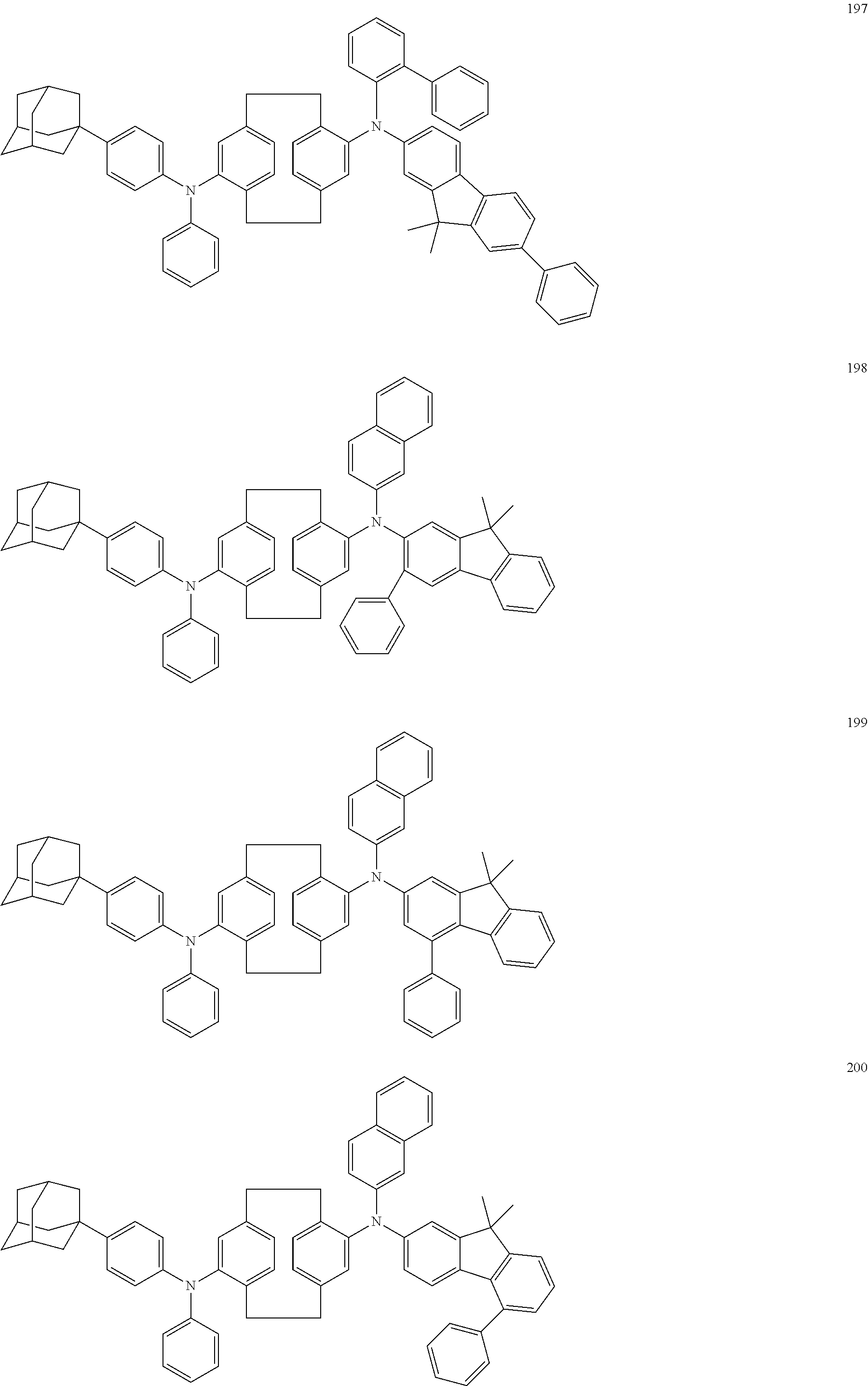 [SMILES: CC1(C)c2cc(-c3ccccc3)ccc2-c2ccc(N(c3cc4ccc3CCc3ccc(c(N(c5ccccc5)c5ccc(C67CC8CC(CC(C8)C6)C7)cc5)c3)CC4)c3ccccc3-c3ccccc3)cc21 ; CC1(C)c2ccccc2-c2cc(-c3ccccc3)c(N(c3ccc4ccccc4c3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc4ccccc4c3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc4ccccc4c3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21] 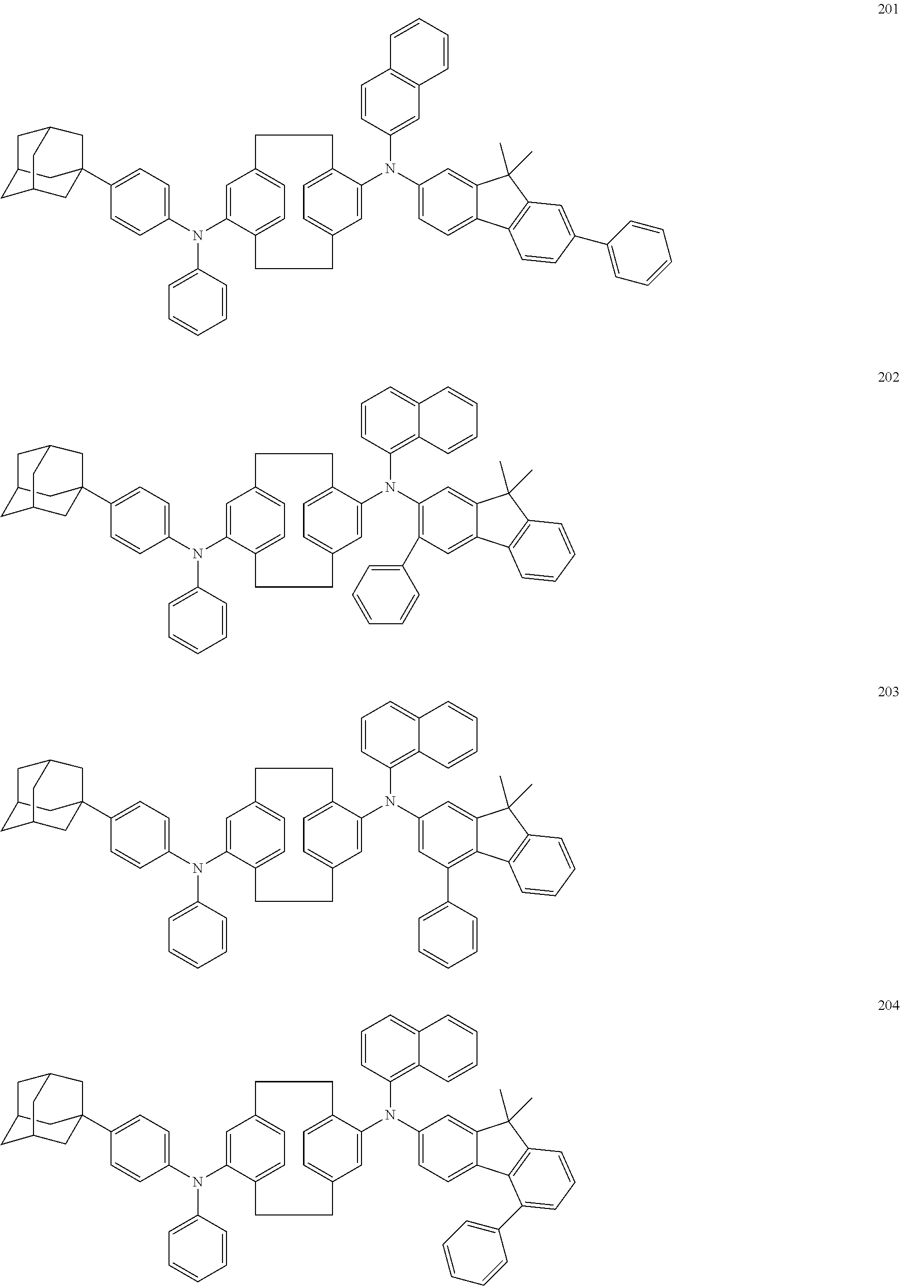 [SMILES: CC1(C)c2cc(-c3ccccc3)ccc2-c2ccc(N(c3ccc4ccccc4c3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2cc(-c3ccccc3)c(N(c3cc4ccc3CCc3ccc(c(N(c5ccccc5)c5ccc(C67CC8CC(CC(C8)C6)C7)cc5)c3)CC4)c3cccc4ccccc34)cc21 ; CC1(C)c2ccccc2-c2c(-c3ccccc3)cc(N(c3cc4ccc3CCc3ccc(c(N(c5ccccc5)c5ccc(C67CC8CC(CC(C8)C6)C7)cc5)c3)CC4)c3cccc4ccccc34)cc21 ; CC1(C)c2cc(N(c3cc4ccc3CCc3ccc(c(N(c5ccccc5)c5ccc(C67CC8CC(CC(C8)C6)C7)cc5)c3)CC4)c3cccc4ccccc34)ccc2-c2c(-c3ccccc3)cccc21] 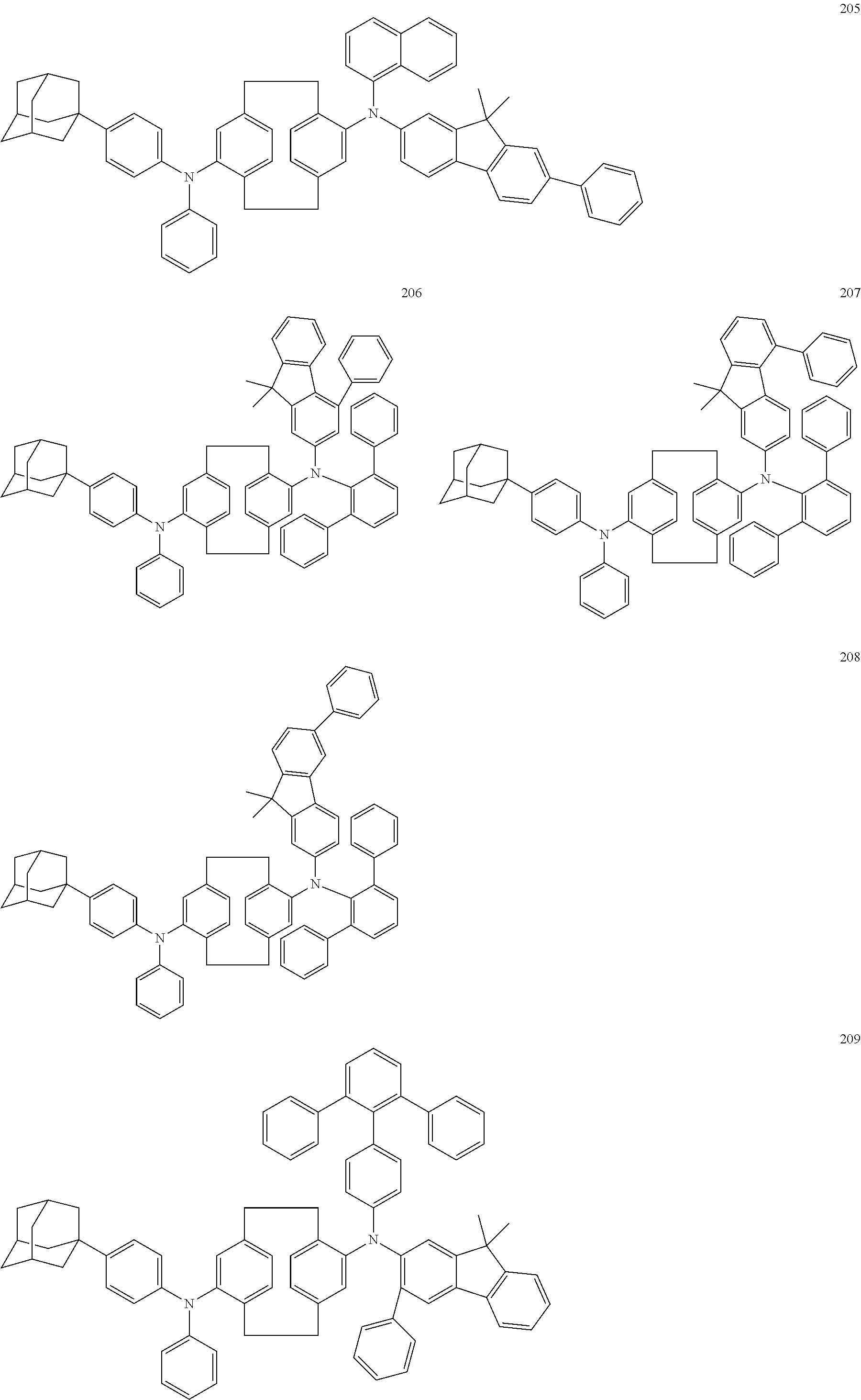 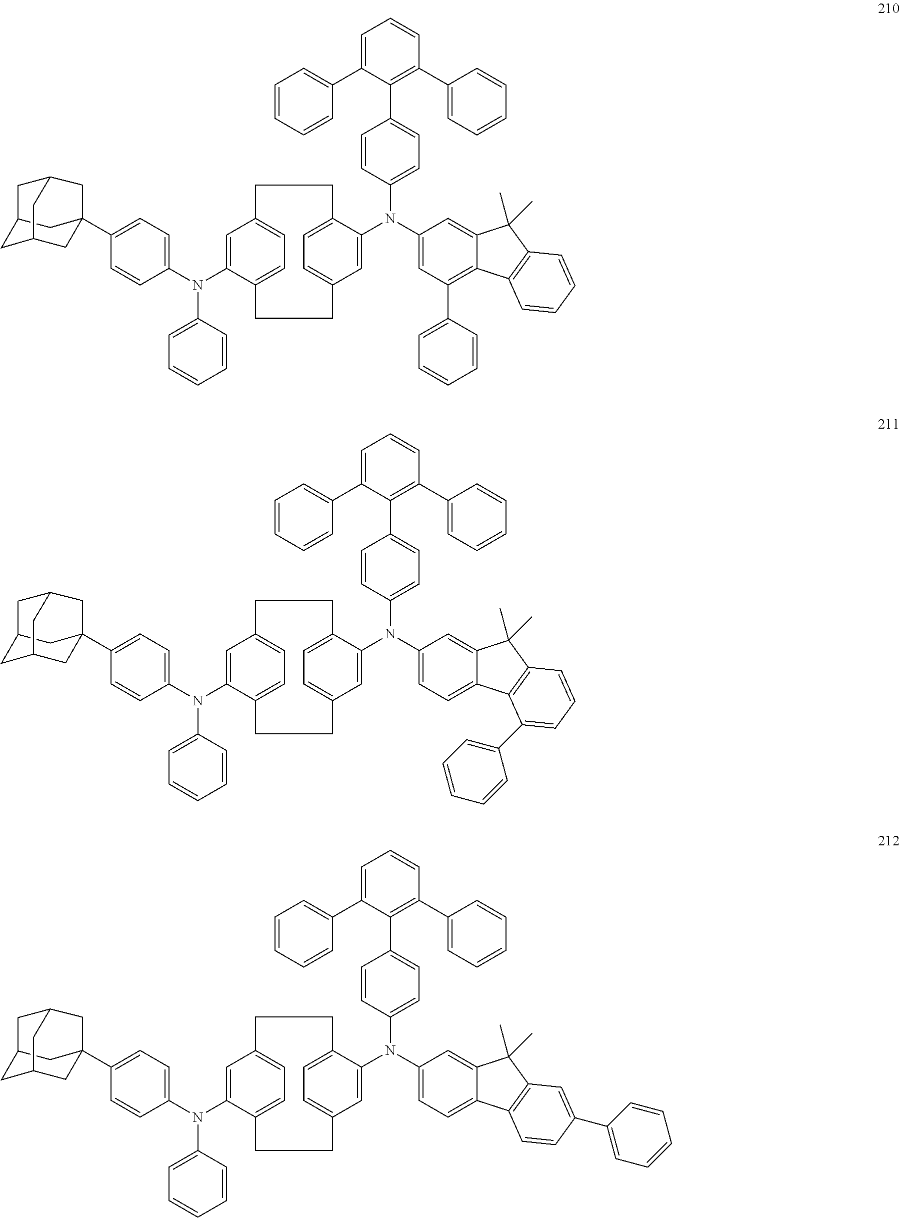 [SMILES: CC1(C)c2ccccc2-c2c(-c3ccccc3)cc(N(c3ccc(-c4c(-c5ccccc5)cccc4-c4ccccc4)cc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc(-c4c(-c5ccccc5)cccc4-c4ccccc4)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21 ; CC1(C)c2cc(-c3ccccc3)ccc2-c2ccc(N(c3ccc(-c4c(-c5ccccc5)cccc4-c4ccccc4)cc3)c3cc4ccc3CCc3ccc(c(N(c5ccccc5)c5ccc(C67CC8CC(CC(C8)C6)C7)cc5)c3)CC4)cc21] 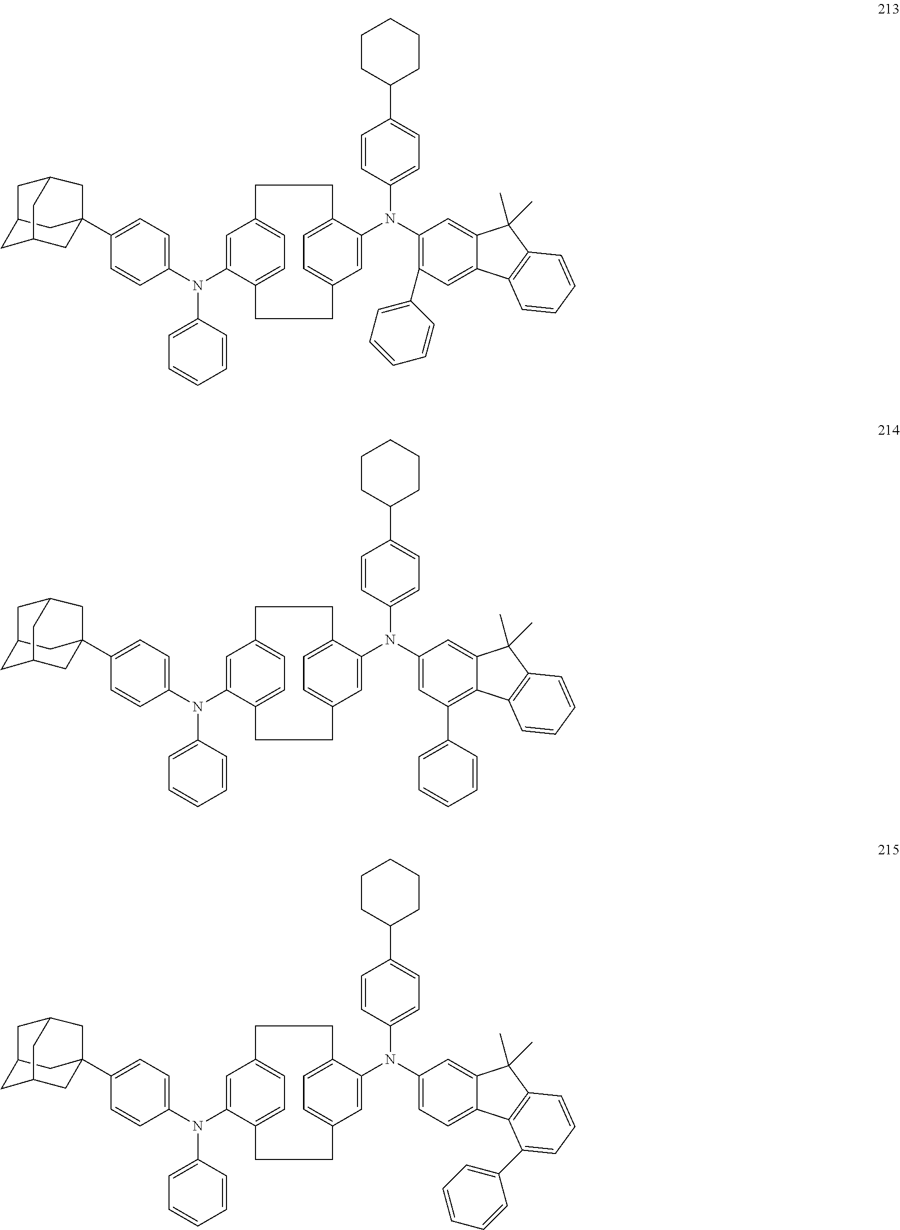 [SMILES: CC1(C)c2ccccc2-c2cc(-c3ccccc3)c(N(c3ccc(C4CCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc(C4CCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc(C4CCCCC4)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21] 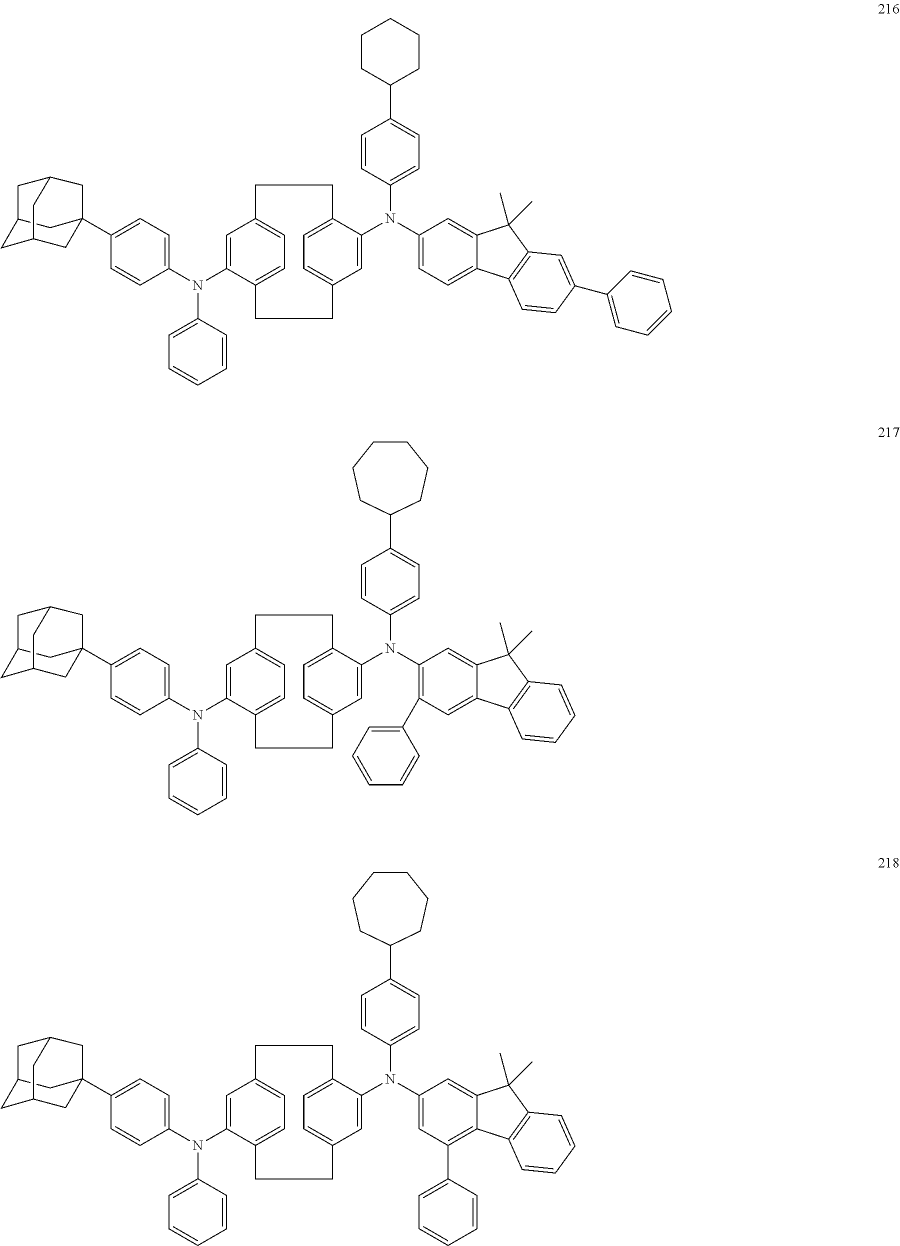 [SMILES: CC1(C)c2cc(-c3ccccc3)ccc2-c2ccc(N(c3ccc(C4CCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2cc(-c3ccccc3)c(N(c3ccc(C4CCCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc(C4CCCCCC4)cc3)c3cc4ccc3CCc3ccc(c(N(c5ccccc5)c5ccc(C67CC8CC(CC(C8)C6)C7)cc5)c3)CC4)cc21] 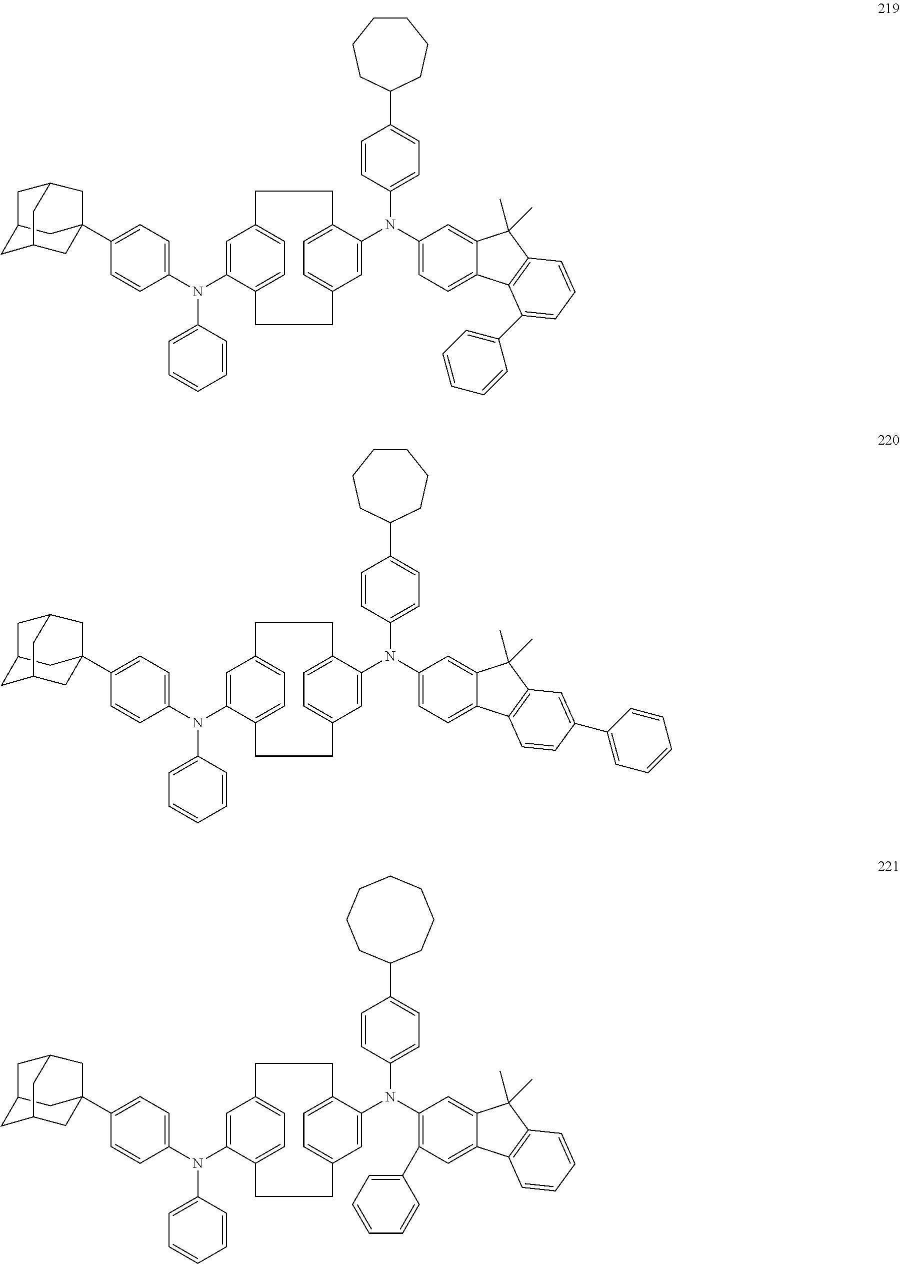 [SMILES: CC1(C)c2cc(N(c3ccc(C4CCCCCC4)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21 ; CC1(C)c2cc(-c3ccccc3)ccc2-c2ccc(N(c3ccc(C4CCCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2cc(-c3ccccc3)c(N(c3ccc(C4CCCCCCC4)cc3)c3cc4ccc3CCc3ccc(c(N(c5ccccc5)c5ccc(C67CC8CC(CC(C8)C6)C7)cc5)c3)CC4)cc21] 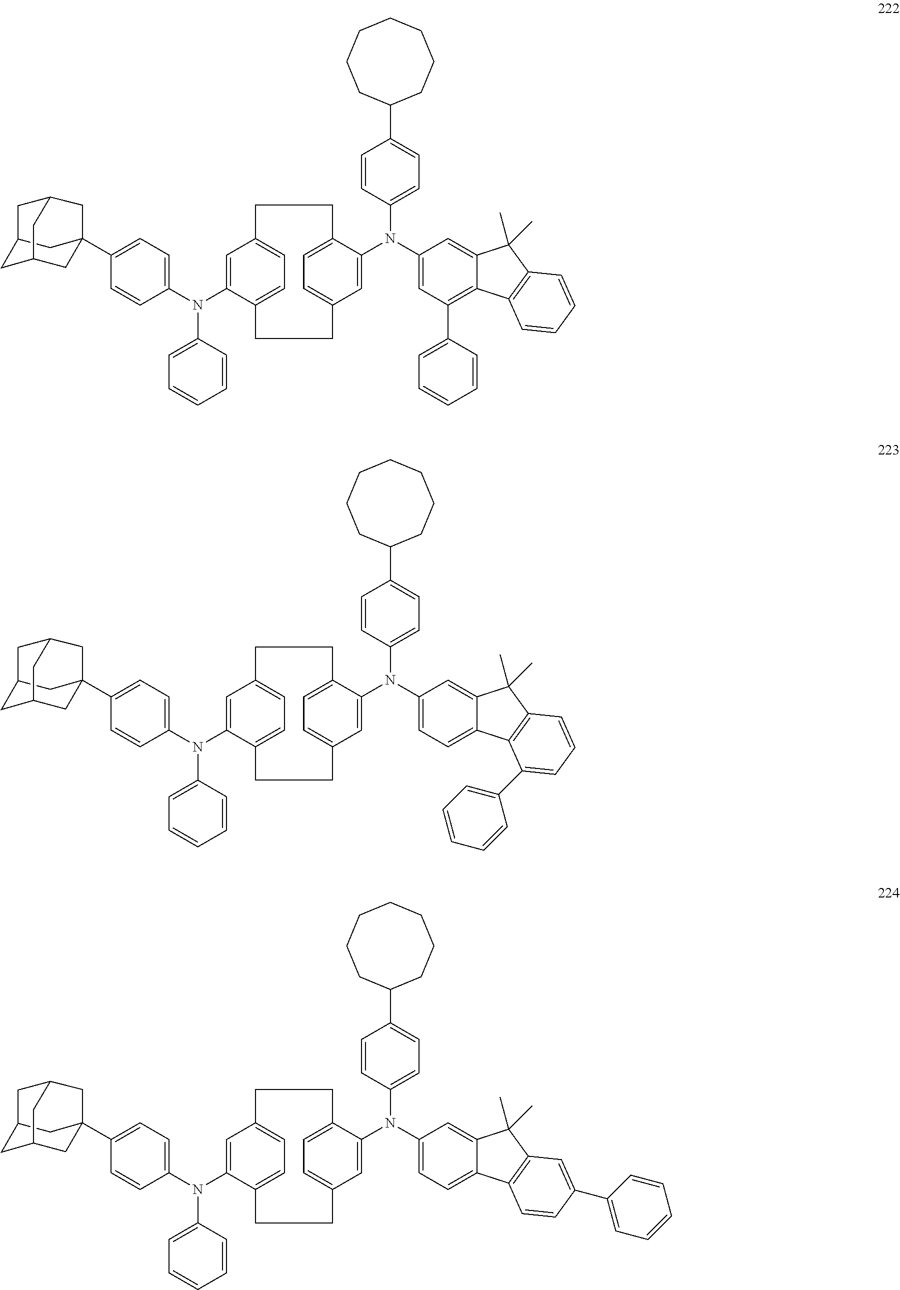 [SMILES: CC1(C)c2ccccc2-c2c(-c3ccccc3)cc(N(c3ccc(C4CCCCCCC4)cc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc(C4CCCCCCC4)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21 ; CC1(C)c2cc(-c3ccccc3)ccc2-c2ccc(N(c3ccc(C4CCCCCCC4)cc3)c3cc4ccc3CCc3ccc(c(N(c5ccccc5)c5ccc(C67CC8CC(CC(C8)C6)C7)cc5)c3)CC4)cc21] 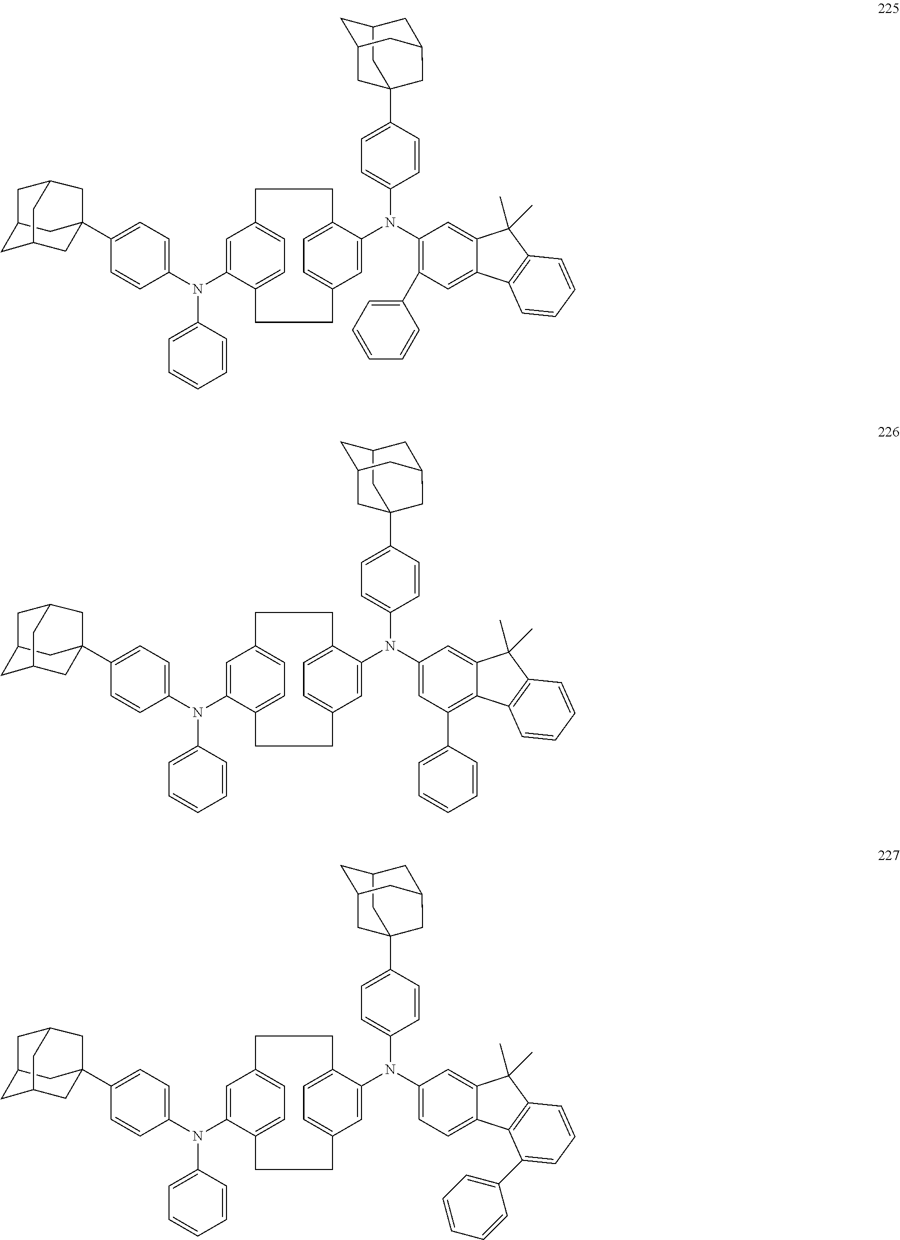 [SMILES: CC1(C)c2ccccc2-c2cc(-c3ccccc3)c(N(c3ccc(C45CC6CC(CC(C6)C4)C5)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc(C45CC6CC(CC(C6)C4)C5)cc3)c3cc4ccc3CCc3ccc(c(N(c5ccccc5)c5ccc(C67CC8CC(CC(C8)C6)C7)cc5)c3)CC4)cc21 ; CC1(C)c2cc(N(c3ccc(C45CC6CC(CC(C6)C4)C5)cc3)c3cc4ccc3CCc3ccc(c(N(c5ccccc5)c5ccc(C67CC8CC(CC(C8)C6)C7)cc5)c3)CC4)ccc2-c2c(-c3ccccc3)cccc21] 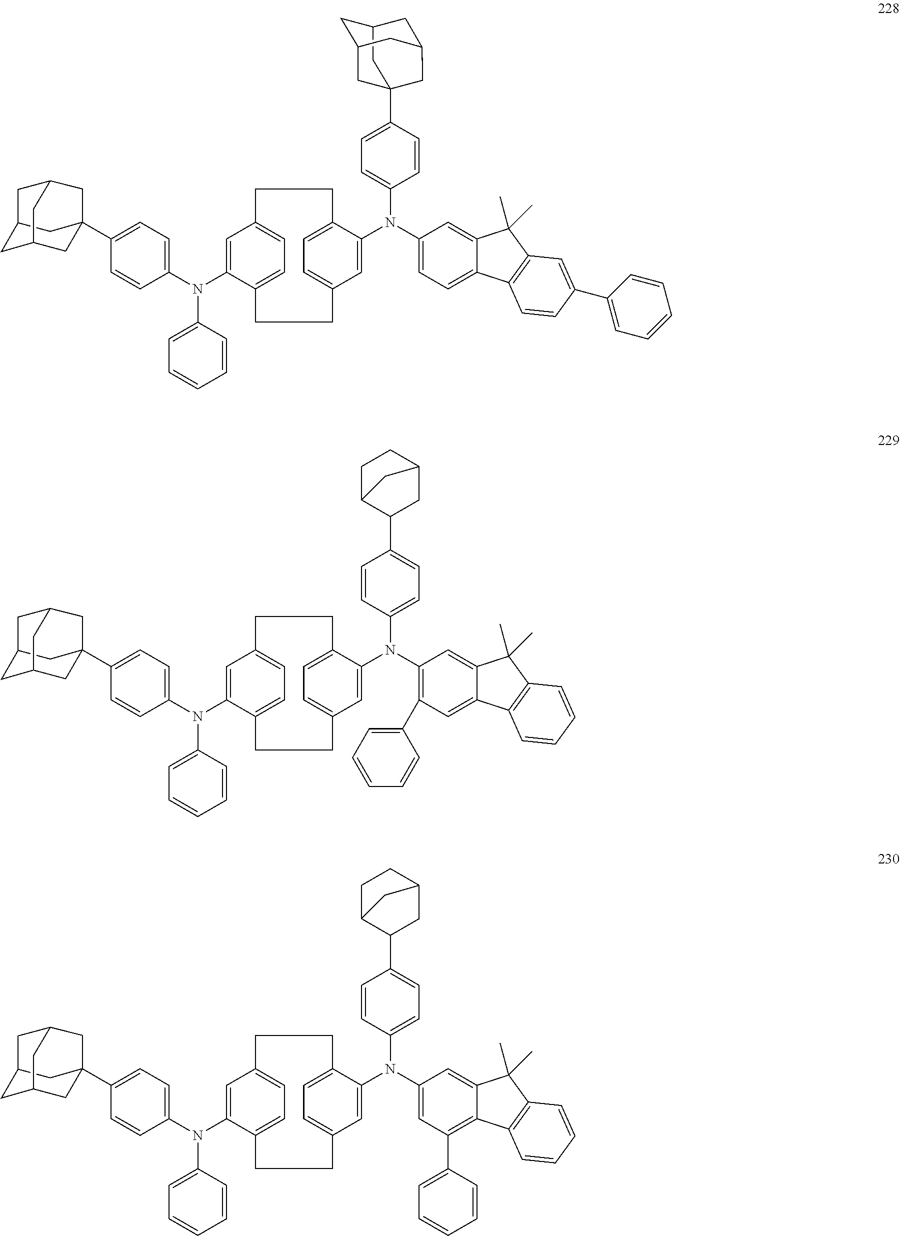 [SMILES: CC1(C)c2cc(-c3ccccc3)ccc2-c2ccc(N(c3ccc(C45CC6CC(CC(C6)C4)C5)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2cc(-c3ccccc3)c(N(c3ccc(C4CC5CCC4C5)cc3)c3cc4ccc3CCc3ccc(c(N(c5ccccc5)c5ccc(C67CC8CC(CC(C8)C6)C7)cc5)c3)CC4)cc21 ; CC1(C)c2ccccc2-c2c(-c3ccccc3)cc(N(c3ccc(C4CC5CCC4C5)cc3)c3cc4ccc3CCc3ccc(c(N(c5ccccc5)c5ccc(C67CC8CC(CC(C8)C6)C7)cc5)c3)CC4)cc21] 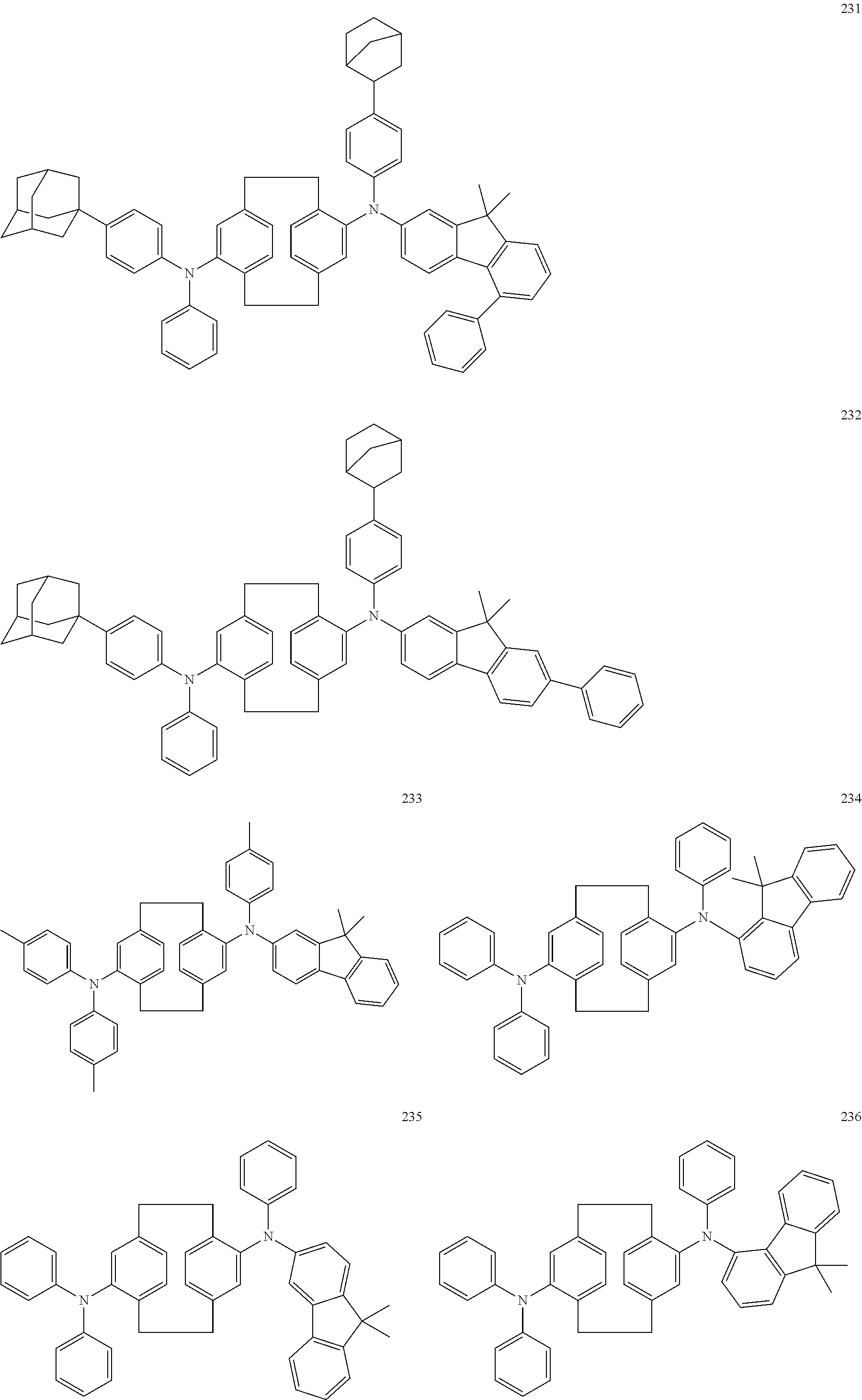 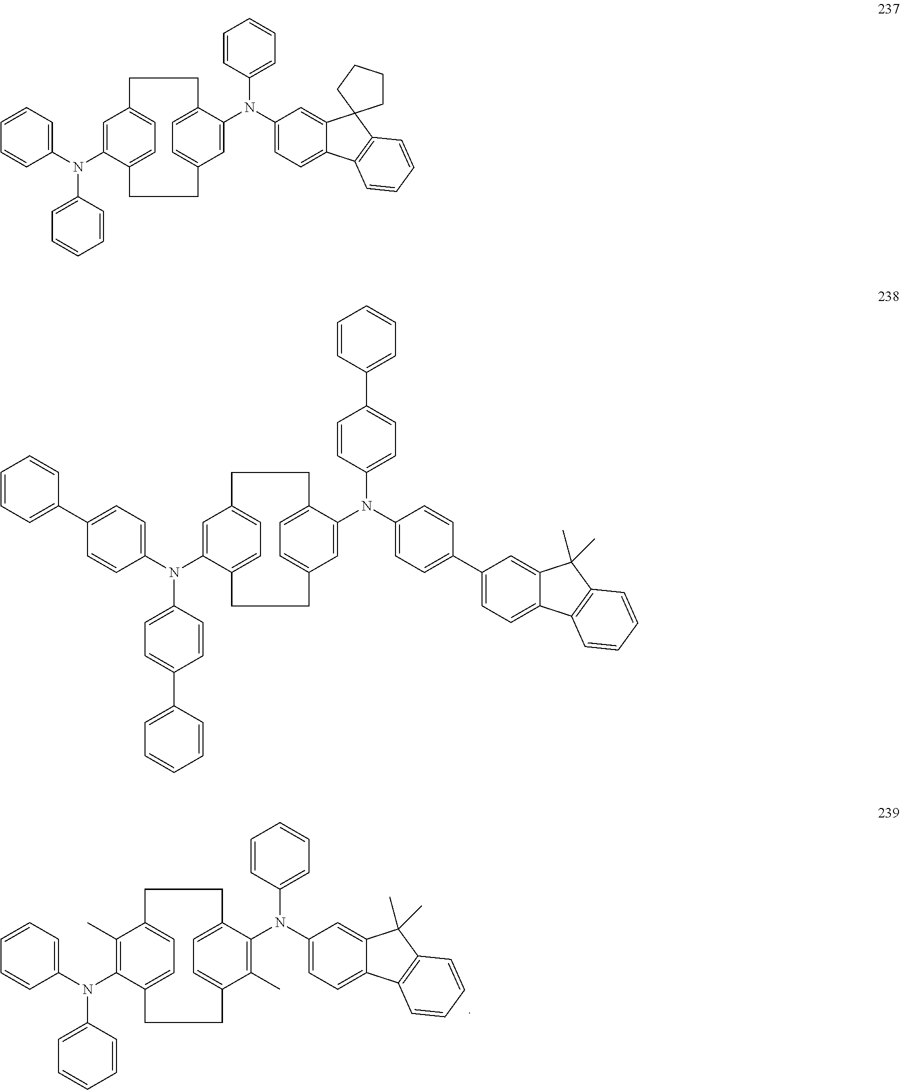 [SMILES: c1ccc(N(c2ccccc2)c2cc3ccc2CCc2ccc(c(N(c4ccccc4)c4ccc5c(c4)C4(CCCC4)c4ccccc4-5)c2)CC3)cc1 ; CC1(C)c2ccccc2-c2ccc(-c3ccc(N(c4ccc(-c5ccccc5)cc4)c4cc5ccc4CCc4ccc(c(N(c6ccc(-c7ccccc7)cc6)c6ccc(-c7ccccc7)cc6)c4)CC5)cc3)cc21 ; Cc1c2ccc(c1N(c1ccccc1)c1ccccc1)CCc1ccc(c(N(c3ccccc3)c3ccc4c(c3)C(C)(C)c3ccccc3-4)c1C)CC2]
